(19) United States
(12) Patent Application Publication
(10) Pub. No.: US 2011/0065916 A1
(43) Pub. Date: Mar. 17, 2011

(54) METHOD OF MAKING IMIDAZOAZEPINONE COMPOUNDS

(76) Inventors: Mark Spyvee, Hampstead, NH (US); Boris M. Seletsky, Andover, MA (US); Shawn Schiller, Haverhill, MA (US); Francis Fang, Andover, MA (US)

(21) Appl. No.: 12/739,286

(22) PCT Filed: Nov. 26, 2008
(86) PCT No.: PCT/US08/13162

§ 371 Date: Jun. 14, 2010

Related U.S. Application Data
(60) Provisional application No. 60/990,228, filed on Nov. 26, 2007.
(60) Provisional application No. 60/990,829, filed on Nov. 28, 2007.

(51) Int. Cl. C07D 487/20

(52) U.S. Cl. 540/543

(57) ABSTRACT

A method of making a compound of Formula I: is carried out by (a) providing a compound of Formula (II) or (III): wherein ring A is C3-14 aryl or C3-14 heteroaryl such as phenyl or furanyl, and then (b) combining the compound of Formula (II) or (III) with an acid to produce a compound of Formula I.

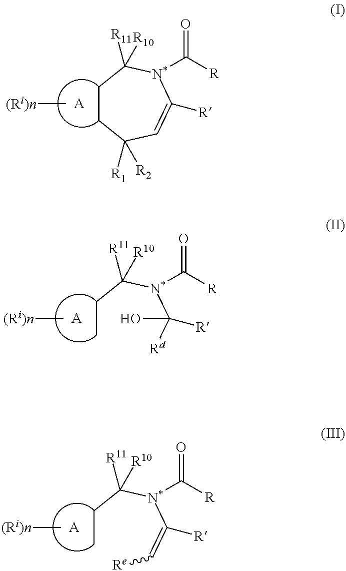

### BACKGROUND OF THE INVENTION

Upon encountering antigen, naive CD4+ T helper precursor (Thp) cells are differentiated into two distinct subsets, Type 1 T helper (Th1) and Type 2 T helper (Th2). These differentiated Th cells are defined both by their distinct functional abilities and by unique cytokine profiles. Specifically, Th1 cells produce interferon-gamma, interleukin (IL)-2, and tumor necrosis factor (TNF)-beta, which activate macrophages and are responsible for cell-mediated immunity and phagocyte-dependent protective responses. In contrast, Th2 cells are known to produce IL-4, IL-5, IL-6, IL-9, IL-10 and IL-13, which are responsible for strong antibody production, eosinophil activation, and inhibition of several macrophage functions, thus providing phagocyte-independent protective responses. Accordingly, Th1 and Th2 cells are associated with different immunopathological responses.

In addition, the development of each type of Th cell is mediated by a different cytokine pathway. Specifically, it has been shown that IL-4 promotes Th2 differentiation and simultaneously blocks Th1 development. In contrast, IL-12, IL-18 and IFN-gamma are the cytokines critical for the development of Th1 cells. Accordingly, the cytokines themselves form a positive and negative feedback system that drives Th polarization and keeps a balance between Th1 and Th2.

Th1 cells are involved in the pathogenesis of a variety of organ-specific autoimmune disorders, Crohn's disease, Helicobacter pylori-induced peptic ulcer, acute kidney allograft rejection, and unexplained recurrent abortions. In contrast, allergen-specific Th2 responses are responsible for atopic disorders in genetically susceptible individuals. Moreover, Th2 responses against still unknown antigens predominate in Omenn's syndrome, idiopathic pulmonary fibrosis, and progressive systemic sclerosis.

There remains a high unmet medical need to develop new treatments that are useful in treating the various conditions associated with imbalanced Th1/Th2 cellular differentiation. For many of these conditions the currently available treatment options are inadequate. Accordingly, the Th1/Th2 paradigm provides the rationale for the development of strategies for the therapy of allergic and autoimmune disorders.

### SUMMARY OF THE INVENTION

A first aspect of the invention is a method of making a compound of Formula I:

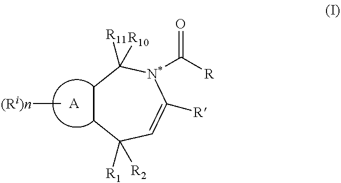

comprising the steps of:
(a) providing a compound of Formula (II) or (III):

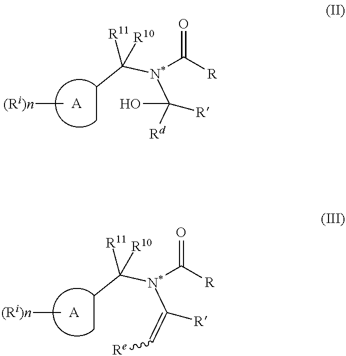

wherein:

ring A is C₃₋₁₄ aryl or C₃₋₁₄heteroaryl

n is an integer from 0 to 4 (e.g., 0, 1, 2, 3 or 4; 0 to 1, 0 to 2; 0 to 3),

each occurrence of Rⁱ is independently selected from the group consisting of hydrogen, hydroxyl, C₁₋₁₀ alkoxy, benzyloxy, benzyl, halo, amino, (C₁₋₆ alkyl)amino, (C₁₋₆alkyl)(C₁₋₆alkyl)amino, phenoxyl, and phenyl; or two adjacent R′, taken together, are —O—(CH₂)—O— or —O—CH₂—CH₂—O— and Rⁱ is attached to the A ring as valence permits;

R and R′ are each independently hydrogen, C₁₋₁₀ alkyl, C₂₋₁₀ alkenyl, C₂₋₁₀ alkynyl, C₁₋₁₀ alkoxy, C₁₋₁₀ alkylsulfonyl, C₁₋₁₀ haloalkyl, C₁₋₁₀ aminoalkyl, amino, (C₁₋₆ alkyl)amino, (C₁₋₆alkyl)(C₁₋₆alkyl)amino, C₃₋₁₀ cycloalkyl, C₃₋₁₀ cycloalkenyl, C₃₋₁₀ cycloalkynyl, C₃₋₁₀ heterocycle, C₃₋₁₄ aryl, or C₃₋₁₄ heteroaryl, or R and R′ taken together form with N* a C₃₋₁₀ cycloalkyl, C₃₋₁₀ cycloalkenyl, C₃₋₁₀ cycloalkynyl, C₄₋₁₀ heterocyclyl, C₃₋₁₄ aryl, or C₃₋₁₄ heteroaryl ring system, which ring system is unsubstituted or substituted from one to four times with substituents independently selected from the group consisting of halo, oxygen, hydroxyl, sulfuryl, amino, nitro, cyano, C₁₋₁₀ haloalkyl, C₁₋₁₀ alkyl, C₃₋₁₀ spirocyclyl, C₃₋₁₀ spiroheterocyclyl, C₂₋₁₀ alkenyl, C₂₋₁₀ alkynyl, C₁₋₁₀ alkoxy, C₁₋₁₀ aminoalkyl, C₁₋₁₀ thioalkyl, C₃₋₁₀ heterocyclyl, C₃₋₁₀ cycloalkyl, C₃₋₁₄ aryl, and C₃₋₁₄ heteroaryl,

R¹ and R² are independently hydrogen, C₁₋₁₀ alkyl, C_{2.10} alkenyl, C₂₋₁₀ alkynyl, or taken together are C₂₋₁₀ alkenyl or C₂₋₁₀ alkenylenidene, or R¹ and R² taken together form C₃₋₁₀ cycloalkyl or C₃₋₁₀ heterocyclyl,

R¹⁰ and R¹¹ are independently selected from the group consisting of hydrogen, oxygen, hydroxyl, C₁₋₁₀ alkyl, C₂₋₁₀ alkenyl, C₂₋₁₀ alkynyl, C₁₋₁₀ alkoxy, C₁₋₁₀ alkylsulfonyl, C₁₋₁₀ haloalkyl, C₁₋₁₀ aminoalkyl, amino, (C₁₋₆ alkyl)amino, (C₁₋₆alkyl)(C₁₋₆alkyl)amino, C₃₋₁₀ cycloalkyl, C₃₋₁₀ cycloalkenyl, C₃₋₁₀ cycloalkynyl, C₃₋₁₀ heterocycle, C₃₋₁₄ aryl and C₃₋₁₄ heteroaryl, or taken together form C₂₋₁₀ alkenyl, C₃₋₁₀cycloalkyl, C₃₋₁₀heterocyclyl

R^{d} is C₂₋₁₀ alkenyl or C₂₋₁₀ alkynyl,

R^{e} is C₂₋₁₀ alkenyl or C₂₋₁₀ alkynyl, wherein R^{e} is positioned cis or trans to the double bond; and

(b) combining said compound of Formula (II) or (III) with an acid to produce a compound of Formula I.

In some embodiments, the present invention provides a method of making a compound of Formula (Ia)

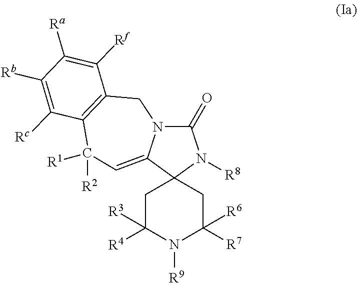

comprising the steps of:

(a) providing a compound of Formula (IIa) or (IIIa):

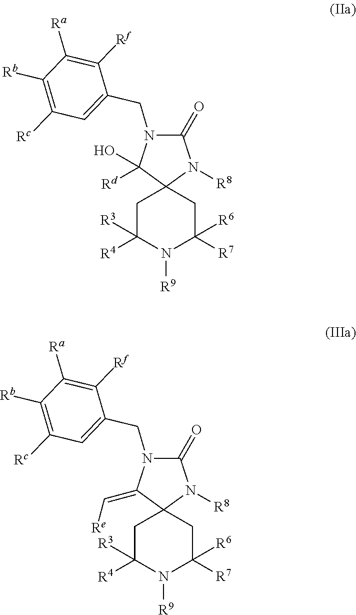

wherein:

R¹ and R² are independently hydrogen, C₁₋₁₀ alkyl, C₂₋₁₀ alkenyl, C₂₋₁₀ alkynyl, or taken together are C₂₋₁₀ alkenyl or C₂₋₁₀ alkenylenidene, or form a C₃₋₁₀ cycloalkyl or C₃₋₁₀ heterocyclyl,

each of R³, R⁴, R⁶, and R⁷ is independently selected from hydrogen and methyl, or R³ and R⁶ taken together is —(CH₂CH₂)—,

R^{d} and R^{e} are independently C₂₋₁₀ alkenyl (e.g., C₃₋₁₀ alkenyl) or C₂₋₁₀ alkynyl (e.g., C₃₋₁₀ alkynyl), and R^{e} is positioned cis or trans to the double bond,

each of R^{a}, R^{b}, R^{c} and R^{f} is independently selected from the group consisting of hydrogen, hydroxyl, C₁₋₁₀ alkoxy, benzyloxy, benzyl, halo, amino, (C₁₋₆ alkyl)amino, (C₁₋₆alkyl)(C₁₋₆alkyl)amino, phenoxy, and phenyl; or one pair selected from R^{a} and R^{b}, and R^{b} and R^{c}, taken together, is —O—(CH₂)—O— or —O—CH₂—CH₂—O—,

R⁹ is hydrogen or X—R⁵, wherein X is C₁₋₁₀ alkylene, C₂₋₁₀ alkenylene, C₂₋₁₀ alkynylene, and R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furanyl, thienyl, pyrazolyl, quinoxalinyl, or naphthyl,

wherein said R⁵ substituted with between 0 and 5 substituents independently selected from the group consisting of C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl and halo,

R⁸ is hydrogen, methyl, ethyl, propyl, (C₁₋₃ alkoxy)C₁₋₃ alkyl, (C₁₋₃ alkylthio)C₁₋₃ alkyl, C₁₋₃ hydroxyalkyl, phenyl, benzyl, furanyl, pyrrolyl, imidazolyl, pyrazolyl, pyrrolyl, isothiazolyl, isooxazolyl, pyridyl, and thienyl,

wherein R⁸ is substituted with between 0 and 3 substituents independently selected from methyl, ethyl, halo, hydroxyl, C₁₋₃ alkoxy, C₁₋₃ alkylthio, (C₁₋₃ alkoxy)C₁₋₃ alkyl, (C₁₋₃ alkylthio)C₁₋₃ alkyl, C₁₋₃ hydroxyalkyl, (C₁₋₃ mercaptoalkyl)phenyl, benzyl, furanyl, imidazolyl, pyrazolyl, pyrrolyl, thiazolyl, isothiazolyl, oxazolyl, isooxazolyl, pyridyl, thienyl, indolyl, benzpyrazolyl, benzimidazolyl, benzofuranyl, benzoxazolyl, benzisoxazolyl, isobenzofuranyl, benzothiophenyl, isobenzothiophenyl, indolinyl, quinolinyl, isoquinolinyl, quinazolinyl, or quinoxalinyl, and

(b) combining said compound of Formula (IIa) or (IIa) with an acid to produce a compound of Formula (Ia).

In other embodiments of the invention, the compound of Formula I is a compound of Formula (Ib), (Ic), or (Id):

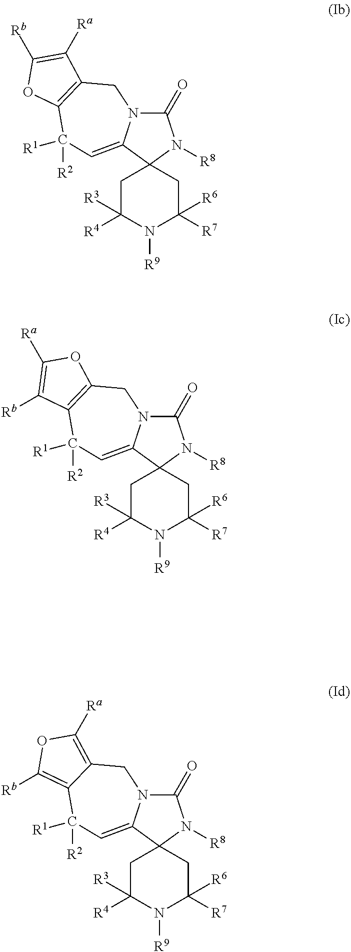

and likewise the compounds of Formula (II) or Formula (III) are compounds of Formula (IIb-d) or (IIIb-d):

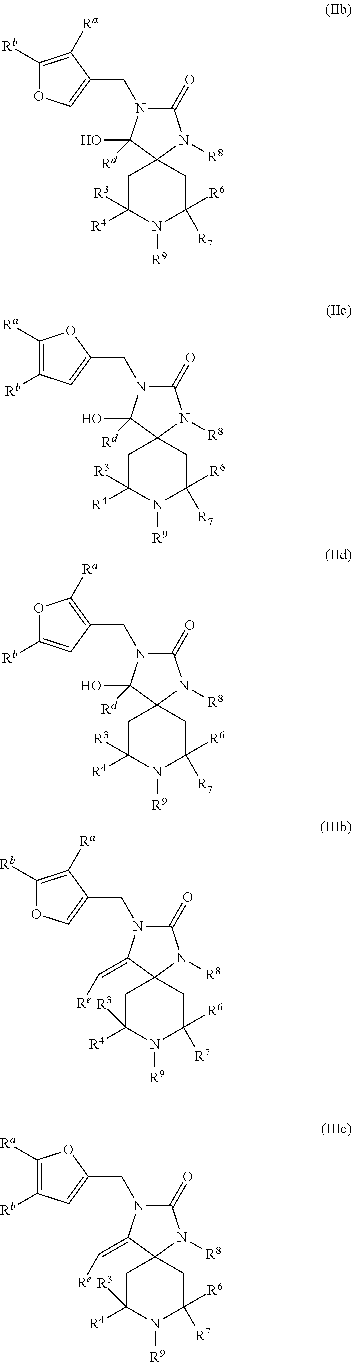

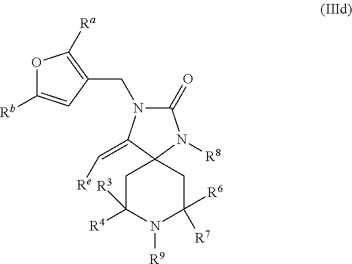

wherein:

each of R³, R⁴, R⁶, and R⁷ are independently selected from hydrogen and methyl, or R³ and R⁶ taken together is —(CH₂CH₂)—,

R^{d} and R^{e} are independently C₂₋₁₀ alkenyl or C₂₋₁₀ alkynyl, and R^{e} is positioned cis or trans to the double bond,

each of R^{a} and R^{b} is independently selected from the group consisting of hydrogen, hydroxyl, C₁₋₁₀ alkoxy, benzyloxy, benzyl, halo, amino, (C₁₋₆ alkyl)amino, (C₁₋₆alkyl)(C₁₋₆alkyl)amino, phenoxy, and phenyl; or one pair selected from R^{a} and R^{b}, and R^{b} and R^{c}, taken together, is —O—(CH₂)—O— or —O—CH₂—CH₂—O—,

R⁹ is hydrogen or X—R⁵, wherein X is C₁₋₁₀ alkylene, C₂₋₁₀ alkenylene, C₂₋₁₀ alkynylene, and R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furanyl, thienyl, pyrazolyl, quinoxalinyl, or naphthyl,

wherein said R⁵ substituted with between 0 and 5 substituents independently selected from the group consisting of C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl and halo,

R⁸ is hydrogen, methyl, ethyl, propyl, (C₁₋₃ alkoxy)C₁₋₃ alkyl, (C₁₋₃ alkylthio)C₁₋₃ alkyl, C₁₋₃ hydroxyalkyl, phenyl, benzyl, furanyl, pyrrolyl, imidazolyl, pyrazolyl, pyrrolyl, isothiazolyl, isooxazolyl, pyridyl, and thienyl,

wherein R⁸ is substituted with between 0 and 3 substituents independently selected from methyl, ethyl, halo, hydroxyl, C₁₋₃ alkoxy, C₁₋₃ alkylthio, (C₁₋₃ alkoxy)C₁₋₃ alkyl, (C₁₋₃ alkylthio)C₁₋₃ alkyl, C₁₋₃ hydroxyalkyl, (C₁₋₃ mercaptoalkyl)phenyl, benzyl, furanyl, imidazolyl, pyrazolyl, pyrrolyl, thiazolyl, isothiazolyl, oxazolyl, isooxazolyl, pyridyl, thienyl, indolyl, benzpyrazolyl, benzimidazolyl, benzofuranyl, benzoxazolyl, benzisoxazolyl, isobenzofuranyl, benzothiophenyl, isobenzothiophenyl, indolinyl, quinolinyl, isoquinolinyl, quinazolinyl, or quinoxaliny.

In some embodiments, the combining step (b) is carried out in a solvent. In some embodiments, the solvent is selected from the group consisting of tetrahydrofuran, acetonitrile, methylene chloride, ether, methanol, water and combinations thereof.

In some embodiments, the acid of step (b) is selected from the group consisting of, trifluoromethanesulfonic acid, haloacetic acid, trifluoroacetic acid, monofluoroacetic acid, difluoroacetic acid, mono, di-, or trichloroacetic acid, phosphoric acid, sulfuric acid, camphor sulfonic acid, formic acid, acetic acid, tartic acid, haloacetic acid, dibenzoyltartaric acid, hydrochloric acid, hydroiodic acid, hydrofloric acid, and hydrobromic acid.

In some embodiments, the acid is a Lewis acid selected from the group consisting of trimethylsilyl trifluoromethanesulfonate, trimethylsilyl chloride, titanium tetrachloride, gold(III) chloride, boron trifluoride, aluminium trichloride, iron(III) chloride and niobium chloride.

In some embodiments, wherein R⁸ in the compound of Formula Ia is not H and R⁸ in the compound of Formula (IIa) and (IIIa) is H, said method further comprising the step of:

(c) combining the compound of Formula Ia with a compound of R⁸*—Y and a base to produce said compound of Formula Ia, wherein:

Y is bromo, chloro, iodo, triflyl (i.e., trifluoromethylsulfonyl), tosyl (i.e., 4-methylphenylsulfonyl), or mesyl (i.e., methanesulfonyl); and

R⁸* is hydrogen or X—R⁵, wherein X is C₁₋₁₀ alkyl, C₁₋₁₀ alkenyl, C₁₋₁₀ alkynyl, and R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranyl, thiazolyl, thiadiazolyl, furanyl, thienyl, pyrazolyl, quinoxalinyl, or naphthyl. In some embodiments, the base is selected from the group consisting of sodium hydride, lithium hexamethyldisilazide, sodium hexamethyldisilazide, potassium hexamethyldisilazide and potassium tert-butoxide.

In some embodiments, wherein R⁹ in said compound of Formula (Ia) is —X—R⁵ and R⁹ in said compound of Formula (IIa) and Formula (IIIa) is H, said method further comprising the step of: (c) combining the compound of Formula (Ia) with Z-X—R⁵ and a base to produce said compound of Formula (Ia), wherein: Z is bromo, chloro, iodo, triflyl (i.e., trifluoromethylsulfonyl), tosyl (i.e., 4-methylphenylsulfonyl), or mesyl (i.e., methanesulfonyl). In some embodiments, the base is Diaza(1,3)bicyclo[5.4.0] undecane.

In some embodiments, R⁹ in said compound of Formula (Ia) is —X—R⁵ and R⁹ in said compound of Formula (IIa) and Formula (IIIa) is H, said method further comprising the step of: (c) combining the compound of formula (Ia) with R⁵—C(═O)H and a reducing agent to produce said compound of Formula (Ia). In some embodiments, the reducing agent is sodium cyanoborohydride or sodium triacetoxyborohydride. In some embodiments, step (c) is carried out in a solvent. In some embodiments, the solvent is selected from the group of consisting of N-methylpyrrolidone, dichloromethane, toluene, dichloroethane, and tetrahydrofuran.

In some embodiments, the compound of Formula (Ia) is selected from the group consisting of

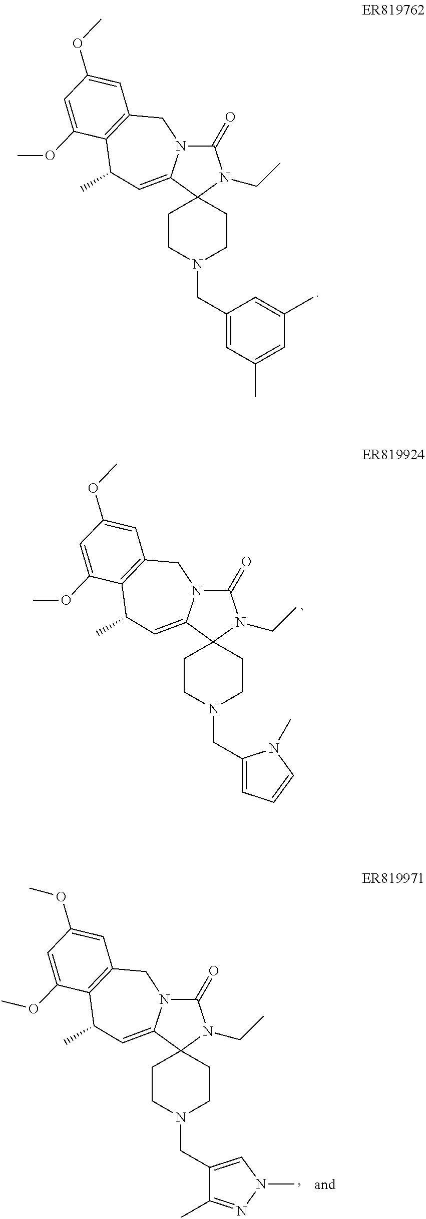

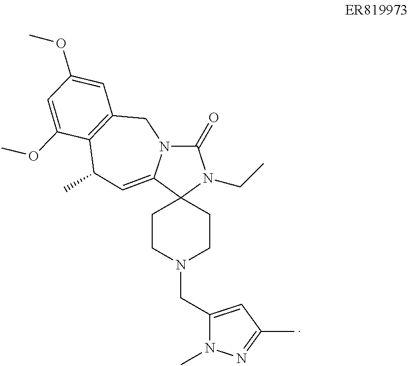

As described herein, the present invention provides or makes by methods as described above compounds of Formula X:

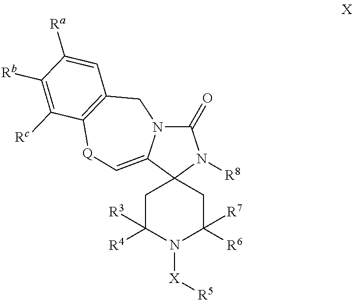

wherein:
Q is —C(R′)(R²)— or —CH═CH— (cis or trans);
R¹ and R² are independently selected from H, C₁₋₃ alkyl, C₂₋₄ alkenyl, or taken together are C₁₋₆ alkylidene or C₂₋₆ alkenylidene;
each of R³, R⁴, R⁶, and R⁷ is independently selected from hydrogen and methyl;
X is methylene, ethylene, or propenylene;
R⁵ is phenyl, quinolinyl, isoquinolinyl, indolyl, furanyl, thienyl, pyrazolyl, quinoxalinyl, naphthyl, or pyrrolyl, and substituted with between 0 and 5 substituents independently selected from C₁₋₃ alkyl, C₁₋₃ alkoxy, hydroxyl, C₁₋₃ alkylthio, cyclopropyl, cyclopropylmethyl, and halo;
R⁸ is H, methyl, ethyl, propenyl, (C₁₋₃ alkoxy)C₁₋₃ alkyl, (C₁₋₃ alkylthio)C₁₋₃ alkyl, C₁₋₃ hydroxyalkyl, phenyl, benzyl, furanyl, pyrrolyl, imidazolyl, pyrazolyl, isothiazolyl, isooxazolyl, pyridyl, or thienyl;
   wherein R⁸ is substituted with between 0 and 3 substituents independently selected from methyl, ethyl, halo, C₁₋₃ alkoxy, C₁₋₃ alkylthio, (C₁₋₃ alkoxy)C₁₋₃ alkyl, (C₁₋₃ alkylthio)C₁₋₃ alkyl, C₁₋₃ hydroxyalkyl, (C₁₋₃ mercaptoalkyl)phenyl, benzyl, furanyl, imidazolyl, pyrazolyl, pyrrolyl, isothiazolyl, isooxazolyl, pyridyl, thienyl, pyranyl, dihydropyranyl, tetrahydropyranyl, and cyclopropyl; and
each of R^{a}, R^{b}, and R^{c} is independently selected from hydrogen, hydroxyl, methoxy, benzyloxy, fluoro, chloro, amino, methylamino, dimethylamino, and phenoxy;
   or one pair selected from R^{a} and R^{b}, and R^{b} and R^{C}, taken together, is —O—(CH₂)—O— or —O—CH₂—CH₂—O—;
      or a pharmaceutically acceptable salt, a C₁₋₆ alkyl ester or amide, or a C₂₋₆ alkenyl ester or amide thereof.

In other embodiments, the present invention provides a pharmaceutical composition comprising a compound of formula I or a subset or example thereof. In certain embodiments, the pharmaceutical composition is useful for treating rheumatoid arthritis or multiple sclerosis.

Other embodiments provide use of a compound of formula I, or a subset or example thereof, in the manufacture of a medicament. In certain embodiments, the present invention provides the use of a compound of formula I, or a subset or example thereof, in the manufacture of a medicament for the treatment of rheumatoid arthritis or multiple sclerosis.

Other aspects of the present invention are disclosed herein.

### DETAILED DESCRIPTION OF CERTAIN EMBODIMENTS OF THE INVENTION

### A. Definitions

Compounds of this invention include those described generally above, and are further illustrated by the embodiments, sub-embodiments, and species disclosed herein. As used herein, the following definitions shall apply unless otherwise indicated.

As described herein, compounds of the invention may optionally be substituted with one or more substituents, such as are illustrated generally above, or as exemplified by particular classes, subclasses, and species of the invention. In general, the term “substituted” refers to the replacement of hydrogen radicals in a given structure with the radical of a specified substituent. Unless otherwise indicated, a substituted group may have a substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds.

The term “stable”, as used herein, refers to compounds that are not substantially altered when subjected to conditions to allow for their production, detection, and preferably their recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week.

The term “alkyl” or “alkyl group,” as used herein, means a straight-chain, (i.e., unbranched) unbranched, branched, or cyclic hydrocarbon chain that is completely saturated. In certain embodiments, alkyl groups contain 1 to 20 carbon atoms. In some embodiments, alkyl groups contain 1 to 10 carbon atoms. In other embodiments, alkyl groups contain 1 to 3 carbon atoms. In still other embodiments, alkyl groups contain 2-5 carbon atoms, and in yet other embodiments alkyl groups contain 1-2, or 2-3 carbon atoms. In certain embodiments, the term “alkyl” or “alkyl group” refers to a cycloalkyl group, also known as carbocycle. Exemplary C₁₋₃ alkyl groups include methyl, ethyl, propyl, isopropyl, and cyclopropyl.

The term “alkenyl” or “alkenyl group,” as used herein, refers to a straight-chain (i.e., unbranched), branched, or cyclic hydrocarbon chain that has one or more double bonds. In some embodiments, alkenyl groups contain 2-20 carbon atoms. In certain embodiments, alkenyl groups contain 2-10 carbon atoms. In certain embodiments, alkenyl groups contain 2-6 carbon atoms, yet another embodiments contain 2-4 carbon atoms. In some embodiments, alkenyl group contain 2-5 carbon atoms. In still other embodiments, alkenyl groups contain 3-4 carbon atoms, and in yet other embodiments alkenyl groups contain 2-3 carbon atoms. According to another aspect, the term alkenyl refers to a straight chain hydrocarbon having two double bonds, also referred to as “diene.” In other embodiments, the term “alkenyl” or “alkenyl group” refers to a cycloalkenyl group. Exemplary C₂₋₄ alkenyl groups include —CH═CH₂, —CH₂CH═CH₂ (also referred to as allyl), —CH═CHCH₃, —CH₂CH₂CH═CH₂, —CH₂CH═CHCH₃, —CH═CH₂CH₂CH₃, —CH═CH₂CH═CH₂, and cyclobutenyl.

The term “alkoxy”, or “alkylthio”, as used herein, refers to an alkyl group, as previously defined, attached to the principal carbon chain through an oxygen (“alkoxy”) or sulfur (“alkylthio”) atom.

As used herein, the term “alkylene” refers to a straight or branched, saturated or unsaturated bivalent hydrocarbon chain. In some embodiments, alkylene groups contain 1-20 carbon atoms. In some embodiments, alkylene groups contain 1-10 carbon atoms. In certain embodiments, alkylene groups contain 1-6 carbon atoms. In other embodiments, alkylene groups contain 2-5, 1-4, 2-4, 1-3, or 2-3 carbon atoms. Exemplary alkylene groups include methylene, ethylene, and propylene. In certain embodiments, alkylene groups have a double bond, referred to herein as “alkenylene.” In other embodiments, alkylene groups have a triple bond, referred to herein as “alkynylene.”

As used herein, the terms “methylene,” “ethylene,” and “propylene” refer to the bivalent moieties —CH₂—, —CH₂ CH₂—, and —CH₂CH₂CH₂—, respectively.

As used herein, the terms ethenylene, propenylene, and butenylene refer to the bivalent moieties —CH═CH—, —CH═CHCH₂—, —CH₂CH═CH—, —CH═CHCH₂CH₂—, CH₂CH═CH₂CH₂—, and —CH₂CH₂CH═CH—, where each ethenylene, propenylene, and butenylene group can be in the cis or trans configuration. In certain embodiments, an ethenylene, propenylene, or butenylene group can be in the trans configuration.

As used herein, the term “alkylidene” refers to a bivalent hydrocarbon group formed by mono or dialkyl substitution of methylene. In some embodiments, an alkylidene group has 1-10 carbon atoms. In certain embodiments, an alkylidene group has 1-6 carbon atoms. In other embodiments, an alkylidene group has, 1-3, 1-4, 1-5, 2-4, 2-5, or 2-6 carbon atoms. Such groups include propylidene (CH₃CH₂CH═), ethylidene (CH₃CH═), methylidene (CH₂═), and isopropylidene (CH₃(CH₃)CH═), and the like.

As used herein, the term “alkenylidene” refers to a bivalent hydrocarbon group having one or more double bonds formed by mono or dialkenyl substitution of methylene. In some embodiments, an alkenylidene group has 2-10 carbon atoms. In certain embodiments, an alkenylidene group has 2-6 carbon atoms. In other embodiments, an alkenylidene group has 2-6, 2-5, 2-4, or 2-3 carbon atoms. According to one aspect, an alkenylidene has two double bonds. Exemplary alkenylidene groups include CH₃CH═C═, CH₂═CHCH═, CH₂═CHCH₂CH═, and CH₂═CHCH₂CH═CHCH═.

As used herein, the term “alkenylidene” refers to a bivalent hydrocarbon group having one or more double bonds formed by mono or dialkenyl substitution of methylene. In some embodiments, an alkenylidene group has 2-10 carbon atoms. In certain embodiments, an alkenylidene group has 2-6 carbon atoms. In other embodiments, an alkenylidene group has 2-6, 2-5, 2-4, or 2-3 carbon atoms. According to one aspect, an alkenylidene has two double bonds. Exemplary alkenylidene groups include CH₃CH═C═, CH₂═CHCH═, CH₂═CHCH₂CH═, and CH₂═CHCH₂CH═CHCH═.

The term “spirocycle,” as used herein, represents an alkenylene or alkylene group in which both ends of the alkenylene or alkylene group are attached to the same carbon of the parent molecular moiety to form a bicyclic group. In some embodiments, it contains 3-10 carbons. In certain embodiments, it contains 4-6 carbon atoms. In some embodiments, it contains 3-6 carbon atoms. Exemplary spiroheterocycle groups taken together with its parent group include, but are not limited to 2-azaspiro[4.5]decan-3-one, 1,3-diazaspiro[4.5]decan-2-one, 1-oxa-3-azaspiro[4.5]decan-2-one, 2-oxa-4-azaspiro[5.5]undecan-3-one.

The term “spiroheterocycle,” as used herein, represents a heteroalkenylene or heteroalkylene group in which both ends of the heteroalkenylene or heteroalkylene group are attached to the same carbon of the parent molecular moiety to form a bicyclic group. In some embodiments, it contains 3-10 carbons. In certain embodiments, it contains 4-6 carbon atoms. In some embodiments, it contains 3-6 carbon atoms. Exemplary spiroheterocycle groups taken together with its parent group include, but are not limited to 1,3,8-triazaspiro[4.5]decan-2-one, and 1,3,8-triazaspiro[4.5]decane-2,4-dione, 1,8,10-triazaspiro[5.5]undecan-9-one, 2,4,8-triazaspiro[5.5]undecan-3-one, 2-oxa-4,9-diazaspiro[5.5]undecan-3-one, 2-oxa-4,8-diazaspiro[5.5]undecan-3-one, 8-oxa-1,10-diazaspiro[5.5]undecan-9-one, 2-oxa-4,8-diazaspiro[5.5]undecan-3-one, and 8-oxa-1,10-diazaspiro[5.5]undecan-9-one.

The “spirocycle” or “spiroheterocycle” groups of the present invention can be optionally substituted with one or more substituents selected from the group consisting of alkyl, aryl, arylalkoxyalkyl, arylalkyl, aryloxyalkyl, or X—R⁵, wherein X is methylene, ethylene, propylene, ethenylene, propenylene, or butenylene; and R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furanyl, thienyl, pyrazolyl, quinoxalinyl, or naphthyl, wherein said R⁵ substituted with between 0 and 5 substituents independently selected from the group consisting of C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl; and halo.

As used herein, the term “C₁₋₆ alkyl ester or amide” refers to a C₁₋₆ alkyl ester or a C₁₋₆ alkyl amide where each C₁₋₆ alkyl group is as defined above. Such C₁₋₆ alkyl ester groups are of the formula (C₁₋₆ alkyl)OC(═O)— or (C₁₋₆ alkyl)C(═O)O—. Such C₁₋₆ alkyl amide groups are of the formula (C₁₋₆ alkyl)NHC(═O)— or (C₁₋₆ alkyl)C(═O)NH—.

As used herein, the term “C₂₋₆ alkenyl ester or amide” refers to a C₂₋₆ alkenyl ester or a C₂₋₆ alkenyl amide where each C₂₋₆ alkenyl group is as defined above. Such C₂₋₆ alkenyl ester groups are of the formula (C₂₋₆ alkenyl)OC(═O)— or (C₂₋₆ alkenyl)C(═O)O—. Such C₂₋₆ alkenyl amide groups are of the formula (C₂₋₆ alkenyl)NHC(═O)— or (C₂₋₆ alkenyl)C(═O)NH—.

The term “alkynyl” or “alkynyl group,” as used herein, refers to a straight-chain (i.e., unbranched) or branched hydrocarbon chain that has one or more triple bonds. In certain embodiments, alkynyl groups contain 2-6 carbon atoms. In still other embodiments, alkynyl groups contain 2-5 carbon atoms, and in yet other embodiments alkynyl groups contain 2-4 or 2-3 carbon atoms. In other embodiments, the term “alkynyl” or “alkynyl group” refers to a cycloalkynyl group. Exemplary C₂₋₆ alkynyl groups include —C≡CH, —CH₂C≡CH (also referred to as vinyl), —C≡CCH₃, —CH₂CH₂C≡CH, —CH₂C≡CCH₃, —C≡CHCH₂CH₃, —CH₂CH₂CH₂C≡CH, —C≡CCH₂CH₂CH₃, —CH₂C≡CCH₂CH₂CH₃, —CH₂CH₂C≡CCH₃, —CH₂CH₂CH₂CH₂C≡CH, —C≡CCH₂CH₂CH₂CH₃, —CH₂C≡CCH₂CH₂CH₃, —CH₂CH₂C≡CCH₂CH₃, —CH₂CH₂CH₂C≡CCH₃, cyclobutynyl, cyclobutynemethyl, cyclopentynyl, cyclopentynemethyl, and cyclohexynyl.

“Cycloalkyl”, as used herein alone or as part of another group, refers to groups having 3 to 10 carbon atoms. In some embodiments, the cycloalkyl employed in the invention have 3 to 8 carbon atoms. Suitable cycloalkyls include, but are not limited to cyclopropyl, cyclobutyl, cyclopentyl, cyclohexyl, cycloheptyl and the like, which, as in the case of other aliphatic, heteroaliphatic or heterocyclic moieties, may optionally be substituted with the same groups as set forth in connection with alkyl and loweralkyl above.

“Heterocycloalkyl” or “heterocycle”, as used herein alone or as part of another group, refers to a non-aromatic 3-, 4-, 5-, 6-, 7-, or 8-membered ring or a polycyclic group, including, but not limited to a bi- or tri-cyclic group comprising fused six-membered rings having between one and four heteroatoms independently selected from oxygen, sulfur and nitrogen, wherein (i) the nitrogen and sulfur heteroatoms may be optionally oxidized, (ii) the nitrogen heteroatom may optionally be quaternized, and (iv) may form a Spiro ring or be fused with an cycloalkyl, aryl, heterocyclic ring, benzene or a heteroaromatic ring. In some embodiments, the heterocycle employed in the invention have 3 to 10 carbon atoms. Representative heterocycles include, but are not limited to, 1,4-dioxa-8-azaspiro[4.5]decane, morpholine, azetidine, azepine, aziridine, diazepine, 1,3-dioxolane, dioxane, dithiane, furan, imidazole, imidazoline, imidazolidine, isothiazole, isothiazoline, isothiazolidine, isoxazole, isoxazoline, isoxazolidine, morpholine, oxadiazole, oxadiazoline, oxadiazolidine, oxazole, oxazoline, oxazolidine, piperazine, piperidine, pyran, pyrazine, pyrazole, pyrazoline, pyrazolidine, pyridine, pyrimidine, pyridazine, pyrrole, pyrroline, pyrrolidine, tetrahydrofuran, tetrahydrothiophene, tetrazine, tetrazole, thiadiazole, thiadiazoline, thiadiazolidine, thiazole, thiazoline, thiazolidine, thiophene, thiomorpholine, thiomorpholine sulfone, thiopyran, triazine, triazole, trithiane, benzimidazole, benzothiazole, benzothiadiazole, benzothiophene, benzoxadiazole, benzoxazole, benzofuran, benzopyran, benzothiopyran, benzodioxine, 1,3-benzodioxole, cinnoline, indazole, indole, indoline, indolizine, naphthyridine, isobenzofuran, isobenzothiophene, isoindole, isoindoline, isoquinoline, phthalazine, purine, pyranopyridine, quinoline, quinolizine, quinoxaline, quinazoline, tetrahydroisoquinoline, tetrahydroquinoline, thiopyranopyridine, and the like. These rings include quaternized derivatives thereof and may be optionally substituted with the same groups as set forth in connection with alkyl and loweralkyl above.

“Aryl” as used herein alone or as part of another group, refers to a monocyclic carbocyclic ring system or a bicyclic carbocyclic fused ring system having one or more aromatic rings. In some embodiments, the aryl employed in the invention have 3 to 14 carbon atoms. Representative examples of aryl include, azulenyl, indanyl, indenyl, naphthyl, phenyl, tetrahydronaphthyl, and the like. The term “aryl” is intended to include both substituted and unsubstituted aryl unless otherwise indicated and these groups may be optionally substituted with the same groups as set forth in connection with alkyl and loweralkyl above.

“Heteroaryl” as used herein alone or as part of another group, refers to a cyclic, aromatic hydrocarbon in which one or more carbon atoms have been replaced with heteroatoms such as O, N, and S. If the heteroaryl group contains more than one heteroatom, the heteroatoms may be the same or different. In some embodiments, the heteroaryl employed in the invention have 3 to 14 carbon atoms. Examples of heteroaryl groups include pyridyl, pyrimidinyl, imidazolyl, thienyl, furanyl, pyrazinyl, pyrrolyl, pyranyl, isobenzofuranyl, chromenyl, xanthenyl, indolyl, isoindolyl, indolizinyl, triazolyl, pyridazinyl, indazolyl, purinyl, quinolizinyl, isoquinolyl, quinolyl, phthalazinyl, naphthyridinyl, quinoxalinyl, isothiazolyl, and benzo[b]thienyl. In some embodiments, heteroaryl groups are five and six membered rings and contain from one to three heteroatoms independently selected from O, N, and S. The heteroaryl group, including each heteroatom, can be unsubstituted or substituted with from 1 to 4 substituents, as chemically feasible. For example, the heteroatom N or S may be substituted with one or two oxo groups, which may be shown as ═O.

“Amine” or “amino group”, as used herein alone or as part of another group, refers to the radical —NH₂. An “optionally substituted” amines refers to —NH₂ groups wherein none, one or two of the hydrogens is replaced by a suitable substituent. Disubstituted amines may have substituents that are bridging, i.e., form a heterocyclic ring structure that includes the amine nitrogen.

The term “alkylamino” refers to a group having the structure —NHR′ wherein R′ is alkyl, as defined herein. The term “aminoalkyl” refers to a group having the structure NH2R′—, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

“Haloalkyl”, as used herein alone or as part of another group, refers to an alkyl group, as defined above, having one, two, or three halogen atoms attached thereto and is exemplified by such groups as chloromethyl, bromoethyl, trifluoromethyl, and the like.

“Haloacetic acid”, as used herein, has a formula X—CH₃₋ₙCOOH. X is an halogen atom, such as F, Cl, Br, I. n is 1, 2, or 3. Examples include trifluoroacetic acid, monofluoroacetic acid, difluoroacetic acid, mono, di-, or trichloroacetic acid.

Unless indicated otherwise, nomenclature used to describe chemical groups or moieties as used herein follow the convention where, reading the name from left to right, the point of attachment to the rest of the molecule is at the right-hand side of the name. For example, the group “(C₁₋₃ alkoxy)C₁₋₃ alkyl,” is attached to the rest of the molecule at the alkyl end. Further examples include methoxyethyl, where the point of attachment is at the ethyl end, and methylamino, where the point of attachment is at the amine end.

Unless indicated otherwise, where a bivalent group is described by its chemical formula, including two terminal bond moieties indicated by “—,” it will be understood that the attachment is read from left to right. By way of example, when X is —CH₂CH═CH—, X is attached to the nitrogen of the hydantoin core at the Left-hand side methylene and X is attached to R⁵ at the right-hand side methyne.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. In certain embodiment, when the Q group of formula I comprises a double bond, that double bond can be in the cis (E) or trans (Z) conformation. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this invention. Such compounds are useful, for example, as analytical tools or probes in biological assays.

As used herein, the terms “treatment,” “treat,” and “treating” refer to reversing, alleviating, delaying the onset of, inhibiting the progress of, or preventing a disease or disorder as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The following abbreviations may be used in this application: tetrahydrofuran (THF), acetonitrile (ACN), methylene chloride (CH₂Cl₂), ether (Et₂O), methanol (MeOH), water (H₂O), trifluoromethansulfonic acid (TfOH), trifluoroacetic acid (TFA), camphor sulfonic acid (CSA), hydrochloric acid (HCl), hydroiodic acid (HI), hydrofloric Acid (HF), hydrobromic acid (HBr), trimethylsilyl trifluoromethanesulfonate (TMSOTf), trimethylsilyl chloride (TMSCl), titanium tetrachloride (TiCl₄), gold(III) chloride (AuCl₃), boron trifluoride (BF₃), aluminium trichloride (AlCl₃), iron(III) chloride (FeCl₃) and niobium chloride (NbCl₅), lithium hexamethyldisilazide (LHMDS) potassium tert-butoxide (KO′Bu), sodium hydride (NaH), Diaza(1,3)bicyclo[5.4.0] undecane (DBU), sodium cyanoborohydride (NaBH₃CN), Sodium triacetoxyborohydride (NaBH(OAc)₃), N-methylpyrrolidone (NMP), sodium hexamethyldisilazide (NaHMDS), potassium hexamethyldisilazide (KHMDS).

### B. Compounds

In one embodiment, the present invention provides a compound of formula X:

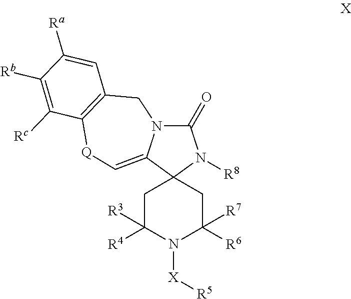

wherein:
Q is —C(R¹)(R²)— or —CH═CH— (cis or trans);
R¹ and R² are independently selected from H, C₁₋₃ alkyl, C₂₋₄ alkenyl, or taken together are C₁₋₆ alkylidene or C₂₋₆ alkenylenidene;
each of R³, R⁴, R⁶, and R⁷ is independently selected from hydrogen and methyl;
X is methylene, ethylene, or propenylene;
R⁵ is phenyl, quinolinyl, isoquinolinyl, indolyl, furanyl, thienyl, pyrazolyl, quinoxalinyl, naphthyl, or pyrrolyl, and substituted with between 0 and 5 substituents independently selected from C₁₋₃ alkyl, C₁₋₃ alkoxy, hydroxyl, C₁₋₃ alkylthio, cyclopropyl, cyclopropylmethyl, and halo;
R⁸ is H, methyl, ethyl, propenyl, (C₁₋₃ alkoxy)C₁₋₃ alkyl, (C₁₋₃ alkylthio)C₁₋₃ alkyl, C₁₋₃ hydroxyalkyl, phenyl, benzyl, furanyl, pyrrolyl, imidazolyl, pyrazolyl, isothiazolyl, isooxazolyl, pyridyl, and thienyl;
   wherein R⁸ is substituted with between 0 and 3 substituents independently selected from methyl, ethyl, halo, C₁₋₃ alkoxy, C₁₋₃ alkylthio, (C₁₋₃ alkoxy)C₁₋₃ alkyl, (C₁₋₃ alkylthio)C₁₋₃ alkyl, C₁₋₃ hydroxyalkyl, (C₁₋₃ mercaptoalkyl)phenyl, benzyl, furanyl, imidazolyl, pyrazolyl, pyrrolyl, isothiazolyl, isooxazolyl, pyridyl, thienyl, pyranyl, dihydropyranyl, tetrahydropyranyl, and cyclopropyl; and
each of R^{a}, R^{b}, and R^{c} is independently selected from hydrogen, hydroxyl, methoxy, benzyloxy, fluoro, chloro, amino, methylamino, dimethylamino, and phenoxy;
   or one pair selected from R^{a} and R^{b}, and R^{b} and R^{c}, taken together, is —O—(CH₂)—O— or —O—CH₂—CH₂—O—;
      or a pharmaceutically acceptable salt, a C₁₋₆ alkyl ester or amide, or a C₂₋₆ alkenyl ester or amide thereof.

In certain embodiments, Q is —C(R¹)(R²)—, wherein R¹ and R² are independently selected from H, methyl, ethyl, or taken together are CH₂═, allylidene, propylidene, propenylidene, or ethylidene. In other embodiments, R¹ and R² are independently selected from H and methyl, or taken together are CH₂═. According to another embodiment, R¹ and R² are independently selected from H, methyl, ethyl, or taken together are propylidene, allylidene, or CH₂═. In certain embodiments, each of R¹ and R² is independently selected from H, methyl, and ethyl. In other embodiments, one of R¹ and R² is H, and the other is methyl or ethyl. In still other embodiments, one of R¹ and R² is methyl and the other is H. Yet another aspect provides a compound of formula X wherein one of R¹ and R² is H. According to yet another embodiment, R¹ and R² taken together are propylidene, vinylidene, or CH₂═.

As defined generally above, X is methylene, ethylene, or propenylene. In certain embodiments, X is methylene or ethylene. In other embodiments, X is —CH₂CH═CH— in the trans configuration.

In certain embodiments, each of R³, R⁴, R⁶, and R⁷ is hydrogen.

According to one embodiment, R⁵ is phenyl, quinolinyl, isoquinolinyl, indolyl, quinoxalinyl, or naphthyl, and substituted with between 0 and 3 substituents independently selected from methyl, methoxy, hydroxyl, bromo, fluoro, and chloro. According to another embodiment, R⁵ is phenyl, quinolinyl, isoquinolinyl, indolyl, quinoxalinyl, or naphthyl, and substituted with between 0 and 3 substituents independently selected from hydrogen, fluoro, methyl, methoxy, hydroxyl, and bromo. In certain embodiments, R⁵ is phenyl, quinolinyl, isoquinolinyl, indolyl, furanyl, thienyl, pyrazolyl, quinoxalinyl, or naphthyl, and substituted with between 0 and 3 substituents independently selected from methyl, methoxy, fluoro, and bromo. In other embodiments, R⁵ is phenyl, 4-quinolinyl, 5-quinolinyl, 8-quinolinyl, 5-isoquinolinyl, 3-indolyl, N-methyl-3-indolyl, 5-quinoxalinyl, 1-naphthyl, or 2-naphthyl, and substituted or further substituted with between 0 and 3 substituents independently selected from methyl, methoxy, and bromo. In still other embodiments, R⁵ is phenyl, having the following substituents: fluoro, methyl or hydroxyl at the 2-position; hydrogen, methyl, or methoxy at the 3-position; and hydrogen, methyl, or methoxy at the 5-position. According to another aspect, R⁵ is 2-fluoro-3,5-dimethylphenyl, 2-fluoro-3,5-dimethoxyphenyl, 3,5-dimethylphenyl, 2-hydroxy-3,5-dimethoxyphenyl, 2,3-dimethyl, or 2-methyl-3,5-dimethoxyphenyl.

According to one embodiment, R⁸ is H, methyl, ethyl, methoxyethyl, methylthioethyl, hydroxyethyl, hydroxylpropyl, benzyl, or phenyl, optionally substituted. According to another embodiment, R⁸ is H, methyl, ethyl, hydroxyethyl, benzyl, or phenyl; wherein phenyl is optionally substituted with pyrrolyl or pyrazolyl. In certain embodiments, R⁸ is benzyl, phenyl, (pyrrolyl)phenyl, or (pyrazolyl)phenyl. In other embodiments, R⁸ is H, methyl, ethyl, hydroxyethyl, or methoxyethyl. In still other embodiments, R⁸ is methyl, ethyl, methoxy, ethyl, or hydroxyethyl.

In certain embodiments, each of R^{a}, R^{b}, and R^{c} is independently selected from hydrogen, hydroxyl, methoxy, benzyloxy, fluoro, and chloro. In other embodiments, each of R^{a}, R^{b}, and R^{c} is independently selected from hydrogen, methoxy, and fluoro. In still other embodiments, R^{c} is methoxy or fluoro. According to another embodiment, R^{a} and R^{c} are methoxy or fluoro.

According to another aspect, the present invention provides a compound of formula Ib, wherein:
Q is —C(R¹)(R²)—;
R¹ and R² are independently selected from H, methyl, ethyl, or taken together are CH₂=, allylidene, propylidene, propenylidene, or ethylidene;
each of R³, R⁴, R⁶, and R⁷ is hydrogen;
X is methylene, ethylene, or propenylene;
R⁵ is phenyl, quinolinyl, isoquinolinyl, indolyl, quinoxalinyl, or naphthyl, and substituted with between 0 and 3 substituents independently selected from methyl, methoxy, hydroxyl, bromo, fluoro, and chloro;
R⁸ is H, methyl, ethyl, methoxyethyl, methylthioethyl, hydroxyethyl, hydroxylpropyl, benzyl, or phenyl, optionally substituted (as described in paragraph [0030]); and
each of R^{a}, R^{b}, and R^{c} is independently selected from hydrogen, hydroxyl, methoxy, benzyloxy, fluoro, and chloro.

According to another aspect, the present invention provides a compound of formula Ib wherein:
Q is —C(R′)(R²)—;
R¹ and R² are independently selected from H and methyl, or taken together are CH₂═;
each of R³, R⁴, R⁶, and R⁷ is hydrogen;
X is methylene, ethylene, or propenylene;
R⁵ is phenyl, quinolinyl, isoquinolinyl, indolyl, quinoxalinyl, or naphthyl, and substituted with between 0 and 3 substituents independently selected from hydrogen, fluoro, methyl, methoxy, hydroxyl, and bromo;
R⁸ is H, methyl, ethyl, hydroxyethyl, benzyl, or phenyl; wherein phenyl is optionally substituted with pyrrolyl or pyrazolyl; and
each of R^{a}, R^{b}, and R^{c} is independently selected from hydrogen, methoxy, and fluoro.

Yet another aspect of the present invention provides a compound of formula X, wherein:
Q is —C(R¹)(R²)—;
R¹ and R² are independently selected from H, methyl, ethyl, or taken together are propylidene, allylidene, or CH₂═;
each of R³, R⁴, R⁶, and R⁷ is hydrogen;
X is methylene or ethylene;
R⁵ is phenyl, quinolinyl, isoquinolinyl, indolyl, furanyl, thienyl, pyrazolyl, quinoxalinyl, or naphthyl, and substituted with between 0 and 3 substituents independently selected from methyl, methoxy, fluoro, and bromo; and
R⁸ is H, methyl, ethyl, hydroxyethyl, benzyl, or phenyl; wherein phenyl is optionally substituted with pyrrolyl or pyrazolyl.

In certain embodiments, the present invention provides a compound of formula Ib, wherein:
Q is —C(R¹)(R²)—;
one of R¹ and R² is H and the other is methyl or ethyl;
each of R³, R⁴, R⁶, and R⁷ is hydrogen;

R⁵ is phenyl, having the following substituents: fluoro, methyl or hydroxyl at the 2-position; hydrogen, methyl, or methoxy at the 3-position; and hydrogen, methyl, or methoxy at the 5-position; and
R⁸ is methyl, ethyl, methoxy, ethyl, or hydroxyethyl.

It will be appreciated that all embodiments, classes and subclasses described above and herein are contemplated both singly and in combination.

Exemplary compounds of formula X are set forth in the Examples section and in Table 1-2, below. Thus particular examples of the compounds of the invention include, but are not limited to:

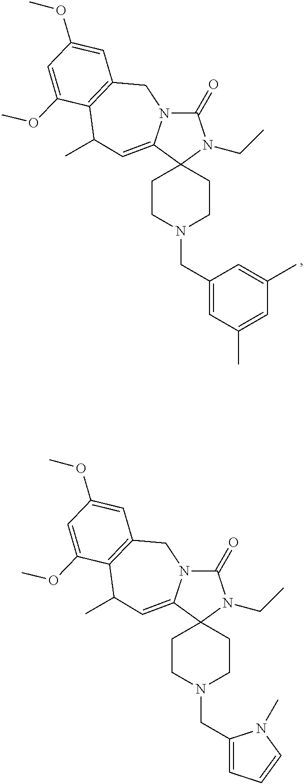

and pharmaceutically acceptable salts thereof.

### C. Methods of Making Compounds of Formula I and Formula Ia

In some embodiments, the present invention provides a method of making a compound of Formula I:

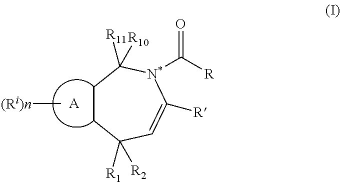

comprising the steps of:

(a) providing a compound of Formula (II) or (III):

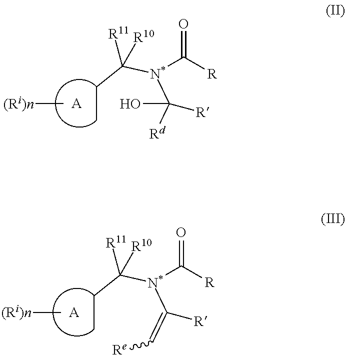

wherein:

ring A is C₃₋₁₄ aryl or C₃₋₄₄heteroaryl

n is an integer from 0 to 4,

each occurrence of Rⁱ is independently selected from the group consisting of hydrogen, hydroxyl, C₁₋₁₀ alkoxy, benzyloxy, benzyl, halo, amino, (C₁₋₆ alkyl)amino, (C₁₋₆alkyl)(C₁₋₆alkyl)amino, phenoxyl, and phenyl; or two adjacent Rⁱ, taken together, is —O—(CH₂)—O— or —O—CH₂—CH₂—O— and Rⁱ is attached to the A ring as valence permits;

R and R′ are each independently hydrogen, C₁₋₁₀ alkyl, C₂₋₁₀ alkenyl, C₂₋₁₀ alkynyl, C₁₋₁₀ alkoxy, C₁₋₁₀ alkylsulfonyl, C₁₋₁₀ haloalkyl, C₁₋₁₀ aminoalkyl, amino, (C₁₋₆ alkyl)amino, (C₁₋₆alkyl)(C₁₋₆alkyl)amino, C₃₋₁₀ cycloalkyl, C₃₋₁₀ cycloalkenyl, C₃₋₁₀ cycloalkynyl, C₃₋₁₀ heterocycle, C₃₋₁₄ aryl, or C₃₋₁₄ heteroaryl,

or R and R′ taken together form with N* a C₃₋₁₀ cycloalkyl, C₃₋₁₀ cycloalkenyl, C₃₋₁₀ cycloalkynyl, C₄₋₁₀ heterocyclyl, C₃₋₁₄ aryl, or C₃₋₁₄ heteroaryl ring system, which ring system is unsubstituted or substituted from one to four times with substituents independently selected from the group consisting of halo, oxygen, hydroxyl, sulfuryl, amino, nitro, cyano, haloalkyl, C₁₋₁₀ alkyl, C₃₋₁₀ spirocyclyl, C₃₋₁₀ spiroheterocyclyl, C₂₋₁₀ alkenyl, C₂₋₁₀ alkynyl, C₁₋₁₀alkoxy, aminoalkyl, C₁₋₁₀ thioalkyl, C₃₋₁₀ heterocyclyl, C₃₋₁₀ cycloalkyl, C₃₋₁₄ aryl, and C₃₋₁₄ heteroaryl,

R¹ and R² are independently hydrogen, C₁₋₁₀ alkyl, C₂₋₁₀ alkenyl, C₂₋₁₀ alkynyl, or taken together are C₂₋₁₀ alkenyl or C₂₋₁₀ alkenylenidene, or R¹ and R² taken together form C₃₋₁₀ cycloalkyl or C₃₋₁₀ heterocyclyl,

R¹⁰ and R¹¹ are independently selected from the group consisting of hydrogen, oxygen, hydroxyl, C₁₋₁₀ alkyl, C₂₋₁₀ alkenyl, C₂₋₁₀ alkynyl, C₁₋₁₀ alkoxy, C₁₋₁₀ alkylsulfonyl, haloalkyl, C₁₋₁₀ aminoalkyl, amino, (C₁₋₆ alkyl)amino, (C₁₋₆alkyl)(C₁₋₆alkyl)amino, C₃₋₁₀ cycloalkyl, C₃₋₁₀ cycloalkenyl, C₃₋₄₀ cycloalkynyl, C₃₋₁₀ heterocycle, C₃₋₁₄ aryl and C₃₋₁₄ heteroaryl, or taken together form C₂₋₁₀ alkenyl, C₃₋₁₀cycloalkyl, C₃₋₁₀heterocyclyl

R^{d} is C₂₋₁₀ alkenyl or C₂₋₁₀ alkynyl,

R^{e} is C₂₋₁₀ alkenyl or C₂₋₁₀ alkynyl, wherein R^{e} is positioned cis or trans to the double bond; and

(b) combining said compound of Formula (II) or (III) with an acid to produce a compound of Formula I.

In some embodiments, R^{e} is positioned cis to the double bond.

In some embodiments, the ring A is selected from the group consisting of phenyl, furanyl, pyrrolyl, pyrazolyl, imidazolyl, thiazolyl, thiophenyl, oxazolyl, isoxazolyl, oxadiazolyl, thiazolyl, pyridinyl, pyrimidinyl, pyrazinyl, pyridazinyl, triazinyl, indolyl, benzothiophenyl, benzofuranyl, isobenzofuranyl, indazyl, and benzimidazolyl. In another embodiments, the ring A is phenyl or furanyl.

In some embodiments, ring A is phenyl or furanyl, n is an integer 0-3, each occurrence of Ri is independently selected from the group consisting of hydrogen, methoxyl, benzyloxy or two adjacent Rⁱ, taken together, is —O—(CH₂)—O— or —O—CH₂—CH₂—O—, R and R′ taken together form with N* a C₄₋₁₀ heterocyclyl, which C₄₋₁₀ heterocyclyl is unsubstituted or substituted from three to sever times with substituents independently selected from the group consisting of C₄₋₆ spirocycle, C₃₋₁₀ spiroheterocycle, R¹ and R² are independently hydrogen, C₁₋₁₀ alkyl, or taken together are C₂₋₆ alkenyl, R¹⁰ and R¹¹ are hydrogen, R^{d} is C₂₋₅ alkenyl or C₂₋₅ alkynyl, R^{e} is C₂₋₅ alkenyl or C₂₋₅ alkynyl, wherein R^{e} is positioned cis or trans to the double bond.

In some embodiments, step (b) is carried out in a solvent. In some embodiments, the solvent is selected from the group consisting of tetrahydrofuran, acetonitrile, methylene chloride, ether, methanol, water and combinations thereof.

In some embodiments, the acid is selected from the group consisting of, trifluoromethansulfonic acid, trifluoroacetic acid, monofluoroacetic acid, difluoroacetic acid, mono, di-, or trichloroacetic acid, phosphoric acid, sulfuric acid, camphor sulfonic acid, formic acid, acetic acid, tartic acid, haloacetic acid, dibenzoyltartaric acid, hydrochloric acid, hydroiodic acid, hydrofloric acid, hydrobromic acid. In some embodiments, the acid is selected from the group consisting of, trifluoromethansulfonic acid, trifluoroacetic acid, camphor sulfonic acid, formic acid, acetic acid, tartic acid, dibenzoyltartaric acid.

In some embodiments, the acid is a Lewis acid selected from the group consisting of trimethylsilyl trifluoromethaneulfonate, trimethylsilyl chloride, titanium tetrachloride, gold(III) chloride, boron trifluoride, aluminium trichloride, iron(III) chloride and niobium chloride. In some embodiments, the acid is a Lewis acid selected from the group consisting of Trimethylsilyl trifluoromethanesulfonate, trimethylsilyl chloride, titanium tetrachloride and dichlorodiisopropoxytitanium.

In certain embodiments, the present invention provides a method of making a compound of Formula (Ia)

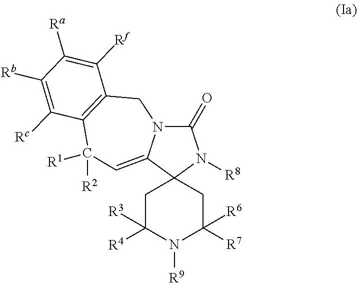

comprising the steps of:

(a) providing a compound of Formula (IIa) or (IIIa):

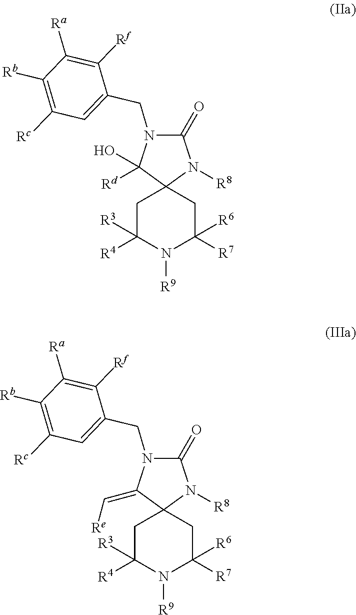

wherein:

R¹ and R² are independently hydrogen, C₁₋₁₀ alkyl, or C₂₋₁₀ alkenyl, C₂₋₁₀ alkynyl, or taken together are C₂₋₁₀ alkenyl or C₂₋₁₀ alkenylenidene, or form C₃₋₁₀ cycloalkyl or C₃₋₁₀ heterocyclyl,

each of R³, R⁴, R⁶, and R⁷ are independently selected from hydrogen and methyl, or R³ and R⁶ taken together is —(CH₂CH₂)—,

R^{d} and R^{e} are independently C₂₋₁₀ alkenyl or C₂₋₁₀ alkynyl, and R^{e} is positioned cis or trans to the double bond,

each of R^{a}, R^{b}, R^{c} and R^{f} is independently selected from the group consisting of hydrogen, hydroxyl, C₁₋₁₀ alkoxy, benzyloxy, benzyl, halo, amino, (C₁₋₆ alkyl)amino, (C₁₋₆alkyl)(C₁₋₆alkyl)amino, phenoxy, and phenyl; or one pair selected from R^{a} and R^{b}, and R^{b} and R^{c}, taken together, is —O—(CH₂)—O— or —O—CH₂—CH₂—O—,

R⁹ is hydrogen or X—R⁵, wherein X is C₁₋₁₀ alkylene, C₂₋₁₀ alkenylene, C₂₋₁₀ alkynlene, and R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furanyl, thienyl, pyrazolyl, quinoxalinyl, or naphthyl,

wherein said R⁵ substituted with between 0 and 5 substituents independently selected from the group consisting of C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl and halo,

R⁸ is hydrogen, methyl, ethyl, propyl, (C₁₋₃ alkoxy)C₁₋₃ alkyl, (C₁₋₃ alkylthio)C₁₋₃ alkyl, C₁₋₃ hydroxyalkyl, phenyl, benzyl, furanyl, pyrrolyl, imidazolyl; pyrazolyl, pyrrolyl, isothiazolyl, isooxazolyl, pyridyl, and thienyl,

wherein R⁸ is substituted with between 0 and 3 substituents independently selected from methyl, ethyl, halo, hydroxyl, C₁₋₃ alkoxy, C₁₋₃ alkylthio, (C₁₋₃ alkoxy)C₁₋₃ alkyl, (C₁₋₃ alkylthio)C₁₋₃ alkyl, C₁₋₃ hydroxyalkyl, (C₁₋₃ mercaptoalkyl)phenyl, benzyl, furanyl, imidazolyl, pyrazolyl, pyrrolyl, thiazolyl, isothiazolyl, oxazolyl, isooxazolyl, pyridyl, thienyl, indolyl, benzpyrazolyl, benzimidazolyl, benzofuranyl, benzoxazolyl, benzisoxazolyl, isobenzofuranyl, benzothiophenyl, isobenzothiophenyl, indolinyl, quinolinyl, isoquinolinyl, quinazolinyl, or quinoxalinyl, and

(b) combining said compound of Formula (IIa) or (IIIa) with an acid to produce a compound of Formula (Ia).

In some embodiments, R¹ and R² are independently hydrogen or C₁₋₁₀ alkyl, or taken together are C₂₋₄ alkenyl, each of R³, R⁴, R⁶, and R⁷ are independently selected from hydrogen and methyl, or R3 and R6 taken together is —(CH₂CH₂)—, R^{d} is —(CH₂)ₘC(Rᵢ)═C(Rᵢᵢ)(Rᵢᵢᵢ) or —(CH₂)ₘC≡C(Rᵢ), wherein each occurrence of Rᵢ, Rᵢᵢ, Rᵢᵢᵢ are independently hydrogen, C₁₋₆alkyl, and m is 0 or 1, R^{c} is —(CH₂)ₚC(Rᵢᵥ)═C(Rᵥ)(Rᵥᵢ), wherein Rᵢᵥ, Rᵥ, Rᵥᵢ are independently hydrogen, C₁₋₆alkyl, and p is 0 or 1, each of R^{a}, R^{b}, R^{c} and R^{f} is independently selected from the group consisting of hydrogen, hydroxyl, methoxyl, benzyloxy, or one pair selected from R^{a} and R^{b}, and R^{b} and R^{c}, taken together, is —O—(CH₂)—O—, R⁹ is hydrogen or X—R⁵, wherein X is C₁₋₁₀ alkyl, C₁₋₁₀ alkenyl, C₁₋₁₀ alkynyl, and R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranyl, thiazolyl, thiadiazolyl, furanyl, thienyl, pyrazolyl, quinoxalinyl, or naphthyl, wherein said R⁵ substituted with between 0 and 5 substituents independently selected from the group consisting of C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl and halo, R⁸ is hydrogen, methyl, ethyl, propyl, (C₁₋₃ alkoxy)C₁₋₃ alkyl, (C₁₋₃ alkylthio)C₁₋₃ alkyl, C₁₋₃ hydroxyalkyl, phenyl, benzyl, furanyl, pyrrolyl, imidazolyl, pyrazolyl, pyrrolyl, isothiazolyl, isooxazolyl, pyridyl, and thienyl, wherein R⁸ is substituted with between 0 and 3 substituents independently selected from methyl, ethyl, halo, hydroxyl, C₁₋₃ alkoxy, C₁₋₃ alkylthio, (C₁₋₃ alkoxy)C₁₋₃ alkyl, (C₁₋₃ alkylthio)C₁₋₃ alkyl, C₁₋₃ hydroxyalkyl, (C₁₋₃ mercaptoalkyl)phenyl, benzyl, furanyl, imidazolyl, pyrazolyl, pyrrolyl, thiazolyl, isothiazolyl, oxazolyl, isooxazolyl, pyridyl, thienyl, indolyl, benzpyrazolyl, benzimidazolyl, benzofuranyl, benzoxazolyl, benzisoxazolyl, isobenzofuranyl, benzothiophenyl, isobenzothiophenyl, indolinyl, quinolinyl, isoquinolinyl, quinazolinyl and quinoxalinyl.

In certain embodiments, the step (b) is carried out in a solvent. In certain embodiments, the solvent comprises a solvent selected from the group consisting of tetrahydrofuran, acetonitrile, methylene chloride, ether, methanol, water and combinations thereof.

In some embodiments, the acid is selected from the group consisting of trifluoromethansulfonic acid, trifluoroacetic acid, phosphoric acid, sulfuric acid, camphor sulfonic acid, formic acid, acetic acid, tartic acid, dibenzoyltartaric acid hydrochloric acid, hydroiodic acid, hydrofloric acid, hydrobromic acid. In some embodiments, the acid is selected from the group consisting of, trifluoromethansulfonic acid, trifluoroacetic acid, camphor sulfonic acid, formic acid, acetic acid, tartic acid, dibenzoyltartaric acid. In some embodiments, the acid is a Lewis acid selected from the group consisting of trimethylsilyl trifluoromethanesulfonate, trimethylsilyl chloride, titanium tetrachloride, gold(III) chloride, boron trifluoride, aluminium trichloride, iron(III) chloride and niobium chloride. In some embodiments, the Lewis acid is Trimethylsilyl trifluoromethanesulfonate, trimethylsilyl chloride, titanium tetrachloride or dichlorodiisopropoxytitanium.

In some embodiments, when R⁸ in the compound of Formula Ia is not H and R⁸ in the compound of Formula (IIa) and (IIIa) is H, said method further comprising the step of (c) combining the compound of Formula Ia with a compound of R⁸*—Y and a base to produce said compound of Formula Ia, wherein: Y is bromo, chloro, iodo, triflyl (i.e., trifluoromethylsulfonyl), tosyl (i.e., 4-methylphenylsulfonyl), or mesyl (i.e., methanesulfonyl); and R⁸* is hydrogen or X—R⁵, wherein X is C₁₋₁₀ alkyl, C₁₋₁₀ alkenyl, C₁₋₁₀ alkynyl, and R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranyl, thiazolyl, thiadiazolyl, furanyl, thienyl, pyrazolyl, quinoxalinyl, or naphthyl. In some embodiments, Y is bromo, chloro, or iodo and R⁸* is hydrogen or X—R⁵, wherein X is C₁₋₁₀ alkyl, C₁₋₁₀ alkenyl, C₁₋₁₀ alkynyl, and R⁵ is phenyl. In some embodiments, the base is selected from the group consisting of sodium hydride, lithium hexamethyldisilazide, sodium hexamethyldisilazide, potassium hexamethyldisilazide and potassium tert-butoxide.

In some embodiments, R⁹ in said compound of Formula (Ia) is —X—R⁵ and R⁹ in said compound of Formula (IIa) and Formula (IIIa) is H, said method further comprising the step of: (c) combining the compound of Formula (Ia) with Z-X—R⁵ and a base to produce said compound of Formula (Ia), wherein: Z is bromo, chloro, iodo, triflyl (i.e., trifluoromethylsulfonyl), tosyl (i.e., 4-methylphenylsulfonyl), or mesyl (i.e., methanesulfonyl). In some embodiments, the base is Diaza(1,3)bicyclo[5.4.0] undecane.

In some embodiments, when R⁹ in said compound of Formula (Ia) is —X—R⁵ and R⁹ in said compound of Formula (IIa) and Formula (IIIa) is H, said method further comprising the step of: (c) combining the compound of formula (Ia) with R5-C(═O)H and a reducing agent to produce said compound of Formula (Ia). In some embodiments, the reducing agent is sodium cyanoborohydride or sodium triacetoxyborohydride. In some embodiments, step (c) is carried out in a solvent. Any suitable solvent or solvent system can be used (see, e.g., U.S. Pat. Nos. 7,256,314; 7,227,028, and 6,469,200, the disclosures of which are incorporated herein by reference). In some embodiments, the solvent is selected from the group of consisting of N-methylpyrrolidone, dichloromethane, toluene, dichloroethane, and tetrahydrofuran.

In some embodiments, R1 and R2 are independently hydrogen or C1-3 alkyl, R3, R4, R6, and R7 are hydrogen, Rd is —(CH₂)mC(Ri)═C(Rii)(Riii) or —(CH2)mC≡C(Ri), wherein each occurrence of Ri, Rii, Riii are independently hydrogen, C1-3alkyl, and m is 0 or 1, R^{e} is —(CH2)pC(Rᵢᵥ)═C(Rᵥ)(Rᵥᵢ), wherein Rᵢᵥ, Rᵥ, Rᵥᵢ are independently hydrogen, C₁₋₃alkyl, and p is 0 or 1, each of R^{a}, R^{b}, R^{c} and R^{f} is independently hydrogen or C₁₋₃ alkoxy, R⁹ is hydrogen or X—R⁵, wherein X is C₁₋₃ alkylene, and R⁵ is phenyl, pyrrolyl, pyrazolyl, wherein said R⁵ substituted with 1 or 2 substituents of C₁₋₃ alkyl, R⁸ is hydrogen, methyl, ethyl, or propyl.

Exemplary compounds of formula (Ia) and formula (I) are set forth in the Examples section and in Tables below. Thus particular examples of the compounds of the invention include, but are not limited to:

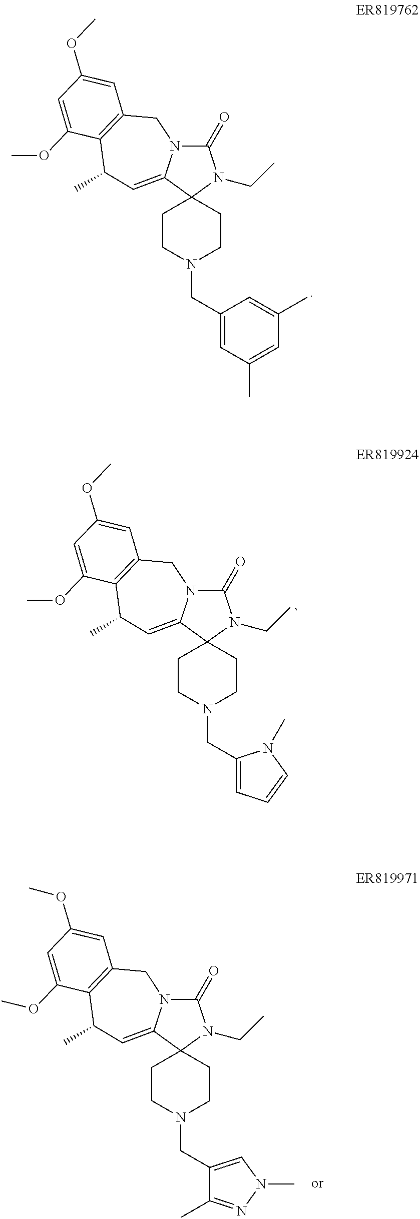

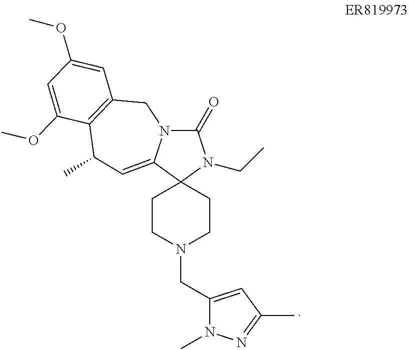

### E. Uses, Formulation and Administration

Pharmaceutically acceptable compositions. The compounds and compositions described herein are generally useful for the inhibition of Th1 cell formation. In particular, these compounds, and compositions thereof, are useful as inhibitors, directly or indirectly, of the T-bet signalling pathway. Thus, the compounds and compositions of the invention are therefore also particularly suited for the treatment of diseases and disease symptoms that are mediated by Th1 cells and/or T-bet signalling pathway.

In one particular embodiment, the compounds and compositions of the invention are inhibitors, directly or indirectly, of the T-bet signalling pathway, and thus the compounds and compositions are particularly useful for treating or lessening the severity of disease or disease symptoms associated with the T-bet signalling pathway.

The term “patient” or “subject”, as used herein, means an animal, preferably a mammal, and most preferably a human, patient or subject.

In certain embodiments, the present invention provides a composition comprising a compound of formula X. In other embodiments, the present invention provides a composition comprising any of the compounds set forth in Tables 1 and 2. According to another aspect, the present invention provides a composition comprising a compound selected from ER-819724, ER-819755, ER-819750, ER-819749, ER-819735. According to yet another aspect, the present invention provides a composition comprising a compound selected from ER-819543, ER-819549, ER-819543, ER-819701, ER-819544, ER-819594, ER-819647, ER-819657, ER-819659, and ER-819592. In other embodiments, the present invention provides a composition comprising a compound selected from ER-819595, ER-819597, ER-819641, ER-819673, ER-819651, ER-819583, ER-819604, ER-819593, ER-819658, and ER-819648. In still other embodiments, the present invention provides a composition comprising a compound selected from ER-819602, ER-819689, ER-819646, ER-819655, ER-819703, ER-819667, ER-819601, ER-819605, ER-819652, ER-819688, ER-819603, ER-819642, and ER-819628. Yet another embodiment provides a composition comprising a compound selected from ER 819-891, ER— ER-819772, ER-819771, ER-819770, ER-819769, ER-819768, and ER-819767. In certain embodiments, the present invention provides a composition comprising a compound selected from ER-819556, ER-819557, ER-819558, and ER-819752. Yet another embodiment provides a composition comprising a compound selected from ER-819877, ER-819878, ER-819879, ER-819882, and ER-819763.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, cyclodextrins, sodium carboxymethylcellulose, polyacrylates, waxes, polyethylene-polyoxypropylene-block polymers, polyethylene glycol and wool fat.

Pharmaceutically acceptable salts of the compounds of this invention include those derived from pharmaceutically acceptable inorganic and organic acids and bases. Examples of suitable acid salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, formate, fumarate, glucoheptanoate, glycerophosphate, glycolate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, nitrate, oxalate, palmoate, pectinate, persulfate, 3-phenylpropionate, phosphate, picrate, pivalate, propionate, salicylate, succinate, sulfate, tartrate, thiocyanate, tosylate and undecanoate. Other acids, such as oxalic, while not in themselves pharmaceutically acceptable, may be employed in the preparation of salts useful as intermediates in obtaining the compounds of the invention and their pharmaceutically acceptable acid addition salts.

Salts derived from appropriate bases include alkali metal (e.g., sodium and potassium), alkaline earth metal (e.g., magnesium), ammonium and N+(C₁₋₄ alkyl)₄ salts. This invention also envisions the quaternization of any basic nitrogen-containing groups of the compounds disclosed herein. Water or oil-soluble or dispersible products may be obtained by such quaternization.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, the pharmaceutically acceptable compositions of this invention are formulated for oral administration.

The amount of the compounds of the present invention that may be combined with the carrier materials to produce a composition in a single dosage form will vary depending upon the host treated, and the particular mode of administration. Preferably, the compositions should be formulated so that a dosage of between 0.01-100 mg/kg body weight/day of the inhibitor can be administered to a patient receiving these compositions. In certain embodiments, the compositions of the present invention provide a dosage of between 0.01 mg and 50 mg is provided. In other embodiments, a dosage of between 0.1 and 25 mg or between 5 mg and 40 mg is provided.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Compounds of Formula I, Formula Ia, or Formula Ib are useful as T-bet inhibitors, both in vitro and in vivo. T-bet (T-box expressed in T cells) is a Th1 specific transcription factor that is a key regulator of the Th1/Th2 balance. See S. J. Szabo, et al., Cell, 100:655-669 (2000). T-bet is selectively induced in Th1 cells and can transactivate the interferon-gamma gene, induce interferon-gamma production, redirect polarized Th2 cells into the Th1 pathway. T-bet also controls IFN-gamma production in CD8+ T cells, as well as in cells of the innate immune system, e.g., NK cells and dendritic cells. Accordingly, direct or indirect inhibitors of the T-bet signalling pathway (including compounds that inhibit T-bet expression) are therapeutically useful in balancing over-active Th1 responses, and therefore be of value in treating Th1-mediated diseases, such as: rheumatoid arthritis and multiple sclerosis. In some embodiments, such as where R⁹ is hydrogen, compounds of Formula I or Formula Ia are also useful as intermediates for making other compounds of Formula I or Formula Ia wherein R⁹ is X—R⁵, In some embodiments, such as where R⁸ is H in compounds of Formula I or Formula Ia, those compounds are also useful as intermediates for making other compounds of Formula I, Formula Ia, where R⁸ is not H.

According to one embodiment, the invention relates to a method of inhibiting the formation of Th1 cells in a biological sample comprising the step of contacting said biological sample with a compound of this invention, or a composition comprising said compound.

According to another embodiment, the invention relates to a method of directly or indirectly inhibiting activity of the T-bet signalling pathway in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

According to one embodiment, the invention relates to a method of inhibiting the formation of Th1 cells in a patient comprising the step of administering to said patient a compound of this invention, or a composition comprising said compound.

Specifically, the present invention relates to a method of treating or lessening the severity of rheumatoid arthritis or multiple sclerosis, wherein said method comprises administering to a patient in need thereof a composition according to the present invention.

In certain embodiments, the present invention provides a method for treating rheumatoid arthritis or multiple sclerosis by administering a compound of formula I. In other embodiments, the present invention provides a method for treating a T-bet-mediated disease, as described herein, by administering any of compounds 1-70 set forth in Tables 1 and 2. According to another aspect, the present invention provides a method for treating rheumatoid arthritis or multiple sclerosis by administering a compound selected from ER-819724, ER-819755, ER-819750, ER-819749, ER-819735. According to yet another aspect, the present invention provides a method for treating rheumatoid arthritis or multiple sclerosis by administering a compound selected from ER-819543, ER-819549, ER-819543, ER-819701, ER-819544, ER-819594, ER-819647, ER-819657, ER-819659, and ER-819592. In other embodiments, the present invention provides a method for treating rheumatoid arthritis or multiple sclerosis by administering a compound selected from ER-819595, ER-819597, ER-819641, ER-819673, ER-819651, ER-819583, ER-819604, ER-819593, ER-819658, and ER-819648. In still other embodiments, the present invention provides a method for treating rheumatoid arthritis or multiple sclerosis by administering a compound selected from ER-819602, ER-819689, ER-819646, ER-819655, ER-819703, ER-819667, ER-819601, ER-819605, ER-819652, ER-819688, ER-819603, ER-819642, and ER-819628. Yet another embodiment provides a method for treating rheumatoid arthritis or multiple sclerosis by administering a compound selected from ER 819-891, ER-819772, ER-819771, ER-819770, ER-819769, ER-819768, and ER-819767. In certain embodiments, the present invention provides a method for treating rheumatoid arthritis or multiple sclerosis by administering a compound selected from ER-819556, ER-819557, ER-819558, and ER-819752. Yet another embodiment provides a method for treating rheumatoid arthritis or multiple sclerosis by administering a compound selected from ER-819877, ER-819878, ER-819879, ER-819882, and ER-819763.

In order that the invention described herein may be more fully understood, the following examples are set forth. It should be understood that these examples are for illustrative purposes only and are not to be construed as limiting this invention in any'manner. For example, in the claims below, where compounds are identified by a number “ER-xxxxxx” herein, the compound is intended to be inclusive of that compound as both a free base (or salt-free) and any pharmaceutically acceptable salts thereof (e.g., as identified in the definitions above), even if that compound is specified as “salt free” or as a particular salt in the Examples below. Additionally, where structures of compounds are depicted in connection with a number “ER-xxxxxx” herein, and that structure contains a methyl group depicted by a sinusoidal or “wavy” line, that the compound is intended to be inclusive of that compound as both a racemic mixture and enantiomerically pure compounds.

### EXAMPLES

### Chemical Compounds

Microwave assisted reactions were carried out using an Emrys Liberator instrument supplied by Biotage Corporation. Solvent removal was carried out using either a Biichi rotary evaporator or a Genevac centrifugal evaporator. Analytical and preparative chromatography was carried out using a Waters autopurification instrument using either normal phase or reverse phase HPLC columns, under either acidic, neutral, or basic conditions. Compounds were estimated to be >90% pure, as determined by area percent of ELSD chromatograms. NMR spectra were recorded using a Varian 300 MHz spectrometer.

General methods and experiments for preparing compounds of the present invention are set forth below. In certain cases, a particular compound is described by way of example. However, it will be appreciated that in each case a series of compounds of the present invention were prepared in accordance with the schemes and experiments described below.

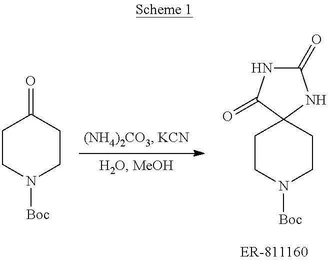

ER-811160. As depicted in Scheme 1 above, a solution of potassium cyanide (22.5 g, 0.335 mol) in water (50 mL) was added dropwise over 5 minutes to a solution of 1-Boc-piperidone (32.48 g, 0.1598 mol) and ammonium carbonate (33.8 g, 0.351 mol) in water (90 mL) and methanol (110 mL). An off-white precipitate began to form soon after addition was complete. The reaction flask was sealed and the suspension stirred at room temperature for 72 hours. The resultant pale yellow precipitate was filtered and was washed with small portions of water to give ER-811160 (37.1 g, 86%) as a colorless solid.

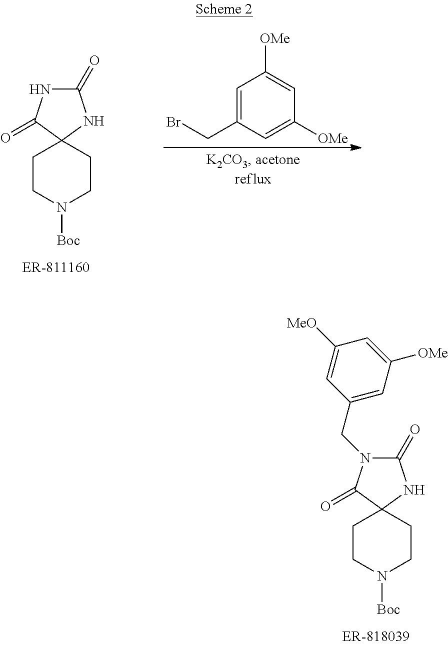

ER-818039. As depicted in Scheme 2 above, a suspension of ER-811160 (30.0 g, 0.111 mol), 3,5-Dimethoxybenzyl bromide (30.9 g, 0.134 mol), and potassium carbonate (18.5 g, 0.134 mol) in acetone (555 mL) was heated under reflux overnight. The reaction solution was cooled to room temperature, filtered and concentrated in vacuo. The crude orange product was dissolved in a minimal amount of MTBE (250 mL). A small amount of hexanes was added (50 mL) and the product was allowed to precipitate out (2 hours) as a colorless solid which was isolated by vacuum filtration. The filter cake was washed with small amounts of MTBE, and dried in vacuo to provide ER-818039 (39.6 g, 85%).

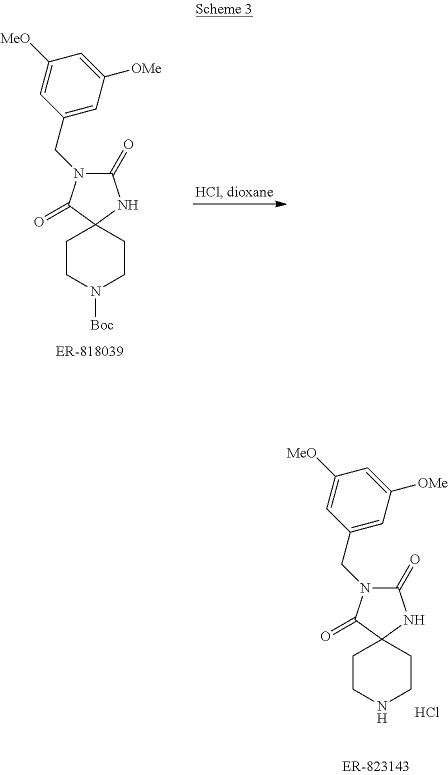

ER-823143. As depicted in Scheme 3 above, to a 1-neck round-bottom flask containing ER-818039 (2.15 g, 0.00512 mol) was slowly added a solution of 4N HCl in 1,4-Dioxane (3.8 mL, 0.049 mol). The starting material slowly dissolved over 20 minutes and a colorless precipitate formed after 30 minutes. MTBE (3 ml) was then added. After 2 hours, the reaction was filtered and washed with MTBE, which provided ER-823143 (1.81 g, 99%) as a colorless solid.

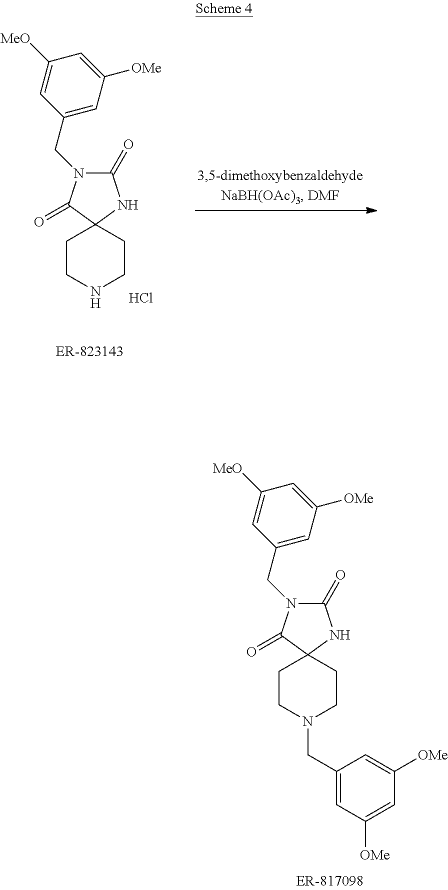

ER-817098: As depicted in Scheme 4 above, to a suspension of ER-823143 (41.5 mg, 0.000117 mol) and 4 Å molecular sieves in 1,2-dimethoxyethane (0.5 mL, 0.004 mol) under an atmosphere of nitrogen was added 3,5-dimethoxybenzaldehyde (21.3 mg, 0.000128 mol) followed by triethylamine (16.2 μL, 0.000117 mol). The reaction was stirred for 1 hour. Sodium triacetoxyborohydride (34.6 mg, 0.000163 mol) was added, and the reaction was stirred overnight. Flash chromatography using ethyl acetate as eluent yielded ER-817098 (45.3 mg, 83%) as a colorless solid.

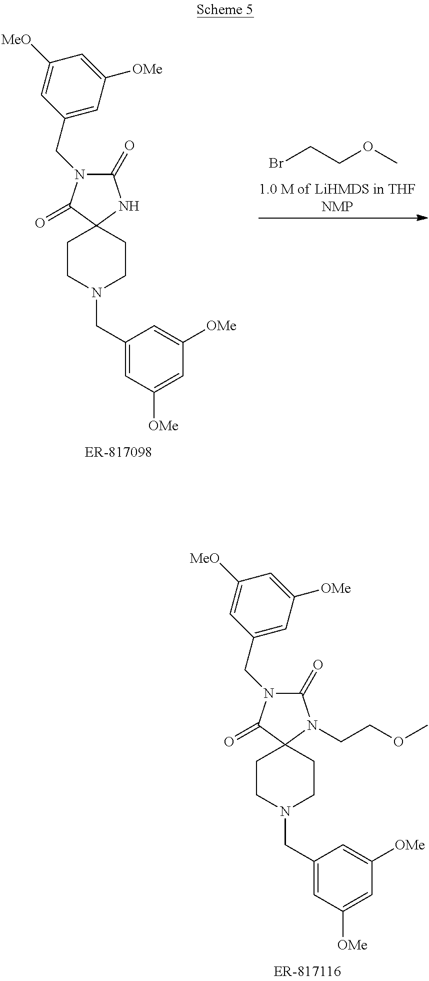

ER-817116: As depicted in Scheme 5 above, to a solution of ER-817098-00 (50.0 mg, 0.000106 mol) and 1-bromo-2-methoxyethane (15.6 μL, 0.000160 mol) in N-methylpyrrolidinone (1.0 mL, 0.010 mol) was added 1.0 M lithium hexamethyldisilazide solution in tetrahydrofuran (0.16 mL). The temperature was increased to at 80° C. and the reaction mixture stirred overnight. The reaction mixture was cooled to room temperature, quenched with water and then extracted several times with MTBE. The MTBE extracts were combined and washed with water (2×) and brine (1×). The organic layer was dried over magnesium sulfate, filtered, and concentrated in vacuo. Flash chromatography using ethyl acetate as eluent provided ER-817116 (32.2 mg, 58%) as colorless oil.

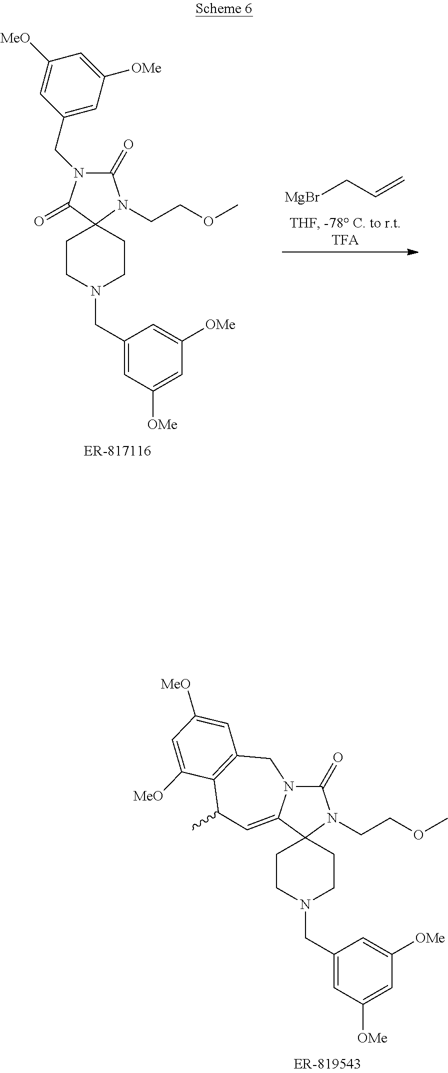

ER-819543: As depicted in Scheme 6 above, to a solution of ER-817116-00 (91.6 mg, 0.000174 mol) in tetrahydrofuran (1.8 mL, 0.022 mol) at −78° C. was slowly added a solution of 1.0 M allylmagnesium bromide in ether (0.35 mL). The reaction mixture was warmed to room temperature and stirred overnight. Mass spectroscopic analysis showed 25% conversion to product; consequently, the reaction mixture was re-cooled to −78° C. and an additional 1.35 mL of 1.0 M of allylmagnesium bromide in ether was added. The reaction mixture was warmed to room temperature and stirred for 4 hours. The reaction mixture was then cooled to 0° C. and was treated dropwise with trifluoroacetic acid (2.00 mL, 0.0260 mol) and then concentrated in vacuo. Triethylamine was then added to neutralize residual TFA. Ethyl acetate was added and the crude reaction product purified by flash chromatography (eluent: 100% Ethyl acetate) to provide ER-819543 (56.8 mg, 59%) as a colorless solid.

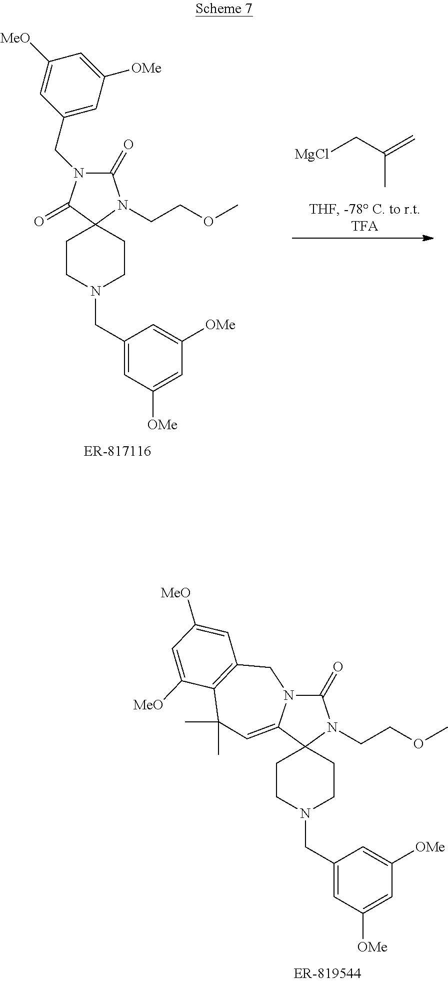

ER-819544: As depicted in Scheme 7 above, to a solution of ER-817116-00 (100.5 mg, 0.0001905 mol) in tetrahydrofuran (1.9 mL, 0.023 mol) at −78° C. was slowly added a 0.5 M solution of 2-methylallylmagnesium chloride in tetrahydrofuran (800 μL). The reaction mixture was warmed to room temperature and stirred for 6 hours. The reaction mixture was cooled to 0° C., treated dropwise with trifluoroacetic acid (1.00 mL, 0.0130 mol), and then concentrated in vacuo. Triethylamine was added to neutralize residual TFA. Ethyl acetate was added and the crude reaction product purified by flash chromatography using ethyl acetate as eluent to provide ER-819544 (66.2 mg, 61%) as a colorless solid.

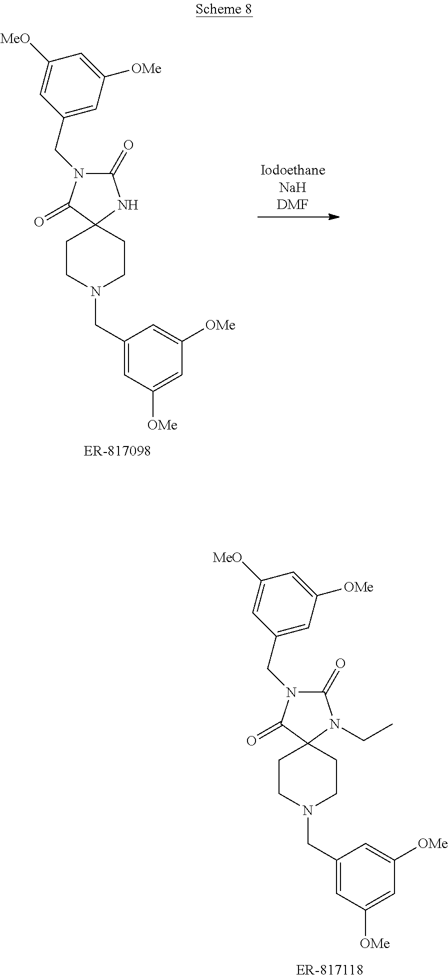

ER-817118: As depicted in Scheme 8 above, to a solution of ER-817098 (2.85 g, 0.00607 mol) in N,N-dimethylformamide (15 mL) was added sodium hydride (364 mg, 0.00910 mol) followed by iodoethane (758 μL, 0.00910 mol). The reaction mixture was stirred overnight. Water was very slowly added and the reaction mixture was extracted several times with MTBE. The MTBE extracts were combined and washed with water (2×) and brine (1×). The organic layer was dried over magnesium sulfate, filtered, and concentrated in vacuo. Flash chromatography using ethyl acetate as eluent provided ER-817098 (2.89 g, 96%) as a colorless oil.

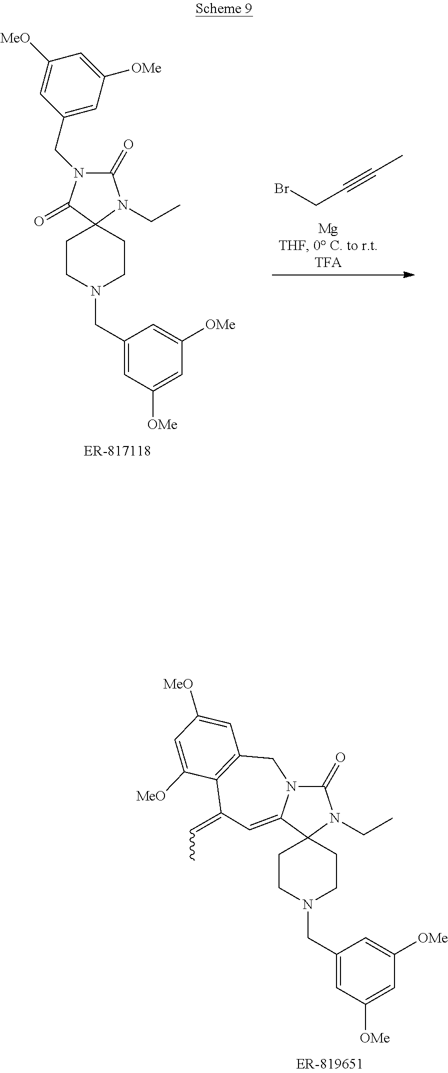

ER-819651: As depicted in Scheme 9 above, to a stirred suspension of 1 M of magnesium in tetrahydrofuran (5.58 mL) was slowly added 1-bromo-2-butyne (414 μL, 0.00459 mol) at 0° C. After stirring for 2 hours (the reaction solution remains black), a solution of ER-817118 (228.4 mg, 0.0004590 mol) in dry THF (10 mL) was slowly added at 0° C. The reaction was warmed to room temperature and was stirred for 4 hours. The reaction mixture was then cooled to −78° C. and treated dropwise with trifluoroacetic acid (0.95 mL, 0.012 mol) to cause the solution to become clear. The reaction mixture was warmed to room temperature and stirred for 1 hour. The reaction mixture was concentrated in vacuo to dryness using a rotary evaporator with a water bath temperature of 40° C. The residual light brown solid was basified with triethylamine (clear solid) and purified by flash chromatography (eluent: 2% EtOH in methylene chloride) to provide impure ER-819651. Subsequent repurification by HPTLC (8% EtOH in Toluene) provided ER-819651 (128.8 mg, 53%) as a colorless solid.

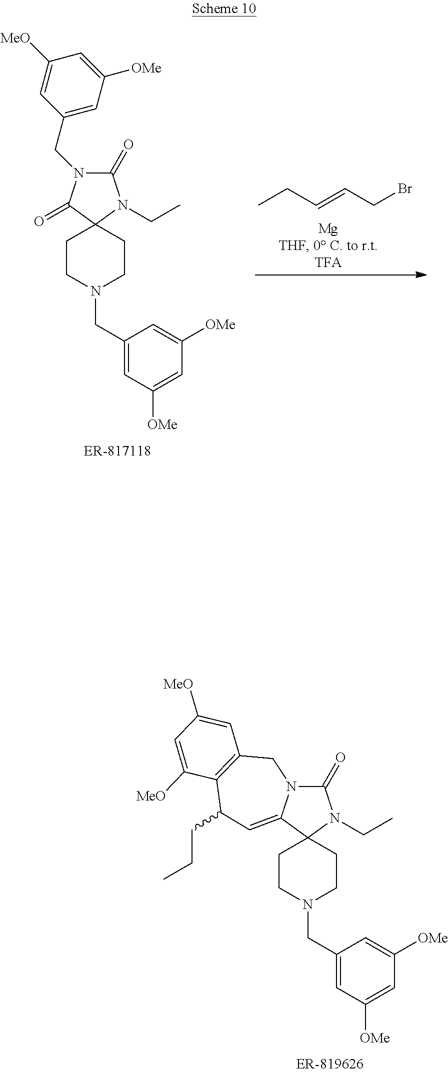

ER-819626: As depicted in Scheme 10 above, to a stirred suspension of 1 M of magnesium in tetrahydrofuran (4.990 mL) was slowly added 1-bromo-2-pentene (485.6 uL, 0.004106 mol) at 0° C. After stirring for 2 hours (the reaction solution remains black), a solution of ER-817118 (204.3 mg, 0.0004106 mol) in dry THF (10 mL) was slowly added at 0° C. The reaction mixture was warmed to room temperature and stirred for 4 hours (reaction solution remains black). The reaction was cooled to −78° C. and treated dropwise with trifluoroacetic acid (0.85 mL, 0.011 mol) to cause the reaction mixture to become clear. The reaction mixture was warmed to room temperature and stirred for 1 hour. The reaction mixture was concentrated in vacuo to dryness using a rotary evaporator with a water bath temperature of 40° C. The crude product (light brown solid) was basified with triethylamine (clear solid) and purified by flash chromatography (eluent: 2% EtOH in methylene chloride) to provide ER-819626 (110.2 mg, 49%) as a white solid.

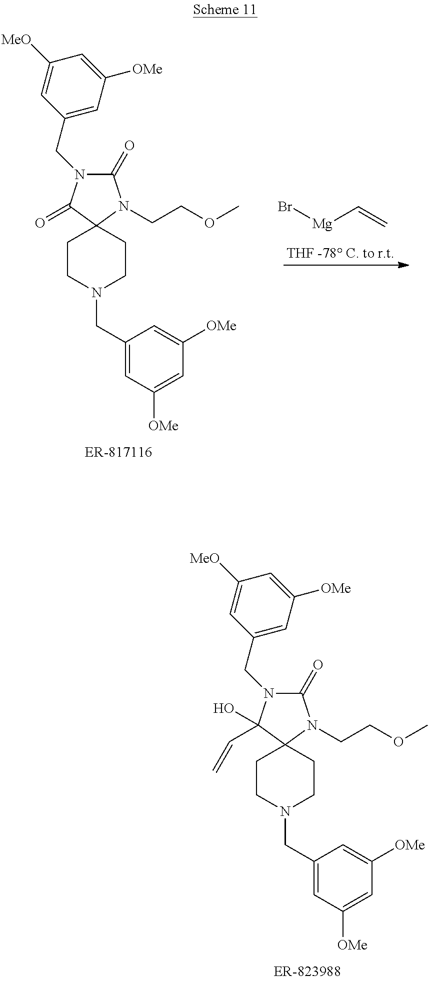

ER-823988: As depicted in Scheme 11 above, to a solution of ER-817116 (1.006 g, 0.0019067 mol) in tetrahydrofuran (7.6 mL, 0.094 mol) was slowly added a 1.0 M solution of vinylmagnesium bromide in tetrahydrofuran (3.8 mL) at −78° C. The reaction mixture was warmed to room temperature and stirred for 1 hour. Mass spectroscopic analysis showed a significant amount of residual starting material; consequently, the reaction mixture was re-cooled to 0° C. and an additional 3.8 mL of 1.0 M vinylmagnesium bromide solution in tetrahydrofuran was added. The reaction mixture was stirred for 2 hours then quenched by dropwise addition of saturated aqueous ammonium hydroxide solution. The mixture was extracted several times with ethyl acetate. The organic extracts were combined and washed with water (2×) and brine. The organic layer was dried over magnesium sulfate, filtered, and concentrated in vacuo. Flash chromatography (eluent: 5% ethanol in ethyl acetate) provided ER-823988 (0.605 g, 57%) as a colorless solid.

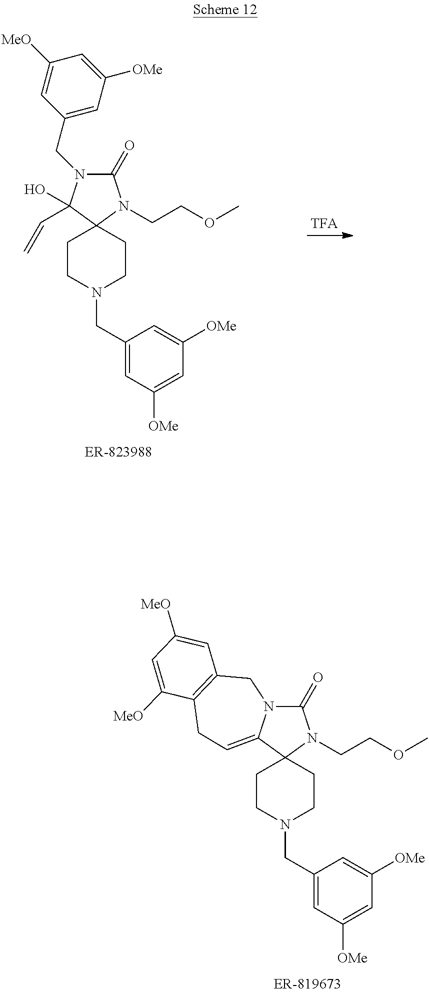

ER-819673: As depicted in Scheme 12 above, ER-823988 (163.1 mg, 0.0002935 mol) was dissolved in trifluoroacetic acid (2.00 mL, 0.0260 mol) at room temperature. The reaction mixture was warmed to 40° C. and stirred for 2 hours then concentrated in vacuo. The residue was dissolved in a small amount of acetone and was treated with a small portion of potassium carbonate until basic. Flash chromatography (eluent: 2% ethanol in ethyl acetate) provided ER-819673 (0.101 g, 64%) as a colorless glassy solid.

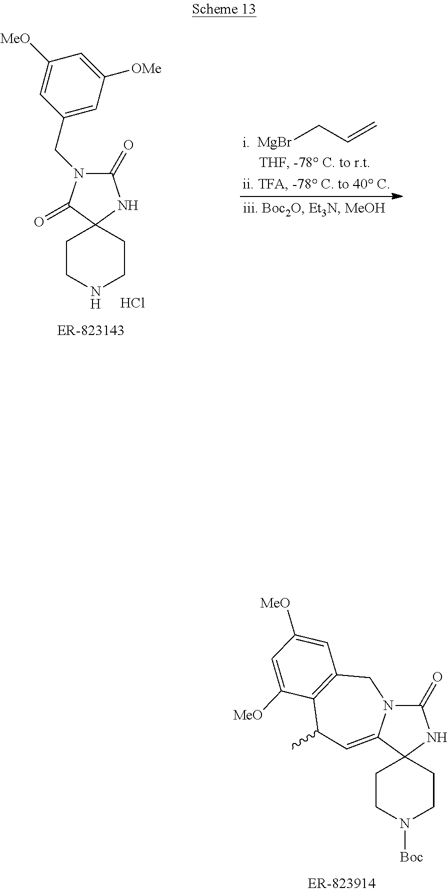

ER-823914: As depicted in Scheme 13 above, to a solution of ER-823143 (5.03 g, 0.0141 mol) in tetrahydrofuran (30.0 mL, 0.370 mol) at −78° C. was slowly added a 1.0 M solution of allylmagnesium bromide in ether (71 mL). The reaction mixture was warmed to room temperature and stirred overnight. The reaction mixture was cooled to −78° C., treated dropwise with trifluoroacetic acid (21.8 mL, 0.283 mol), and then concentrated in vacuo to a small residual volume. Triethylamine was added to neutralize residual TFA and the mixture then concentrated in vacuo to dryness. The residual red oil was dissolved in methanol (138 mL, 3.41 mol) and treated with di-tert-butyldicarbonate (3.34 g, 0.0148 mol) followed by triethylamine (2.38 mL, 0.0169 mol) and stirred overnight at room temperature. The reaction mixture was concentrated in vacuo and purified by flash chromatography (eluent: 50% hexanes in ethyl acetate) to provide ER-823914 (3.25 g, 52%) as a colorless solid.

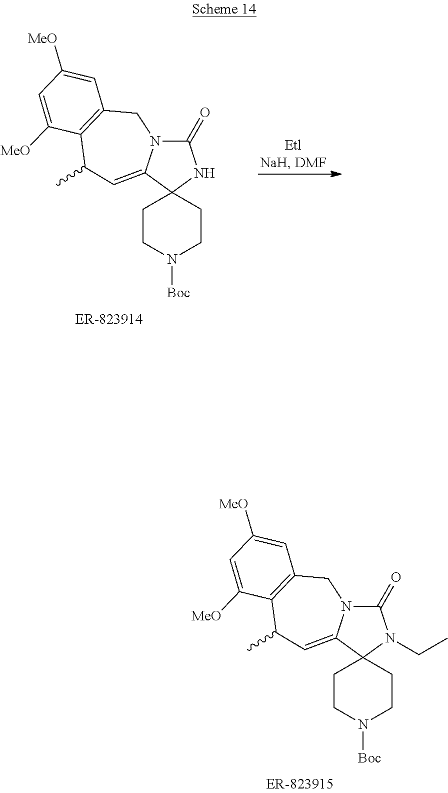

ER-823915: To a solution of ER-823914 (2.20 g, 0.00496 mol) in N,N-Dimethylformamide (12.4 mL, 0.160 mol) was added sodium hydride (298 mg, 0.00744 mol) followed by iodoethane (607 μL, 0.00744 mol). The reaction mixture was stirred overnight then quenched with water and extracted several times with MTBE. The MTBE extracts were combined and washed with water and brine. The organic layer was dried over magnesium sulfate, filtered, and concentrated in vacuo. Flash chromatography (eluent: 40% hexanes in ethyl acetate) provided ER-823915 (0.80 g, 34%) as a colorless foam.

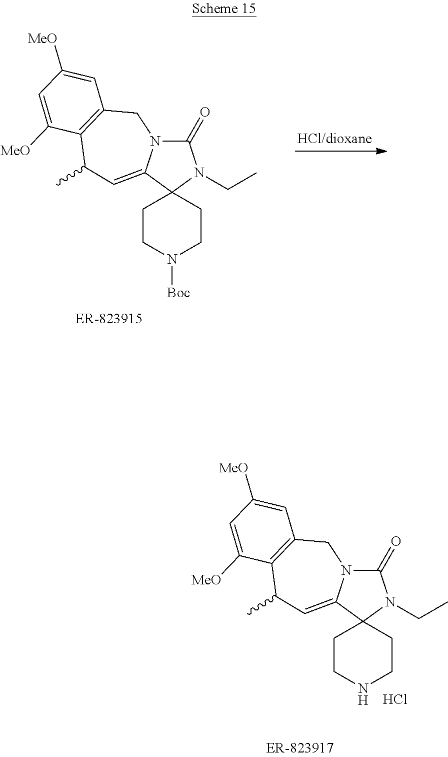

ER-823917: As depicted in Scheme 15 above, ER-823915 (799.2 mg, 0.001695 mol) was dissolved in a solution of 4 M hydrogen chloride in 1,4-dioxane (10 mL). The reaction mixture was stirred overnight and then concentrated in vacuo to provide ER-823917 (0.69 g, quantitative) as an orange solid.

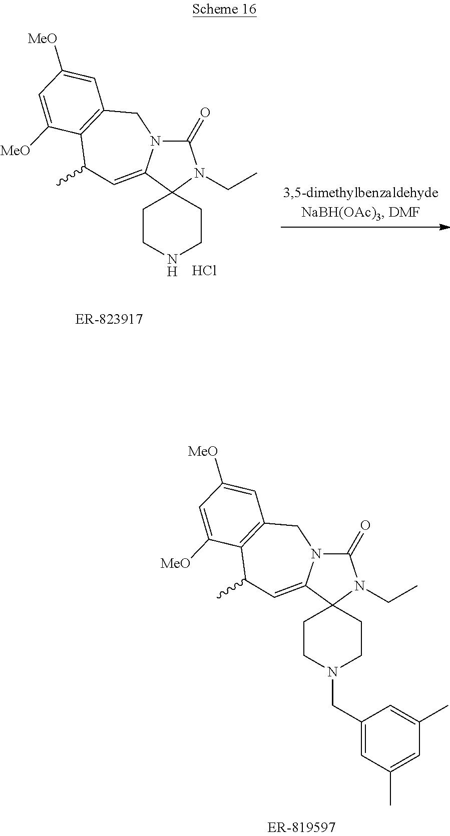

ER-819597: As depicted in Scheme 16 above, ER-823917 (100.0 mg, 0.0002451 mol), 4 Å molecular sieves, and 3,5-dimethylbenzaldehyde (50.9 mg, 0.000368 mol) were dissolved/suspended in N,N-dimethylformamide (1.0 mL, 0.013 mol). After stirring for 30 minutes, sodium triacetoxyborohydride (76.6 mg, 0.000343 mol) was added. The reaction mixture was stirred overnight. Water was added until a white precipitate formed. The precipitate was collected by filtration washing several times with water. The filtrate was then dried in vacuo to provide ER-819597 (108.0 mg, 90%) as a colorless solid.

ER-819689, ER-819688, ER-819604, ER-819595, ER-819594, ER-819593, ER-819592, ER-819582, and ER-819777 were prepared in substantially the same manner as for ER-819597. In some instances the desired product could be precipitated from the reaction mixture; in other cases the reaction mixture would be quenched with water then extracted with a suitable water-immiscible solvent, followed by chromatographic purification.

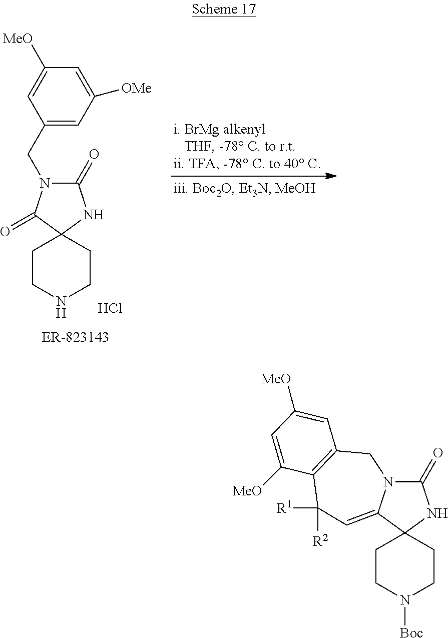

Scheme 17 above depicts a general cyclization method. As depicted in Scheme 17 above, to a solution of ER-823143 (0.0141 mol) in tetrahydrofuran (30.0 mL) at −78° C. was slowly added a 1.0 M solution of an alkenyl magnesium bromide in ether (71 mL). The reaction mixture was warmed to room temperature and stirred overnight. The reaction mixture was cooled to −78° C. and treated dropwise with trifluoroacetic acid (0.283 mol). The reaction solution was concentrated in vacuo to a small volume then treated with triethylamine to neutralize the residual TFA. The crude product was concentrated in vacuo to dryness. The resultant residue was then dissolved in methanol (138 mL) and treated with di-tert-butyldicarbonate (0.0148 mol) followed by triethylamine (0.0169 mol). The reaction mixture was stirred overnight then concentrated in vacuo. Purification by flash chromatography provided the desired product.

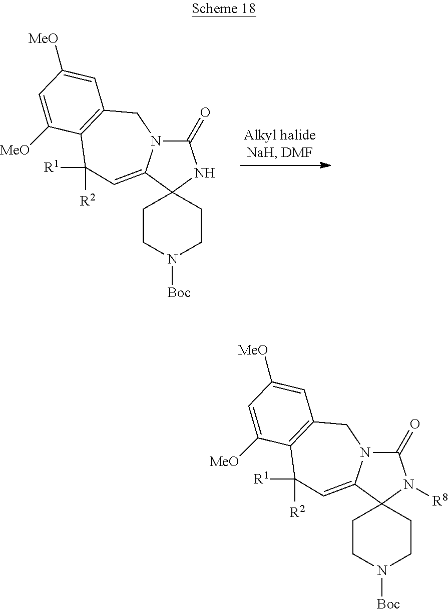

Scheme 18 above depicts a general method for introducing the R⁸ group. As depicted in Scheme 18 above, to a solution of starting material (0.00496 mol) in N,N-dimethylformamide (12.4 mL) was added sodium hydride (0.00744 mol) followed by an alkyl halide (0.00744 mol). The reaction mixture was stirred overnight then quenched with water and extracted several times with MTBE. The MTBE extracts were combined and washed with water and brine. The organic layer was dried over magnesium sulfate, filtered, and concentrated in vacuo. Flash chromatography provided the desired product.

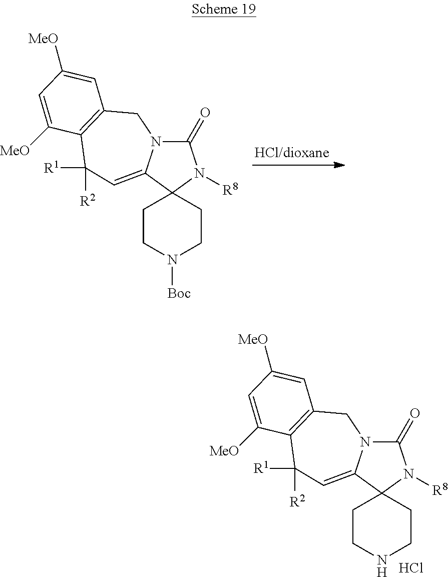

As depicted in Scheme 19 above, starting material (0.001695 mol) was dissolved in 4 M of hydrogen chloride in 1,4-dioxane (10 mL). The reaction mixture was stirred overnight and then concentrated in vacuo to provide the desired product.

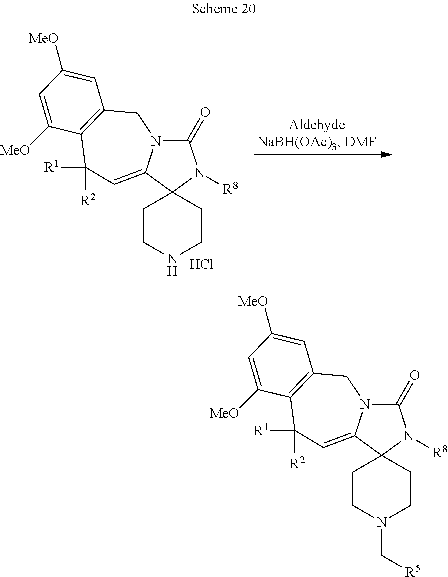

Scheme 20 above depicts a general method for introducing the —X—R⁵ group, where X is —CH₂—. As depicted in Scheme 20 above, starting material (0.0002451 mol), 4 Å molecular sieves, and aldehyde (0.000368 mol) were dissolved/suspended in N,N-dimethylformamide (1.0 mL). After stirring for 30 minutes, sodium triacetoxyborohydride (0.000343 mol) was added. The reaction mixture was stirred overnight then quenched with water. In some cases the desired product would precipitate upon quenching the reaction with water, in which case it could be isolated by filtration and subsequently purified by flash chromatography. In other cases the desired product could be extracted using a suitable water-immiscible organic solvent and then subsequently purified by either flash chromatography or reverse phase preparative HPLC.

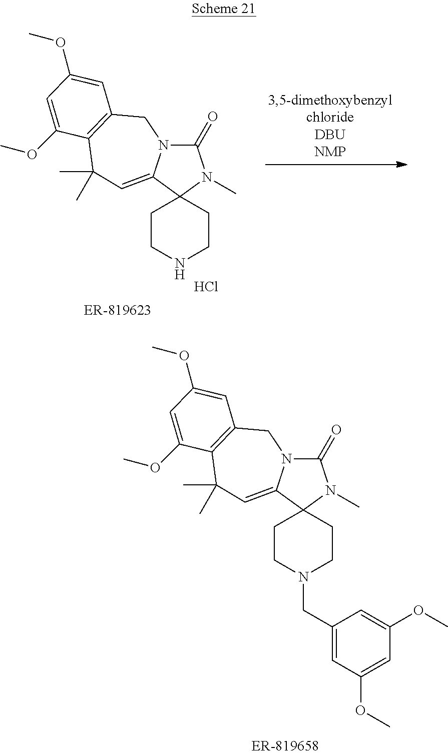

ER-819658: As depicted in Scheme 21 above, a 2 mL microwave reactor vial was charged with ER-819623 (71.6 mg, 0.000176 mol), 3,5-dimethoxybenzyl chloride (41.1 mg, 0.000220 mol), N-methylpyrrolidinone (700.0 μL) and 1,8-diazabicyclo[5.4.0]undec-7-ene (60.0 μL, 0.000401 mol). The reaction mixture was sealed and was heated at 180° C. for 60 seconds in the microwave. Purification by reverse phase HPLC provided ER-819658 (54.9 mg, 60%).

ER-819637 and ER-819627 were prepared in substantially the same manner as ER-819658.

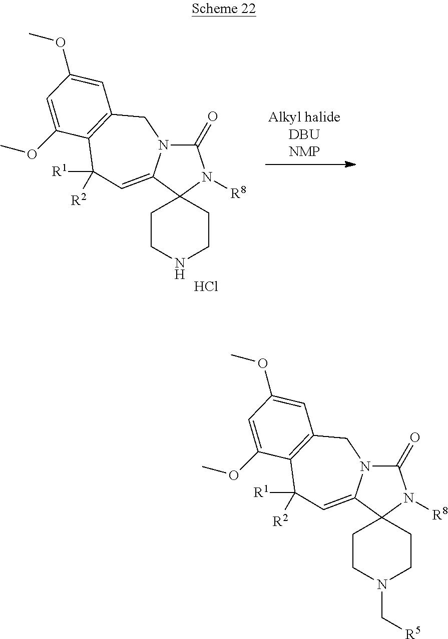

Scheme 22 above depicts another general method for introducing the —X—R⁵ group, where X is —CH₂—. As depicted in Scheme 22 above, a 2 mL microwave reactor vial was charged with starting material (0.000176 mol), an alkyl halide (0.000220 mol), N-methylpyrrolidinone (700.0 μL) and 1,8-diazabicyclo[5.4.0]undec-7-ene (0.000401 mol). The reactor vial was sealed and heated at 180° C. for 60 seconds in the microwave. Purification by reverse phase HPLC provided the desired product.

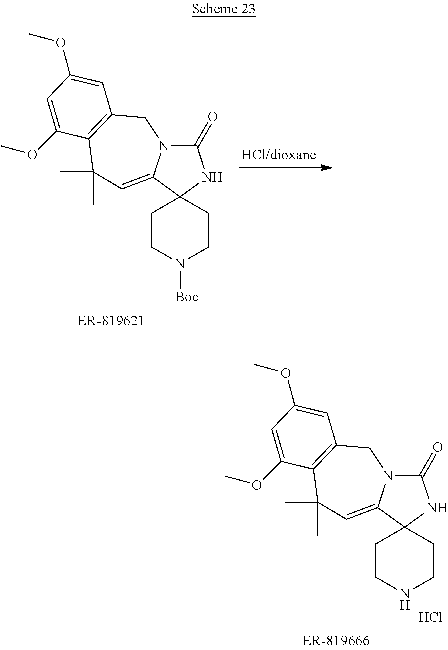

ER-819666: As depicted in Scheme 23 above, to a flask containing ER-819621 (2.30 g, 0.00503 mol) was added a 4 M solution of hydrogen chloride in 1,4-dioxane (15.0 mL). The reaction mixture was stirred at room temperature for 30 minutes then concentrated in vacuo to provide ER-819666 (1.98 g, quantitative).

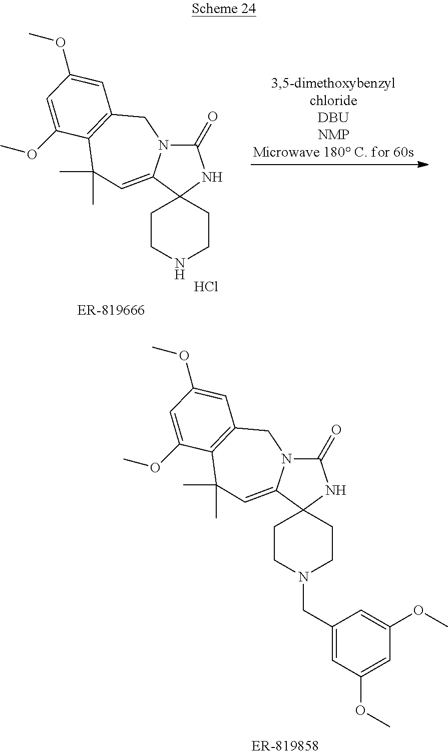

ER-819585: As depicted in Scheme 24 above, a 2 mL microwave reactor vial containing a stir bar was charged with ER-819666 (653.4 mg, 0.001659 mol), 3,5-dimethoxybenzyl chloride (377.6 mg, 0.002023 mol), N-methylpyrrolidinone (5.00 mL, 0.0518 mol) and 1,8-diazabicyclo[5.4.0]undec-7-ene (560.0 μL, 0.003745 mol). The reactor vial was sealed and heated at 180° C. for 60 seconds in the microwave. Purification by reverse phase HPLC provided ER-819585 (52.1 mg, 68%).

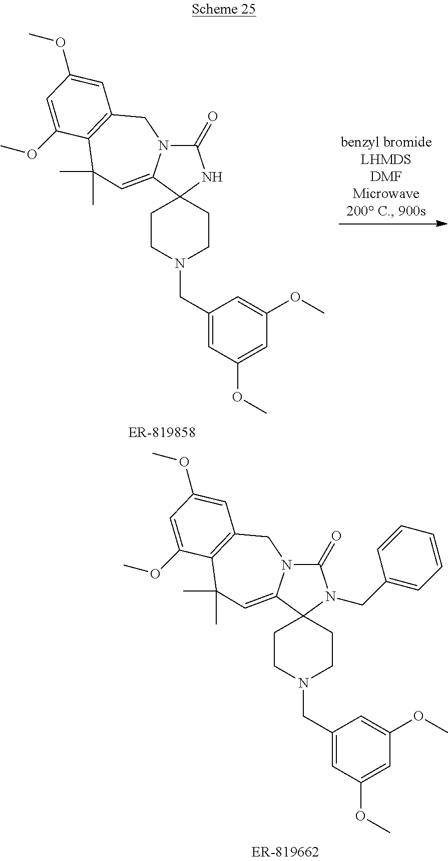

ER-819621: As depicted in Scheme 25 above, a 2 mL microwave reactor vial equipped with a stir bar was charged with ER-819585 (70.0 mg, 0.000138 mol), N,N-dimethylformamide (830.0 μL, 0.01072 mol), benzyl bromide (40.0 μL, 0.000336 mol) and a 1.00 M solution of lithium hexamethyldisilazide in tetrahydrofuran (350.0 μL). The reactor vial was sealed and heated at 200° C. for 900 sec in the microwave. Purification by preparative reverse phase HPLC provided ER-819662 (35.14 mg, 43%).

ER-819663, ER-819661, ER-819659, ER-819650, ER-819647, ER-819641 were prepared in substantially the same manner as ER-819662.

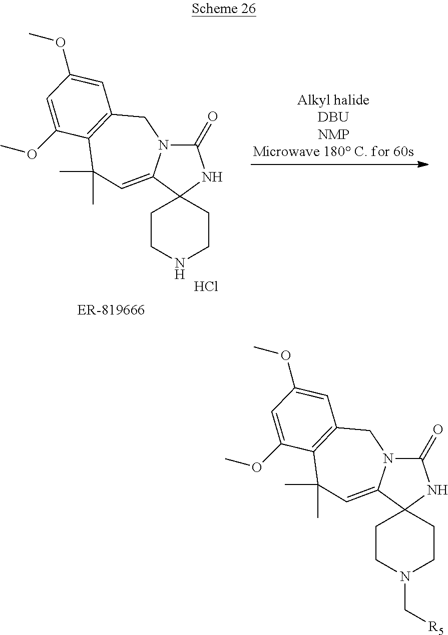

Scheme 26 above depicts a general method for introducing the —X—R⁵ group, where X is —CH₂—. As depicted in Scheme 26 above, a 2 mL microwave reactor vial containing a stir bar was charged with ER-819666 (0.001659 mol), an alkyl halide (0.002023 mol), N-methylpyrrolidinone (5.00 mL) and 1,8-diazabicyclo[5.4.0]undec-7-ene (0.003745 mol). The reactor vial was sealed and heated at 180° C. for 60 seconds in the microwave. Purification by preparative reverse phase HPLC provided the desired product.

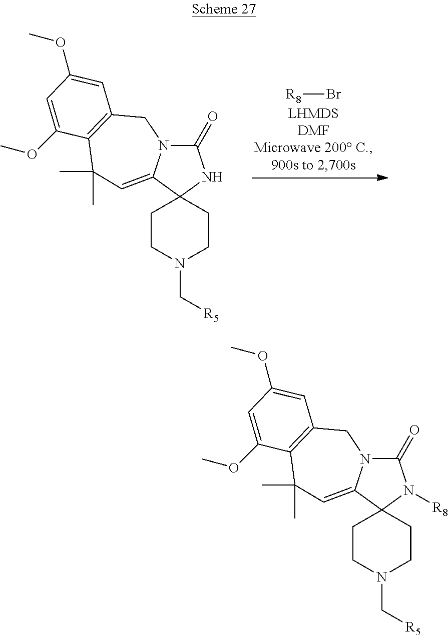

Scheme 27 above depicts a general method for introducing the R⁸ group. As depicted in Scheme 27 above, a 2 mL microwave reactor vial equipped with a stir bar was charged with starting material (0.000138 mol), N,N-dimethylformamide (830 μL), R⁸-bromide (0.000336 mol) and a 1.00 M solution of lithium hexamethyldisilazide in tetrahydrofuran (350 μL). The reactor vial was sealed and heated at 200° C. for up to 2700 sec in the microwave. Purification by preparative reverse phase HPLC provided the desired product.

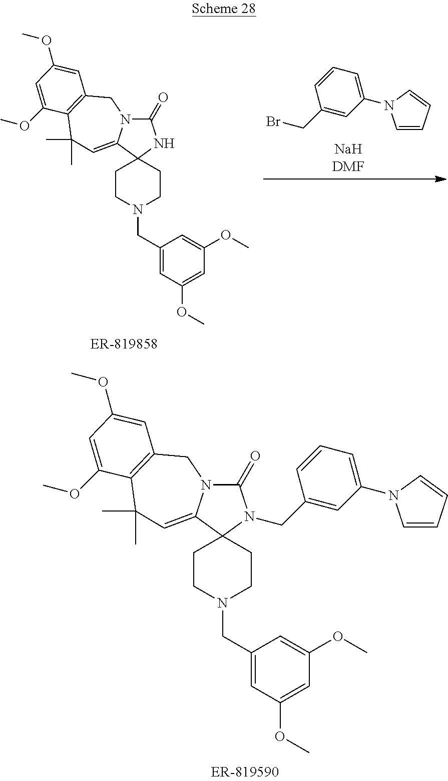

ER-819590: As depicted in Scheme 28 above, to a solution of ER-819585 (31.6 mg, 0.0000622 mol) and 1-[3-(bromomethyl)phenyl]-1H-pyrrole (18.2 mg, 0.0000747 mol) in N,N-dimethylformamide (500 μL, 0.007 mol) was added sodium hydride (2.99 mg, 0.0000747 mol). The reaction mixture was stirred overnight then quenched cautiously with water (1 mL), and extracted several times with ethyl acetate. The organic extracts were combined, washed with water and brine, dried over magnesium sulfate, filtered, and concentrated in vacuo. Flash chromatography (eluent: 50% ethyl acetate in hexanes) provided ER-819590 (18.8 mg, 46%) as a colorless solid.

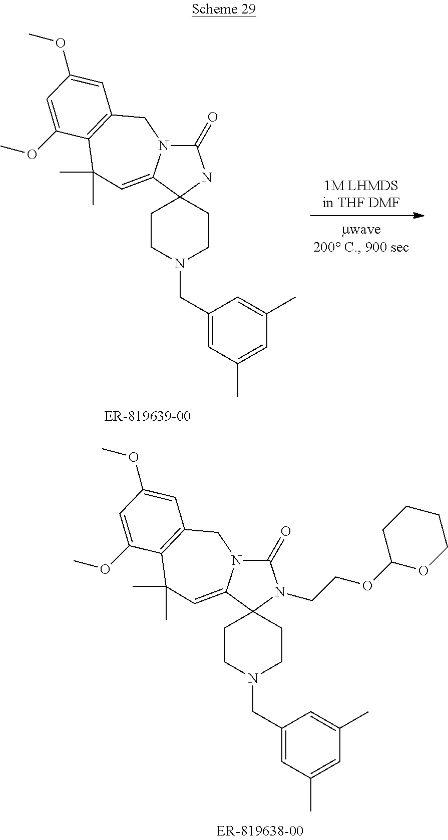

ER-819638: As depicted in Scheme 29 above, a 2 mL microwave reactor vial was charged with ER-819639 (102.3 mg, 0.0002151 mol), 2-(2-bromoethoxy)tetrahydro-2H-pyran (80.0 pt, 0.000530 mol), N,N-dimethylformamide (1000.0 μL) and a 1.00 M solution of lithium hexamethyldisilazide in tetrahydrofuran (530.0 μL). The reactor vial was sealed and heated at 200° C. for 900 sec in the microwave. The reaction was not complete; consequently, additional 2-(2-bromoethoxy)tetrahydro-2H-pyran (80 μL, 2.5 eq) and 1.00 M lithium hexamethyldisilazide solution in tetrahydrofuran (530 μL, 2.4 eq) were added and the vial reheated at 200° C. for 900 sec. Purification by preparative reverse phase HPLC provided ER-819638 (57.8 mg, 44.5%).

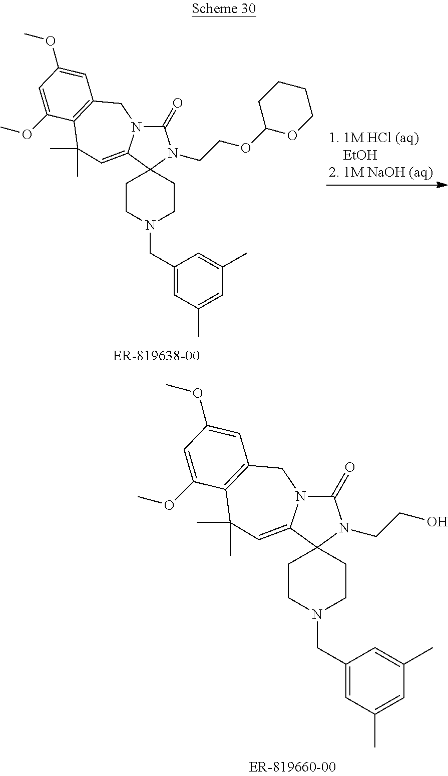

ER-819660: As depicted in Scheme 30 above, a solution of ER-819638 (57.8 mg, 0.0000957 mol) in ethanol (0.539 mL, 0.00922 mol) was treated with 1M hydrochloric acid (0.970 mL) and stirred at room temperature for 3 hours. The reaction mixture was neutralized by dropwise addition of 1 M aqueous sodium hydroxide (0.970 mL). Purification by preparative reverse phase HPLC provided ER-819660 (29.06 mg, 58.4%).

ER-819657 and ER-819642 were prepared in substantially the same manner as ER-819660.

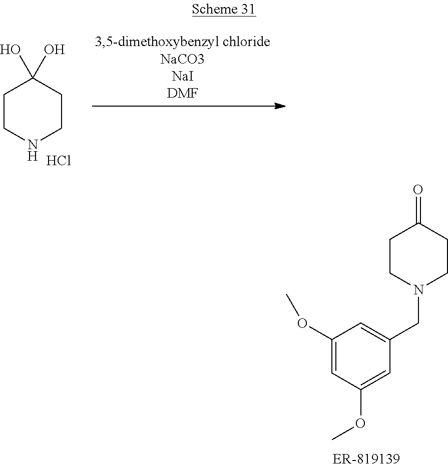

ER-819139: As depicted in Scheme 31 above, a 2 L round bottom flask was charged with 4-piperidone monochloride monohydrate (46.5 g, 0.302 mol) and N,N-dimethylformamide (600 mL). To the resulting suspension were added sodium carbonate (58.3 g, 0.550 mol), sodium iodide (28.9 g, 0.193 mol) and 3,5-dimethoxybenzyl chloride (51.4 g, 0.275 mol) under nitrogen. The resulting beige suspension was then heated to 90° C. and left to stir overnight under nitrogen. The reaction mixture became cloudy and golden yellow. The reaction mixture was filtered and then the resultant orange filtrate concentrated to a minimum amount of solvent by high vacuum rotavap. Saturated aqueous ammonium chloride solution (300 mL) was added and the mixture extracted with MTBE (250 mL extractions). The combined organic phases were dried (anhydrous Na₂SO₄) and concentrated to give a reddish brown oil ER-823139 (quantitative yield assumed).

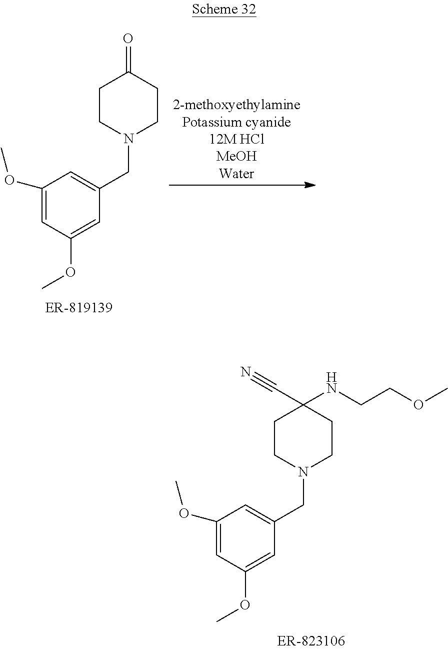

ER-823106: As depicted in Scheme 32 above, to a suspension of ER-823139 in water (2.8 mL) and methanol (3.0 mL) was added 2-methoxyethylamine (1.36 mL, 0.0157 mol). To the resultant brown suspension was added dropwise a 12M solution of aqueous hydrochloric acid (1.31 mL). The reaction mixture was heated to 40° C. and a solution of potassium cyanide (1.02 g, 0.0157 mol) in water (2.3 mL, 0.13 mol) was added dropwise. A significant amount of starting material was still not dissolved. Thus, additional methanol (3.0 mL, 0.074 mol) and water (2.8 mL, 0.16 mol) were added and the suspension was stirred at room temperature for 18 hr. The reaction mixture was then extracted with ethyl acetate (2×). The combined organics were washed with water, brine, dried over sodium sulfate, filtered and concentrated in vacuo to give yellow-brown crude product ER-823106 (4.70 g, 99%).

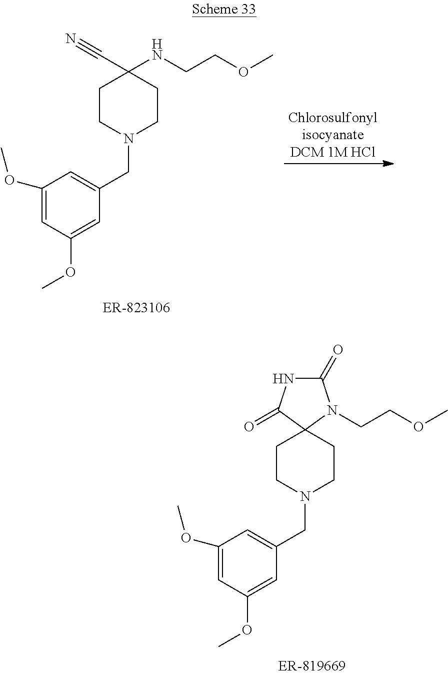

ER-819669: As depicted in Scheme 33 above, to a solution of ER-823106 (0.48 g, 0.0014 mol) in methylene chloride (2.0 mL) at room temperature was added chlorosulfonyl isocyanate (0.125 mL, 0.001440 mol) dropwise slowly. The internal temperature increased to 30° C. so an ice bath was then employed to keep the temperature between 16° C. and 25° C. The mixture was stirred at room temperature for 1 hr then concentrated in vacuo to give pale yellow foam. To the residue was added 1M hydrochloric acid (4.0 mL). The resulting suspension was stirred for 10 min at room temperature, than heated at 110° C. for 1 hr. The reaction mixture was then cooled to 0° C., neutralized with 5 M aqueous sodium hydroxide (˜1.2 mL). A light yellow milky precipitate formed, which was extracted with ethyl acetate (5×—until little/no product in last extract by TLC). The combined organics were washed with brine, dried over sodium sulfate, filtered and concentrated to give a dark yellow oil. The oil was purified by flash chromatography using DCM/Ethyl acetate (1:1), DCM/Ethyl acetate/MeOH (9:9:1) and Ethyl acetate/MeOH (9:1) to give ER-819669 (17 mg, 31%).

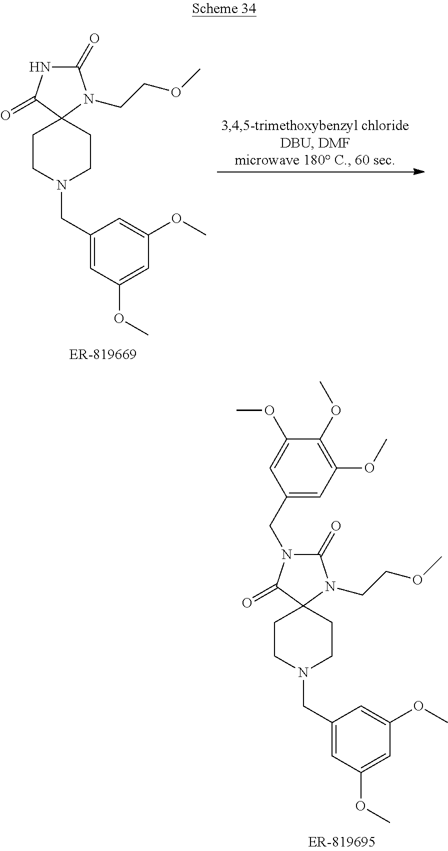

ER-819695: As depicted in Scheme 34 above, a solution of ER-819669 (110 mg, 0.00029 mol), 1,8-diazabicyclo[5.4.0]undec-7-ene (87.2 μL, 0.000583 mol) and 3,4,5-trimethoxybenzyl chloride (107 mg, 0.000495 mol) in N,N-dimethylformamide (1.1 mL) was heated at 180° C. for 60 seconds in the microwave. Purification by preparative reverse phase HPLC provided ER-819695 (129 mg, 79%) as colorless oil.

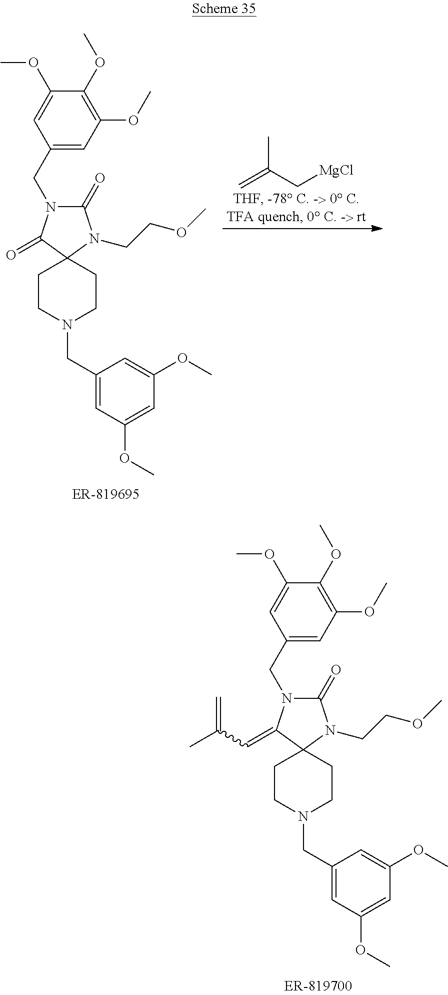

ER-819700: As depicted in Scheme 35 above, to a solution of ER-819695 (118 mg, 0.000212 mol) in tetrahydrofuran (4 mL, 0.05 mol) at −78° C. was added a 0.5 M solution of 2-methylallylmagnesium chloride in tetrahydrofuran (4.232 mL) dropwise over 3 min keeping internal temperature below at −50° C. The cooling bath was removed, and the reaction mixture allowed to warm to 0° C. After 2 h at 0° C., TLC (9:1 Ethyl acetate-MeOH, ninhydrin stain, UV) showed complete reaction. The reaction mixture was quenched by slow careful addition of trifluoroacetic acid (0.978 mL, 0.0127 mol) at 0° C. to give yellow solution. The reaction mixture was then warmed to room temperature, stirred for 10 min and then concentrated in vacuo using a rotary evaporator with a water bath temperature of 30° C. The resultant yellow residue was dissolved in ethyl acetate, and treated cautiously with an excess of saturated aqueous sodium bicarbonate solution. The biphasic mixture was stirred until gas evolution ceased. The organic layer was separated and the aqueous layer was re-extracted with ethyl acetate. The combined organic extracts were dried over Na₂SO₄, filtered, and concentrated in vacuo. Purification by preparative TLC ethyl acetate/MeOH (9:1) gave ER-819700 (85 mg, 67%).

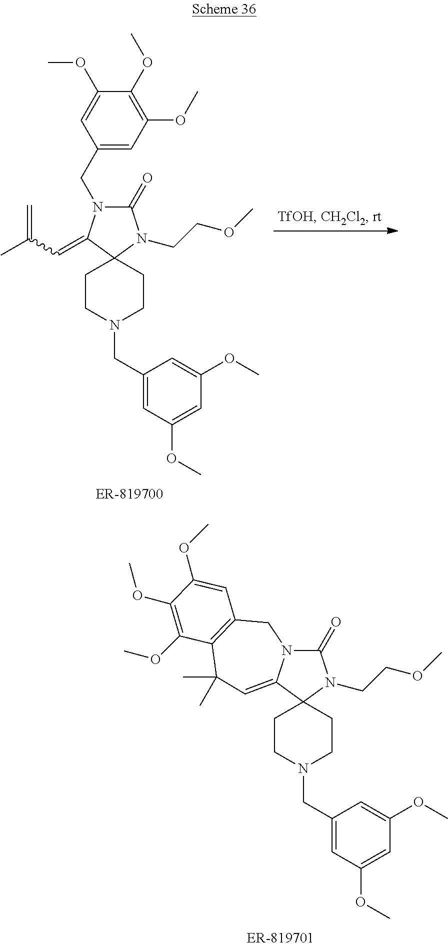

ER-819701: As depicted in Scheme 36 above, to a solution of ER-819700 (45 mg, 0.000076 mol) in methylene chloride (2.25 mL) was added trifluoromethanesulfonic acid (20 μL, 0.0002 mol) dropwise at room temperature. After 40 min the reaction was quenched with sat. NaHCO₃ (color changed from dark yellow to almost colorless), vigorously stirred for 20 min at room temperature, extracted with methylene chloride (3×). The combined extracts were dried over Na2SO4, filtered, concentrated in vacuo. Purification by flash chromatography using 100% ethyl acetate followed by ethyl acetate/methanol (19:1) afforded ER-819701 (26 mg, 58%).

ER-819655, ER-819672, ER-819698, ER-819704 were prepared in substantially the same manner as ER-819701.

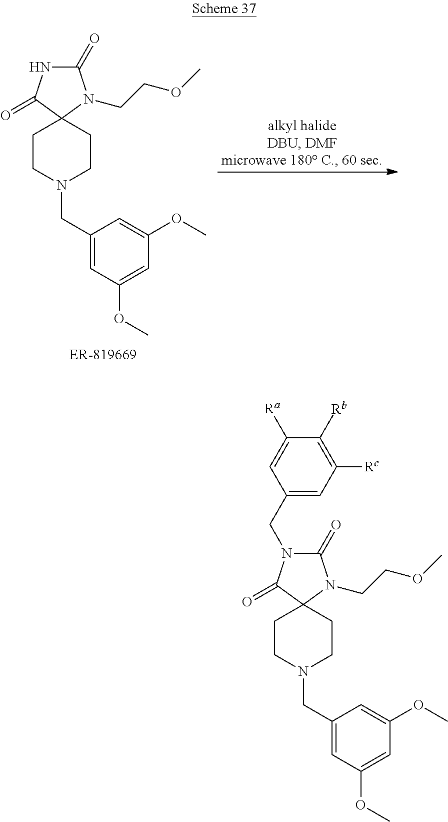

Scheme 37 above depicts a general method for introducing various R^{a}, R^{b}, and R^{c} groups. As depicted in Scheme 37 above, a solution of ER-819669 (0.00029 mol), 1,8-diazabicyclo[5.4.0]undec-7-ene (87.2 μL, 0.000583 mol) and an alkyl halide (0.000495 mol) in N,N-dimethylformamide (1.1 mL) was heated at 180° C. for 60 seconds in the microwave. Purification by preparative reverse phase HPLC provided the desired product.

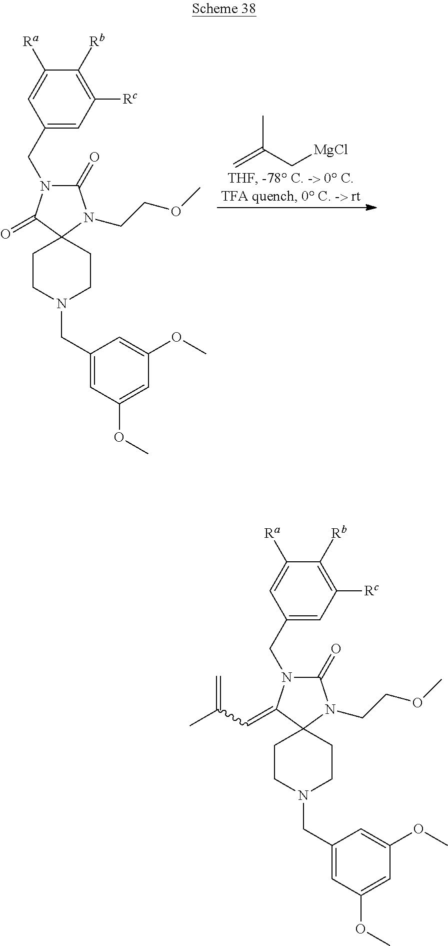

As depicted in Scheme 38 above, to a solution of starting material (0.000212 mol) in tetrahydrofuran (4 mL) at −78° C. was added a 0.5 M solution of 2-methylallylmagnesium chloride in tetrahydrofuran (4.232 mL) dropwise over 3 min keeping internal temperature below at −50° C. The cooling bath was removed to allow the reaction mixture to warm to 0° C. After stirring for 2 hrs at 0° C., the reaction mixture was quenched by slow careful addition of trifluoroacetic acid (0.978 mL, 0.0127 mol). The reaction mixture was then warmed to room temperature, stirred for 10 min and then concentrated in vacuo using a rotary evaporator with the water bath temperature set at 30° C. The resultant residue was dissolved in ethyl acetate, and excess saturated aqueous sodium bicarbonate was added cautiously. The biphasic mixture was stirred until gas evolution ceased. The organic layer was separated; the aqueous layer was extracted with ethyl acetate. The combined organic extracts were dried over Na₂SO₄, filtered, and concentrated in vacuo. Purification by preparative TLC with ethyl acetate/methanol (9:1) afforded the desired product.

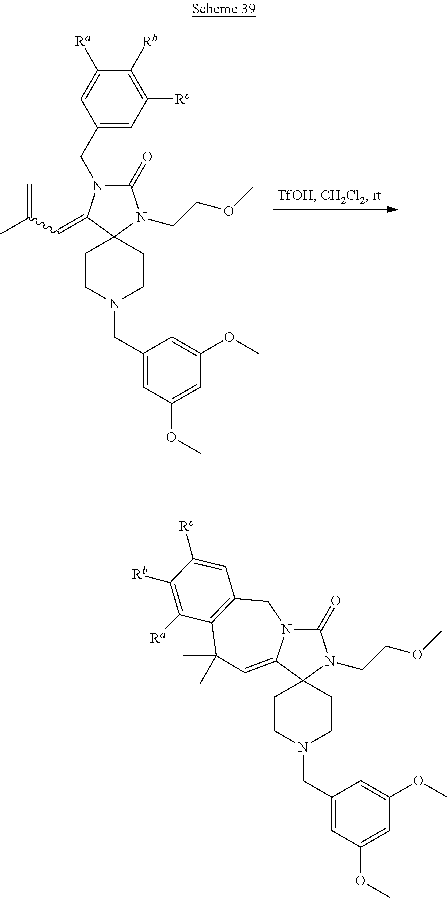

As depicted in Scheme 39 above, to a solution of starting material (0.000076 mol) in methylene chloride (2.25 mL) was added trifluoromethanesulfonic acid (20 μL, 0.0002 mol) dropwise at room temperature. After 40 min the reaction was quenched with an excess of saturated aqueous sodium bicarbonate, vigorously stirred for 20 min at room temperature, and extracted with methylene chloride (3×). The combined extracts were dried over Na₂SO₄, filtered, and concentrated in vacuo. Purification by flash chromatography using 100% ethyl acetate followed by ethyl acetate/methanol (19:1) afforded the desired product.

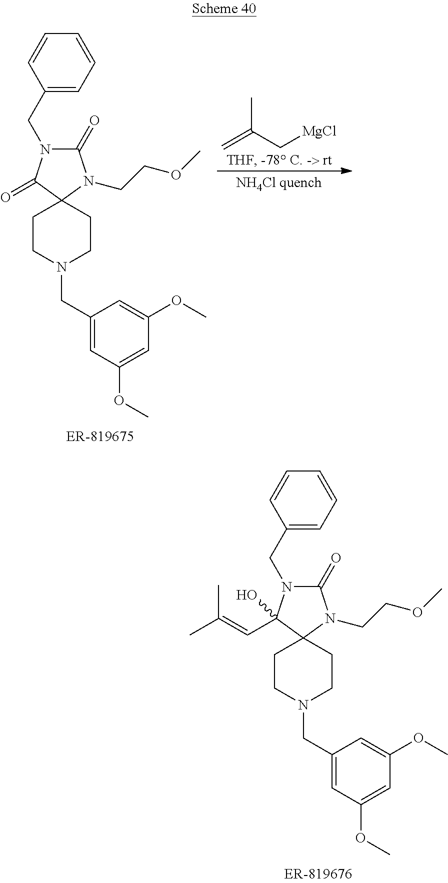

ER-819676: As depicted in Scheme 40 above, to a solution of ER-819675 (80.0 mg, 0.000171 mol) in tetrahydrofuran (2 mL, 0.03 mol) at −78° C. was added a 0.5 M solution of 2-methylallylmagnesium chloride in tetrahydrofuran (3.422 mL) dropwise over 3 min keeping internal temperature below −60° C. The reaction mixture was allowed to warm slowly to −35° C. (over approximately 1.5 hours). The reaction was quenched with saturated aqueous ammonium chloride solution, and extracted with ethyl acetate (2×). The combined extracts were dried over Na₂SO₄, and concentrated in vacuo. The crude product was purified by flash chromatography eluting with ethyl acetate/methanol (19:1) to afford ER-819676 (85 mg, 95%).

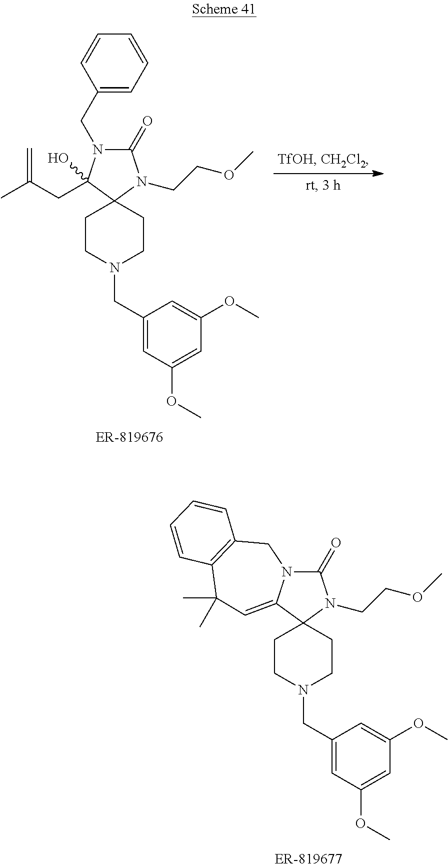

ER-819677: As depicted in Scheme 41 above, to a solution of ER-819676 (56 mg, 0.00011 mol) in methylene chloride (5000 μL) was added trifluoromethanesulfonic acid (90 μL, 0.001 mol) dropwise at room temperature to give yellow solution. After 3 h, the reaction was quenched with saturated aqueous sodium bicarbonate solution, vigorously stirred for 20 min at room temperature and extracted with methylene chloride (3×). The combined extracts were dried with Na₂SO₄, filtered and concentrated in vacuo. Purification by preparative TLC using ethyl acetate/methanol (9:1) as eluent afforded ER-819677 (22 mg, 40%).

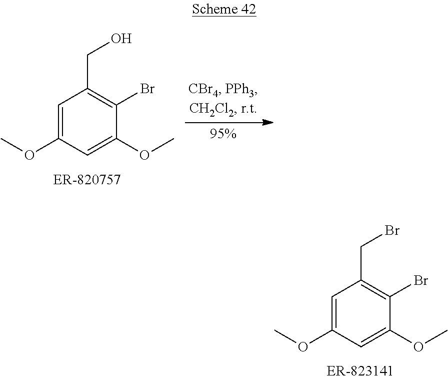

ER-823141: As depicted in Scheme 42 above, ER-820757 (1.62 g, 6.556 mmol) was dissolved in methylene chloride (80 mL). Triphenylphosphine (3.44 g, 13.1 mmol) and carbon tetrabromide (4.35 g, 13.1 mmol) were added and the mixture stirred overnight at room temperature. Concentration in vacuo followed by flash chromatography using ethyl acetate/heptane (1:9) as eluent afforded ER-823141 (1.93 g, 95%) as a light grey solid.

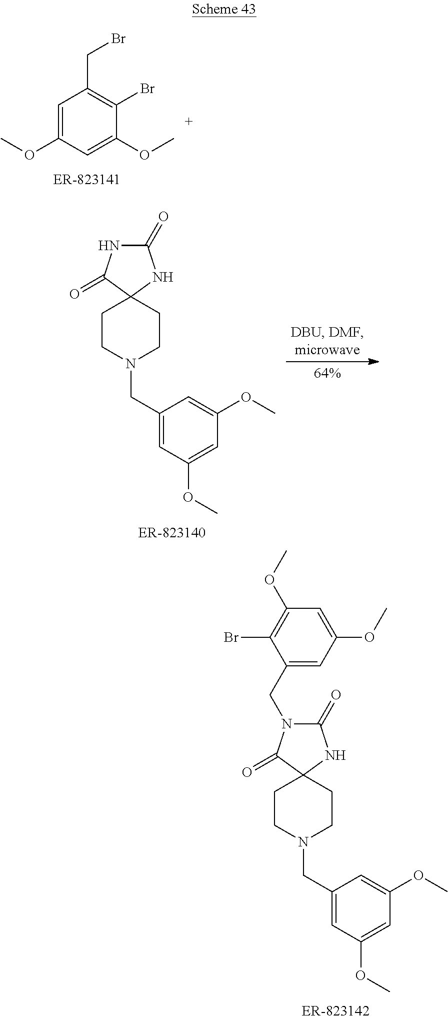

ER-823142: As depicted in Scheme 43 above, a 5 mL microwave reactor vial, equipped with a magnetic stir bar, was charged with ER-823140 (200.0 mg, 0.6263 mmol), N,N-dimethylformamide (2.0 mL), ER-823141 (388 mg, 1.25 mmol) and 1,8-diazabicyclo[5.4.0]undec-7-ene (211 μL, 1.41 mmol) to give a light yellow solution. The reaction mixture was heated at 180° C. for 90 seconds in the microwave. Ethyl acetate (5.0 mL) was added followed by a saturated aqueous ammonium chloride solution (2.5 mL) and water (2.5 mL). The organic layer was isolated and the aqueous layer extracted (2×) with ethyl acetate (5.0 mL). The combined organic extracts were washed with saturated aqueous sodium chloride solution (5.0 mL). The organic layer was dried with sodium sulfate, filtered and concentrated in vacuo. The residue was purified by flash chromatography (0-2.5% methanol/ethyl acetate) to give ER-823142 (218 mg, 63%) as a colorless solid.

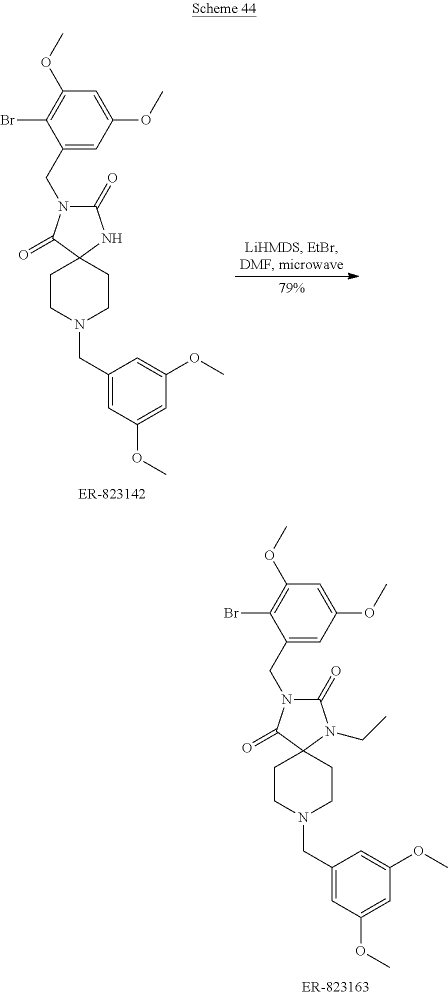

ER-823163: As depicted in Scheme 44 above, a 5 mL microwave reactor vial, equipped with a magnetic stir bar, was charged with ER-823142 (100.0 mg, 0.1823 mmol), N,N-dimethylformamide (1.00 mL), 1 M lithium hexamethyldisilazide solution in tetrahydrofuran (0.43 mL), and ethyl bromide (0.032 mL, 0.438 mmol). The mixture was heated at 170° C. for 150 seconds in the microwave. The reactor mixture was cooled to room temperature and treated with MTBE (2 mL). Saturated aqueous ammonium chloride solution (1 mL) was added and the mixture was stirred for 10 minutes. The organic layer was isolated and the aqueous layer back extracted with MTBE (2×2 mL). The combined organic layers were washed with saturated aqueous sodium chloride solution (2 mL). The organic layer was dried with sodium sulfate, filtered and concentrated in vacuo. The crude material was purified by flash chromatography (ethyl acetate) to give ER-823163 (83 mg, 79%) as a light yellow solid.

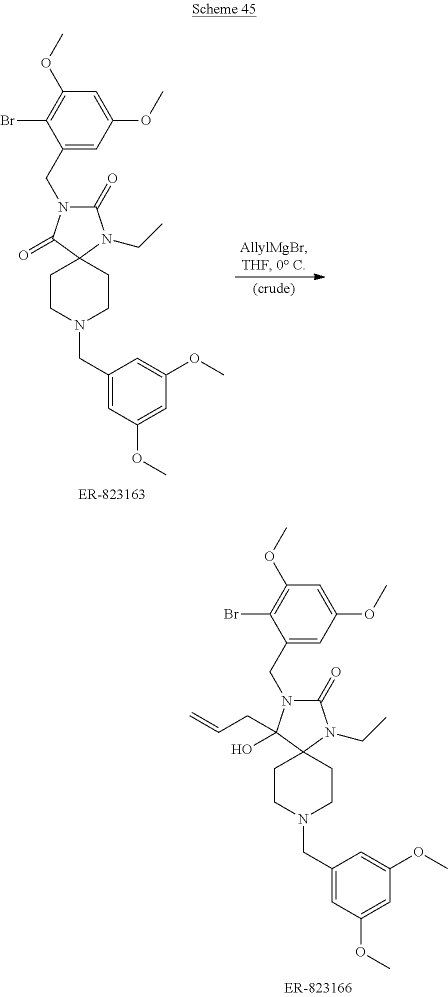

ER-823166: As depicted in Scheme 45 above, ER-823163 (153.0 mg, 0.2654 mmol) was dissolved in anhydrous tetrahydrofuran (1.5 mL) and the solution cooled to 0° C. A 1.0 M solution of allylmagnesium bromide in ether (1.327 mL) was added and the mixture stirred at 0° C. for 1.5 hours. Saturated aqueous ammonium chloride solution (1.5 mL) was added and the mixture was stirred for 10 minutes. The mixture was extracted (2×) with MTBE (7 mL) The combined organic layers were washed with saturated aqueous sodium chloride solution (3 mL). The organic layer was dried with sodium sulfate, filtered and concentrated in vacuo to afford crude ER-823166 (160 mg) which was used immediately without purification.

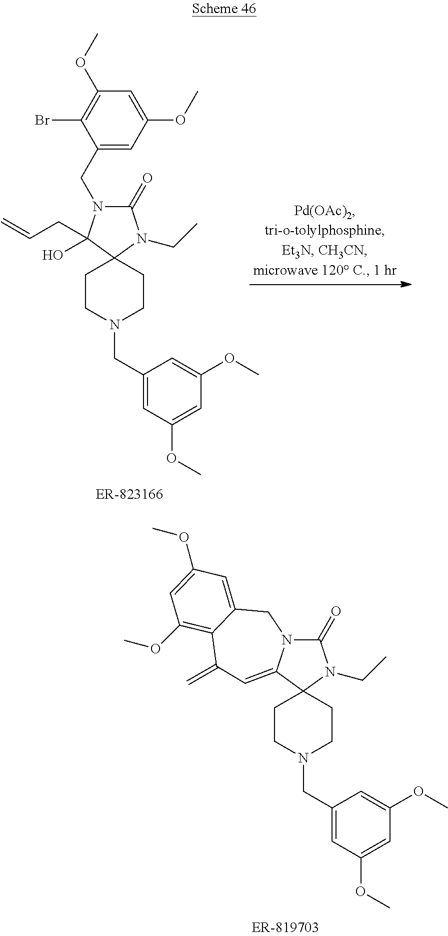

ER-819703: As depicted in Scheme 46 above, to a solution of ER-823166 (110.0 mg, 0.1778 mmol) in acetonitrile (2.5 mL) under an atmosphere of nitrogen in a 5 mL microwave reactor vial was added palladium acetate (20.0 mg, 0.0889 mmol), tri-o-tolylphosphine (27.6 mg, 0.0907 mmol) and triethylamine (99.1 μL, 0.711 mmol). The mixture was heated at 120° C. for 60 minutes in the microwave. The reaction mixture was filtered through a short pad of Celite and silica gel, and the pad subsequently washed with ethyl acetate/methanol (9:1). The filtrate was concentrated in vacuo. Purification of the resultant residue by preparative reverse phase HPLC provided ER-819703 (10 mg, 12%).

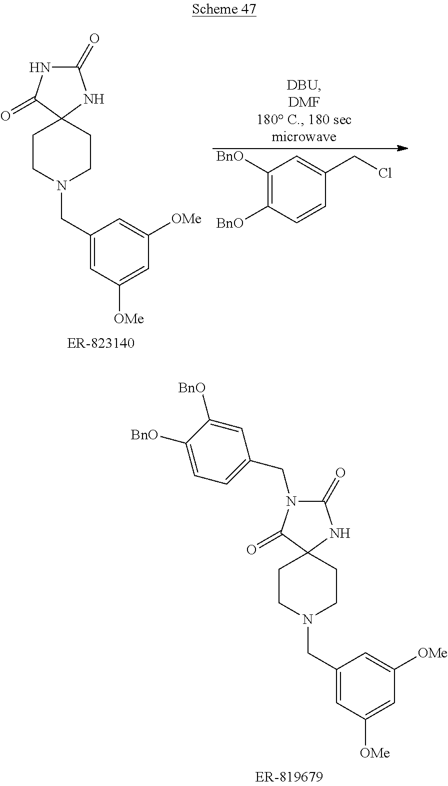

ER-819679: As depicted in Scheme 47 above, a 5-mL microwave reactor vial was charged with a magnetic stir-bar, ER-823140 (505.0 mg, 0.001581 mol), and N,N-dimethylformamide (3.5 mL). The mixture was stirred for a few minutes to dissolve all the solid, giving a clear, faintly yellow solution. 3,4-dibenzyloxybenzyl chloride (910.8 mg, 0.002688 mol) was added, and the solution was stirred to dissolve. 1,8-diazabicyclo[5.4.0]undec-7-ene (475 4, 0.00318 mol) was then added via syringe. The solution rapidly took on a slightly greenish tint after the 1,8-diazabicyclo[5.4.0]undec-7-ene was added, but the color did not darken further. The clear solution was stirred to mix, the tube was sealed with a septum cap, and the reactor vial heated in the microwave at 180° C. for 90 sec., and then allowed to stand at room temperature overnight. TLC and mass spectroscopic analysis indicated a small amount of ER-823140 remaining. Consequently, the reactor vial was heated in the microwave again for 90 sec at 180° C. The clear, amber solution was diluted with ethyl acetate (80 mL) and washed with water (2×30 mL), saturated aqueous sodium bicarbonate solution (30 mL), water (30 mL), and saturated brine (30 mL), dried over anhydrous magnesium sulfate, filtered, and concentrated in vacuo to give ER-819679 (1.02 g, 104%) as a light tan solid. ¹H NMR (CDCl₃) indicated sufficient purity for use in the next step without further purification.

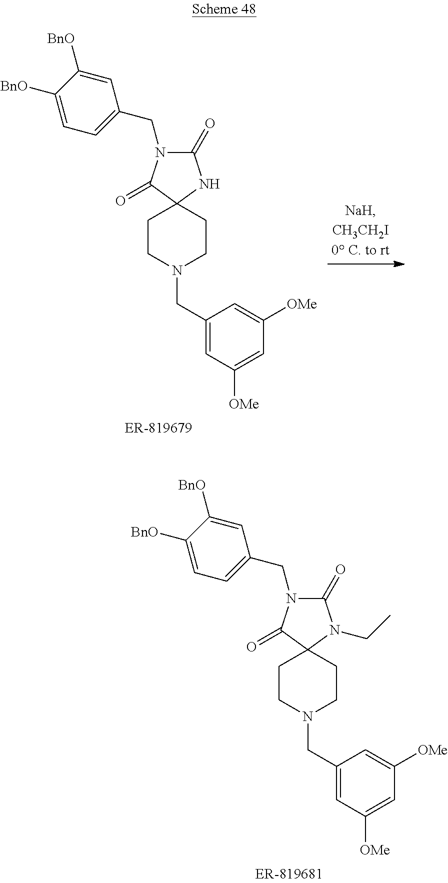

ER-819681: As depicted in Scheme 48 above, ER-819679 (0.6204 g, 0.0009979 mol) was dissolved in N,N-dimethylformamide (5.0 mL, 0.064 mol) at room temperature, and the solution was cooled in an ice-water bath under nitrogen. Sodium hydride (47.9 mg, 0.00120 mol) was added all at once, and the mixture stirred for 40 min. Iodoethane (100 μL, 0.001250 mol) was added via syringe. The resultant cloudy solution was stirred with ice-water bath cooling for 2.3 h, and the bath was then removed. Stirring was continued at room temperature overnight. The reaction solution was diluted with ethyl acetate (80 mL) and water (25 mL), and the phases separated. The ethyl acetate phase was washed with water (2×25 mL), and saturated brine (30 mL), dried over anhydrous magnesium sulfate, filtered, and concentrated in vacuo to give an off-white film. This film was rinsed with heptanes (3×˜2 mL), and the heptanes was decanted by pipette. The solid was re-dried under vacuum to give ER-819681 (648.0 mg, 100%) as a semi-solid foam that melted with warming.

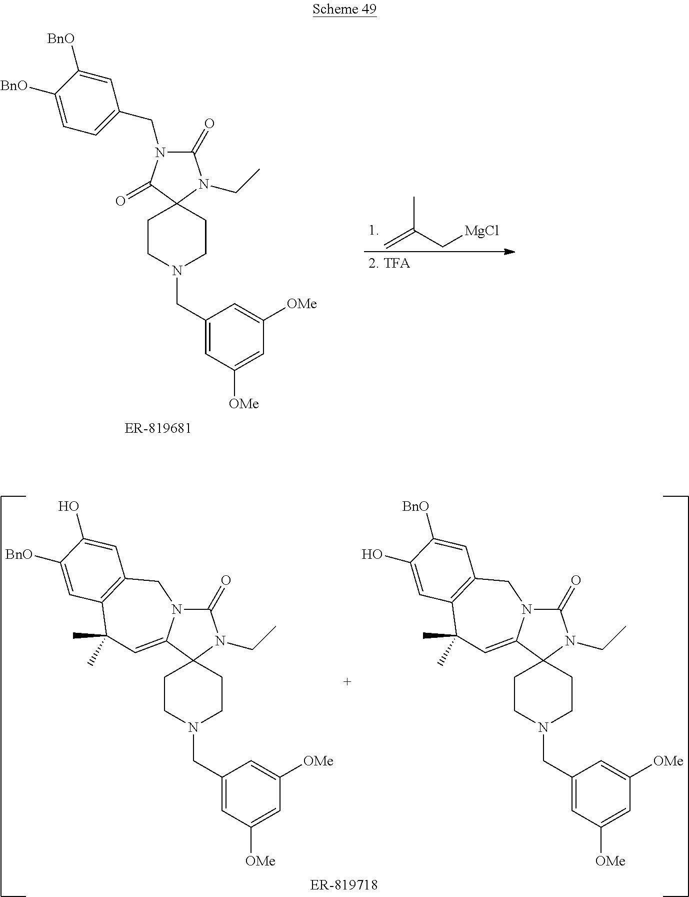

ER-819718: As depicted in Scheme 49 above, ER-819681 (200.3 mg, 0.0003083 mol) was dissolved in tetrahydrofuran (3.0 mL) under nitrogen, and the solution cooled to −78° C. in a dry ice/acetone bath. A 0.5 M solution of 2-methylallylmagnesium chloride in tetrahydrofuran (2.0 mL) was added via syringe over ca. 3 min, and the solution was allowed to stir at −78° C. for 5 min, and then the bath was removed, and the solution was stirred at room temperature for 2.5 h. The solution was re-cooled to −78° C. and quenched with 0.1 mL trifluoroacetic acid. This solution was then concentrated in vacuo to give a yellow foam. The flask was cooled to −78° C. in a dry ice/acetone bath and 3.0 mL of trifluoroacetic acid was added. The trifluoroacetic acid solidified, so the flask was removed from the bath, and allowed to warm to room temperature. After 3 hours, 1 mL of methylene chloride was added to help dissolve the solid. After ˜7 hours total at room temperature, the red solution was concentrated in vacuo using a rotary evaporator with the water bath temperature set to approximately 40° C. The residual red-brown oil was dissolved in a few mL of ethyl acetate (with sonication) and diluted with a total of approximately 80 mL of ethyl acetate. This solution was washed with saturated sodium bicarbonate solution (40 mL), water (40 mL), and saturated brine (40 mL). The organic extract was then dried over anhydrous magnesium sulfate, filtered, and concentrated in vacuo to afford a yellow-brown oil (200.4 mg). Purification by preparative reverse phase HPLC provided ER-819717 (1.0 mg, 1.8%) and ER-819718 (1.2 mg, 2.2%).

Compounds of the present invention were prepared in accordance with the methods described herein and those known to one of ordinary skill in the art. Such compounds include those listed in Table 1 set forth below. Table 1 provides analytical data, including ¹H NMR data, for exemplary compounds of the present invention.

TABLE 1
Analytical Data for Exemplary Compounds of Formula I

| Example # | Structure | ER-# | Analytical Data |
|---|---|---|---|
| 1 | 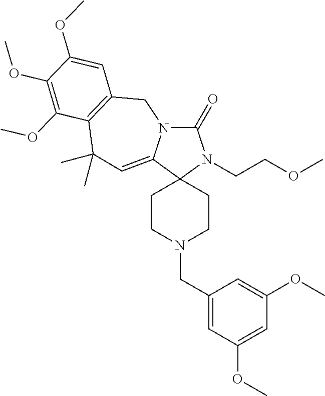 | 819701 Salt free | NMR ¹H (400 MHz, CDCl₃) δ 6.63 (s, 1H), 6.51 (d, J = 2.3 Hz, 2H), 6.38 (t, J = 2.2 Hz 1H), 4.70 (s, 1H), 4.68 (s, 2H), 3.85 (s, 3H), 3.84 (s, 3H), 3.81 (s, 6H), 3.80 (s, 3H), 3.54 (s, 2H), 3.51 (t, J = 6.2 Hz, 2H), 3.38 (t, J = 6.6 Hz, 2H), 3.35 (s, 3H), 2.78-2.75 (m, 2H), 2.54 (t, J = 10.9 Hz, 2H), 2.01-1.93 (m, 2H), 1.69 (s, 6H), 1.65-1.62 (m, 2H) |
| 2 | 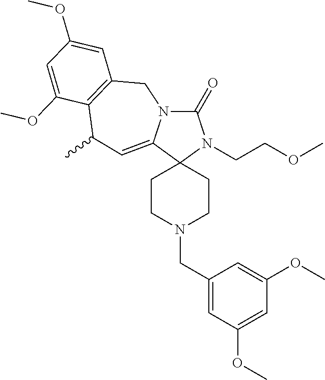 | 819543 Salt free | NMR ¹H (400 MHz, DMSO) δ 6.48-6.46 (m, 3H), 6.38 (d, J = 2.6 Hz, 1H), 6.35 (t, J = 2.3 Hz, 1H), 5.04 (d, J = 8.5 Hz, 1H), 4.56 (dd, J = 14.1 Hz, 2H) 4.06-4.01 (m, 1H), 3.74 (s, 3H), 3.72 (s, 3H), 3.70 (s, 6H), 3.46 (s, 2H), 3.35 (t, J = 6.74 Hz, 2H), 3.26-3.16 (m, 2H), 3.20 (s, 3H), 2.70-2.60 (m, 2H), 2.49-2.39 (m, 2H), 1.89-1.78 (m 2H), - 1.54-1.50 (m, 1H), 1.40-1.36 (m, 1H), 1.26 (d, J = 7.3 Hz, 3H), |
| 3 | 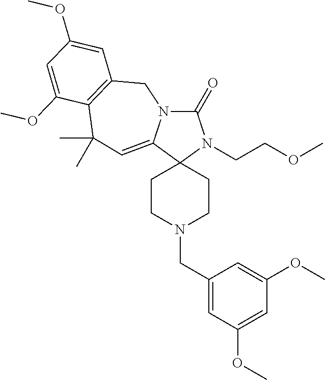 | 819544 Salt free | NMR ¹H (400 MHz, CDCl₃) δ 6.52-6.50 (m, 2H), 6.46-6.45 (m, 2H), 6.38-6.37 (m, 1H), 4.69 (s, 1H), 4.62 (s, 2H), 3.80 (s, 6H), 3.79 (s, 3H), 3.76 (s, 3H), 3.53-3.50 (m, 4H), 3.40-3.37 (m, 2H), 3.35 (s, 3H), 2.78-2.75 (m, 2H), 2.58-2.55 (m, 2H), 2.01-1.97 (m, 2H), 1.66 (s, 6H), 1.67- 1.62 (m, 2H) |
| 4 | 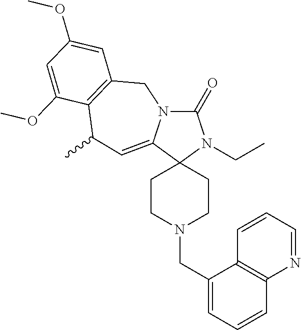 | 819592 Salt free | NMR ¹H (400 MHz, DMSO) δ 8.89-8.87 (m, 1H), 8.70 (d, J = 8.8 Hz, 1H), 7.92 (d, J = 8.2 Hz, 1H), 7.67 (t, J = 7.9 Hz, 1H), 7.56- 7.53 (m, 2H), 6.48-6.47 (m, 1H), 6.38- 6.37 (m, 1H), 5.10 (d, J = 8.2 Hz, 1H), 4.56 (dd, J = 14.2 Hz, 2H), 4.09-4.04 (m, 1H), 3.99 (s, 2H), 3.75 (s, 3H), 3.72 (s, 3H), 3.12-3.03 (m, 2H), 2.78-2.55 (m, 4H), 1.83-1.71 (m, 2H), 1.57-1.53 (m, 1H), 1.40-1.37 (m, 1H), 1.28 (d, J = 7.3 Hz, 3H), 1.00 (t, J = 6.9 Hz, 3H) |
| 5 | 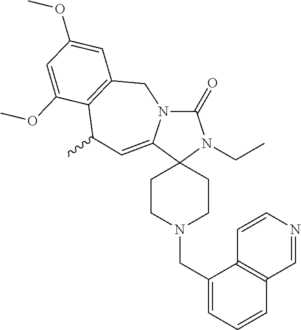 | 819593 Salt free | NMR ¹H (400 MHz, DMSO) δ 8.85-8.84 (m, 1H), 8.33 (d, J = 8.2 Hz, 1H), 7.98-7.93 (m, 1H), 7.87 (s, 1H), 7.75-7.73 (m, 1H), 7.51-7.48 (m, 1H), 6.47 (s, 1H), 6.38 (s, 1H), 5.05 (d, J = 8.2 Hz, 1H), 4.55, (dd, J = 14.2 Hz, 2H), 4.05-4.01 (m, 1H), 3.74 (s, 5H), 3.72 (s, 3H), 3.18-3.11 (m, 2H), 2.75-2.52 (m, 4H), 1.91-1.82 (m, 2H), 1.58-1.55 (m, 1H), 1.43-1.40 (m, 1H), 1.26 (d, J = 7.3 Hz, 3H), 1.03 (t, J = 6.7 Hz, 3H) |
| 6 | 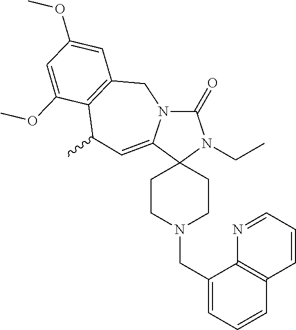 | 819594 Salt free | NMR ¹H (400 MHz, DMSO) δ 8.91-8.90 (m, 1H), 8.36-8.34 (m, 1H), 7.87-7.85 (m, 2H), 7.59 (t, J = 7.8 Hz, 1H), 7.54-7.51 (m, 1H), 6.48-6.47 (m, 1H), 6.38-6.37 (m, 1H), 5.07 (d, J = 8.5 Hz, 1H), 4.55 (dd, J = 14.2 Hz, 2H), 4.25 (s, 2H), 4.06-4.02 (m, 1H), 3.74 (s, 3H), 3.72 (s, 3H), 3.19- 3.12 (m, 2H), 2.86-2.60 (m, 4H), 1.96- 1.85 (m, 2H), 1.60-1.57 (m, 1H), 1.45- 1.42 (m, 1H), 1.26 (d, J = 7.3 Hz, 3H), 1.04 (t, J = 6.9 Hz, 3H) |
| 7 | 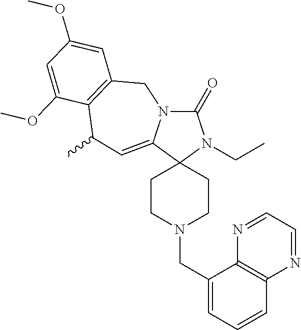 | 819595 Salt free | NMR ¹H (400 MHz, DMSO) δ 8.96-8.95 (m, 2H), 8.00-7.93 (m, 2H), 7.87-7.83 (m, 1H), 6.48-6.47 (m, 1H), 6.38-6.37 (m, 1H), 5.06 (d, J = 8.5 Hz, 1H), 4.55, (dd, J = 14.1 Hz, 2H), 4.24 (s, 2H), 4.05-4.01 (m, 1H), 3.74 (s, 3H), 3.72 (s, 3H), 3.19-3.11 (m, 2H), 2.79-260 (m, 4H), 1.95-1.84 (m, 2H), 1.59-1.56 (m, 1H), 1.44-1.41 (m, 1H), 1.26 (d, J = 7.0 Hz, 3H), 1.03 (t, J = 7.0 Hz, 3H), |
| 8 | 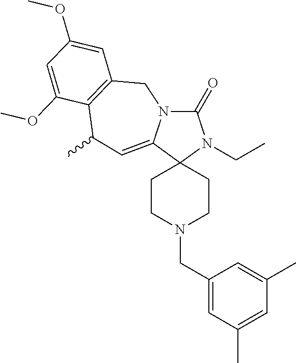 | 819597 Salt free | NMR ¹H (400 MHz, DMSO) δ 6.89 (s, 2H), 6.85 (s, 1H), 6.48-6.47 (m, 1H), 6.38-6.37 (m, 1H), 5.00 (d, J = 8.5 Hz, 1H), 4.55 (dd, J = 14.2 Hz, 2H), 4.04-4.00 (m, 1H), 3.74 (s, 3H), 3.72 (s, 3H), 3.44 (s, 2H), 3.17- 3.08 (m, 2H), 2.68-2.57 (m, 2H), 2.51- 2.38 (m, 2H), 2.23 (s, 6H), 1.88-1.75 (m, 2H), 1.56-1.52 (m, 1H), 1.40-1.37 (m, 1H), 1.26 (d, J = 7.0 Hz, 3H), 1.02 (t, J = 7.0 Hz, 3H), |
| 9 | 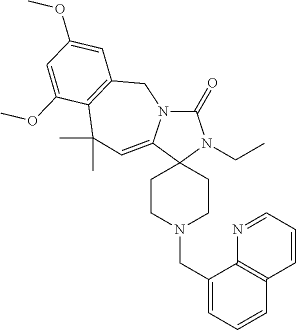 | 819604 Salt free | NMR ¹H (400 MHz, DMSO) δ 8.91-8.92 (m, 1H), 8.37- 835 (m, 1H), 7.89-7.82 (m, 2H), 7.62-7.51 (m, 2H), 6.50-6.49 (m, 1H), 6.38- 6.37 (m, 1H), 4.56 (s, 1H), 4.44 (s, 2H), 4.28 (s, 2H), 3.71 (s, 3H), 3.70 (s, 3H), 3.19-3.16 (m, 2H), 2.85-2.80 (m, 2H), 2.65-2.59 (m, 2H), 1.98-1.90 (m, 2H), 1.58-1.52 (m, 2H), 1.50 (s, 6H), 1.08-1.03 (m, 3H) |
| 10 | 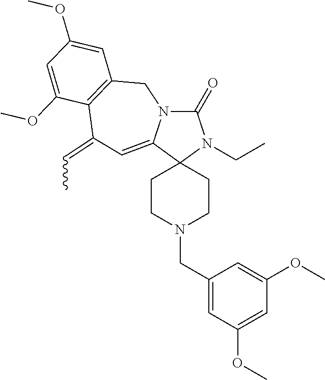 | 819651 Salt free | NMR ¹H (400 MHz, CD₃OD) δ 6.56-6.49 (m, 4H), 6.44-6.42 (m, 1H), 5.73-5.72; 5.60-5.59 (2m, 1H), 5.70-5.68; 5.54-5.52 (2m, 1H), 4.54 (dd, J = 13.6 Hz, 2H), 3.80-3.65 (m, 14H), 3.21-3.18 (m, 2H), 2.89-2.61 (m, 4H), 2.10-1.94 (m, 2H), 1.76-1.74 (m, 2H), 1.50-1.48; 1.32-1.28 (2m, 3H), 1.18-1.12 (m, 3H) |
| 11 | 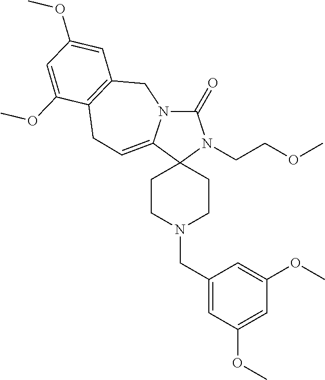 | 819673 Salt free | NMR ¹H (400 MHz, CD₃OD) δ 6.55-6.54 (m, 2H), 6.50-6.49 (m, 1H), 6.46-6.45 (m, 1H), 6.41 (br, lH), 5.08 (t, J = 6.2 Hz, 1H), 4.73 (s, 2H), 3.79 (s, 3H), 3.77 (s, 9H), 3.61-3.57 (m, 4H), 3.45 (t, J = 6.2 Hz, 2H), 3.33-3.31 (m, 2H), 2.91-2.82 (m, 2H), 2.66-2.56 (m, 2H), 2.15 (s, 3H), 2.01-1.96 (m, 2H), 1.60-1.56 (m, 2H), |
| 12 | 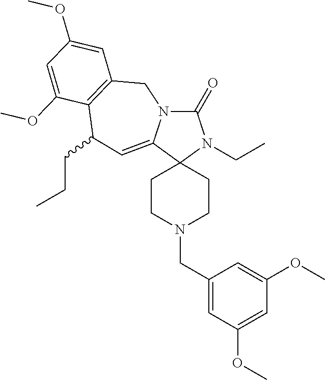 | 819626 Salt free | NMR ¹H (400 MHz, CD₃OD) δ 6.71-6.62 (m, 3H), 6.47-6.46 (m, 1H), 3.69-6.38 (m, 1H), 4.79-4.78 (m, 2H), 4.38 (br, 1H), 4.12- 4.10 (m, 1H), 3.82-3.56 (m, 16H),3.64- 3.56 (m, 2H), 3.48-3.45 (m, 2H), 2.58- 2.43 (m, 2H), 2.22-2.05 (m, 2H), 1.43- 1.41 (m, 4H), 1.18-1.15 (m, 6H) |
| 13 | 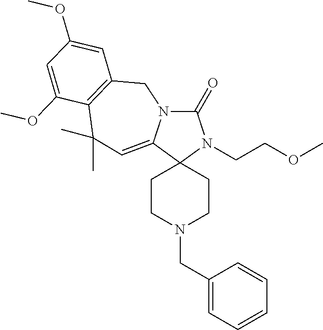 | 819641 Salt free | NMR ¹H (400 MHz, CD₃OD) δ 7.37-7.28 (m, 5H), 6.51 (d, J = 2.6 Hz, 1H), 6.43 (d, J = 2.6 Hz, 1H), 4.67 (s, 1H), 4.54 (s, 2H), 3.78 (s, 3H), 3, 76 (s, 3H), 3.69 (s, 2H), 3.51- 3.48 (m, 2H), 3.39-3.35 (m, 2H), 3.32 (s, 3H), 2.85-2.82 (m, 2H), 2.70-2.61 (m, 2H), 2.09-2.01 (m, 2H), 1.65-1.60 (m, 2H), 1.59 (s, 6H) |
| 14 | 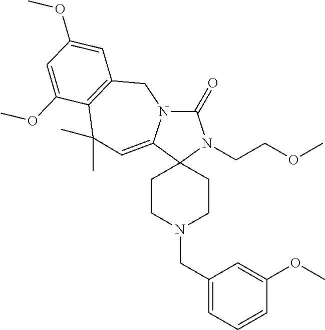 | 819647 Salt free | NMR ¹H (400 MHz, CD₃OD) δ 7.27 (t, J = 7.9 Hz, 1H), 6.93-6.91 (m, 2H), 6.88-6.86 (m, 1H), 6.52 (d, J = 2.6 Hz, 1H), 6.43 (d, J = 2.9 Hz, 1H), 4.68 (s, 1H), 4.54 (s, 2H), 3.81 (s, 3H), 3.78 (s, 3H), 3.76 (s, 3H), 3.66 (s, 2H), 3.52-3.48 (m, 2H), 3.39- 3.36 (m, 2H), 3.33 (s, 3H), 2.85-2.81 (m, 2H), 2.69-2.62 (m, 2H), 2.09-2.01 (m, 2H), 1.64-1.60 (m, 2H), 1.59 (s, 6H) |
| 15 | 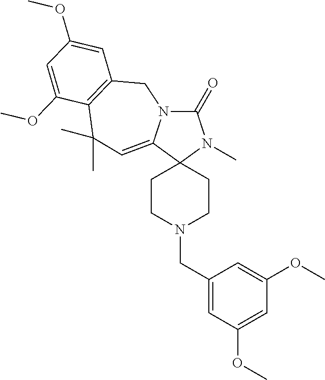 | 819658 Salt free | NMR ¹H (400 MHz, CD₃OD) δ 6.54-6.53 (m, 2H), 6.51-6.50 (m, 1H), 6.44-6.42 (m, 2H), 4.67 (s, 1H), 4.55 (s, 2H), 3.79 (s, 6H), 3.78 (s, 3H), 3.76 (s, 3H), 3.62 (s, 2H), 2.85 (s, 3H), 2.83-2.77 (m, 2H), 2.75- 2.69 (m, 2H), 2.14-2.06 (m, 2H), 1.67- 1.61 (m, 2H). 1.60 (s, 6H) |
| 16 | 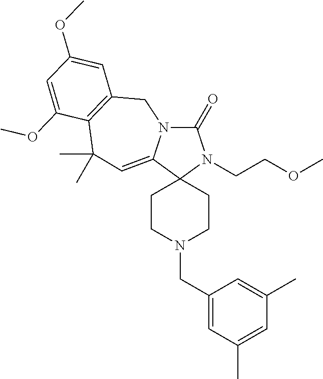 | 819659 Salt free | NMR ¹H (400 MHz, CD₃OD) δ 6.96 (s, 3H), 6.51 (d, J = 2.6 Hz, 1H), 6.43 (d, J = 2.6 Hz, 1H), 4.63 (s, 1H), 4.54 (s, 2H), 3.77 (s, 3H), 3.46 (s, 3H), 3.62 (s, 2H), 3.51-3.48 (m, 2H), 3.39-3.36 (m, 2H), 3.32 (s, 3H), 2.83-2.77 (m, 2H), 2.69-2.62 (m, 2H), 2.31 (s, 6H), 2.11-2.01 (m, 2H), 1.64- 1.59 (m, 2H), 1.57 (s, 6H), |
| 17 | 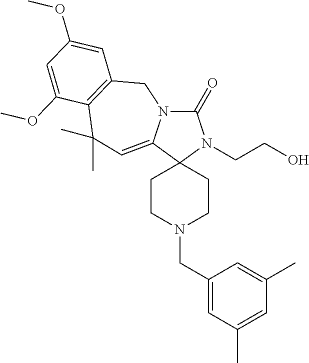 | 819660 Salt free | NMR ¹H (400 MHz, CD₃OD) δ 6.96 (s, 3H), 6.50 (d, J = 2.6 Hz, 1H), 6.43 (d, J = 2.6 Hz, 1H), 4.64 (s, 1H), 4.54 (s, 2H), 3.77 (s, 3H), 3.75 (s, 3H), 3.66-3.62 (m, 2H), 3.33-3.30 (m, 2H), 2.83-2.80 (m, 2H), 2.69-2.61 (m, 2H), 2.31 (s, 6H), 2.07- 1.99 (m, 2H), 1.66-1.62 (m, 2H), 1.57 (s, 6H) |
| 18 | 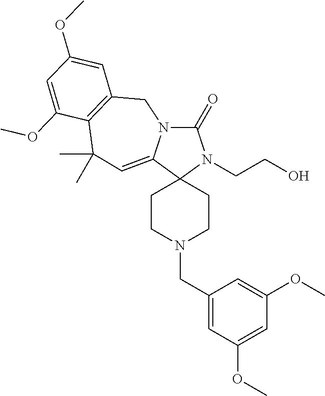 | 819657 Salt free | NMR ¹H (400 MHz, CD₃OD) δ 6.53-6.52 (m, 2H), 6.51-6.50 (m, 1H), 6.44-6.42 (m, 2H), 4.70 (s, 1H), 4.55 (2H), 3.79 (s, 6H), 3.77 (s, 3H), 3.76 (s, 3H), 3.65 (t, J = 6.4 Hz, 2H), 3.62 (s, 2H), 3.33-3.31 (m, 2H), 2.85-2.82 (m, 2H), 2.70-2.64 (m, 2H), 2.08-2.00 (m, 2H), 1.67-1.64 (m, 2H), 1.61 (s, 6H) |
| 19 | 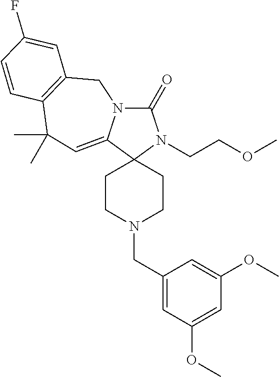 | ER-819672 Salt free | NMR ¹H (400 MHz, CDCl₃) δ 7.32-2.27 (m, 1H), 7.02-6.99 (m, 2H), 6.51 (d, J = 2.3 Hz, 2H), 6.38 (t, J = 2.3 Hz, 1H), 4.82 (s, 2H), 4.78 (s, 1H), 3.81 (s, 6H), 3.52 (s, 2H), 3.52-3.48 (m, 2H), 3.39-3.35 (m, 2H), 3.34 (s, 3H), 2.77-2.72 (m, 2H), 2.54- 2.47 (m, 2H), 1.99-1.91 (m, 2H), 1.62- 1.57-(m, 2H), 1.55 (s, 6H) |
| 20 | 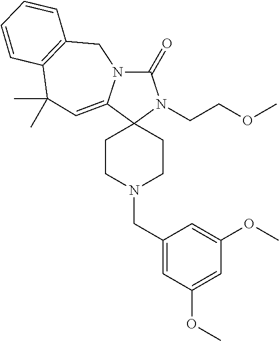 | 819677 Salt free | NMR ¹H (400 MHz, CDCl₃) δ 7.37-7.34 (m, 1H), 7.31-7.27 (m, 2H), 7.23-7.19 (m, 1H), 6.51 (d, J = 2.3 Hz, 2H), 6.38 (t, J = 2.3 Hz, 1H), 4.88 (s, 2H), 4.78 (s, 1H), 3.81 (s, 6H), 3.54-3.48 (m, 4H), 3.39-3.34 (m, 2H), 3.33 (s, 3H), 2.78-2.72 (m, 2H), 2.56-2.49 (m, 2H), 1.99-1.91 (m, 2H), 1.64-1.58 (m, 2H), 1.57 (s, 6H) |
| 21 | 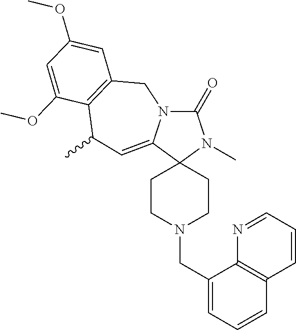 | 819689 Salt free | NMR ¹H (400 MHz, DMSO) δ 8.92-8.90 (m, 1H), 8.36-8.34 (m, 1H), 7.87-7.83 (m, 2H), 7.61-7.60 (m, 1H), 7.53-7.51 (m, 1H), 6.47 (m, 1H), 6.38 (m, 1H), 5.06 (d, J = 8.5 Hz, 1H), 4.57 (dd, J = 14.3 Hz, 2H), 4.24 (s, 2H), 4.06-4.02 (m, 1H), 3.74 (s, 3H), 3.71 (s, 3H), 2.81-2.63 (m, 4H), 2.73 (s, 3H), 2.00-1 .92 (m, 2H), 1.59- 1.56 (m, 1H), 1.44-1.40 (m, 1H), 1.26 (d, J = 7.3 Hz, 3H) |
| 22 | 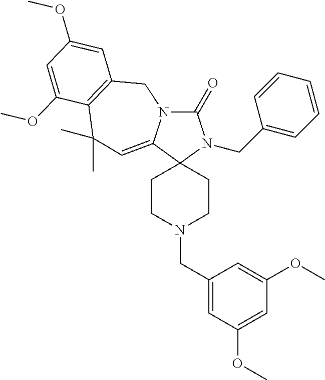 | 819662 Salt free | M/Z (ES+) Calc: 597.3 Found: 598.3 (M + H) Analytical HPLC: Method A1 Xterra MS C18 (4.6 × 100 mm) 5 um Retention time: 9.98 min |
| 23 | 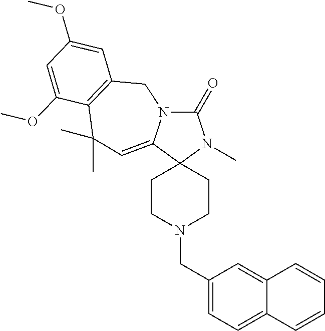 | 819627 TFA salt | M/Z (ES+) Calc: 511.3 Found: 512.4 (M + H) Analytical HPLC: Method A2 Xterra MS C18 (4.6 × 100 mm) 5 um Retention time:6.80 min |
| 24 | 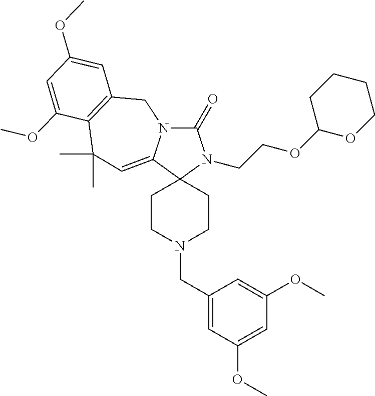 | 819661 Salt free | M/Z (ES+) Calc: 635.4 Found: 636.4 (M + H) Analytical HPLC: Method A1 Xterra MS C18 (4.6 × 100 mm) 5 um Retention time: 9.54 min |
| 25 | 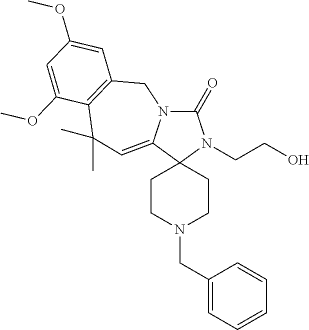 | 819642 Salt free | M/Z (ES+) Calc: 491.3 Found: 492.4 (M + H) Analytical HPLC: Method A1 Xterra MS C18 (4.6 × 100 mm) 5 um Retention time: 7.28 min |
| 26 | 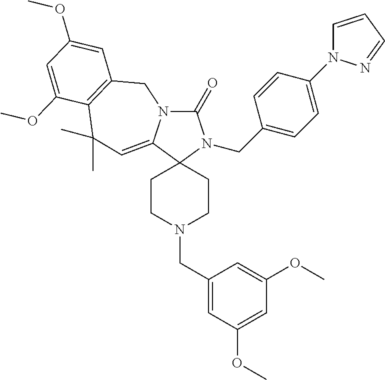 | 819663 Salt free | M/Z (ES+) Calc: 663.3 Found: 664.7 (M + H) Analytical HPLC: Method A1 Xterra MS C18 (4.6 × 100 mm) 5 um Retention time: 9.60 min |
| 27 | 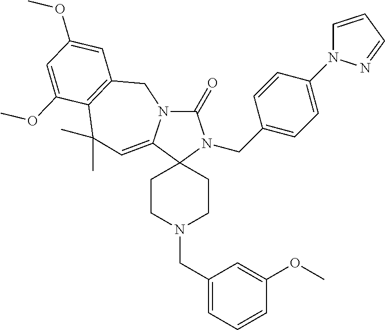 | 819650 Salt free | M/Z (ES+) Calc: 633.3 Found: 634.4 (M + H) Analytical HPLC: Method A1 Xterra MS C18 (4.6 × 100 mm) 5 um Retention time: 9.72 min |
| 28 | 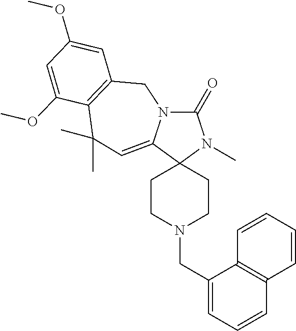 | 819637 TFA salt | M/Z (ES+) Calc: 551.3 Found: 512.3 (M + H) Analytical HPLC: Method A2 Xterra MS C18 (4.6 × 100 mm) 5 um Retention time: 7.17 min |
| 29 | 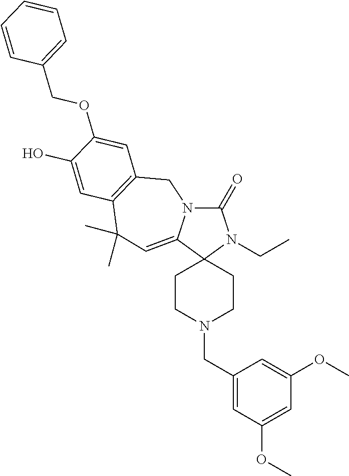 | 819718 TFA salt | M/Z (ES+) Calc: 597.3 Found: 598.4 (M + H) |
| 30 | 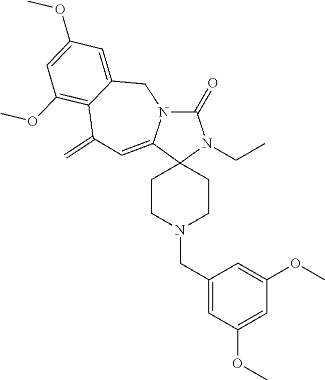 | 819703 TFA salt | M/Z (ES+) Calc: 519.3 Found: 520.4 (M + H) |
| 31 | 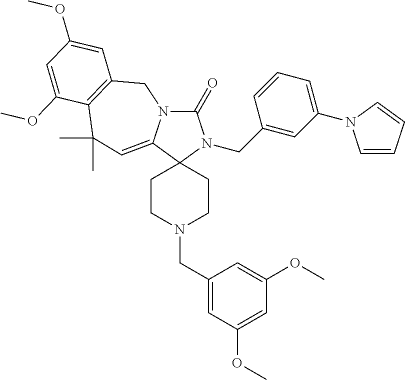 | 819590 Salt free | NMR ¹H (400 MHz, DMSO) δ 7.45 (s, 1H), 7.40-7.32 (m, 2H), 7.27 (m, 2H), 7.05 (d, J = 7.6 Hz, 1H), 6.49 (d, J = 2.3 Hz, 1H), 6.42 (s, 2H), 6.34-6.30 (m, 2H), 6.23 (s, 2H), 4.62-4.40 (m, 4H), 3.75-3.62 (m, 12H), 3.43 (s, 2H), 2.64- 2.55 (m, 2H), 2.50-2.42 (m, 2H), 1.73- 1.83 (m, 2H), 1.50-1.43 (m, 2H), 1.38 (s, 6H) |
| 32 | 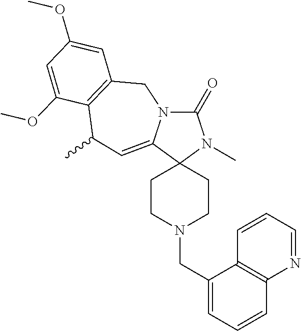 | 819688 Salt free | NMR ¹H (400 MHz, DMSO) δ 8.88-8.87 (m, 1H), 8.70-8.68 (m, 1H), 7.93-7.91 (m, 1H), 7.69-7.65 (m, 1H), 7.56-7.53 (m, 2H), 6.48-6.47 (m, 1H), 6.38-36.37 (m, 1H), 5.07 (d, J = 9.1 Hz, 1H), 4.65-4.48 (m, 2H), 4.08-4.04 (m, 1H), 3.99 (s, 2H), 3.75 (s, 3H), 3.71 (s, 3H), 2.75-2.58 (m, 7H), 1.89-1.80 (m, 2H), 1.54-1.51 (m, 1H), 1.37-1.34 (m, 1H), 1.27 (d, J = 7.3 Hz, 3H) |

### Biological Examples

HEKT-bet-luc assay: This assay measures a T-bet dependent reporter (luciferase) activity in engineered HEK cells that express a human T-bet and a T-box responsive element driving luciferase reporter. HEKT-bet cells were plated at 2×104/well in 96-well plate and compound was added into cell culture for 24 hours. Luciferase activity was measured by adding 50 μl of Steady-Glo reagent (Promega) and samples were read in Victor V reader (PerkinElmer). The activity of compound was determined by comparing compound treated samples to non-compound treated vehicle controls. The IC₅₀ values were calculated utilizing a maximum value corresponding to the amount of luciferase in the absence of a test compound and a minimum value corresponding to a test compound value obtained at maximum inhibition.

Determination of Normalized HEKT-bet IC50 values: Compounds were assayed in microtiter plates. Each plate included a reference compound which was ER-819544. The un-normalized IC₅₀ value for a particular compound was divided by the IC₅₀ value determined for the reference compound in the same microtiter plate to provide a relative potency value. The relative potency value was then multiplied by the established potency of the reference compound to provide the normalized HEKT-bet IC₅₀ value. In this assay, the established potency for ER-819544 was 0.035 μM. The IC₅₀ values provided herein were obtained using this normalization method.

Exemplary compounds of the present invention were assayed according to the methods set forth above in the HEKT-bet-luc assay described above. Tables 1 and 2 below set forth exemplary compounds of the present invention having an IC₅₀ of up to 5.0 μM as determined by the normalized HEKT-bet-luc assay described above.

TABLE 2
IC₅₀ Values of Exemplary Compounds

| Compound # | Structure | ER-Number | IC₅₀ (μm) |
|---|---|---|---|
| 1 | 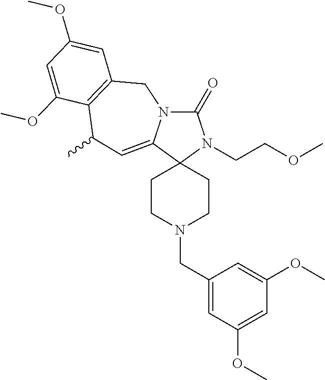 | 819543 | 0.015 |
| 2 | 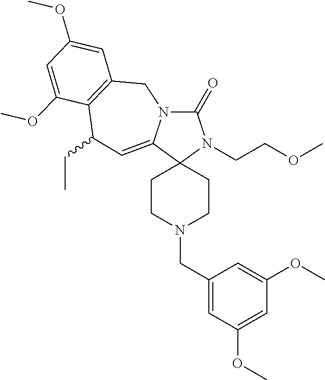 | 819549 | 0.015 |
| 3 | 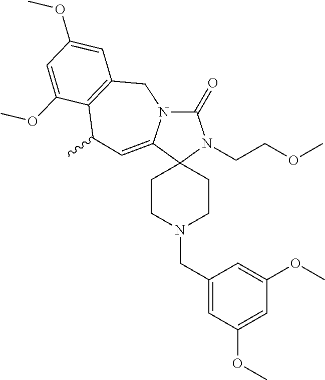 | 819543 | 0.015 |
| 4 | 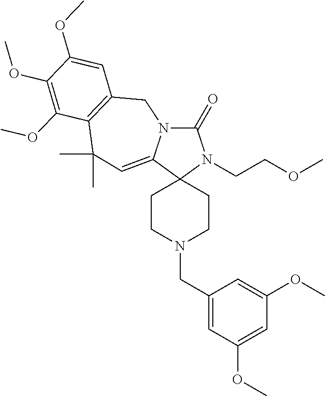 | 819701 | 0.021 |
| 5 | 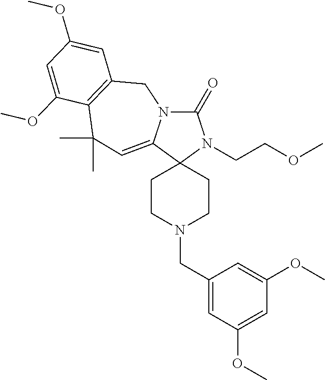 | 819544 | 0.035 |
| 6 | 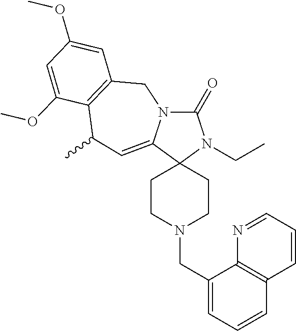 | 819594 | 0.060 |
| 7 | 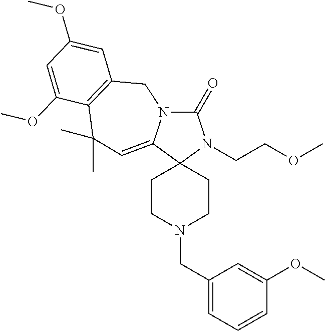 | 819647 | 0.064 |
| 8 | 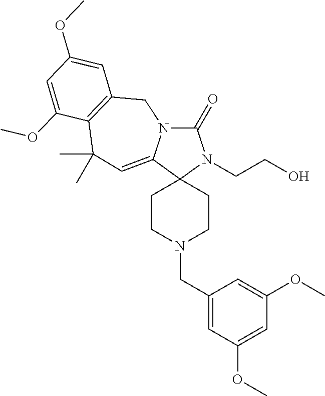 | 819657 | 0.065 |
| 9 | 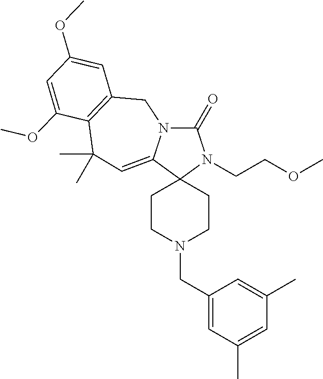 | 819659 | 0.068 |
| 10 | 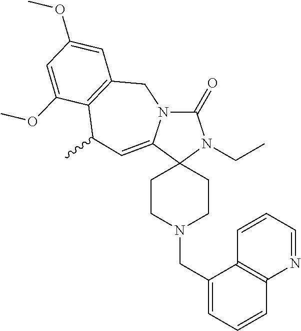 | 819592 | 0.086 |
| 11 | 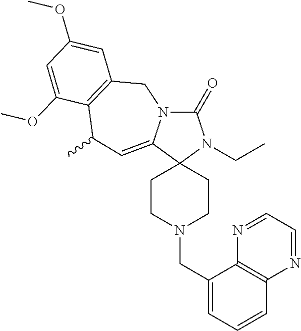 | 819595 | 0.090 |
| 12 THIS IS THE RACEMATE OF 819762 | 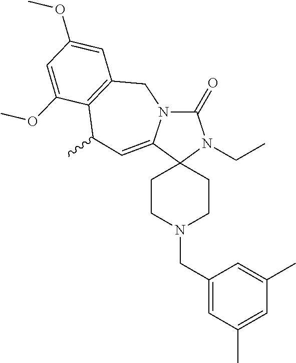 | 819597 | 0.090 |
| 13 | 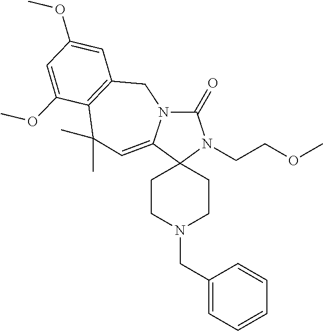 | 819641 | 0.098 |
| 14 | 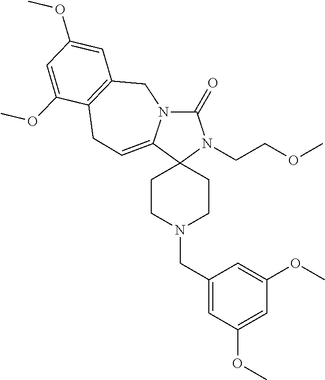 | 819673 | 0.102 |
| 15 | 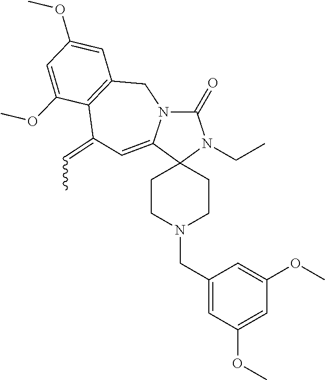 | 819651 | 0.110 |
| 16 | 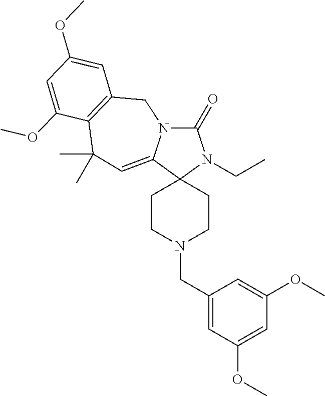 | 819583 | 0.112 |
| 17 | 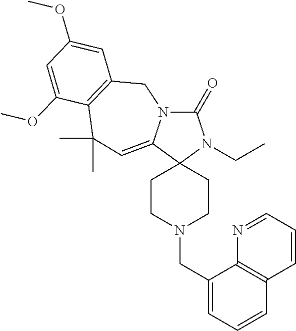 | 819604 | 0.120 |
| 18 | 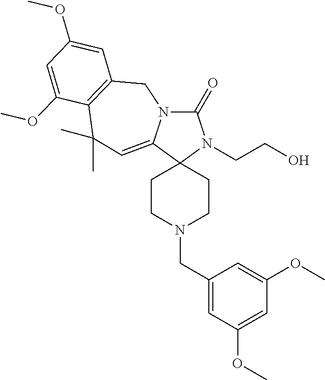 | 819657 | 0.124 |
| 19 | 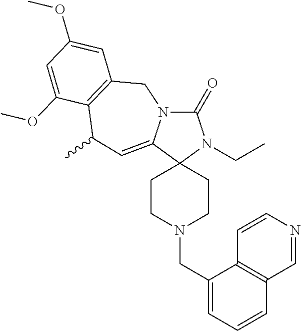 | 819593 | 0.140 |
| 20 | 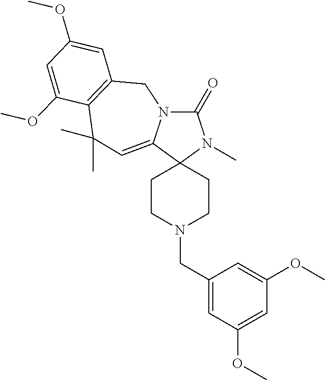 | 819658 | 0.141 |
| 21 | 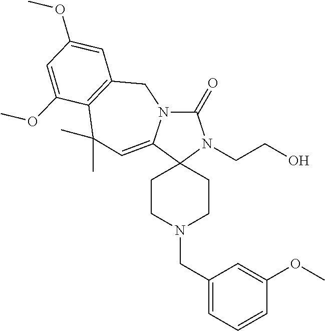 | 819648 | 0.147 |
| 22 | 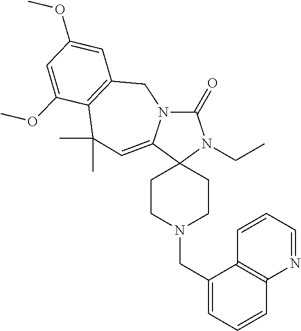 | 819602 | 0.150 |
| 23 | 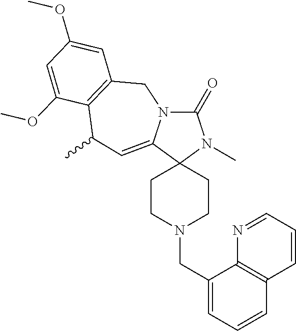 | 819689 | 0.169 |
| 24 | 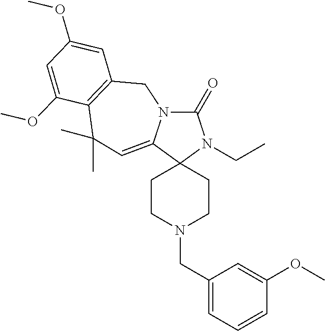 | 819646 | 0.184 |
| 25 | 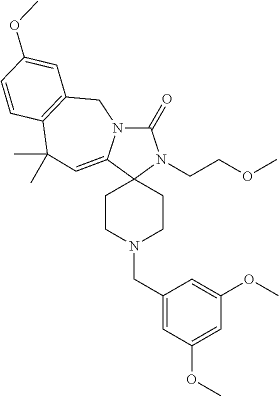 | 819655 | 0.204 |
| 26 | 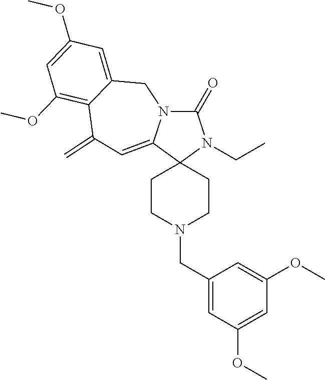 | 819703 | 0.247 |
| 27 | 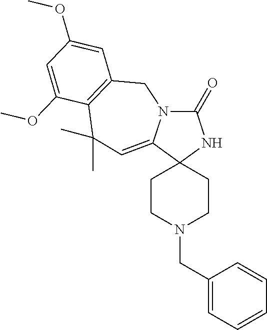 | 819667 | 0.250 |
| 28 | 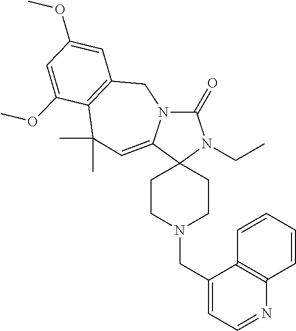 | 819601 | 0.260 |
| 29 | 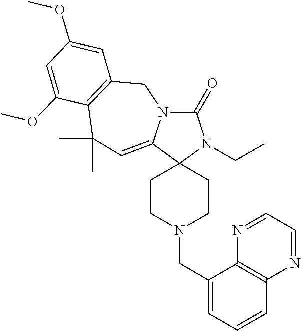 | 819605 | 0.260 |
| 30 | 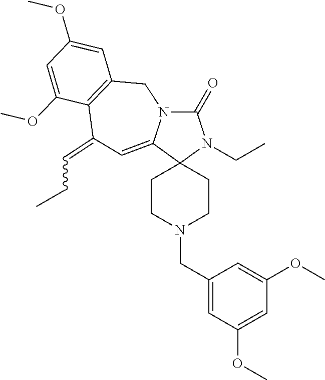 | 819652 | 0.270 |
| 31 | 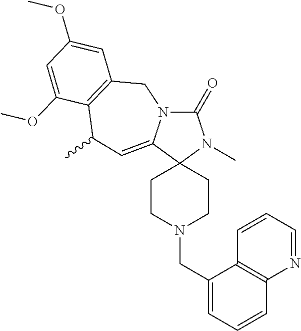 | 819688 | 0.288 |
| 32 | 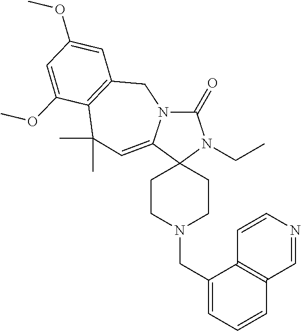 | 819603 | 0.340 |
| 33 | 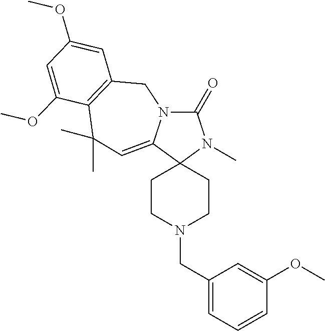 | 819628 | 0.360 |
| 34 | 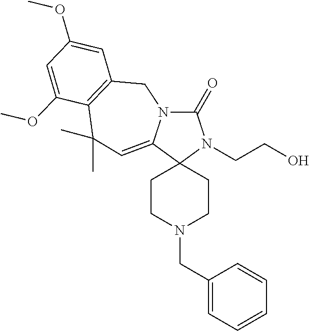 | 819642 | 0.365 |
| 35 | 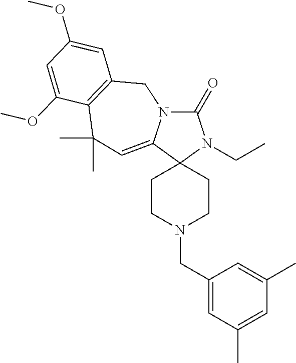 | 819607 | 0.500 |
| 36 | 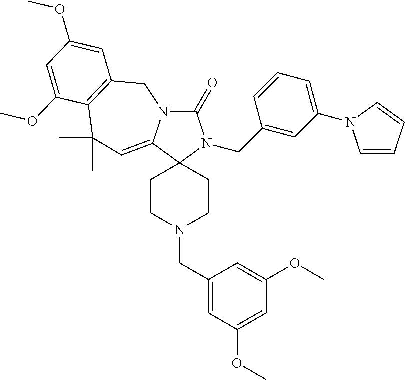 | 819590 | 0.514 |
| 37 | 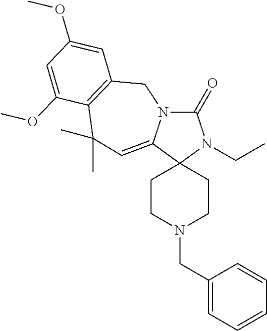 | 819640 | 0.542 |
| 38 | 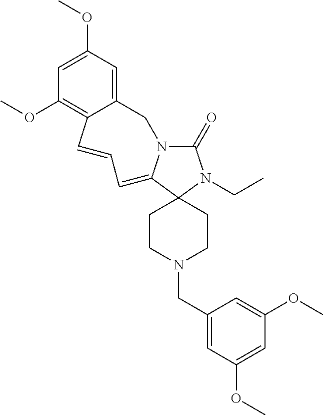 | 819702 | 0.600 |
| 39 | 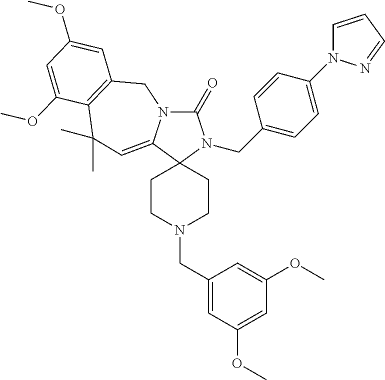 | 819663 | 0.637 |
| 40 | 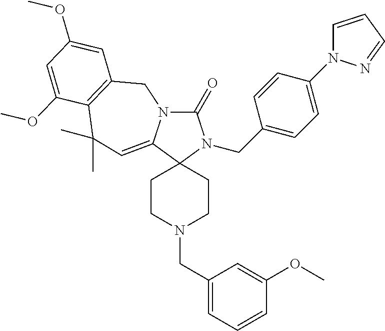 | 819650 | 0.669 |
| 41 | 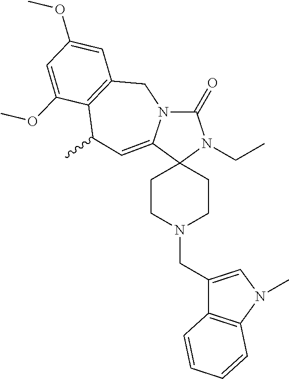 | 819596 | 0.720 |
| 42 | 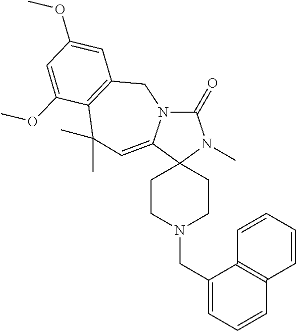 | 819637 | 0.734 |
| 43 | 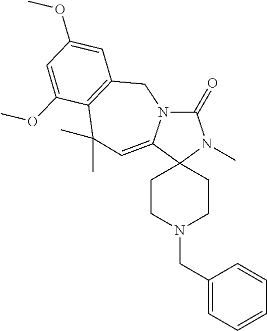 | 819629 | 0.840 |
| 44 | 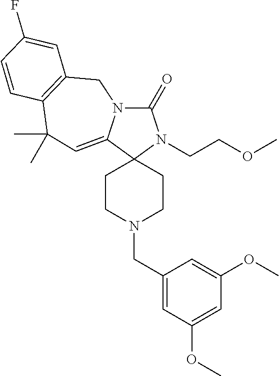 | 819672 | 0.877 |
| 45 | 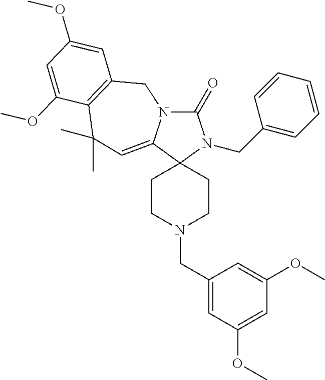 | 819662 | 0.898 |
| 46 | 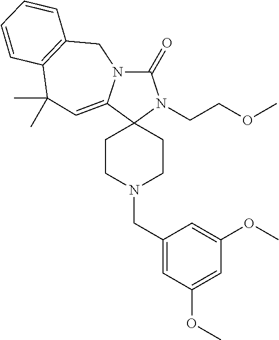 | 819677 | 1.024 |
| 47 | 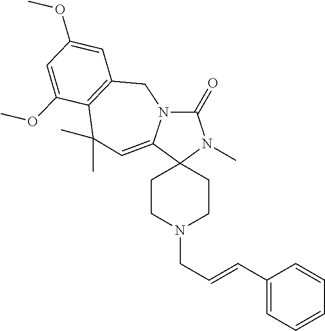 | 819634 | 1.150 |
| 48 | 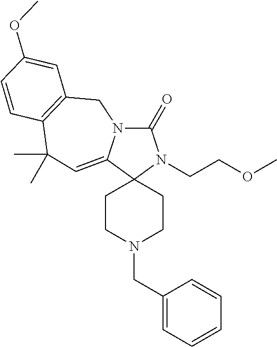 | 819613 | 1.310 |
| 49 | 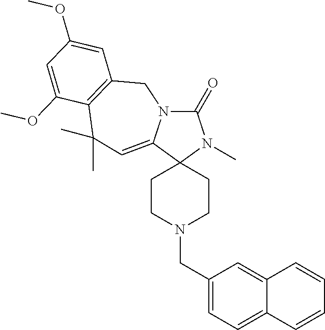 | 819627 | 1.600 |
| 50 | 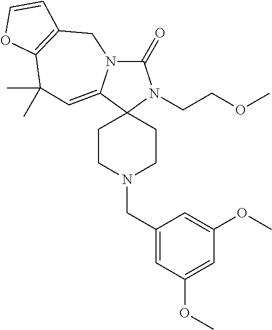 | 819698 | 1.983 |
| 51 | 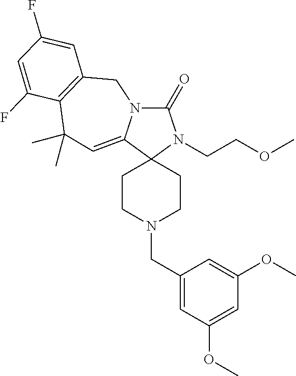 | 819704 | 2.759 |
| 52 | 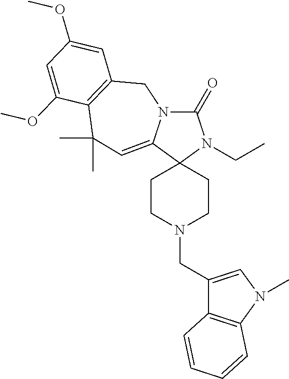 | 819606 | 2.870 |
| 53 | 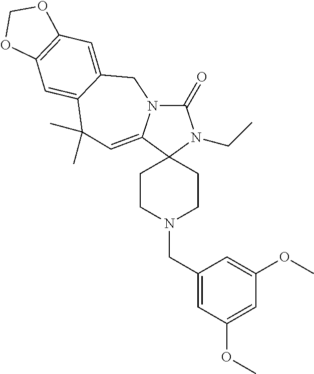 | 819708 | 3.599 |
| 54 | 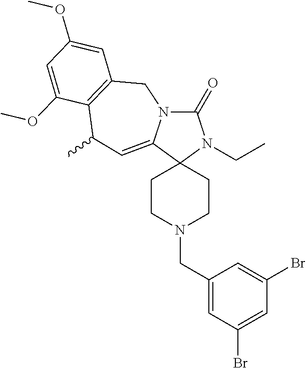 | 819599 | 4.710 |
| 55 | 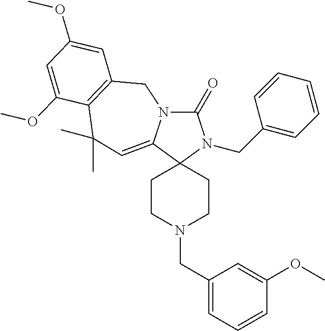 | 819649 | 4.945 |
| 56 | 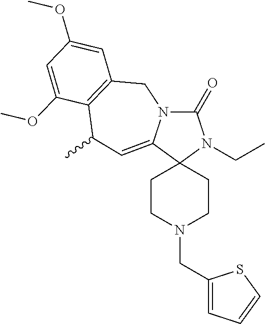 | 819556 | 0.166 |
| 57 | 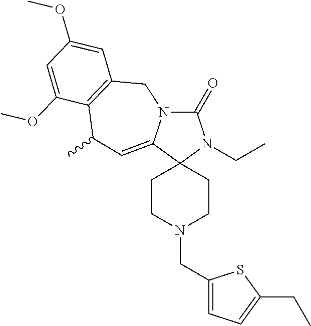 | 819557 | 0.51 |
| 58 | 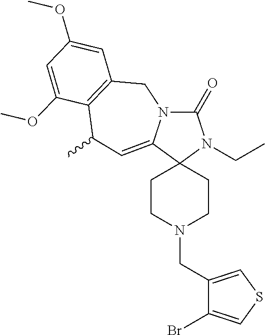 | 819558 | 0.74 |
| 59 | 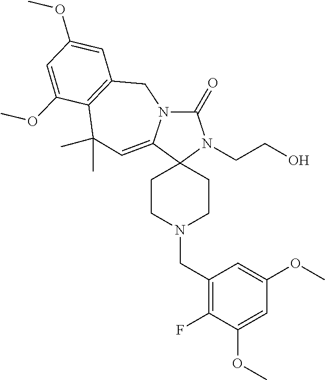 | 819724 | 0.104 |
| 60 | 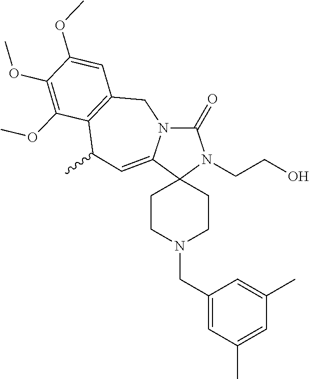 | 819735 | 0.140 |
| 61 | 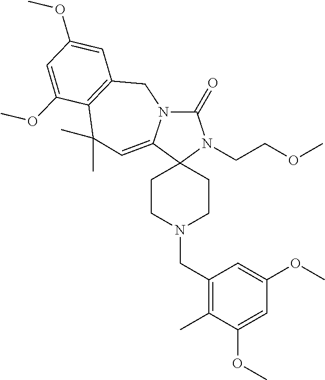 | 819749 | 0.044 |
| 62 | 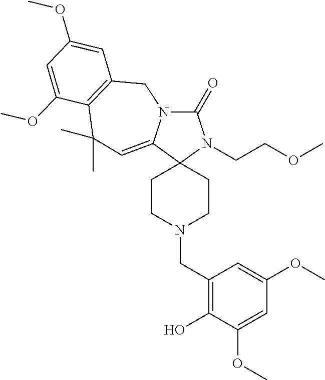 | 819750 | 0.041 |
| 63 | 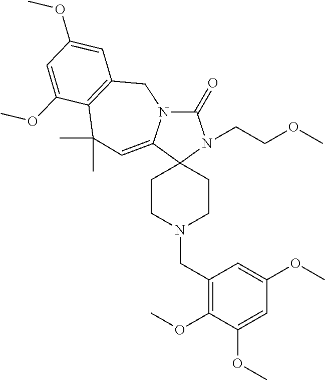 | 819752 | 0.071 |
| 64 | 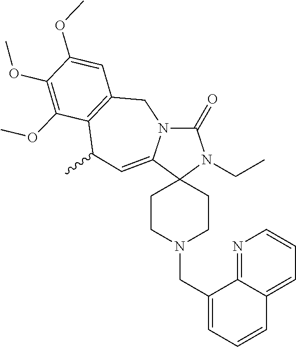 | 819755 | 0.053 |
| 65 | 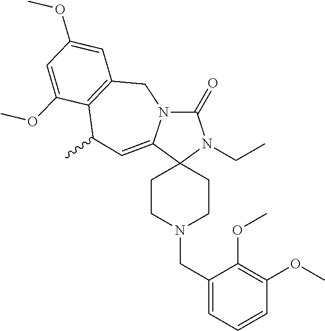 | 819767 | 0.148 |
| 66 | 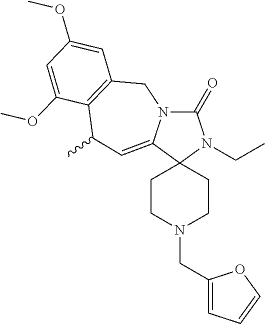 | 819768 | 0.183 |
| 67 | 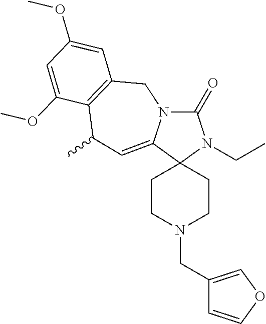 | 819769 | 0.190 |
| 68 | 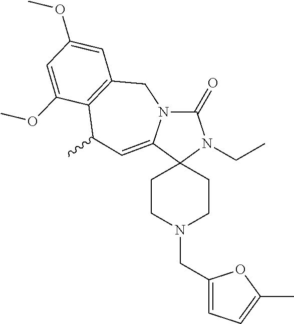 | 819770 | 0.267 |
| 69 | 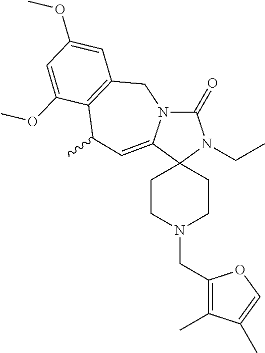 | 819771 | 0.205 |
| 70 | 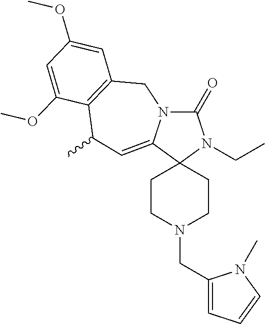 | 819772 | 0.103 |

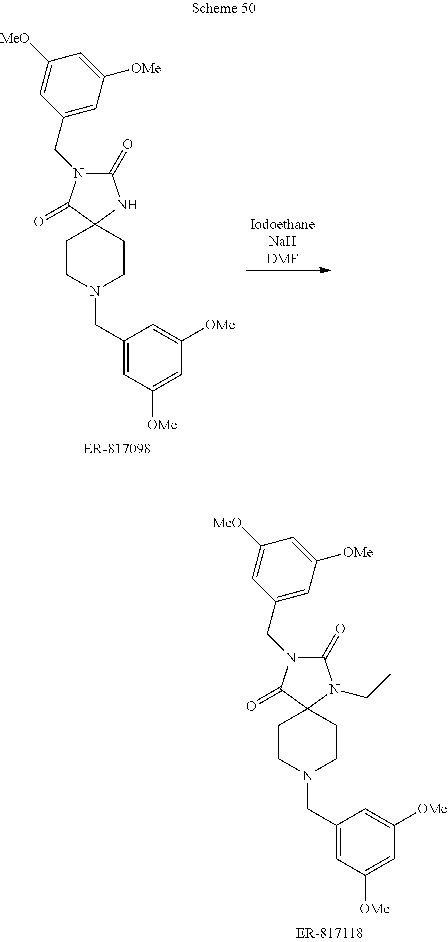

ER-817118: ER-817098 was prepared according to Scheme 1-4. As depicted in Scheme 50 above, to a solution of ER-817098 (2.85 g, 0.00607 mol), in N,N-dimethylformamide (15 mL) was added sodium hydride (364 mg, 0.00910 mol) followed by iodoethane (758 μL, 0.00910 mol). The reaction mixture was stirred overnight. Water was very slowly added and the reaction mixture was extracted several times with MTBE. The MTBE extracts were combined and washed with water (2×) and brine (1×). The organic layer was dried over magnesium sulfate, filtered, and concentrated in vacuo. Flash chromatography using ethyl acetate as eluent provided ER-817118 (2.89 g, 96%) as a colorless oil.

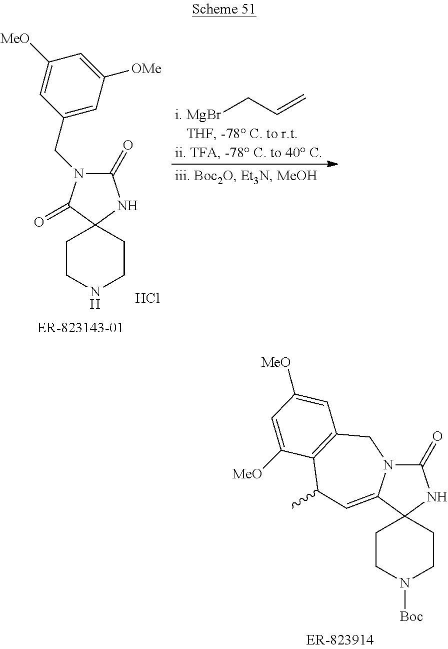

ER-823914: As depicted in Scheme 51 above, to a solution of ER-823143-01 (5.03 g, 0.0141 mol) in tetrahydrofuran (30.0 mL, 0.370 mol) at −78° C. was slowly added 1.0 M of allylmagnesium bromide in ether (71 mL). The reaction mixture was warmed to room temperature and stirred overnight. The reaction mixture was cooled to −78° C., treated dropwise with trifluoroacetic acid (21.8 mL, 0.283 mol), and then concentrated in vacuo to a small residual volume. Triethylamine was added to neutralize residual TFA and the mixture then concentrated in vacuo to dryness. The residual red oil was dissolved in methanol (138 mL, 3.41 mol) and treated with di-tert-butyldicarbonate (3.34 g, 0.0148 mol) followed by triethylamine (2.38 mL, 0.0169 mol) and stirred overnight at room temperature. The reaction mixture was concentrated in vacuo and purified by flash chromatography (eluent: 50% hexanes in ethyl acetate) to provide ER-823914 (3.25 g, 52%) as a colorless solid.

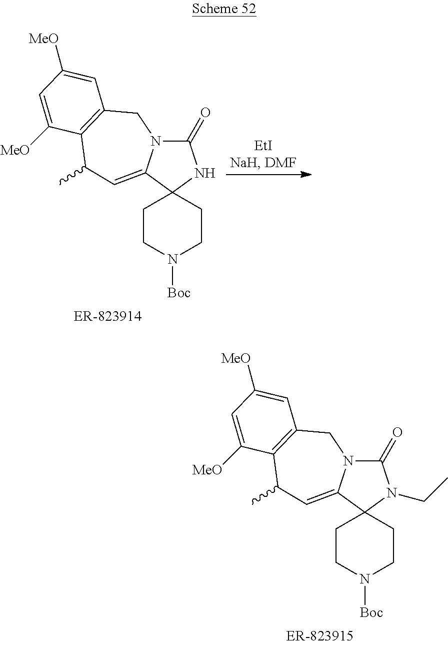

ER-823915: As depicted in Scheme 52 above, to a solution of ER-823914 (2.20 g, 0.00496 mol) in N,N-Dimethylformamide (12.4 mL, 0.160 mol) was added sodium hydride (298 mg, 0.00744 mol) followed by iodoethane (607 μL, 0.00744 mol). The reaction mixture was stirred overnight then quenched with water and extracted several times with MTBE. The MTBE extracts were combined and washed with water and brine. The organic layer was dried over magnesium sulfate, filtered, and concentrated in vacuo. Flash chromatography (eluent: 40% hexanes in ethyl acetate) provided ER-823915 (0.80 g, 34%) as a colorless foam.

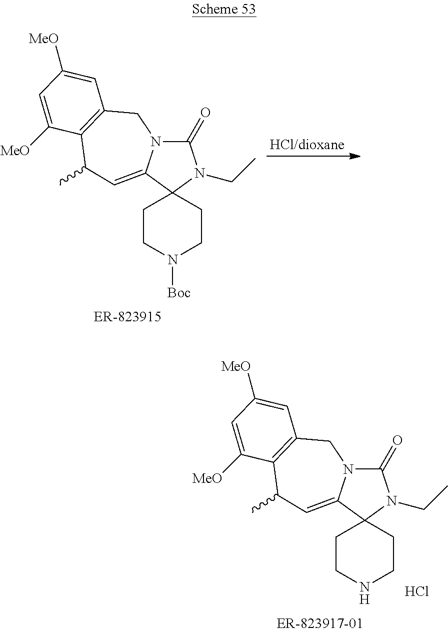

ER-823917-01: As depicted in Scheme 53 above, ER-823915 (799.2 mg, 0.001695 mol) was dissolved in a solution of 4 M hydrogen chloride in 1,4-dioxane (10 mL). The reaction mixture was stirred overnight and then concentrated in vacuo to provide ER-823917-01 (0.69 g, quantitative) as an orange solid.

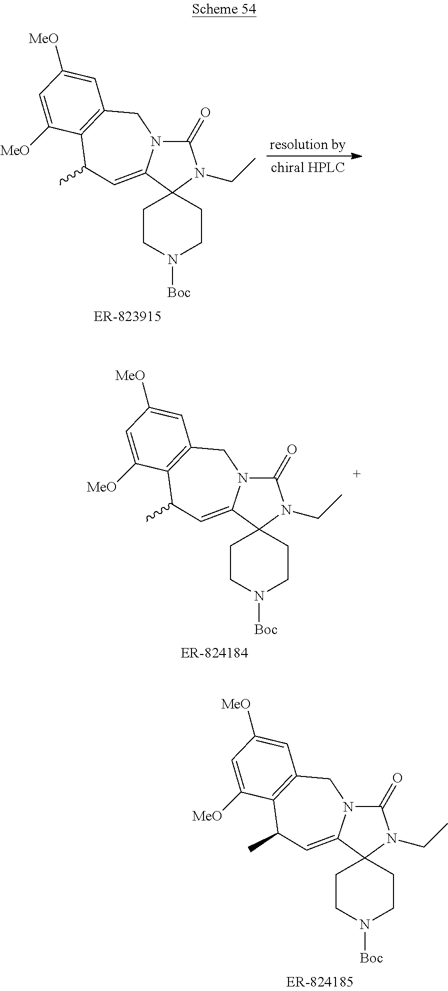

ER-824184 & ER-824185: As depicted in Scheme 55 above, a solution of ER-823915 (200 mg) in acetonitrile (1 ml) was injected onto a CHIRALPAK® AS-H SFC column (30 mm×250 mm, 5 micron particle size) and eluted with 95:5 n-heptane:i-propanol at a flow rate of 40 ml/min. Eluted fractions were detected using a UV detector with the wavelength set at 290 nm. The first eluting fraction was isolated and concentrated by rotary evaporation in vacuo to afford ER-824184; the second eluting fraction was isolated and concentrated by rotary evaporation in vacuo to afford ER-824185.

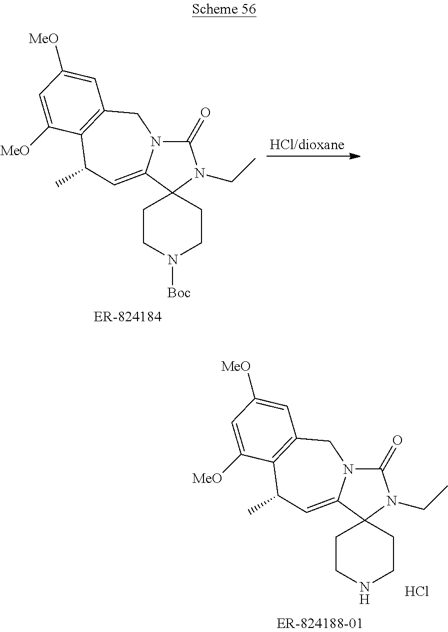

ER-824188-01: As depicted in Scheme 56 above, ER-824184 (25.33 g, 0.05371 mol) was dissolved in a solution of 4 M hydrogen chloride in 1,4-dioxane (135 mL). The reaction mixture was stirred overnight and then concentrated in vacuo to provide ER-824188-01 (21.9 g, quantitative) as an orange solid. Single crystal X-ray diffraction analysis of ER-824188-01 showed the absolute configuration of the stereocenter to be S, as depicted in Scheme 56.

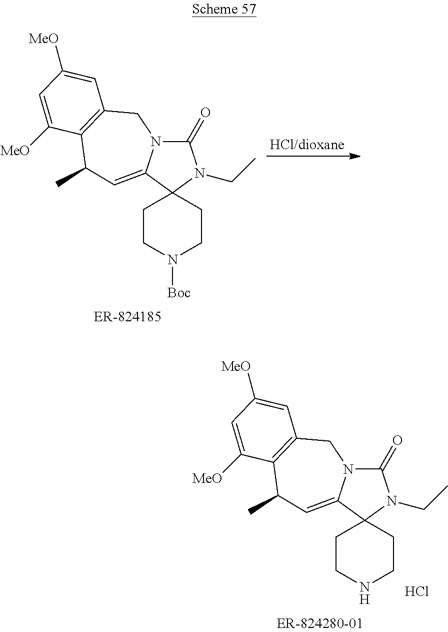

ER-824280-01: As depicted in Scheme 57 above, ER-824185 (457.2 mg, 0.0009695 mol) was dissolved in a solution of 4 M hydrogen chloride in 1,4-dioxane (2.5 mL). The reaction mixture was stirred overnight and then concentrated in vacuo to provide ER-824280-01 (383.2 mg, 97%) as an orange solid. Single crystal X-ray diffraction analysis of a Mosher amide derivative of ER-824188-01 showed the absolute configuration of the stereocenter to be R, as depicted in Scheme 56.

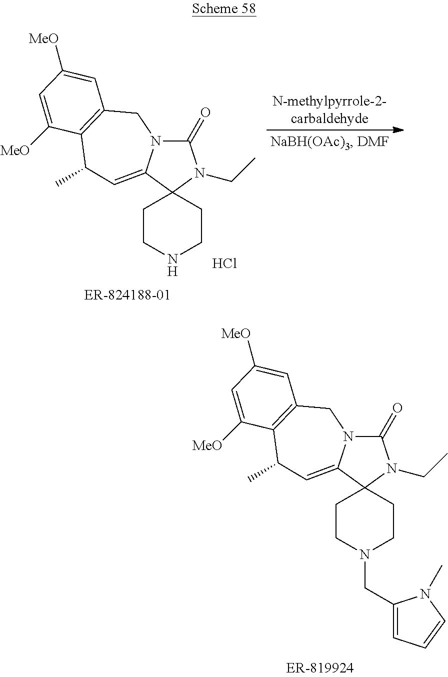

ER-819924: As depicted in Scheme 58 above, ER-824188-01 (62.4 mg, 0.000153 mol) and N-methylpyrrole-2-carbaldehyde (0.000229 mol) were dissolved/suspended in N,N-dimethylformamide (0.62 mL). After stirring for 30 minutes, sodium triacetoxyborohydride (47.8 mg, 0.000214 mol) was added. The reaction mixture was stirred overnight then purified by reverse phase chromatography to afford ER-819924 (71.1 mg, 83.4%) as an oil.

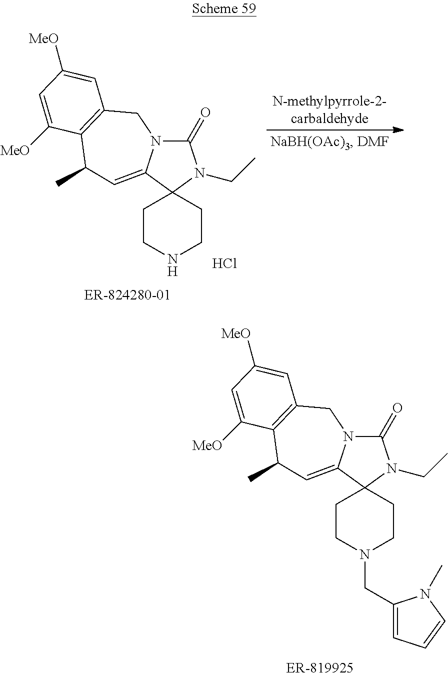

ER-819925: As depicted in Scheme 59 above, ER-824280-01 (59.5 mg, 0.000146 mol and N-methylpyrrole-2-carbaldehyde (0.000219 mol) were dissolved/suspended in N,N′-dimethylformamide (0.60 mL). After stirring for 30 minutes, sodium triacetoxyborohydride (45.6 mg, 0.000204 mol) was added. The reaction mixture was stirred overnight then purified by reverse phase chromatography to afford ER-819925 (51.9 mg, 76.6%) as an oil.

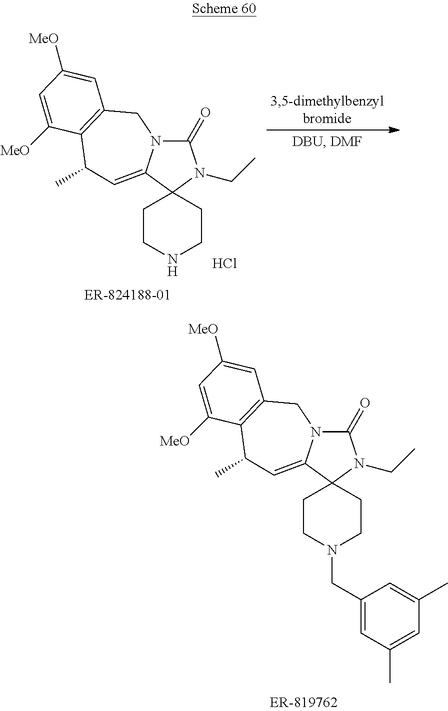

ER-819762: As depicted in Scheme 61 above, a solution of ER-824188-01 (5.7 g, 0.0140 mol), 1,8-diazabicyclo[5.4.0]undec-7-ene (4.4 mL, 0.029 mol) and 3,5-dimethylbenzyl bromide (4.7 g, 0.024 mol) in N,N-dimethylformamide (50 mL) was heated at 97 C overnight. An aqueous work-up and purification by flash chromatography provided ER-819762 (4.86 g, 71%) as colorless solid.

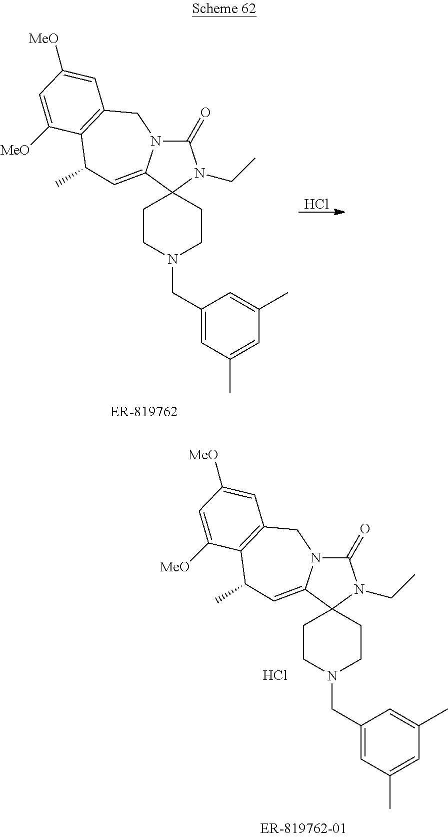

ER-819762-01: As depicted in Scheme 62 above, a solution of ER-819762 (4.77 g, 0.00974 mol), Acetonitrile (10 mL) and 1M HCl in Water (11 mL) was stirred at room temperature for approximately 5 minutes. The solution was concentrated to provide ER-819762-01 (5.1 g, quantitative) as a colorless crystalline solid after lyophilization. Single crystal X-ray diffraction analysis of ER-819762-01 showed the absolute configuration of the stereocenter to be S, as depicted in Scheme 62.

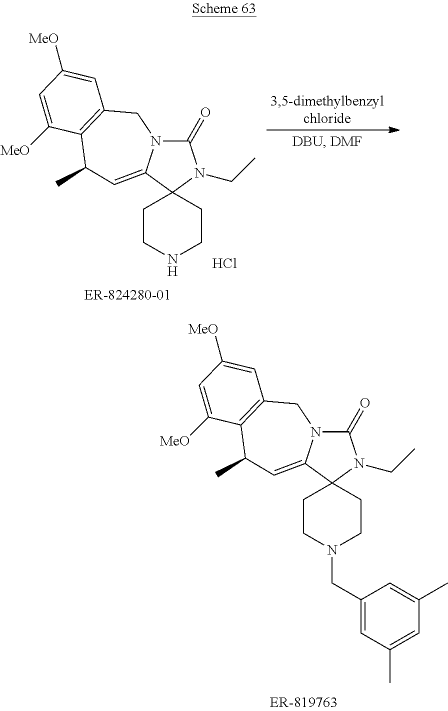

ER-819763: As depicted in Scheme 63 above, a solution of ER-824280-01 (66.9 g, 0.1640 mol), 1,8-diazabicyclo[5.4.0]undec-7-ene (54 mL, 0.361 mol) and 3,5-dimethylbenzyl chloride (42.4 g, 0.213 mol) in N-Methylpyrrolidinone (669 mL) was heated at 72 C for 2 hours. After cooling, water was added to precipitate the desired product. Filtration and drying under vacuum provided ER-819763 (74.4 g, 92%) as colorless solid.

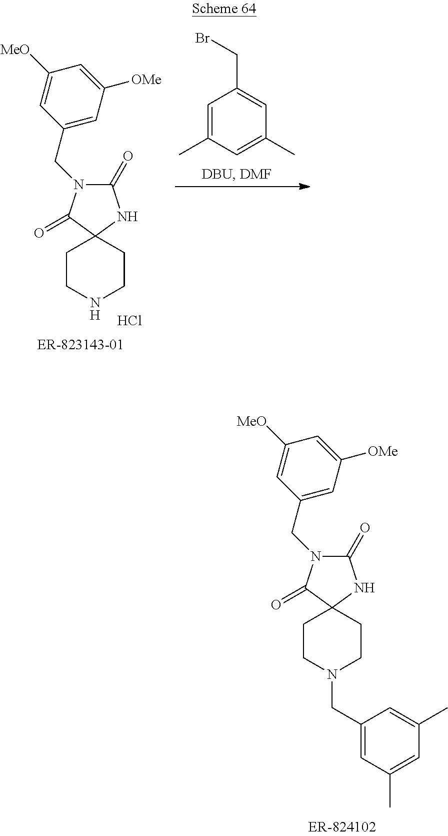

ER-824102: As depicted in Scheme 64 above, to a solution of ER-823143-01 (4.00 g, 0.0112 mol) in N,N-dimethylformamide (25 mL) at room temperature was added alpha-bromomesitylene (3.13 g, 0.0157 mol) followed by DBU (4.37 mL, 0.0292 mol). After stirring for 1 hour, reaction was quenched with half-saturated aq. NH4Cl, diluted with ethyl acetate, and stirred for 1 h to give two clear layers. Organic layer was separated, aq. layer was extracted with ethyl acetate (2×). Combined extracts were dried over Na2SO4, filtered, and concentrated in vacuo. Crystallization from MTBE afforded ER-824102 (4.30 g, 87%) as a colorless solid. (BMS-206)

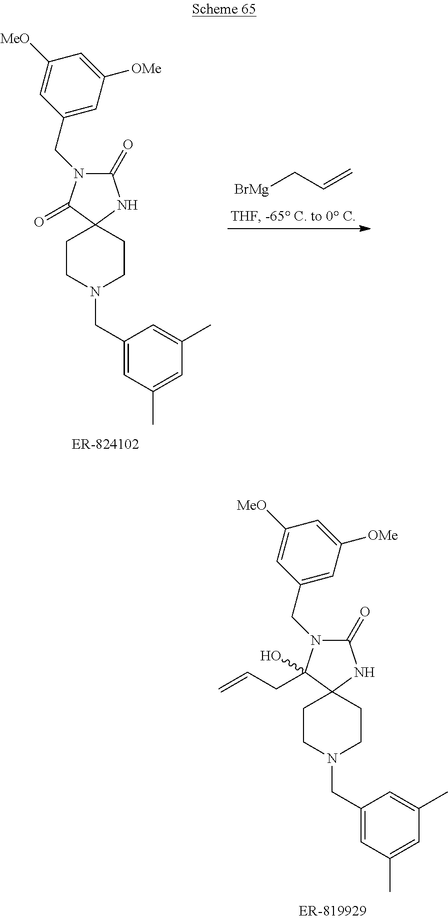

ER-819929: As depicted in Scheme 65 above, to a solution of ER-824102 (3.72 g, 0.0085 mol) in tetrahydrofuran (35 mL) at −65° C. was added 1.0 M allylmagnesium bromide in ether (25.5 mL, 0.0255 mol) over 10 min keeping internal temperature below −50° C. The reaction mixture was allowed to warm to 0° C. After 3 h at 0° C., reaction was quenched with saturated aq. NH4Cl, diluted with ethyl acetate and water, stirred for 10 min to give two clear layers. Organic layer was separated, aq. layer was extracted with ethyl acetate. Combined extracts were washed with water, brine, dried over Na2SO4, filtered, concentrated in vacuo to give crude product ER-819929 (4.15 g, quantitative) as a colorless solid that was used for next step without further purification. (BMS-211)

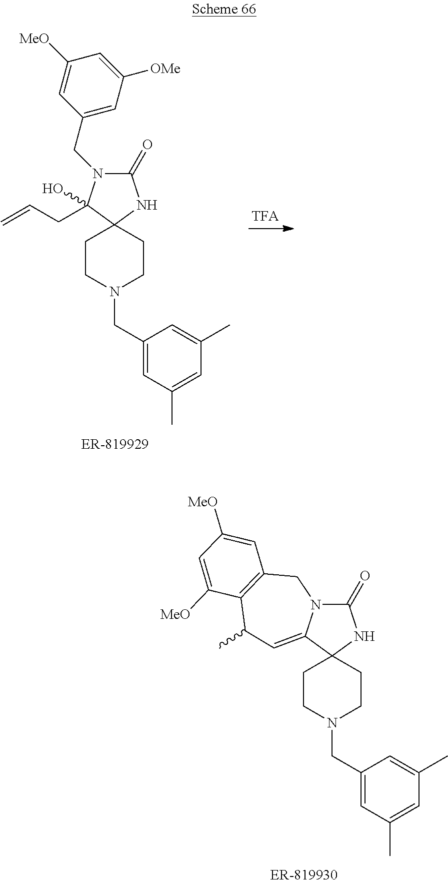

ER-819930: As depicted in Scheme 66 above, a solution of ER-819929 (37 mg, 0.000077 mol) in trifluoroacetic acid (0.5 mL) was stirred at room temperature for 16 hours. Dark brown-red reaction mixture was diluted with EtOAc (5 mL), neutralized with sat aq NaHCO3 (5 mL, careful: gas evolution). Two-layer mixture was stirred for 10 min to give two clear, almost colorless layers. The organic layer was separated; the aq layer was extracted with EtOAc. Combined organic extracts were dried over Na2SO4, filtered, concentrated in vacuo. Purification by flash chromatography eluting with 1:1 Heptane-EtOAc, 1:3 Heptane-EtOAc, 100% EtOAc afforded ER-819930 (26 mg, 73%) as a colorless solid. (BMS-209)

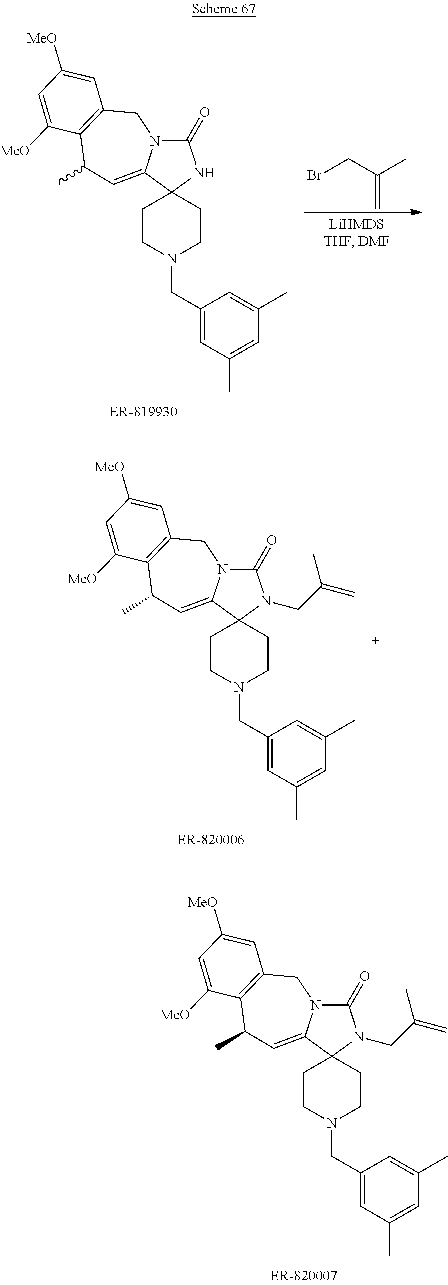

ER-820006 and ER-820007: As depicted in Scheme 67 above, to a solution of ER-819930 (110 mg, 0.000238 mol) and methallyl bromide (72 μL, 0.000715 mol) in DMF (1.5 mL) was added 1.0 M lithium hexamethyldisilazide solution in tetrahydrofuran (0.52 mL, 0.00052 mol). After stirring for 18 h at rt, reaction mixture was diluted with MTBE, quenched with half-saturated aq NH4Cl. Aq. layer was separated, extracted with MTBE. Combined extracts were dried over Na₂SO4, filtered, concentrated in vacuo. Purification by flash chromatography eluting with 3:2 Heptane-EtOAc, 1:1 Heptane-EtOAc furnished racemic product (68 mg, 55%) as a colorless oil. Racemic product (55 mg) was subjected to chiral HPLC on Chiralpak AS column eluting with heptane-isopropanol (9:1) to afford first eluting enantiomer ER-820006 (21 mg, 38%, [α]_{D}=+83.7° (c=0.35, CHCl3) and second eluting enantiomer ER-820007 (23 mg, 42%, [α]_{D}=−74.2° (c=0.38, CHCl3). Absolute stereochemistry was assigned tentatively based on analogy in optical rotation and chiral HPLC retention time with ER-819762/ER-819763 pair of enantiomers. (BMS-232, 242)

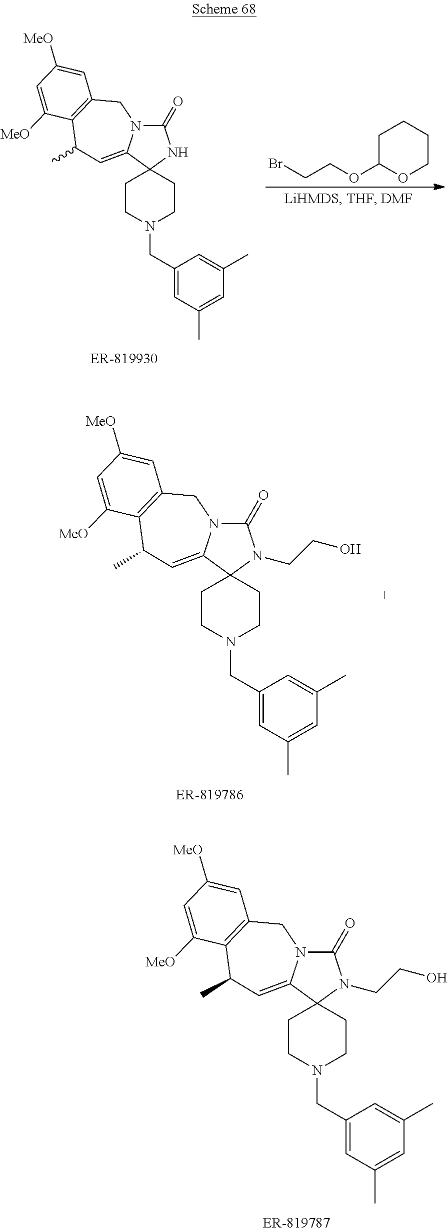

ER-819786 and ER-819787: As depicted in Scheme 68 above, a 5 mL microwave reactor vial equipped with a stir bar was charged with ER-819930 (110 mg, 0.000238 mol), DMF (1.5 mL), 2-(2-bromoethoxy)tetrahydro-2H-pyran (108 μL, 0.000715 mol) and 1.00 M of lithium hexamethyldisilazide in tetrahydrofuran (520 0.00052 mol). The reactor vial was microwaved at 200° C. for 15 min. More 2-(2-bromoethoxy)tetrahydro-2H-pyran (108 μL, 0.000715 mol) and 1.00 M of lithium hexamethyldisilazide in tetrahydrofuran (520 μL, 0.00052 mol) were added, and reaction mixture was heated by microwave irradiation at 200° C. for another 15 min. Purification by preparative reverse phase HPLC provided racemic product (25 mg, 21%) as a colorless glassy oil. Racemic product (17 mg) was subjected to chiral HPLC on Chiralpac AS column eluting with heptane-isopropanol (9:1) to afford first eluting enantiomer ER-819786 (7.2 mg, 42%, [α]_{D}=+72.0° (c=0.1, CHCl3) and second eluting enantiomer ER-819787 (7.5 mg, 44%, [α]_{D}=−73.0° (c=0.1, CHCl3). Absolute stereochemistry was assigned tentatively based on analogy in optical rotation and chiral HPLC retention time with ER-819762/ER-819763 pair of enantiomers. (BMS-230, 247)

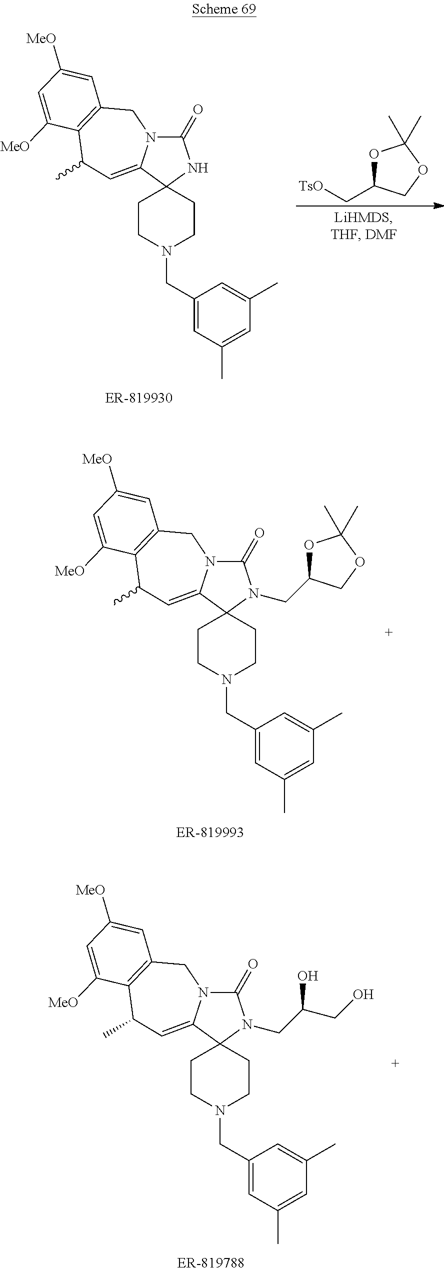

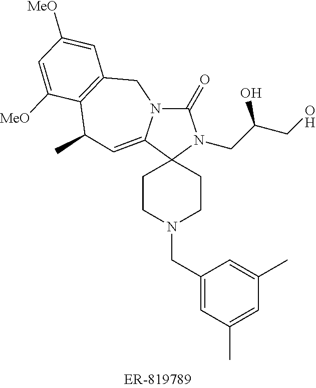

ER-819993 and ER-819994: As depicted in Scheme 69 above, a 5 mL microwave reactor vial equipped with a stir bar was charged with ER-819930 (110 mg, 0.000238 mol), DMF (1.5 mL), ((4S)-2,2-dimethyl-1,3-dioxolan-4-yl)methyl 4-methylbenzenesulfonate (205 mg, 0.000715 mol) and 1.00 M of lithium hexamethyldisilazide in tetrahydrofuran (520 μL, 0.00052 mol). The reactor vial was heated by microwave irradiation at 200° C. for 15 min. More ((4S)-2,2-dimethyl-1,3-dioxolan-4-yl)methyl 4-methylbenzenesulfonate (157 mg, 0.000548 mol) and 1.00 M of lithium hexamethyldisilazide in tetrahydrofuran (477 μL, 0.000477 mol) were added, and reaction mixture was heated by microwave irradiation at 200° C. for another 15 min. Purification by preparative reverse phase HPLC provided acetonide ER-819993 (40 mg, 30%) and diol material (18 mg, 14%) as 1:1 mixtures of diastereomers. Separation of diastereomeric diols by chiral HPLC on Chiralpac AS column eluting with heptane-isopropanol (9:1) afforded the first eluting diastereomer ER-819788 (5.0 mg) and the second eluting diastereomer ER-819789 (5.2 mg). Absolute stereochemistry was assigned tentatively based on analogy in chiral HPLC retention time with ER-819762/ER-819763 pair of enantiomers. (BMS-231, 249)

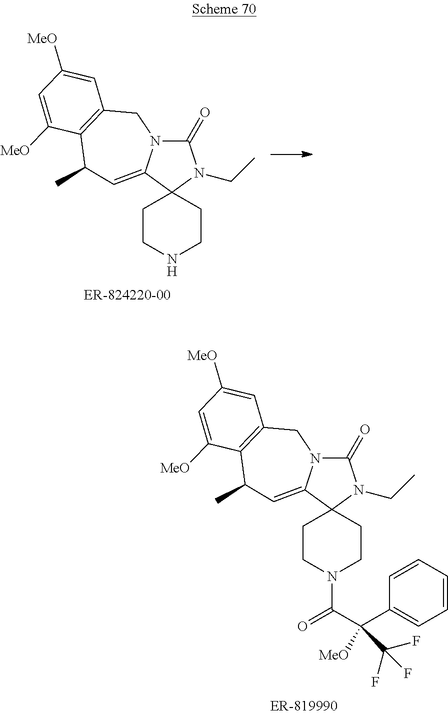

ER-81990: As depicted in Scheme 70 above, a solution of ER-824220-00 (51.8 mg, 0.000139 mol), triethylamine (97 μL, 0.00070 mol), 4-dimethylaminopyridine (3.4 mg, 0.000028 mol) and (R)-(−)-α-Methoxy-α-trifluoromethylphenylacetyl chloride (0.052 mL, 0.00028 mol) in Methylene Chloride (500 μL) was stirred at room temperature for 5 hours. Purification by flash chromatography, followed by crystallization from ethyl acetate/heptane/pentane provided ER-819990 (49.2 mg, 60%) as crystals.

TABLE 3
Analytical Data for Exemplary Compounds of Formula I

| Example # | Structure | ER-# | Analytical Data |
|---|---|---|---|
| 1 | 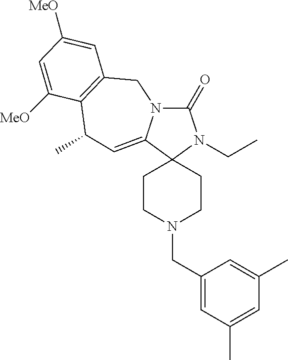 | 819762-01 HCl Salt | NMR ¹H (400 MHz, CD₃OD) δ 7.18 (s, 2H), 7.15 (s, 1H), 6.49 (d, J = 2.6 Hz, 1H), 6.42 (d, J = 2.3 Hz, 1H), 5.18 (br s, 1H), 4.70 (d, J = 14.4 Hz, 1H), 4.62 (d, J = 14.4 Hz, 1H), 4.32 (s, 2H), 4.27-4.19 (m, 1H), 3.80 (s, 3H), 3.78 (s, 3H), 3.50-3.38 (m, 4H), 3.26 (br s, 2H), 2.36 (s, 6H), 2.31-2.17 (m, 2H), 1.97 (br d, J = 14.4 Hz, 1H), 1.79 (br d, J = 14.4 Hz, 1H), 1.38 (d, J = 7.3 Hz, 3H), 1.16 (t, J = 7.0 Hz, 3H) M/Z (ES+) Calc.: 489.30 Found: 490.40 (M + H) Analytical HPLC: Method A1 SunFire MS C18 (4.6 × 100 mm) 5 um Retention Time: 11.21 min Chiral HPLC Method C1 CHIRALPAK IA (0.46 × 25 cm) Retention Time: 6.819 min |
| 2 | 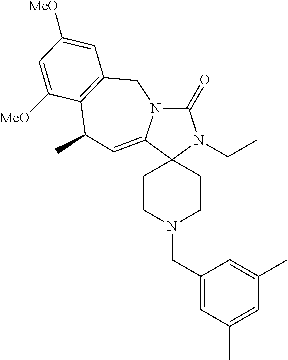 | 819763-00 Salt Free | NMR ¹H (400 MHz, CDCl₃) δ 6.94 (s, 2H), 6.90 (s, 1H), 6.44 (d, J = 2.6 Hz, 1H), 6.41 (d, J = 2.3 Hz, 1H), 5.02 (d, J = 8.5 Hz, 1H), 4.81 (d, J = 14.1 Hz, 1H), 4.58 (d, J = 14.4 Hz, 1H) 4.17-4.09 (m, 1H), 3.79 (s, 3H), 3.78 (s, 3H), 3.49 (s, 2H), 3.51-3.26 (m, 1H), 3.26-3.17 (m, 1H), 2.79-2.76 (m, 1H), 2.71-2.68 (m, 1H), 2.56-2.46 (m, 2H), 2.31 (s, 6H), 2.00-1.86 (m 2H), 1.68-1.58 (m, 2H), 1.35 (d, J = 7.3 Hz, 3H), 1.15 (t, J = 7.2 Hz, 3H) M/Z (ES+) Calc.: 489.30 Found: 490.40 (M + H) Analytical HPLC: Method A1 SunFire MS C18 (4.6 × 100 mm) 5 um Retention Time: 11.16 min Chiral HPLC Method C1 CHIRALPAK IA (0.46 × 25 cm) Retention Time: 4.786 min |
| 3 | 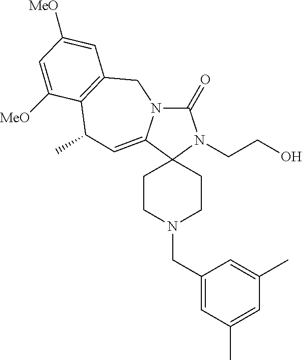 | 819786-01 HCl Salt | NMR ¹H (400 MHz, CDCl₃) δ 12.68 (br s, 1H), 7.25 (s, 2H), 7.10 (s, 1H), 6.42 (d, J = 2.4 Hz, 1H), 6.41 (d, J = 2.4 Hz, 1H), 4.79 (d, J = 14.4 Hz, 1H), 4.75 (d, J = 8.8 Hz, 1H), 4.61 (d, J = 14.4 Hz, 1H), 4.05-4.14 (m, 3H), 3.81 (s, 3H), 3.79 (s, 3H), 3.76 (m, 2H), 3.58 (m, 2H), 3.48 (d, J = 10.0 Hz, 1H), 3.35 (d, J = 10.8 Hz, 1H), 3.11 (q, J = 10 HZ, 2H), 2.82-3.00 (m, 2H), 2.37 (s, 6H), 1.89 (d, J = 14.2 Hz, 1H), 1.73 (d, 13.9 Hz, 1H), 1.34 (d, J = 7.2 Hz, 3H) M/Z (ES+) Calc.: 505.29 Found: 506.40 (M + H) Analytical HPLC: Method A1 SunFire MS C18 (4.6 × 100 mm) 5 um Retention Time: 9.88 min Chiral HPLC Method C1 CHIRALPAK IA (0.46 × 25 cm) Retention Time: 6.687 min |
| 4 | 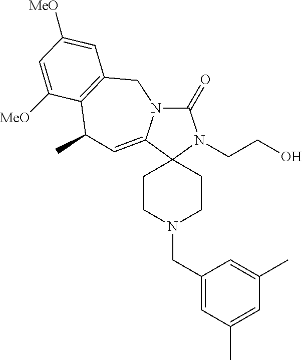 | 819787-01 HCl Salt | NMR ¹H (400 MHz, DMSO) δ 12.67 (br s, 1H), 7.27 (s, 2H), 7.11 (s, 1H), 6.43 (d, J = 2.4 Hz, 1H), 6.41 (d, J = 2.4 Hz, 1H), 4.75- 4.81 (m, 2H), 4.61 (d, J = 14.4 Hz, 1H), 4.10 (br s, 3H), 3.81 (s, 3H), 3.79 (s, 3H), 3.77 (m, 2H), 3.58 (br s, 2H), 3.49 (br s, 1H), 3.35 (br. s, 1H), 2.87-3.11 (m, 4H), 2.37 (s, 6H), 1.89 (br s, 1H), 1.73 (d, 11.2 Hz, 1H), 1.35 (d, J = 5.2 Hz, 3H) M/Z (ES+) Calc.: 505.29 Found: 506.40 (M + H) Analytical HPLC: Method A1 SunFire MS C18 (4.6 × 100 mm) 5 um Retention Time: 9.87 min Chiral HPLC Method C1 CHIRALPAK IA (0.46 × 25 cm) Retention Time: 4.435 min |
| 5 | 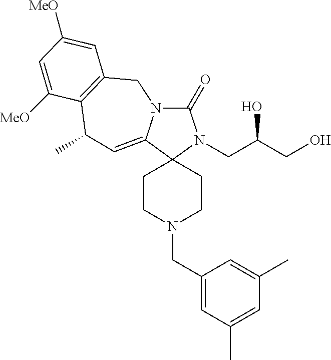 | 819788-01 HCl Salt | NMR¹H (400 MHz, CDCl₃) δ 12.29 (br s, 1H), 7.24 (s, 2H), 7.10 (s, 1H), 6.43 (d, J = 2.4 Hz, 1H), 6.41 (d, J = 2.4 Hz, 1H), 4.80 (d, J = 8.8 Hz, 1H), 4.78 (d, J = 14.4 Hz, 1H), 4.64 (d, J = 14.4 Hz, 1H), 4.04-4.16 (m, 3H), 3.81 (s, 3H), 3.79 (s, 3H), 3.77-3.85 (m, 2H), 3.61 (d, J = 5.6 Hz, 2H), 3.37- 3.54 (m, 3H), 2.91-3.11 (m, 4H), 2.36 (s, 6H), 1.92 (d, J = 11.6 Hz, 1H), 1.72 (d, 13.2 Hz, 1H), 1.37 (d, J = 7.3 Hz, 3H) M/Z (ES+) Calc.: 535.30 Found: 536.39 (M + H) Analytical HPLC: Method A1 SunFire MS C18 (4.6 × 100 mm) 5 um Retention Time: 9.27 min Chiral HPLC Method C1 CHIRALPAK IA (0.46 × 25 cm) Retention Time: 8.471 min |
| 6 | 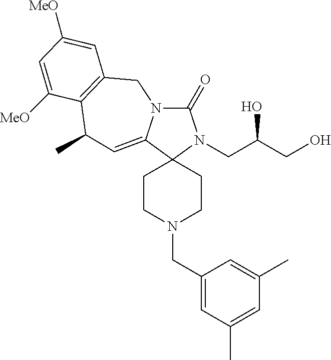 | 819789-01 HCl Salt | NMR ¹H (400 MHz, CDCl₃) δ 12.32 (br s, 1H), 7.24 (s, 2H), 7.11 (s, 1H), 6.44 (d, J = 2.4 Hz, 1H), 6.42 (d, J = 2.4 Hz, 1H), 4.80 (d, J = 8.8 Hz, 1H), 4.80 (d, J = 14.6 Hz, 1H), 4.63 (d, J = 14.4 Hz, 1H), 4.05-4.16 (m, 3H), 3.81 (s, 3H), 3.80 (s, 3H), 3.76-3.79 (m, 2H), 3.60 (d, J = 5.6 Hz, 2H), 3.38- 3.53 (m, 3H), 2.94-3.08 (m, 4H), 2.37 (s, 6H), 1.87 (d, J = 11.2 Hz, 1H), 1.78 (d, 13.2 Hz, 1H), 1.36 (d, J = 7.3 Hz, 3H) M/Z (ES+) Calc.: 535.30 Found: 536.39 (M + H) Analytical HPLC: Method A1 SunFire MS C18 (4.6 × 100 mm) 5 um Retention Time: 9.27 min Chiral HPLC Method C1 CHIRALPAK IA (0.46 × 25 cm) Retention Time: 5.591 min |
| 7 | 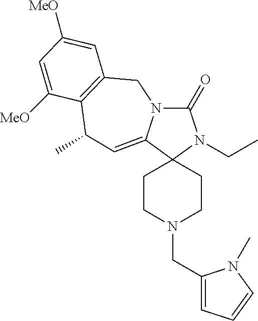 | 819924-01 HCl Salt | NMR ¹H (400 MHz, CD₃OD) δ 6.83-6.81 (m, 1H), 6.45 (d, J = 2.3 Hz, 1H), 6.38 (d, J = 2.3 Hz, 2H), 6.12 (t, J = 3.2 Hz, 1H), 5.17 (br s, 1H), 4.67 (d, J = 14.4 Hz, 1H), 4.59 (d, J = 14.4 Hz, 1H), 4.44 (s, 2H), 4.24-4.17 (m, 1H), 3.74 (t, J = 7.8 Hz, 6H), 3.62- 3.54 (m, 2H), 3.46-3.35 (m, 2H), 2.21 (br s, 2H), 2.62 (s, 3H), 2.23-2.14 (m, 2H), 1.95 (br d, J = 13.8 Hz, 1H), 1.78 (br d, J = 13.5 Hz, 1H), 1.35 (d, J = 7.3 Hz, 3H), 1.13 (t, J = 7.0 Hz, 3H) M/Z (ES+) Calc.: 464.28 Found: 465.39 (M + H) Analytical HPLC: Method A1 SunFire MS C18 (4.6 × 100 mm) 5 um Retention Time: 9.59 min Chiral HPLC Method C1 CHIRALPAK IA (0.46 × 25 cm) Retention Time: 7.790 min |
| 8 | 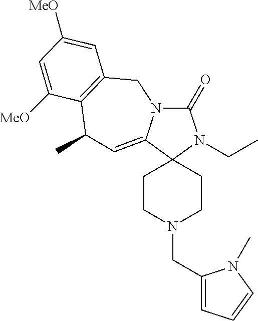 | 819925-00 Salt Free | NMR ¹H (400 MHz, CD₃OD) δ 6.83-6.81 (m, 1H), 6.45 (d, J = 2.3 Hz, 1H), 6.38 (d, J = 2.3 Hz, 2H), 6.11 (t, J = 3.2 Hz, 1H), 5.17 (br s, 1H), 4.67 (d, J = 14.4 Hz, 1H), 4.59 (d, J = 14.4 Hz, 1H), 4.34 (s, 2H), 4.24-4.17 (m, 1H), 3.74 (t, J = 7.9 Hz, 6H), 3.67- 3.36 (m, 4H), 3.19 (br s, 2H), 2.62 (s, 3H), 2.27-2.14 (m, 2H), 1.95 (br d, J = 12.6 Hz, 1H), 1.78 (br d, J = 11.4 Hz, 1H), 1.35 (d, J = 7.3 Hz, 3H), 1.13 (t, J = 7.0 Hz, 3H) M/Z (ES+) Calc.: 464.28 Found: 465.39 (M + H) Analytical HPLC: Method A1 SunFire MS C18 (4.6 × 100 mm) 5 um Retention Time: 9.58 min Chiral HPLC Method C1 CHIRALPAK IA (0.46 × 25 cm) Retention Time: 4.821 min |
| 9 | 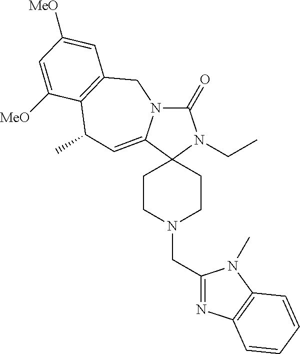 | 819926-01 HCl Salt | NMR ¹H (400 MHz, CD₃OD) δ 7.91-7.87 (m, 1H), 7.85-7.81 (m, 1H), 7.67-7.60 (m, 2H), 6.46 (d, J = 2.3 Hz, 1H), 6.39 (d, J = 2.3 Hz, 1H), 4.80 (s, 1H), 4.68 (d, J = 14.4 Hz, 1H), 4.60 (d, J = 14.1 Hz, 1H), 4.23 (q, J = 7.3, 14.6 Hz, 1H), 4.15 (s, 2H), 3.76 (s, 3H), 3.74 (s, 3H), 3.58-3.41 (m, 4H), 3.33-3.28 (m, 2H), 2.62 (s, 3H), 2.44-2.33 (m, 2H), 1.93 (br d, J = 16.1 Hz, 1H), 1.76 (br d, J = 14.4 Hz, 1H), 1.37 (d, J = 7.3 Hz, 3H), 1.14 (t, J = 7.0 Hz, 3H) M/Z (ES+) Calc.: 515.29 Found: 516.36 (M + H) Analytical HPLC: Method A1 SunFire MS C18 (4.6 × 100 mm) 5 um Retention Time: 8.28 min Chiral HPLC Method C1 CHIRALPAK IA (0.46 × 25 cm) Retention Time: 7.461 min |
| 10 | 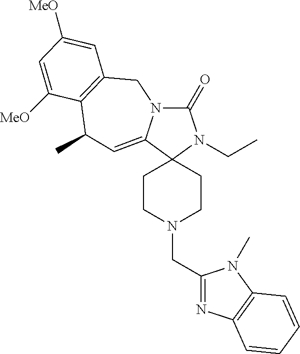 | 819927-01 HCl Salt | NMR ¹H (400 MHz, CD₃OD) δ 7.96-7.92 (m, 1H), 7.90-7.86 (m, 1H), 7.71-7.64 (m, 2H), 6.50 (d, J = 2.3 Hz, 1H), 6.43 (d, J = 2.3 Hz, 1H), 4.86 (s, 1H), 4.72 (d, J = 14.4 Hz, 1H), 4.64 (d, J = 14.1 Hz, 1H), 4.27 (q, J = 7.2, 14.5 Hz, 1H), 4.20 (s, 2H), 3.80 (s, 3H), 3.78 (s, 3H), 3.68-3.39 (m, 4H), 3.37-3.31 (m, 2H), 2.66 (s, 3H), 2.49-2.42 (m, 2H), 1.98 (br d, J = 14.6 Hz, 1H), 1.81 (br d, J = 14.4 Hz, 1H), 1.41(d, J = 7.3 Hz, 3H), 1.18 (t, J = 7.0 Hz, 3H) M/Z (ES+) Calc.: 515.29 Found: 516.36 (M + H) Analytical HPLC: Method A1 SunFire MS C18 (4.6 × 100 mm) 5 um Retention Time: 8.28 min Chiral HPLC Method C1 CHIRALPAK IA (0.46 × 25 cm) Retention Time: 5.670 min |
| 11 | 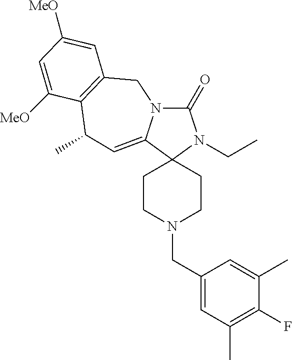 | 819931-00 Salt Free | NMR ¹H (400 MHz, CD₃OD) δ 7.02 (d, J = 7.0 Hz, 2H), 6.47 (d, J = 2.3 Hz, 1H), 6.41 (d, J = 2.3 Hz, 1H), 5.10 (d, J = 8.8 Hz, 1H), 4.68 (d, J = 14.4 Hz, 1H), 4.57 (d, J = 14.4 Hz, 1H), 4.19-4.12 (m, 1H), 3.79 (s, 3H), 3.77 (s, 3H), 3.51 (s, 2H), 3.28-3.18 (m, 2H), 2.82-2.73 (m, 2H), 2.65-2.54 (m, 2H), 2.24 (d, J = 1.8 Hz, 6H), 2.07-1.90 (m, 2H), 1.69 (br d, J = 12.0 Hz, 1H), 1.54 (br d, J = 13.5 Hz, 1H), 1.34 (d, J = 7.3 Hz, 3H), 1.14 (t, J = 7.0 Hz, 3H) M/Z (ES+) Cale.: 507.29 Found: 508.42 (M + H) Analytical HPLC: Method A1 SunFire MS C18 (4.6 × 100 mm) 5 um Retention Time: 11.22 min Chiral HPLC Method C1 CHIRALPAK IA (0.46 × 25 cm) Retention Time: 6.958 min |
| 12 | 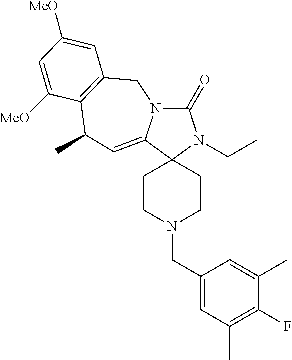 | 819943-00 Salt Free | NMR ¹H (400 MHz, CDCl₃) δ 6.92 (d, J = 7.0 Hz, 2H), 6.42 (d, J = 2.3 Hz, 1H), 6.39 (d, J = 2.3 Hz, 1H), 4.99 (d, J = 8.5 Hz, 1H), 4.79 (d, J = 14.1 Hz, 1H), 4.56 (d, J = 14.4 Hz, 1H), 4.15-4.08 (m, 1H), 3.77 (d, J = 3.2 Hz, 6H), 3.42 (s, 2H), 3.29-3.24 (m, 1H), 3.24-3.15 (m, 1H), 2.75-2.65 (m, 2H), 2.53-2.43 (m, 2H), 2.23 (d, J = 2.1 Hz, 6H), 1.97-1.83 (m, 2H), 1.66- 1.56 (m, 2H), 1.33 (d, J = 7.3 Hz, 3H), 1.13 (t, J = 7.0 Hz, 3H) M/Z (ES+) Calc.: 507.29 Found: 508.36 (M + H) Analytical HPLC: Method A1 SunFire MS C18 (4.6 × 100 mm) 5 um Retention Time: 11.20 min Chiral HPLC Method C1 CHIRALPAK IA (0.46 × 25 cm) Retention Time: 4.684 min |
| 13 | 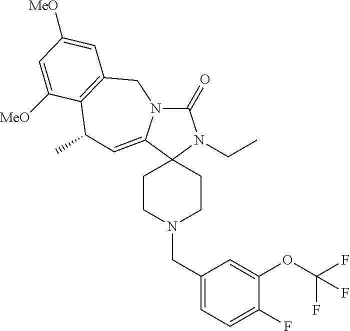 | 819933-00 Salt Free | NMR ¹H (400 MHz, CDCl₃) δ 7.29 (br d, J = 6.7, 1H), 7.24-7.21 (m, 1H), 7.12 (t, J = 9.1 Hz, 1H), 6.42 (d, J = 2.3 Hz, 1H), 6.39 (d, J = 2.3 Hz, 1H), 4.98 (d, J = 8.5 Hz, 1H), 4.79 (d, J = 14.4 Hz, 1H), 4.56 (d, J = 14.4 Hz, 1H), 4.15-4.08 (m, 1H), 3.77 (d, J = 2.3 Hz, 6H), 3.52 (s, 2H), 3.31-3.24 (m, 1H), 3.24-3.15 (m, 1H), 2.73-2.62 (m, 2H), 2.57-2.48 (m, 2H), 1.73-1.83 (m, 2H), 1.68-1.55 (m, 2H), 1.33 (d, J = 7.0 Hz, 3H), 1.15 (t, J = 7.0 Hz, 3H) M/Z (ES+) Calc.: 563.24 Found: 564.30 (M + H) Analytical HPLC: Method A1 SunFire MS C18 (4.6 × 100 mm) 5 um Retention Time: 11.45 min Chiral HPLC Method C1 CHIRALPAK IA (0.46 × 25 cm) Retention Time: 5.872 min |
| 14 | 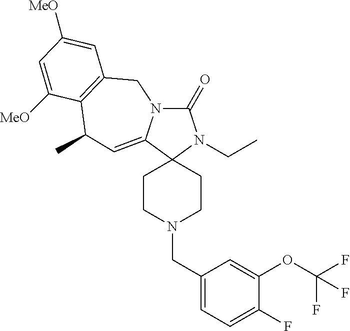 | 819945-00 Salt Free | NMR ¹H (400 MHz, CDCl₃) δ 7.29 (br d, J = 7.3, 1H), 7.24-7.21 (m, 1H), 7.12 (t, J = 8.9 Hz, 1H), 6.42 (d, J = 2.3 Hz, 1H), 6.39 (d, J = 2.6 Hz, 1H), 4.98 (d, J = 8.5 Hz, 1H), 4.79 (d, J = 14.4 Hz, 1H), 4.56 (d, J = 14.4 Hz, 1H), 4.15-4.08 (m, 1H), 3.77 (d, J = 2.1 Hz, 6H), 3.52 (s, 2H), 3.31-3.24 (m, 1H), 3.22-3.15 (m, 1H), 2.73-2.62 (m, 2H), 2.57-2.48 (m, 2H), 1.97-1.83 (m, 2H), 1.68-1.58 (m, 2H), 1.33 (d, J = 7.30 Hz, 3H), 1.15 (t, J = 7.0 Hz, 3H) M/Z (ES+) Calc.: 563.24 Found: 564.30 (M + H) Analytical HPLC: Method A1 SunFire MS C18 (4.6 × 100 mm) 5 um Retention Time: 11.77 min Chiral HPLC Method C1 CHIRALPAK IA (0.46 × 25 cm) Retention Time: 4.144 min |
| 15 | 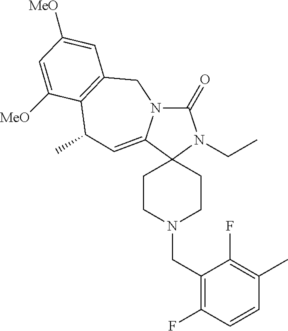 | 819934-00 Salt Free | NMR ¹H (400 MHz, CD₃OD) δ 7.24 (q, J = 8.3, 15.1 Hz, 1H), 6.91 (t, J = 8.8 Hz, 1H), 6.47 (d, J = 2.3 Hz, 1H), 6.41 (d, J = 2.3 Hz, 1H), 5.00 (d, J = 8.8 Hz, 1H), 4.67 (d, J = 14.1 Hz, 1H), 4.57 (d, J = 14.1 Hz, 1H), 4.14-4.06 (m, 1H), 3.79 (s, 3H), 3.78 (s, 3H), 3.76 (s, 2H), 3.29-3.17 (m, 2H), 2.89-2.81 (m, 2H), 2.75-2.70 (m, 2H), 2.27 (s, 3H), 2.06-1.95 (m, 2H), 1.68 (br d, J = 13.5 Hz, 1H), 1.56 (br d, J = 13.5 Hz, 1H), 1.31 (d, J = 7.3 Hz, 3H), 1.13 (t, J = 7.0 Hz, 3H) M/Z (ES+) Calc.: 511.26 Found: 512.39 (M + H) Analytical HPLC: Method A1 SunFire MS C18 (4.6 × 100 mm) 5 um Retention Time: 10.52 min Chiral HPLC Method C1 CHIRALPAK IA (0.46 × 25 cm) Retention Time: 6.734 min |
| 16 | 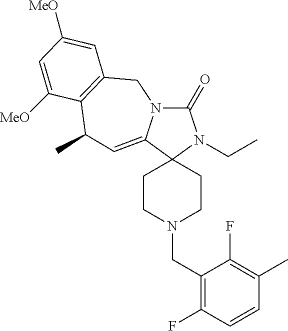 | 819946-00 Salt Free | NMR ¹H (400 MHz, CDCl₃) δ 7.07 (q, J = 8.3, 14.8 Hz, 1H), 6.77 (t, J = 8.1 Hz, 1H), 6.41 (d, J = 2.3 Hz, 1H), 6.38 (d, J = 2.3 Hz, 1H), 4.89 (d, J = 8.5 Hz, 1H), 4.78 (d, J = 14.1 Hz, 1H), 4.54 (d, J = 14.4 Hz, 1H), 4.10- 4.02 (m, 1H), 3.77 (d, J = 2.6 Hz, 6H), 3.68 (s, 2H), 3.29-3.21 (m, 1H), 3.19- 3.10 (m, 1H), 2.81-2.71 (m, 2H), 2.61 (q, J = 12.0, 23.1 Hz, 2H), 2.23 (s, 3H), 1.96- 1.83 (m, 2H), 1.65-1.55 (m, 2H), 1.30 (d, J = 7.3 Hz, 3H), 1.10 (t, J = 7.0 Hz, 3H) M/Z (ES+) Calc.: 511.26 Found: 512.39 (M + H) Analytical HPLC: Method A1 SunFire MS C18 (4.6 × 100 mm) 5 um Retention Time: 10.45 min Chiral HPLC Method C1 CHIRALPAK IA (0.46 × 25 cm) Retention Time: 4.799 min |
| 17 | 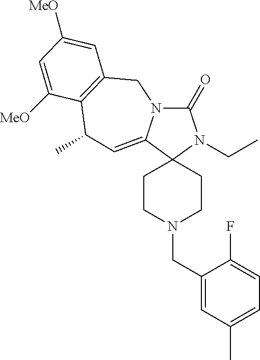 | 819935-00 Salt Free | NMR ¹H (400 MHz, CD₃OD) δ 7.22 (d, J = 7.0 Hz, 1H), 7.15-7.11 (m, 1H), 6.98 (t, J = 9.2 Hz, 1H), 6.48 (d, J = 2.3 Hz, 1H), 6.41 (d, J = 2.6 Hz, 1H), 5.05 (d, J = 8.5 Hz, 1H), 4.68 (d, J = 14.4 Hz, 1H), 4.57 (d, J = 14.4 Hz, 1H), 4.16-4.09 (m, 1H), 3.79 (s, 3H), 3.77 (s, 3H), 3.66 (s, 2H), 3.28-3.20 (m, 2H), 2.87-2.79 (m, 2H), 2.72-2.63 (m, 2H), 2.33 (s, 3H), 2.06-1.94 (m, 2H), 1.69 (br d, J = 13.5 Hz, 1H), 1.56 (br d, J = 13.5 Hz, 1H), 1.32 (d, J = 7.3 Hz, 3H), 1.14 (t, J = 7.0 Hz, 3H) M/Z (ES+) Calc.: 493.27 Found: 494.37 (M + H) Analytical HPLC: Method A1 SunFire MS C18 (4.6 × 100 mm) 5 um Retention Time: 10.48 min Chiral HPLC Method C1 CHIRALPAK IA (0.46 × 25 cm) Retention Time: 6.585 min |
| 18 | 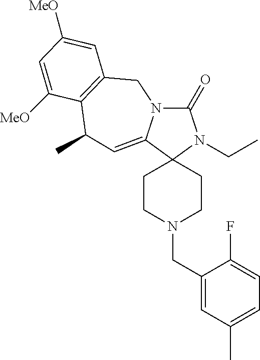 | 819947-00 Salt Free | NMR ¹H (400 MHz, CD₃OD) δ 7.23-7.21 (m, 1H), 7.15-7.11 (m, 1H), 7.98 (t, J = 9.2 Hz, 1H), 6.47 (d, J = 2.3 Hz, 1H), 6.41 (d, J = 2.3 Hz, 1H), 5.05 (d, J = 8.8 Hz, 1H), 4.68 (d, J = 14.4 Hz, 1H), 4.57 (d, J = 14.4 Hz, 1H), 4.16-4.09 (m, 1H), 3.79 (s, 3H), 3.77 (s, 3H), 3.66 (s, 2H), 3.28-3.18 (m, 2H), 2.87-2.79 (m, 2H), 2.72-2.61 (m, 2H), 2.33 (s, 3H), 2.06-1.94 (m, 2H), 1.69 (br d, J = 11.7 Hz, 1H), 1.56 (br d, J = 13.8 Hz, 1H), 1.32 (d, J = 7.3 Hz, 3H), 1.14 (t, J = 7.0 Hz, 3H) M/Z (ES+) Calc.: 493.27 Found: 494.41 (M + H) Analytical HPLC: Method A1 SunFire MS C18 (4.6 × 100 mm) 5 um Retention Time: 10.52 min Chiral HPLC Method C1 CHIRALPAK IA (0.46 × 25 cm) Retention Time: 4.695 min |
| 19 | 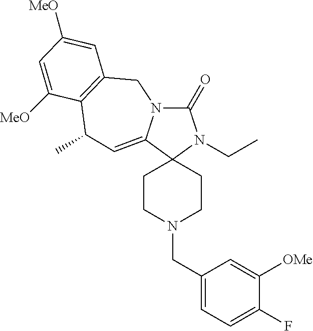 | 819936-00 Salt Free | NMR ¹H (400 MHz, CD₃OD) δ 7.09-7.07 (m, 1H), 7.02-6.97 (m, 1H), 6.88-6.84. (m, 1H), 6.44 (d, J = 2.3 Hz, 1H), 6.38 (d, J = 2.6 Hz, 1H), 5.10 (d, J = 8.8 Hz, 1H), 4.65 (d, J = 14.4 Hz, 1H), 4.55 (d, J = 14.4 Hz, 1H), 4.16-4.09 (m, 1H), 3.84 (s, 3H), 3.75 (d, J = 9.1 Hz, 6H), 3.55 (s, 2H), 3.26-3.19 (m, 2H), 2.81-2.72 (m, 2H), 2.63-2.527 (m, 2H), 2.03-1.90 (m, 2H), 1.67 (br d, J = 13.5 Hz, 1H), 1.54 (br d, J = 13.5 Hz, 1H), 1.31 (d, J = 7.3 Hz, 3H), 1.11 (t, J = 7.0 Hz, 3H) M/Z (ES+) Calc.: 509.27 Found: 510.47 (M + H) Analytical HPLC: Method A1 SunFire MS C18 (4.6 × 100 mm) 5 um Retention Time: 9.71 min Chiral HPLC Method C1 CHIRALPAK IA (0.46 × 25 cm) Retention Time: 6.902 min |
| 20 | 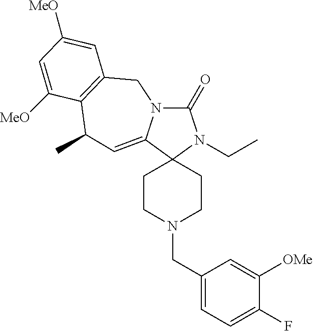 | 819948-00 Salt Free | NMR ¹H (400 MHz, CD₃OD) δ 7.13-7.10 (m, 1H), 7.06-7.01 (m, 1H), 6.91-6.88 (m, 1H), 6.48 (d, J = 2.6 Hz, 1H), 6.41 (d, J = 2.6 Hz, 1H), 5.13 (d, J = 8.8 Hz, 1H), 4.69 (d, J = 14.4 Hz, 1H), 4.58 (d, J = 14.1 Hz, 1H), 4.20-4.13 (m, 1H), 3.88 (s, 3H), 3.80 (s, 3H), 3.77 (s, 3H), 3.58 (s, 2H), 3.28-3.20 (m, 2H), 2.85-2.75 (m, 2H), 2.67-2.56 (m, 2H), 2.06-1.93 (m, 2H), 1.71 (br d, J = 11.7 Hz, 1H), 1.57 (br d, J = 11.7 Hz, 1H), 1.35 (d, J = 7.0 Hz, 3H), 1.15 (t, J = 7.0 Hz, 3H) M/Z (ES+) Calc.: 509.27 Found: 510.47 (M + H) Analytical HPLC: Method A1 SunFire MS C18 (4.6 × 100 mm) 5 um Retention Time: 9.70 min Chiral HPLC Method C1 CHIRALPAK IA (0.46 × 25 cm) Retention Time: 4.781 min |
| 21 | 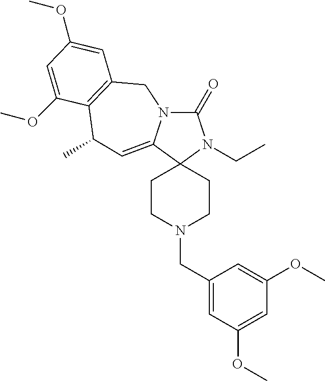 | 829893-00 Salt Free | NMR¹H (400 MHz, CD₃OD) δ 6.54 (d, J = 2.1 Hz, 2H), 6.48 (d, J = 2.3 Hz, 1H), 6.42-6.40 (m, 2H), 5.12 (d, J = 8.5 Hz, 1H), 4.68 (d, J = 14.1 Hz, 1H), 4.58 (d, J = 14.1 Hz, 1H), 4.20-4.12 (m, 1H), 3.80 (s, 3H), 3.78 (d, J = 0.9 Hz, 9H), 3.56 (s, 2H), 3.29-3.20 (m, 2H), 2.85-2.77 (m, 2H), 2.67-2.56 (m, 2H), 2.07-1.95 (m, 2H), 1.72-1.68 (m, 1H), 1.58-1.55 (m, 1H), 1.34 (d, J = 7.3 Hz, 3H), 1.15 (t, J = 7.0 Hz, 3H) M/Z (ES+) Calc.: 521.29 Found: 522.39 (M + H) Analytical HPLC: Method A1 SunFire MS C18 (4.6 × 100 mm) 5 um Retention Time: 10.22 min Chiral HPLC Method C1 CHIRALPAK IA (0.46 × 25 cm) Retention Time: 8.084 min |
| 22 | 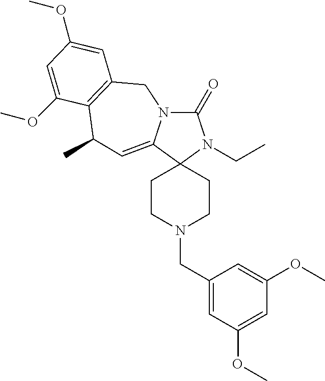 | 819950-00 Salt Free | NMR ¹H (400 MHz, CDCl₃) δ 6.49 (d, J = 2.3 Hz, 2H), 6.42 (d, J = 2.6 Hz, 1H), 6.39 (d, J = 2.6 Hz, 1H), 6.34 (t, J = 2.2 Hz, 1H), 5.00 (d, J = 8.5 Hz, 1H), 4.79 (d, J = 14.4 Hz, 1H), 4.56 (d, J = 14.4 Hz, 1H), 4.15- 4.08 (m, 1H), 3.78 (s, 6H), 3.77 (d, J = 2.1 Hz, 6H), 3.49 (s, 2H), 3.33-3.24 (m, 1H), 3.24-3.15 (m, 1H), 2.77-2.67 (m, 2H), 2.55-2.46 (m, 2H), 1.99-1.85 (m, 2H), 1.66-1.57 (m, 2H), 1.33 (d, J = 7.0 Hz, 3H), 1.14 (t, J = 7.0 Hz, 3H) M/Z (ES+) Calc.: 521.29 Found: 522.38 (M + H) Analytical HPLC: Method A1 SunFire MS C18 (4.6 × 100 mm) 5 um Retention Time: 10.25 min Chiral HPLC Method C1 CHIRALPAK IA (0.46 × 25 cm) Retention Time: 5.601 min |
| 23 | 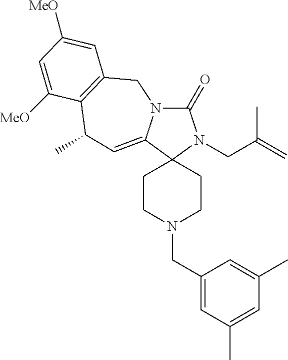 | 820006-01 HCl Salt | NMR ¹H (400 MHz, DMSO) δ 12.85 (br s, 1H), 7.24 (s, 2H), 7.10 (s, 1H), 6.40-6.42 (m, 2H), 4.79-4.85 (m, 3H), 4.54-4.67 (m, 2H), 3.86-4.14 (m, 5H), 3.80 (s, 3H), 3.79 (s, 3H), 3.30-3.49 (m, 2H), 2.82- 3.14 (m, 4H), 2.37 (s, 6H), 1.80 (d, J = 13.7 Hz, 1H), 1.76 (s, 3H), 1.68 (d, 14.2 Hz, 1H), 1.34 (d, J = 7.1 Hz, 3H) M/Z (ES+) Calc.: 515.31 Found: 516.42 (M + H) Analytical HPLC: Method A1 SunFire MS C18 (4.6 × 100 mm) 5 um Retention Time: 12.04 min Chiral HPLC Method C1 CHIRALPAK IA (0.46 × 25 cm) Retention Time: 7.576 min |
| 24 | 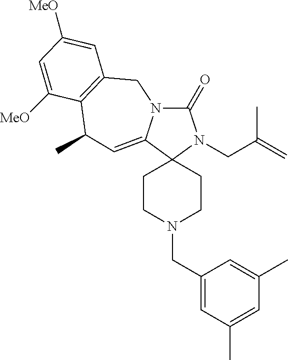 | 820007-01 HCl Salt | NMR ¹H (400 MHz, DMSO) δ 12.84 (br s, 1H), 7.24 (s, 2H), 7.10 (s, 1H), 6.41-6.42 (m, 2H), 4.79-4.85 (m, 3H), 4.54-4.67 (m, 2H), 3.86-4.14 (m, 5H), 3.80 (s, 3H), 3.79 (s, 3H), 3.29-3.46 (m, 2H), 2.82- 3.14 (m, 4H), 2.36 (s, 6H), 1.80 (d, J = 13.9 Hz, 1H), 1.76 (s, 3H), 1.69 (d, 14.4 Hz, 1H), 1.34 (d, J = 7.1 Hz, 3H) M /Z (ES+) Calc.: 515.31 Found: 516.42 (M + H) Analytical HPLC: Method A1 SunFire MS C18 (4.6 × 100 mm) 5 um Retention Time: 12.02 min Chiral HPLC Method C1 CHIRALPAK IA (0.46 × 25 cm) Retention Time: 5.074 min |
| 25 | 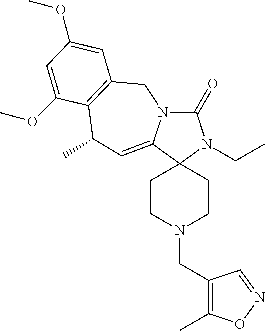 | 819810-01 HCl Salt | NMR ¹H (400 MHz, CD₃OD) δ 8.34 (s, 1H), 6.46 (d, J = 2.6 Hz, 1H), 6.41 (d, J = 2.3 Hz, 1H), 5.13 (d, J = 8.8 Hz, 1H), 4.69 (d, J = 14.4 Hz, 1H), 4.58 (d, J = 14.4 Hz, 1H), 4.21-4.14 (m, 1H), 3.78 (s, 3H), 3.76 (s, 3H), 3.58 (d, J = 2.3 Hz, 2H), 3.30-3.16 (m, 2H), 2.93 (br d, J = 12.0 Hz, 1H), 2.84 (br d, J = 11.1 Hz, 1H), 2.79-2.65 (m, 2H), 2.44 (s, 3H), 2.08-1.94 (m, 2H), 1.75 (br d, J = 13.8 Hz, 1H), 1.59 (br d, J = 11.1 Hz, 1H), 1.34 (d, J = 7.3 Hz, 3H), 1.14 (t, J = 7.0 Hz, 3H) M/Z (ES+) Calc.: 466.26 Found: 568.45 (M + H + 101) Analytical HPLC: Method A1 SunFire MS C18 (4.6 × 100 mm) 5 um Retention Time: 8.00 min |
| 26 | 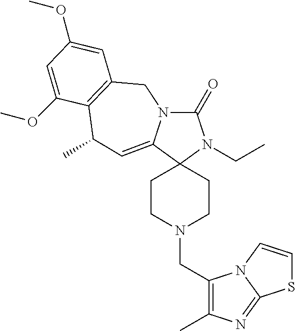 | 811352-00 Salt Free | NMR ¹H (400 MHz, DMSO) δ 7.82 (d, J = 4.4 Hz, 1H), 7.16 (d, J = 4.7 Hz, 1H), 6.49 (d, J = 2.3 Hz, 1H), 6.39 (d, J = 2.3 Hz, 1H), 5.06 (d, J = 8.5 Hz, 1H), 4.64 (d, J = 14.4 Hz, 1H), 4.50 (d, J = 14.4 Hz, 1H), 4.10- 4.02 (m, 1H), 3.76 (s, 3H), 3.74 (s, 3H), 3.31 (s, 2H), 3.17-3.07 (m, 2H), 2.76- 2.52 (m, 4H), 2.21 (s, 3H), 1.86-1.72 (m, 2H), 1.56 (br d, J = 11.1 Hz, 1H), 1.40 (br d, J = 12.3 Hz, 1H), 1.28 (d, J = 7.3 Hz, 3H), 1.03 (t, J = 6.9 Hz, 3H) M/Z (ES+) Calc.: 521.25 Found: 522.34 (M + H) Analytical HPLC: Method A1 SunFire MS C18 (4.6 × 100 mm) 5 um Retention Time: 8.05 min |
| 27 | 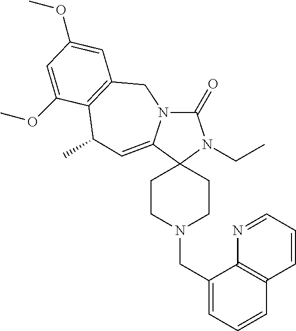 | 819955-01 HCl Salt | (400 MHz, CD₃OD) δ 8.93-8.92 (m, 1H), 8.36-8.33 (m, 1H), 7.91-7.86 (m, 2H), 7.61 (t, J = 7.6 Hz, 1H), 7.55-7.52 (m, 1H), 6.48 (d, J = 2.3 Hz, 1H), 6.41 (d, J = 2.3 Hz, 1H), 5.17 (d, J = 8.5 Hz, 1H), 4.68 (d, J = 14.4 Hz, 1H), 4.58 (d, J = 14.4 Hz, 1H), 4.32 (s, 2H), 4.20-4.12 (m, 1H), 3.80 (s, 3H), 3.77 (s, 3H), 3.30-3.19 (m, 2H), 2.99-2.86 (m, 2H), 2.86-2.74 (m, 2H), 2.10-1.97 (m, 2H), 1.69 (br d, J = 13.5 Hz, 1H), 1.56 (br d, J = 11.7 Hz, 1H), 1.34 (d, J = 7.3 Hz, 3H), 1.13 (t, J = 7.0 Hz, 3H) M/Z (ES+) Calc.: 512.28 Found: 513.40 (M + H) Analytical HPLC: Method A1 SunFire MS C18 (4.6 × 100 mm) 5 um Retention Time: 9.80 min |
| 28 | 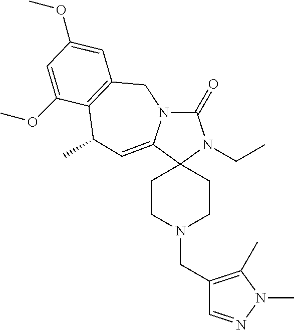 | 819976-01 HCl Salt | (500 MHz, CD₃OD) δ 7.60 (s, 1H), 6.50 (d, J = 2.3 Hz, 1H), 6.43 (d, J = 2.3 Hz, 1H), 5.22 (br s, 1H), 4.71 (d, J = 14.2 Hz, 1H), 4.64 (d, J = 14.2 Hz, 1H), 4.30 (br s, 1H), 4.25 (br s, 1H), 3.83-3.77 (m, 9H), 3.57 (br s, 1H), 3.44 (br s, 2H), 3.22 (br s, 2H), 2.65 (s, 2H), 2.39 (s, 3H) 2.26- 2.13 (m, 2H), 2.00 (br d, J = 11.4 Hz, 1H), 1.82 (br d, J = 13.3 Hz, 1H), 1.39 (d, J = 6.9 Hz, 3H), 1.17 (t, J = 6.9 Hz, 3H) M/Z (ES+) Calc.: 479.29 Found: 480.45 (M + H) Analytical HPLC: Method A1 SunFire MS C18 (4.6 × 100 mm) 5 um Retention Time: 7.22 min |
| 29 | 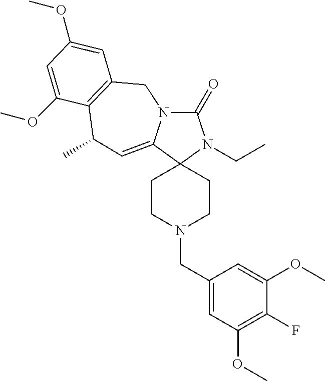 | 824214-00 Salt Free | (400 MHz, CDCl₃) δ 6.60 (d, J = 7.0 Hz, 2H), 6.45 (d, J = 2.6 Hz, 1H), 6.42 (d, J = 2.3 Hz, 1H), 5.03 (d, J = 8.8 Hz, 1H), 4.82 (d, J = 14.1 Hz, 1H), 4.59 (d, J = 14.4 Hz, 1H), 4.18-4.10 (m, 1H), 3.90 (d, J = 0.9 Hz, 6H), 3.80 (d, J = 2.9 Hz, 6H), 3.49 (s, 2H), 3.35-3.18 (m, 2H), 2.78- 2.67 (m, 2H), 2.58-2.47 (m, 2H), 2.01- 1.86 (m, 2H), 1.70-1.60 (m, 2H), 1.37 (d, J = 7.3 Hz, 3H), 1.18 (t, J = 7.0 Hz, 3H) M/Z (ES+) Calc.: 539.28 Found: 540.36 (M + H) Analytical HPLC: Method A1 SunFire MS C18 (4.6 × 100 mm) 5 um Retention Time: 9.52 min |
| 30 | 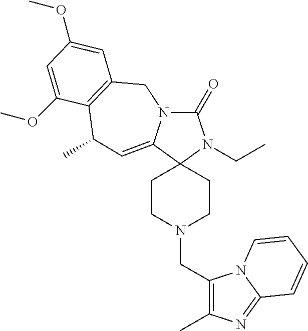 | 811305-01 HCl Salt | (400 MHz, DMSO) δ 9.26 (br s, 1H), 7.90 (br s, 2H), 7.51 (br s, 1H), 6.52 (d, J = 2.1 Hz, 1H), 6.43 (d, J = 2.3 Hz, 1H), 4.97 (br s, 1H), 4.66 (d, J = 14.6 Hz, 1H), 4.55 (d, J = 14.1 Hz, 1H), 4.19-4.11 (m, 1H), 3.79-3.72 (m, 6H), 3.47 (br s, 6H), 3.21 (br d, J = 5.9 Hz, 1H), 2.85 (br s, 1H), 2.67 (s, 2H), 2.54 (s, 3H), 1.79 (br d, J = 13.5 Hz, 1H), 1.59 (br d, J = 13.2 Hz, 1H), 1.36 (d, J = 7.3 Hz, 3H), 1.01 (t, J = 7.0 Hz, 3H) M/Z (ES+) Calc.: 515.29 Found: 516.43 (M + H) Analytical HPLC: Method A1 SunFire MS C18 (4.6 × 100 mm) 5 um Retention Time: 8.23 min |
| 31 | 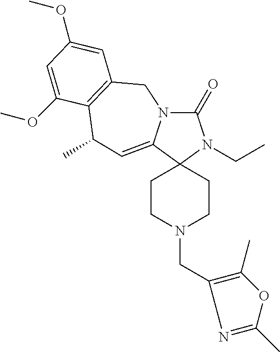 | 811283-01 HCl Salt | M/Z (ES+) Calc.: 480.27 Found: 481.34 (M + H) Analytical HPLC: Method A1 SunFire MS C18 (4.6 × 100 mm) 5 um Retention Time: 7.77 min |
| 32 | 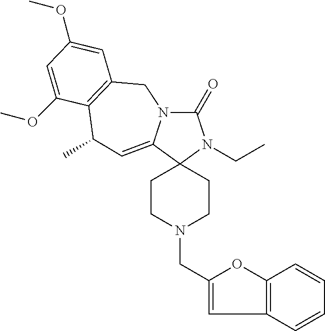 | 811308-01 HCl Salt | (500 MHz, DMSO) δ 7.59 (d, J = 6.9 Hz, 1H), 7.54 (d, J = 7.3 Hz, 1H), 7.29-7.25 (m, 1H), 7.23-7.20 (m, 1H), 6.80 (s, 1H), 6.48 (d, J = 2.3 Hz, 1H), 6.39 (d, J = 2.3 Hz, 1H), 4.99 (d, J = 8.7 Hz, 1H), 4.64 (d, J = 14.6 Hz, 1H), 4.50 (d, J = 14.2 Hz, 1H), 4.04-3.97 (m, 1H), 3.76 (s, 3H), 3.75 (s, 3H), 3.73 (s, 2H), 3.20-3.09 (m, 2H), 2.84-2.59 (m, 4H), 1.92-1.82 (m, 2H), 1.58 (br d, J = 13.3 Hz, 1H), 1.44 (br d, J = 13.3 Hz, 1H), 1.24 (d, J = 7.3 Hz, 3H), 1.03 (t, J = 3.0 Hz, 3H) M/Z (ES+) Calc.: 501.26 Found: 603.38 (M + H + 101) Analytical HPLC: Method A1 SunFire MS C18 (4.6 × 100 mm) 5 um Retention Time: 10.12 min |
| 33 | 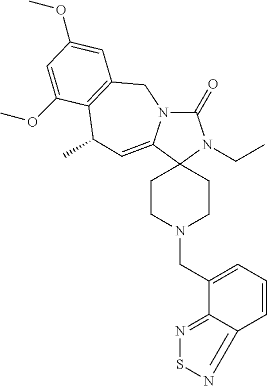 | 811332-01 HCl Salt | (500 MHz, CD₃OD) δ 8.20 (d, J = 9.2 Hz, 1H), 7.93 (d, J = 6.4 Hz, 1H), 7.82-7.79 (m, 1H), 6.50 (d, J = 2.3 Hz, 1H), 6.43 (d, J = 2.3 Hz, 1H), 5.30 (br s, 1H), 4.96 (br s, 2H), 4.71 (d, J = 14.6 Hz, 1H), 4.64 (d, J = 14.6 Hz, 1H), 4.27 (br s, 1H), 3.81 (s, 3H), 3.78 (s, 3H) , 3.66 (br s, 4H), 3.19 (br s, 2H), 2.21 (br s, 2H), 1.99 (br s, 1H), 1.82 (br s, 1H), 1.40 (br d, J = 6.4 Hz, 3H), 1.15 (br s, 3H) M/Z (ES+) Calc.: 519.23 Found: 621.40 (M + H + 101) Analytical HPLC: Method A1 SunFire MS C18 (4.6 × 100 mm) 5 um Retention Time: 10.32 min |
| 34 | 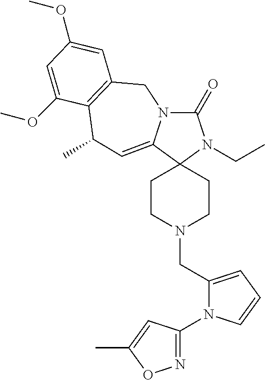 | 820017-01 HCl Salt | (500 MHz, CD₃OD) δ 7.42 (br s, 1H), 6.67 (br s, 1H), 6.65 (s, 1H), 6.50 (d, J = 2.3 Hz, 1H), 6.43 (d, J = 2.7 Hz, 2H), 5.30 (br s, 1H), 4.73-4.63 (m, 3H), 4.28 (br s, 1H), 3.80 (s, 3H), 3.78 (s, 3H), 3.57 (br s, 3H), 3.20 (br s, 2H), 2.65 (s, 3H), 2.51 (s, 2H), 2.19 (br s, 2H), 2.01 (br s, 1H), 1.84 (br s, 1H), 1.41 (br d, J = 6.9 Hz, 3H), 1.20-1.12 (m, 3H) M/Z (ES+) Calc.: 531.28 Found: 532.42 (M + H) Analytical HPLC: Method A1 SunFire MS C18 (4.6 × 100 mm) 5 um Retention Time: 10.27 min |
| 36 | 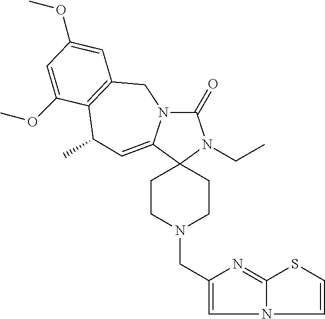 | 811309-01 HCl Salt | (400 MHz, CD₃OD) δ 8.22 (s, 1H), 8.04- 8.03 (m, 1H), 7.53-7.51 (m, 1H), 6.49 (d, J = 2.6 Hz, 1H), 6.42 (d, J = 2.3 Hz, 1H), 4.76-4.55 (m, 3H), 4.26-4.21 (m, 1H), 3.82-3.76 (m, 8H), 3.67 (br s, 2H), 3.59 (d, J = 6.7 Hz, 2H), 3.29 (br s, 2H), 2.48- 2.32 (m, 2H), 2.02 (br s, 1H), 1.86 (br s, 1H), 1.38 (d, J = 7.3 Hz, 3H), 1.17 (t, J = 7.0 Hz, 3H) M/Z (ES+) Calc.: 507.23 Found: 508.36 (M + H) Analytical HPLC: Method A1 SunFire MS C18 (4.6 × 100 mm) 5 um Retention Time: 6.95 min |
| 37 | 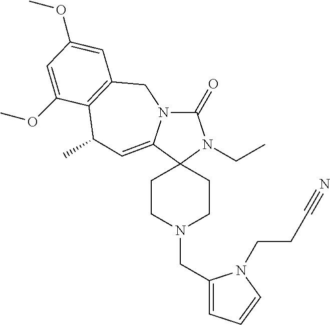 | 820008-00 Salt Free | (400 MHz, CDCl₃) δ 6.72-6.70 (m,1H), 6.45 (d, J = 2.3 Hz, 1H), 6.42 (d, J = 2.3 Hz, 1H), 6.09 (t, J = 3.2 Hz, 1H), 6.05- 6.04 (m, 1H), 5.02 (d, J = 8.8 Hz, 1H), 4.82 (d, J = 14.4 Hz, 1H), 4.59 (d, J = 14.4 Hz, 1H), 4.25 (t, J = 7.2 Hz, 2H), 4.20- 4.12 (m, 1H), 3.80 (d, J = 2.9 Hz, 6H), 3.52 (s, 2H), 3.31-3.22 (m, 1H), 3.22- 3.13 (m, 1H), 2.94 (t, J = 7.0 Hz, 2H), 2.79-2.68 (m, 2H), 2.56-2.46 (m, 2H), 1.91-1.77 (m, 2H), 1.73-1.62 (m, 2H), 1.37 (d, J = 7.3 Hz, 3H), 1.17 (t, J = 7.0 Hz, 3H) M/Z (ES+) Calc.: 503.29 Found: 504.39 (M + H) Analytical HPLC: Method A1 SunFire MS C18 (4.6 × 100 mm) 5 um Retention Time: 9.56 min |
| 38 | 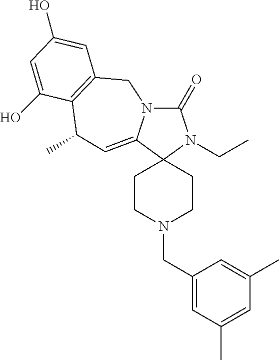 | 819888-00 Salt Free | (400 MHz, CDCl₃) δ 6.94 (s, 2H), 6.91 (s, 1H), 6.60 (s, 1H), 6.28 (s, 1H), 5.05 (d, J = 8.3 Hz, 1H), 4.81 (d, J = 14.2 Hz, 1H), 4.59 (d, J = 14.2 Hz, 1H), 4.11-4.03 (m, 1H), 3.53 (s, 2H), 3.28-3.17 (m, 2H), 2.79-2.74 (m, 2H), 2.56-2.53 (m, 2H), 2.30 (s, 6H), 1.96-1.90 (m, 2H), 1.70- 1.59 (m, 2H), 1.40 (d, J = 7.1 Hz, 3H), 1.11 (t, J = 6.8 Hz, 3H) M/Z (ES+) Calc.: 461.27 Found: 462.37 (M + H) Analytical HPLC: Method A1 SunFire MS C18 (4.6 × 100 mm) 5 um Retention Time: 6.57 min |
| 39 | 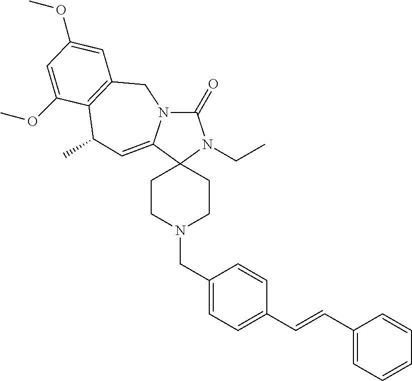 | 819814-01 HCl Salt | (400 MHz, CD₃OD) δ 7.71 (d, J = 7.9 Hz, 2H), 7.58 (d, J = 8.5 Hz, 4H), 7.36 (t, J = 7.6 Hz, 2H), 7.32-7.20 (m, 3H), 6.49 (d, J = 2.3 Hz, 1H), 6.42 (d, J = 2.6 Hz, 1H), 5.23 (d, J = 8.8 Hz, 1H), 4.71 (d, J = 14.4 Hz, 1H), 4.63 (d, J = 14.4 Hz, 1H), 4.46 (s, 2H), 4.29-4.22 (m, 1H), 3.79 (s, 3H), 3.77 (s, 3H), 3.61-3.47 (m, 4H), 3.28-3.15 (m, 2H), 2.35-2.17 (m, 2H), 1.99 (br d, J = 13.8 Hz, 1H), 1.80 (br d, J = 14.1 Hz, 1H), 1.40 (d, J = 7.3 Hz, 3H), 1.16 (t, J = 7.0 Hz, 3H) M/Z (ES+) Calc.: 563.31 Found: 564.36 (M + H) Analytical HPLC: Method A1 SunFire MS C18 (4.6 × 100 mm) 5 um Retention Time: 12.04 min |
| 40 | 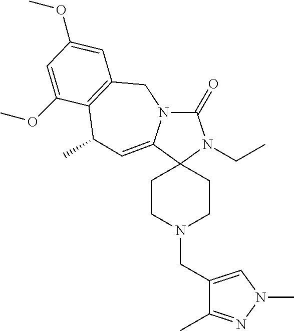 | 819971-00 Salt Free | (400 MHz, CD₃OD) δ 7.46 (s, 1H), 6.46 (d, J = 2 Hz, 1H), 6.40 (d, J = 2 Hz, 1H), 5.07 (d, J = 7 Hz, 1H), 4.68 (d, J = 14 Hz, 1H), 4.56 (d, J = 14 Hz, 1H), 4.10-4.05 (m, 1H), 3.80-3.72 (m, 9H), 3.43 (br s, 2H), 3.22-3.17 (m, 2H), 2.68-2.48 (m, 2H), 2.20 (s, 3H), 2.03-1.86 (m, 2H), 1.75-1.50 (m, 2H), 1.33 (d, J = 7 Hz, 3H), 1.12 (t, J = 6.9 Hz, 3H) M/Z (ES+) Calc.: 479.29 Found: 480.45 (M + H) Analytical HPLC: Method A1 SunFire MS C18 (4.6 × 100 mm) 5 um Retention Time: 7.15 min |
| 41 | 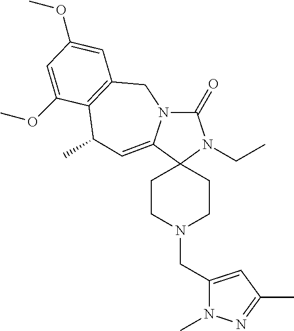 | 819973-00 Salt Free | (400 MHz, CD₃OD) δ 6.45 (d, J = 2 Hz, 1H), 6.40 (d, J = 2 Hz, 1H), 5.95 (s, 1H), 5.11 (d, J = 7 Hz, 1H), 4.68 (d, J = 14 Hz, 1H), 4.55 (d, J = 14 Hz, 1H), 4.22-4.10 (m, 1H), 3.80-3.69 (m, 9H), 3.60-6.49 (m, 2H), 3.30-3.12 (m, 2H), 2.84-2.49 (m, 2H), 2.15 (s, 3H), 2.02-1.83 (m, 2H), 1.70-1.42 (m, 2H), 1.33 (d, J = 4 Hz, 3H), 1.10 (t, J = 6.9 Hz, 3H) M/Z (ES+) Calc.: 479.29 Found: 480.45 (M + H) Analytical HPLC: Method A1 SunFire MS C18 (4.6 × 100 mm) 5 um Retention Time: 7.98 min |

### Analytical Methods:

### Method A1

Solvent A: 0.2% Et₃N in water

Solvent B: 0.2% Et₃N in acetonitrile

Flow rate: 2.0 ml/min

Linear Gradient:

| time | | |
|---|---|---|
| (min) | % A | % B |
| 0 | 70 | 30 |
| 2 | 70 | 30 |
| 9 | 5 | 95 |
| 14 | 5 | 95 |

### Method C1

Mobile Phase: 0.1% Et₂NH in ethanol

Flow rate: 1.0 ml/min

Isocratic.

TABLE 4
IC₅₀ Values of Exemplary Compounds

| Example | | | |
|---|---|---|---|
| # | Structure | ER-Number | IC₅₀ (μm) |
| 42 | 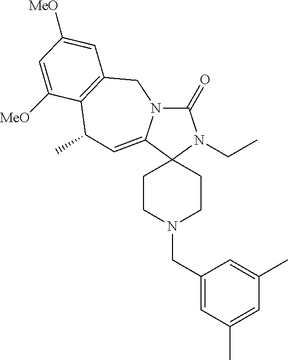 | ER-819762 | 0.04 |
| 43 | 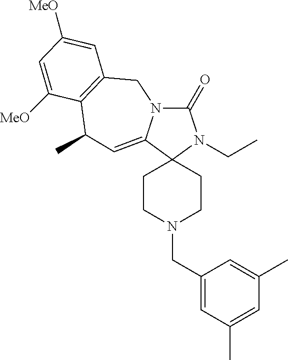 | ER-819763 | 0.55 |
| 44 | 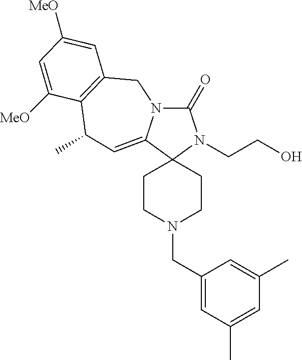 | ER-819786 | 0.03 |
| 45 | 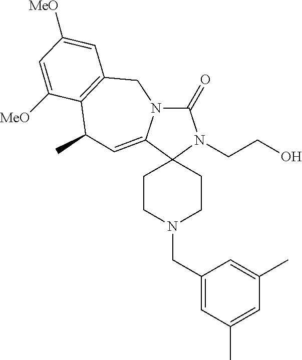 | ER-819787 | 0.17 |
| 46 | 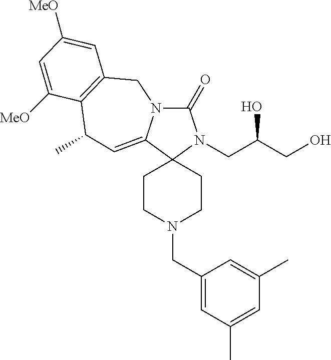 | ER-819788 | 0.03 |
| 47 | 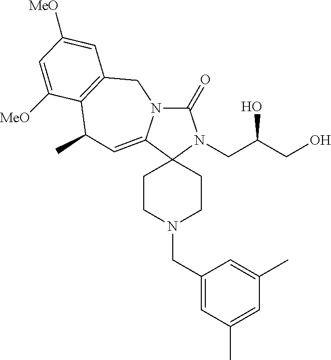 | ER-819789 | 0.17 |
| 48 | 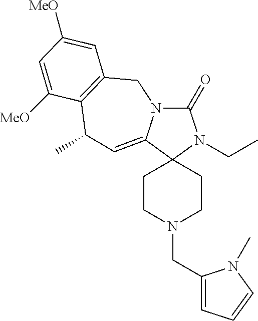 | ER-819924 | 0.05 |
| 49 | 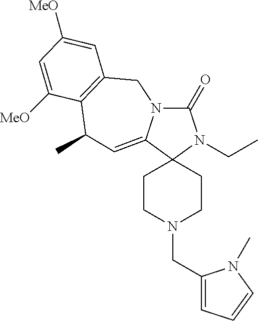 | ER-819925 | 0.32 |
| 50 | 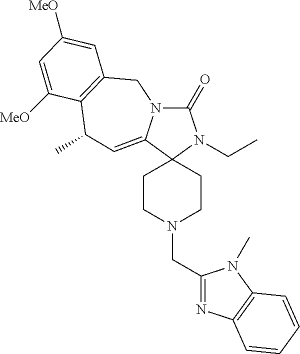 | ER-819926 | 0.04 |
| 51 | 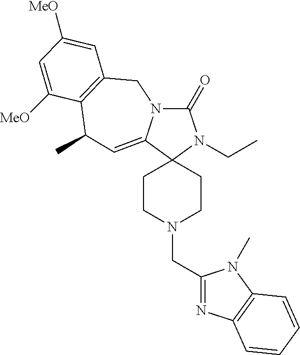 | ER-819927 | 0.21 |
| 52 | 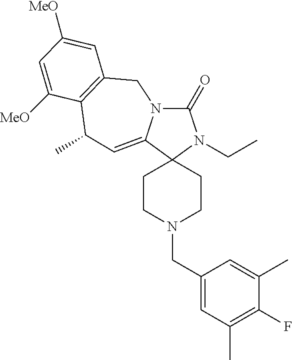 | ER-819931 | 0.07 |
| 53 | 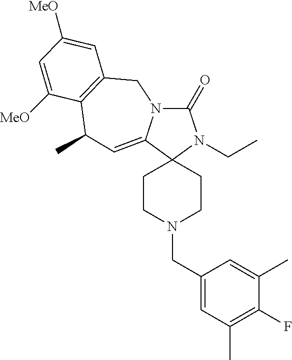 | ER-819943 | >10 |
| 54 | 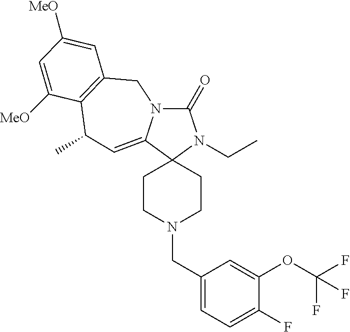 | ER-819933 | 1.15 |
| 55 | 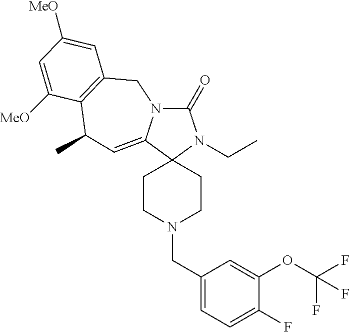 | ER-819945 | >10 |
| 56 | 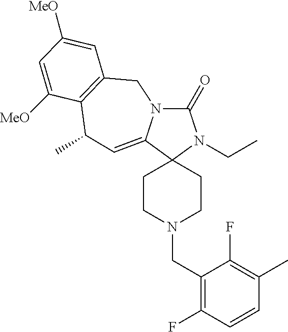 | ER-819934 | 0.10 |
| 57 | 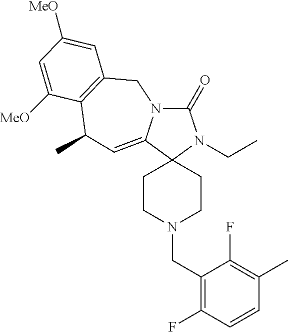 | ER-819946 | 2.97 |
| 58 | 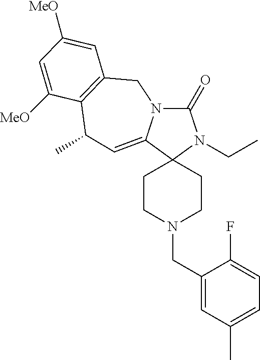 | ER-819935 | 0.13 |
| 59 | 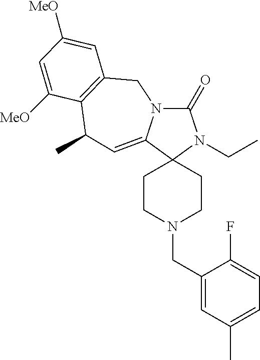 | ER-819947 | 2.6 |
| 60 | 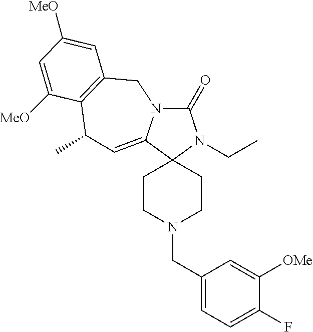 | ER-819936 | 0.12 |
| 61 | 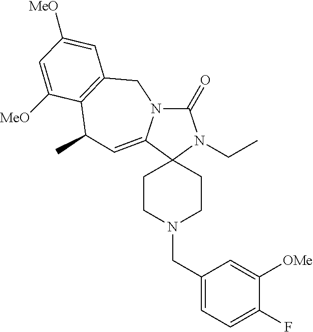 | ER-819948 | >10 |
| 62 | 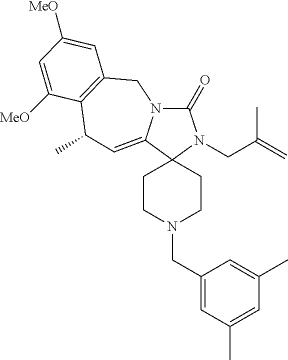 | ER-820006 | 0.06 |
| 63 | 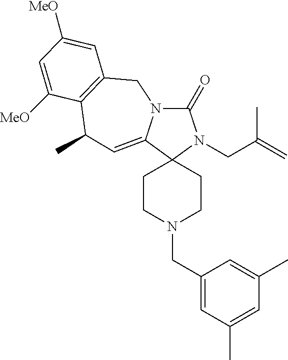 | ER-820007 | 1.26 |
| 64 | 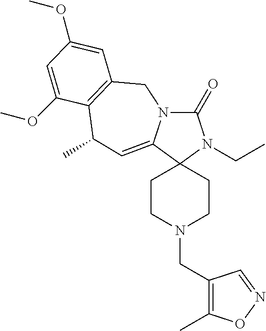 | ER-819810-01 | 0.012 |
| 65 | 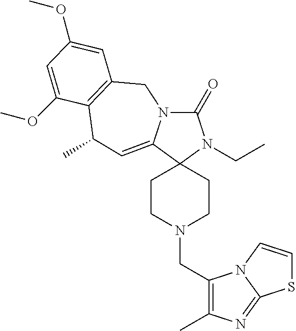 | ER-811352-02 | 0.013 |
| 66 | 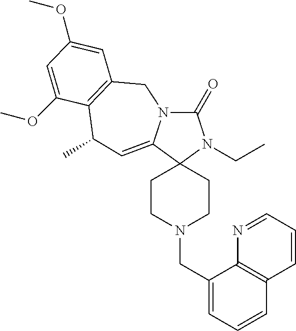 | ER-819955-01 | 0.020 |
| 67 | 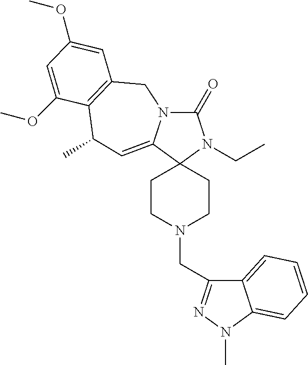 | ER-819800-01 | 0.023 |
| 68 | 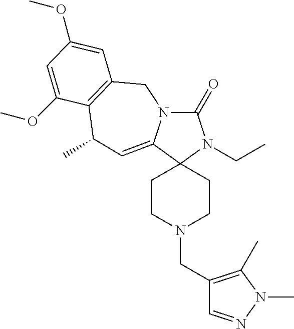 | ER-819976-01 | 0.023 |
| 69 | 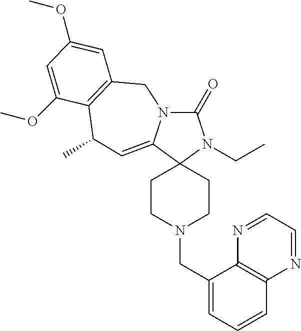 | ER-819953-01 | 0.026 |
| 70 | 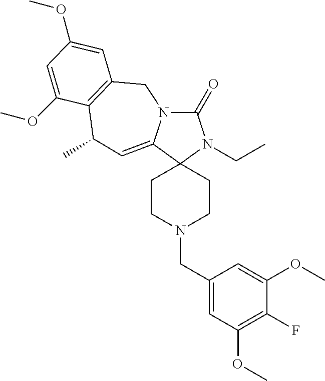 | ER-824214-00 | 0.029 |
| 71 | 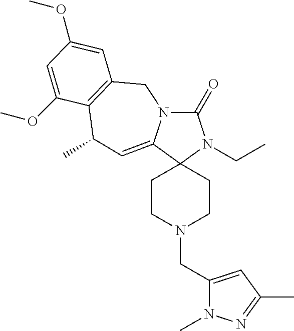 | ER-819973-01 | 0.030 |
| 72 | 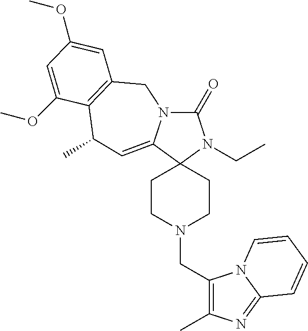 | ER-811305-01 | 0.031 |
| 73 | 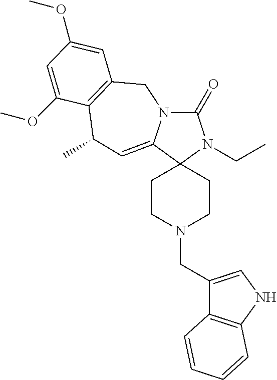 | ER-819783-01 | 0.035 |
| 74 | 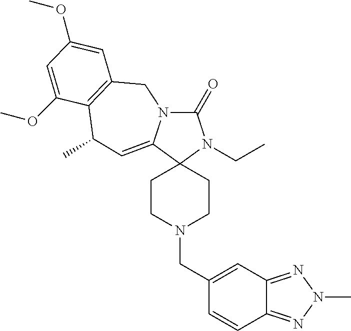 | ER-819847-01 | 0.035 |
| 75 | 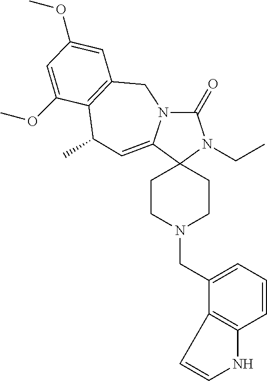 | ER-811300-01 | 0.035 |
| 76 | 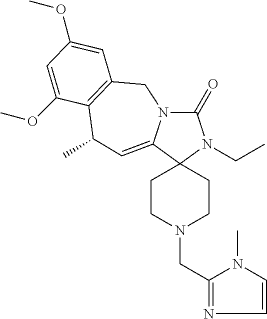 | ER-811278-01 | 0.037 |
| 77 | 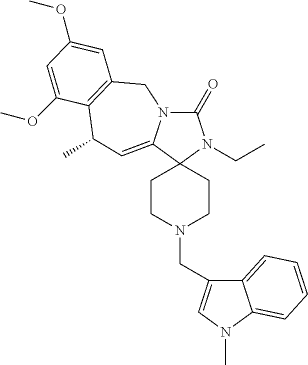 | ER-819804-01 | 0.040 |
| 78 | 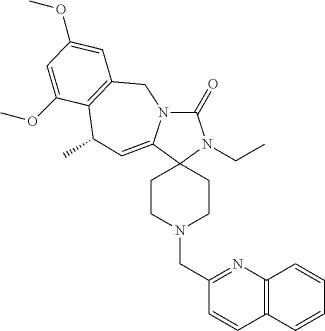 | ER-811323-01 | 0.042 |
| 79 | 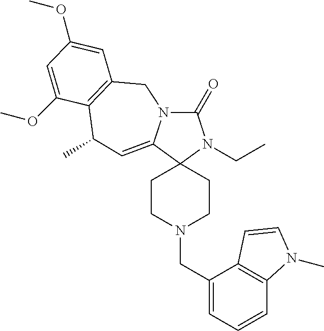 | ER-811349-01 | 0.043 |
| 80 | 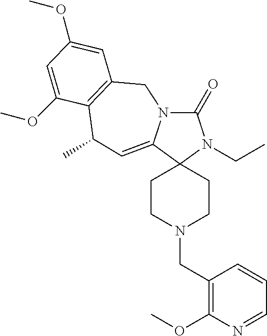 | ER-819833-01 | 0.047 |
| 81 | 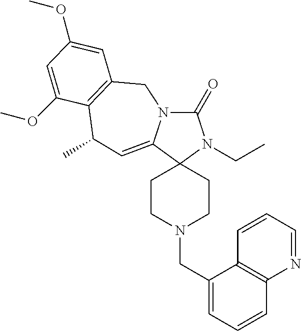 | ER-819954-01 | 0.047 |
| 82 | 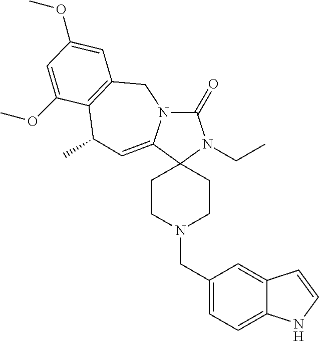 | ER-819966-01 | 0.048 |
| 83 | 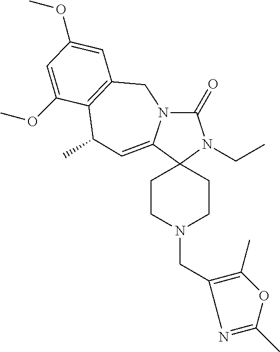 | ER-811283-01 | 0.053 |
| 84 | 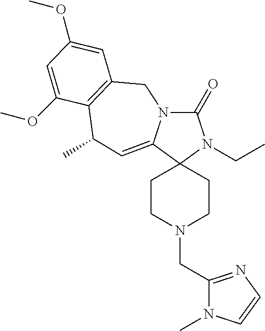 | ER-819957-01 | 0.055 |
| 85 | 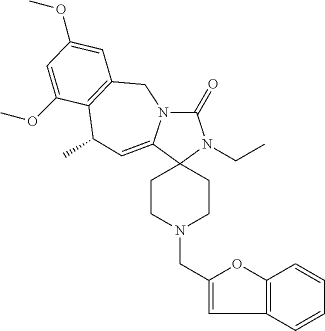 | ER-811308-01 | 0.056 |
| 86 | 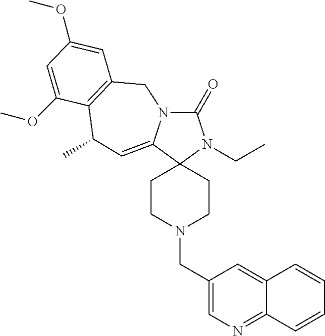 | ER-819837-01 | 0.056 |
| 87 | 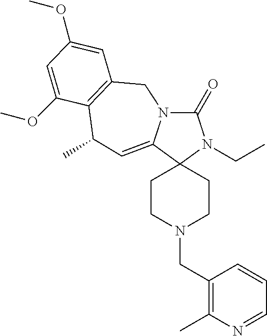 | ER-819832-01 | 0.057 |
| 88 | 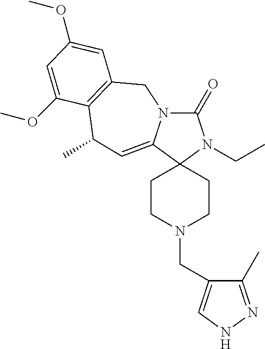 | ER-819826-01 | 0.067 |
| 89 | 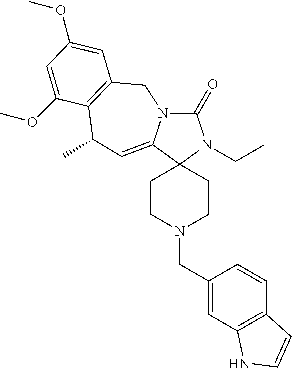 | ER-819844-01 | 0.067 |
| 90 | 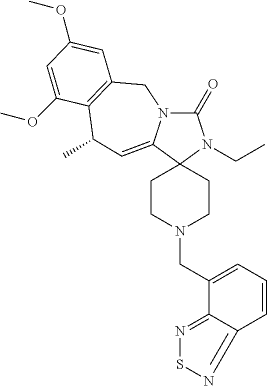 | ER-811332-01 | 0.067 |
| 91 | 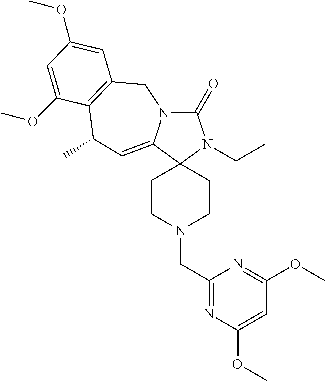 | ER-820004-01 | 0.067 |
| 92 | 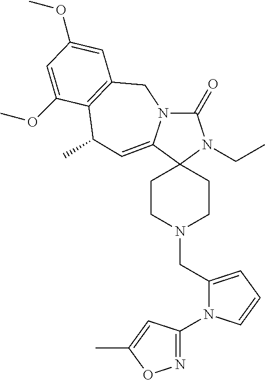 | ER-820017-01 | 0.069 |
| 93 | 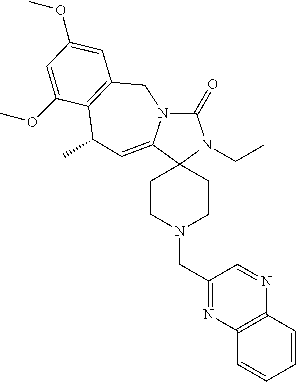 | ER-811297-01 | 0.074 |
| 94 | 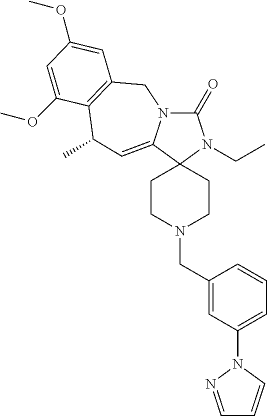 | ER-811317-01 | 0.074 |
| 95 | 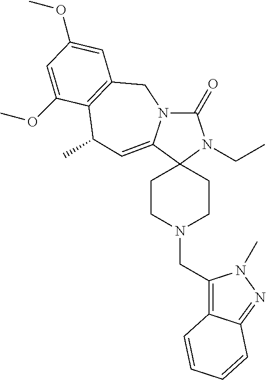 | ER-811312-01 | 0.077 |
| 96 | 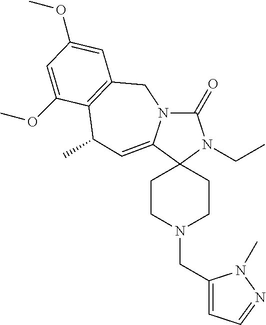 | ER-819958-01 | 0.079 |
| 97 | 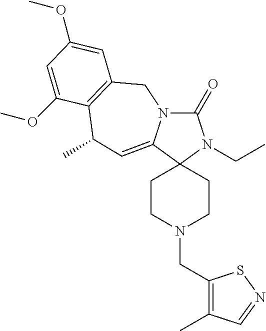 | ER-819842-01 | 0.081 |
| 98 | 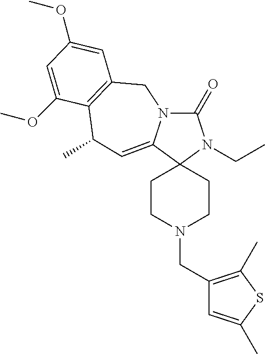 | ER-811365-01 | 0.084 |
| 99 | 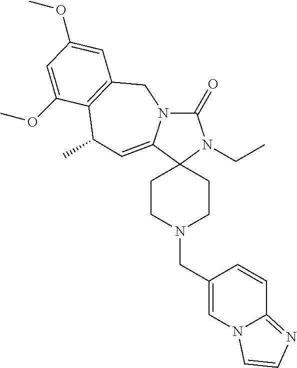 | ER-811284-01 | 0.088 |
| 100 | 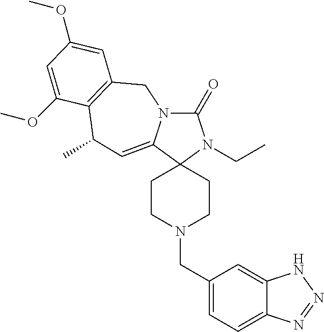 | ER-819820-01 | 0.090 |
| 101 | 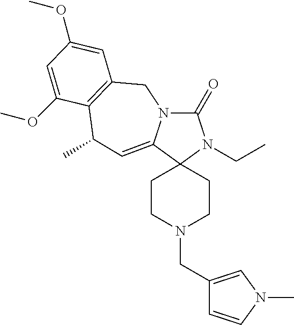 | ER-819961-01 | 0.096 |
| 102 | 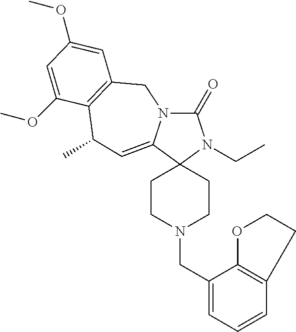 | ER-811306-01 | 0.10 |
| 103 | 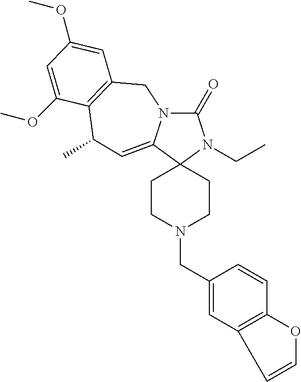 | ER-811304-01 | 0.10 |
| 104 | 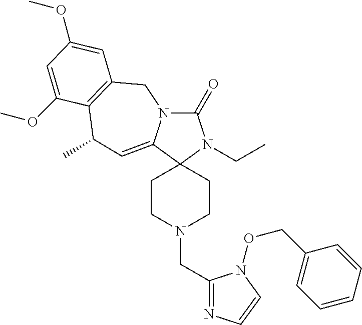 | ER-820009-00 | 0.11 |
| 105 | 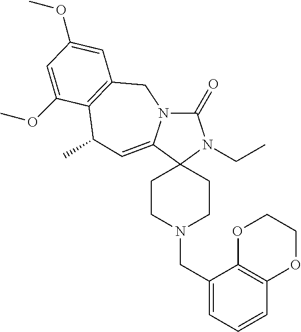 | ER-811291-01 | 0.12 |
| 106 | 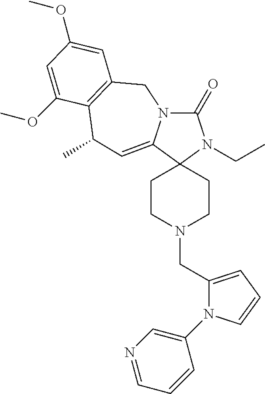 | ER-819979-01 | 0.12 |
| 107 | 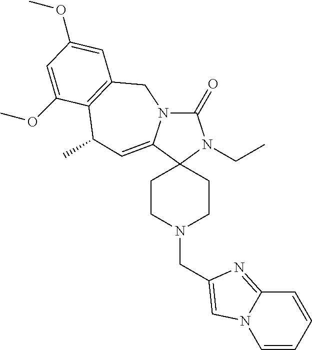 | ER-811292-01 | 0.13 |
| 108 | 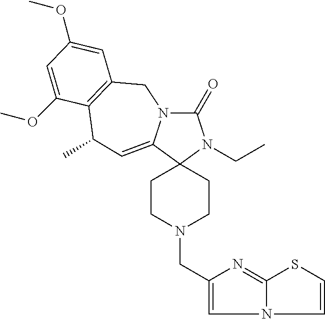 | ER-811309-01 | 0.13 |
| 109 | 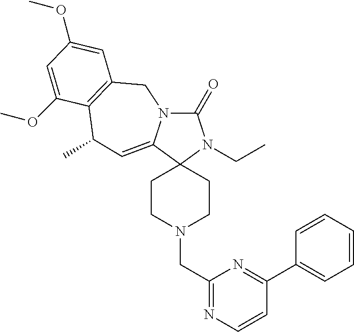 | ER-819985-01 | 0.13 |
| 110 | 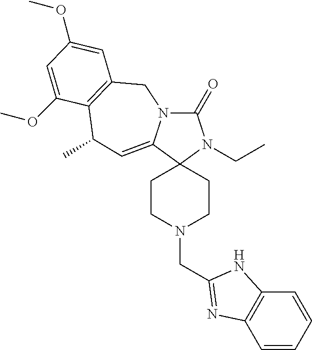 | ER-819965-01 | 0.13 |
| 111 | 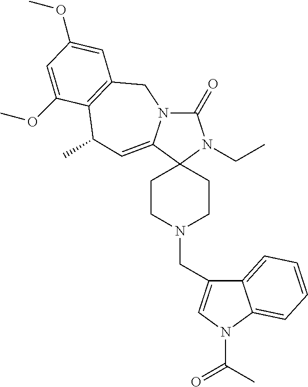 | ER-819808-01 | 0.14 |
| 112 | 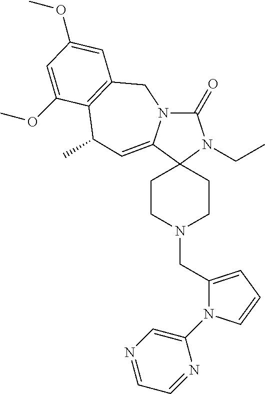 | ER-820020-02 | 0.18 |
| 113 | 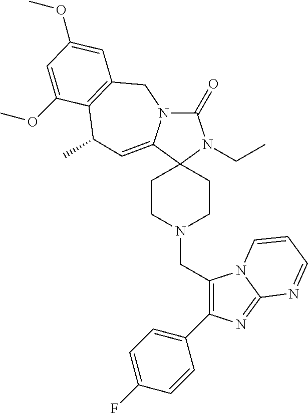 | ER-811346-02 | 0.20 |
| 114 | 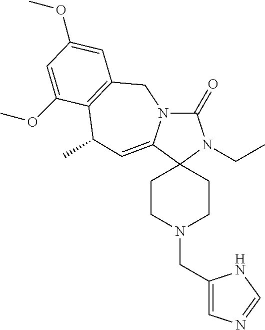 | ER-819780-01 | 0.20 |
| 115 | 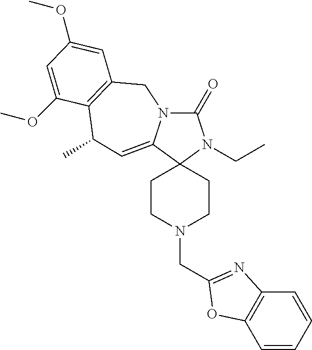 | ER-819981-01 | 0.20 |
| 116 | 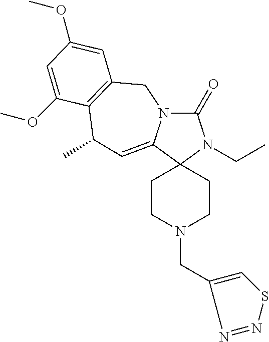 | ER-811279-01 | 0.21 |
| 117 | 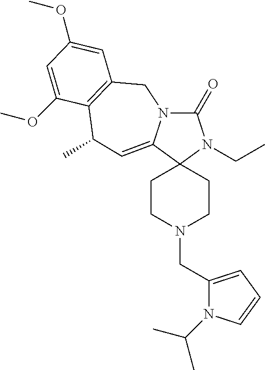 | ER-811358-01 | 0.22 |
| 118 | 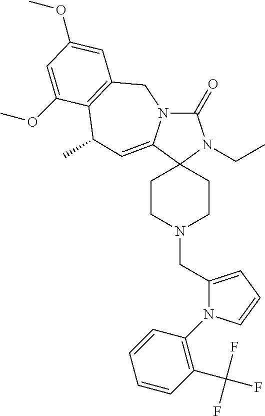 | ER-819849-01 | 0.22 |
| 119 | 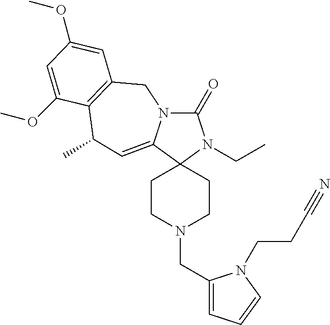 | ER-820008-00 | 0.24 |
| 120 | 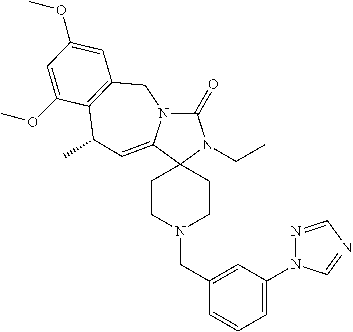 | ER-811302-01 | 0.25 |
| 121 | 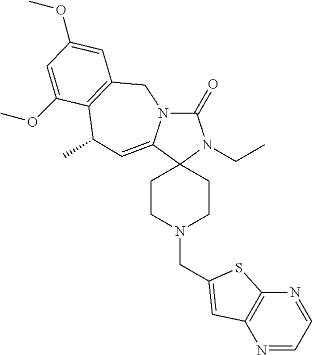 | ER-811301-01 | 0.26 |
| 122 | 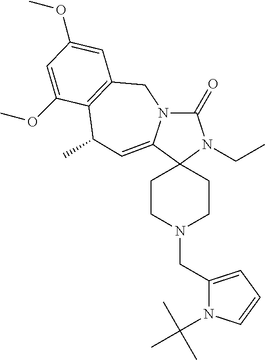 | ER-811359-01 | 0.27 |
| 123 | 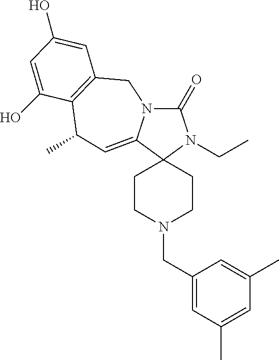 | ER-819888-00 | 0.30 |
| 124 | 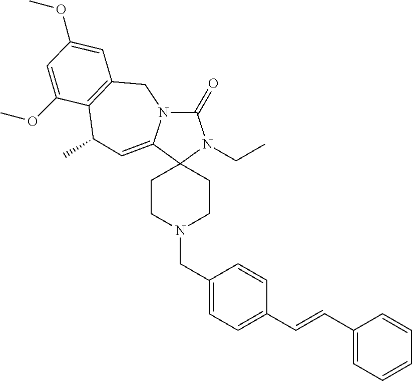 | ER-819814-01 | 0.30 |
| 125 | 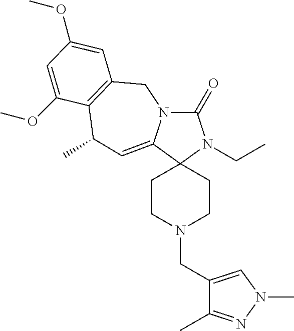 | ER-819971-01 | 0.034 |

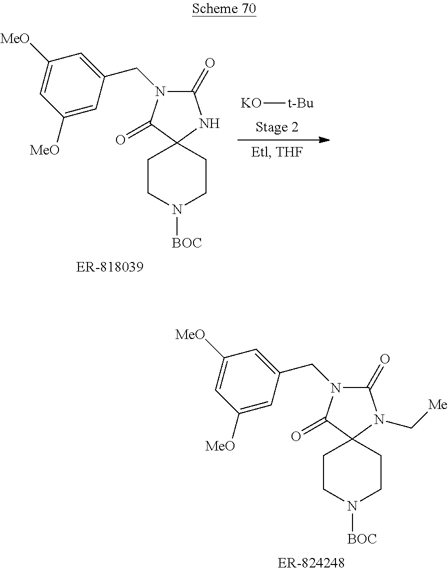

ER-824248 ER 818039 was prepared according to Scheme 1 and 2. As depicted in Scheme 70 above, ER-818039 (1 wt, 1 eq) is charged to a dry inerted reactor. Anhydrous THF (4.45 wts, 5.0 vols) is charged to the reactor. The solution is heated to 50-55° C. Potassium tert-butoxide 20% wt/wt in THF (1.6 wts, 1.2 eq) is added over a period of 20 min keeping the temperature below 55° C.-60° C. The solution is stirred for 15-20 min then Iodoethane (0.45 wts, 1.2 eq) is charged over a period of 15-20 min keeping the temperature below 55° C. The reaction is stirred for 8-12 h and monitored for completion by TLC (Hept:EtOAc, 1:1) and HPLC. Once the reaction is completed cool the reactor to 20-25° C., then quench with water (4 wts) followed by brine (4 wts), then add EtOAc (4.51 wts, 5 vols) stir for 10-15 min then allow to partition. Separate the aqueous layer and back wash with EtOAc (4.51 wts, 5 vols) if necessary. The organics are combined and concentrated to dryness in vacuo not exceeding 30° C. The oil crude ER-824248 (1.07 wts, 100%) is taken without purification to the next step.

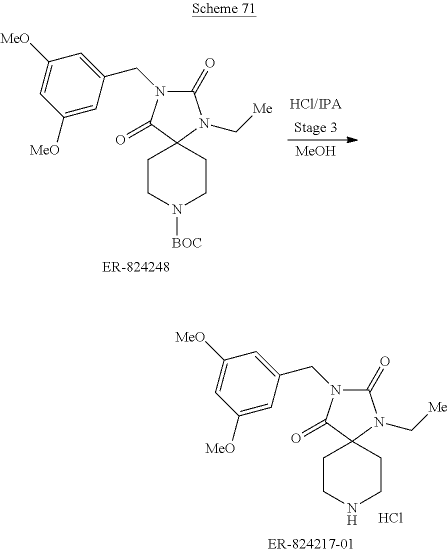

ER-824217-01. As depicted in Scheme 71 above, ER-824248 (1 wt, 1 eq) is charged to reactor. Anhydrous methanol (2.0 wts, 2.5 vols) is added. While stirring charge 5-6 M hydrogen chloride in IPA (0.74 wts, 0.81 vols, 2.0 eq). The reaction is stirred at room temperature and monitored by TLC (EtOAc) and HPLC. After 15-20 minutes solid precipitate start to form. The reaction is stirred for 1-3 h Once the reaction is completed, charge MTBE (1.85 wts, 2.5 vols), cool to 0° C. and let stir for 1-2 h then filter, wash the cake with MTBE (1.48 wts, 2 vols) then dry the fine white powder at rt using a Buchner funnel under house vacuum overnight to get ER-824217-01 (0.78 wt, 92%).

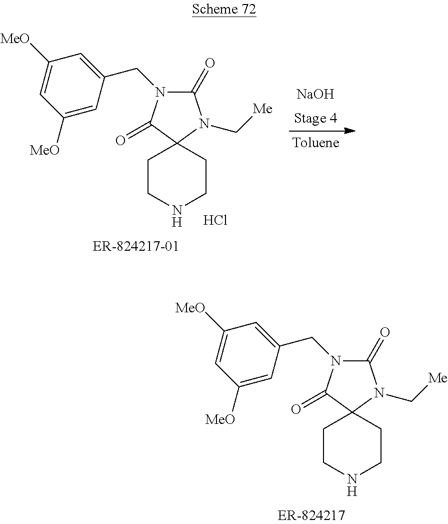

ER-824217. As depicted in Scheme 72 above, ER-824217-01 (1 wt, 1 eq) is charged to a reactor. Toluene ACS grade (4.32 wts, 5.0 vols) is added. The resulting mixture is stirred at 20-25° C. and 1N aqueous sodium hydroxide (3.1 wts, 1.2 eq) in portions. After the addition is completed, stir for 30-40 min. The stirring is then stopped and the layers are allowed to separate. Separate the aqueous layer check by TLC (EtOAc) and back extract if necessary with Toluene (5 vols), concentrate the organic phase in vacuo not exceeding 30° C. Charge MTBE (3.7 wts, 5 vols) and heat to 55° C. until solution is homogeneous (20-40 min). Cool down to 0-5° C. (˜1.0° C./min), crystallization occurs between 35-32° C. When the temperature reaches 0-5° C. stir for 3-4h then filter off the crystalline material. Dry the white powder at it using a Buchner funnel under house vacuum for 8-12 h to get ER-824217-00 (0.62 wts, 78%)

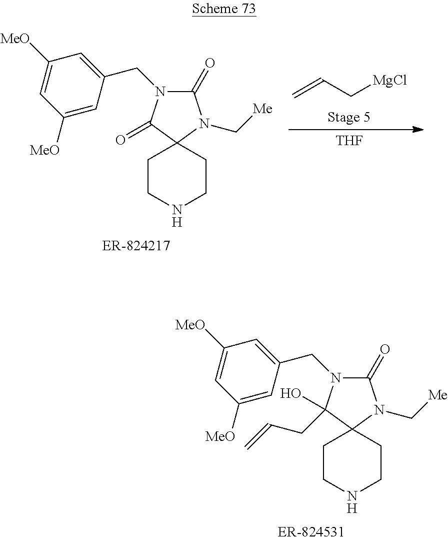

ER-824531. As depicted in Scheme 73 above, ER-824217 (1 wt, 1 eq) is charged to a reactor. Anhydrous THF (7.12 wts, 8.0 vols) is charged under inert atmosphere. Cool the reaction mixture to 0-5° C. 2.0M Allylmagnesium chloride in THF (2.86 wts, 2.88 vols, 2 eq) is added such a rate by keeping the temperature below 15° C. Allow the reaction to warm to rt. The progress of the reaction is monitored by TLC (10% methanol in DCM) and HPLC. After the reaction is completed (1-2 h) charge NH4Cl saturated solution (5.0 wts) then charge EtOAc (5.41 wts, 6 vols). Stir for 10-15 min then allow to partition. Separate the aqueous layer, check by TLC and back wash with EtOAc (4.51 wts, 5 vols) if necessary. The organics are combined and concentrated in vacuo not exceeding 30° C. Azeotrope with MTBE (3.7 wts, 5 vols). Charge MeCN (7.86 wts, 10 vols) to the reactor containing the product. Stir and heat to 65-70° C. then cool down to 0-5° C. (0.5° C./min). Stir for 1-2 h, filter and dry the white solid at rt using a Buchner funnel under house vacuum to give ER-824531 (0.89 wts, 80%)

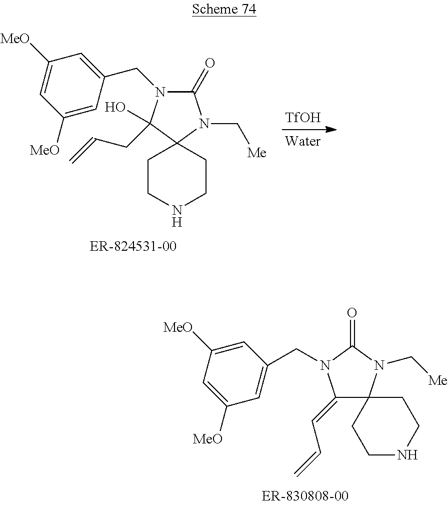

ER-830808-00. As depicted in Scheme 74 above, ER-824531 (1 wt, 1 eq) is charged to a reactor. Water (10.0 vols) is added. To the white slurry mixture is added Trifluoromethanesulfonic acid hydrate (0.25 vols, 1.0 eq) at rt, a white precipitate was formed, stir for 2 h then filter and dry the white solid at rt using a Buchner funnel under house vacuum to give ER-830808-00 (wts, %).

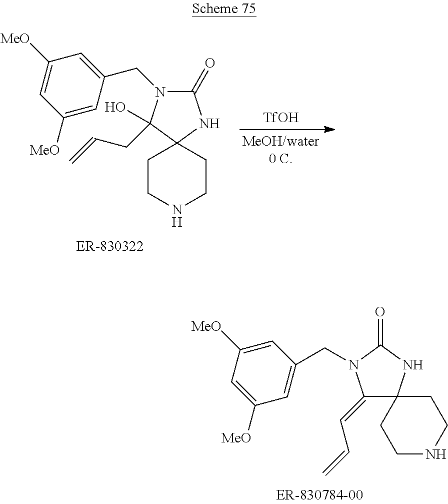

ER-830784-00 As depicted in Scheme 75 above, ER-830322 (1 wt, 1 eq) is charged to a reactor. Methanol (5 vols) is added followed by water (5 vols), the slurry is stirred and cooled ° C. Trifluormethanesulfonic acid (0.48 wt, 1.05 eq) is added. The slurry become clear solution. Check the completion of the reaction by TLC or HPLC). Once the reaction is completed cool to rt and charge 1 N NaOH (10 vols), stir for 1-2 h and then filter the white solid, dry at rt using a Buchner funnel under house vacuum to give ER-830784-00 (wt, %)

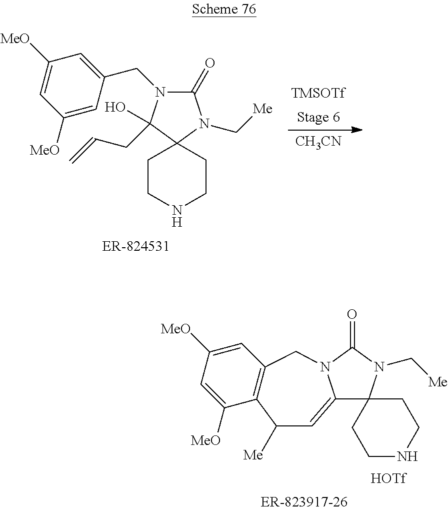

ER-823917-26. As depicted in Scheme 76 above, ER-824531 (1 wt, 1 eq) is charged to a reactor. Anhydrous ACN (Acetonitrile) (7.86 wts, 10.0 vols) is added. To white slurry mixture is added Trimethylsilyl trifluoromethanesulfonate (0.60 wts, 0.488 vols, 1.05 eq) at 20-25° C. keeping the temperature below 50° C. The progress of the reaction is monitored by TLC (10% methanol in DCM) and HPLC. After the reaction is completed (10 min) reduce under vacuo and not exceeding 30° C. the volume of ACN to 1-2 vols then charge MTBE (3.7 wts, 5 vols) cool to 0-5° C. and stir for 1-2 h. Filter the yellow/orange solid and wash the cake with MTBE (3.7 wts, 5 vols). Dry the solid at rt using a Buchner funnel under house vacuum overnight to afford ER-823917-26 (1.13 wts, 85%). The solid is carried forward to the next stage.

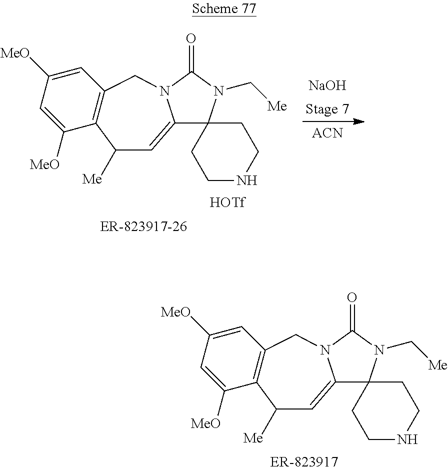

ER-823917. As depicted in Scheme 77 above, The solid ER-823917-26 (1 wt, 1 eq) is transferred to a reactor. Charge ACN (1.57 wts, 2 vols), while stirring charge 0.5M NaOH (2 wts, 2 vols), stir for 10-15 min till all clear solution then charge the remaining 0.5M NaOH (6 wts, 6 vols). Stirr the slurry for 1-2 h. Filter, wash the cake with water (4 vols) and dry at rt using a Buchner funnel under house vacuum. ER-823917 (0.64 wt, 90%) is obtained as white solid.

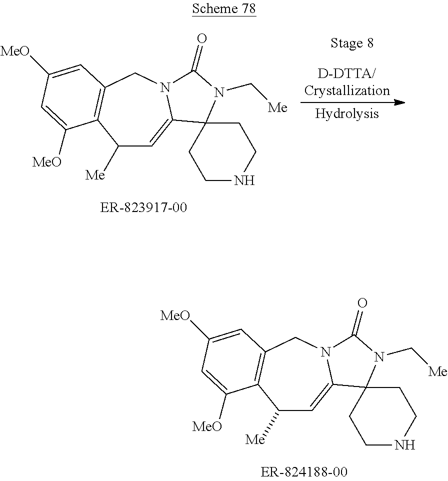

ER-824188-00. As depicted in Scheme 78 above,

(1) Crystallization step: Di-p-toluoyl-D-tartaric acid (D-DPTTA) (1.0 eq, 1.04 wt) is charged into a reactor followed by IPA (5 vols). The mixture is stirred for 10-15. IPA solution (or slurry)(5 vols) of ER-823917-00 (1.0 eq, 1 wt) is added to the reactor in 5 min with stirring followed by 2 more volumes of IPA rinse. Mixture is stirred for 10-15 min then water (2.4 vols) is charged to the reactor within 1 min. After water addition clear solution should be formed then crystallization should start within 15-20 min. The solution is stirred at RT for 16-24 hours. Crystallization process is monitored by HPLC of mother liquor sample. (Target: Area % of ER-824220/ER-824188>=95%).

After the crystallization is completed, the mixture is filtered. The cake containing ER-824188 D-DPTTA salt is washed three times with IPA/water (1/1 v/v, 3×1 vol). The washed solution is combined with mother liquor and stored for ER-824220 recovery. Filter cake is dried under high vacuum for 16 hours then transferred into a reactor for free-base/crystallization.

(2) Free basing of ER-824188: ER-824188 D-DPTTA salt in a reactor is stirred with methanol (4 vols) for 5 min. 1 N NaOH aqueous solution (2.5 vols) is added into the mixture within 1 min with stirring. The mixture is stirred for 10-15 min till clear solution. Water (10 vols) is added. Crystallization starts within the first 2 min of water addition. The mixture is stirred for 4-5 hour then filtered. The cake is washed 3 times with water (1 vol each time) then dried under high vacuum until constant weight to provide ER-824188-00 (38-44%).

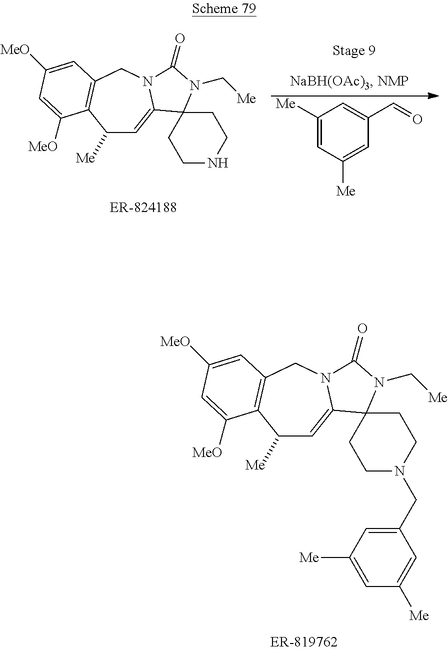

ER-819762. As depicted in Scheme 79 above, ER-824188-00 (1 wt, 1 eq) is charged to an inerted reactor. Anhydrous NMP (8.0 wts, 8 vols) is added. To the stirred solution is added 3,5-dimethylbenzaldehyde (0.397 wts, 0.398 vols, 1.1 eq) at rt. The solution is stirred at rt for 1-2 h. NaBH(OAc)₃ (0.721 wts, 1.2 eq) is added at once at rt (note: delayed exotherm) The solution is stirred at rt. The reaction progress is monitored by TLC (5% MeOH in DCM) and HPLC. Once the reaction is completed (1-3 h), heat the solution to 65-70° C. then charge water (8 wts). Cool to 15-20° C. (˜1° C./min) till a white precipitate is formed. Stir for another 1 h then filter at 15-20° C., wash the cake with water (2.0 wts). The white solid ER-819762 (1.12 wts, 85%) is dried under house vacuum to a constant weight.

Recrystallization: ER-819762 (1 wt, 1 eq) is added to a reaction flask, IPA (6.28 wts, 8 vols) is added, the slurry is stirred and heated to 70-75° C. till become solution, cool down (˜1° C./min) to 0-5° C. then stir for another extra 2 h. Filter using Buchner funnel under house vacuum, wash the cake with IPA (2 vols), transfer the white powder into a round bottom flask and dry under house vacuum (10-30 Ton) for 8-12 h to give ER-819762 (0.88 wt, 88%).

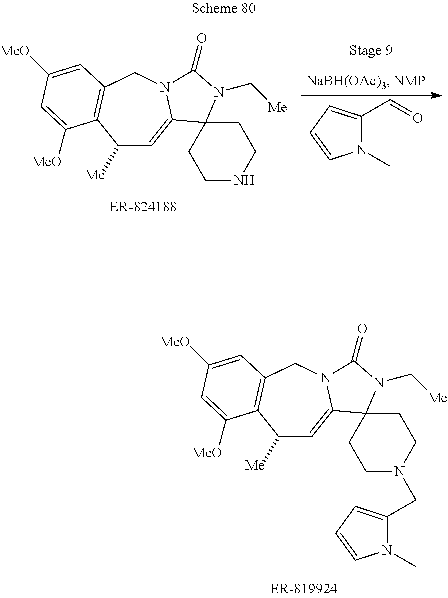

ER-819924. As depicted in Scheme 80 above, ER-824188-00 (1 wt, 1 eq) is charged to an inerted reactor. Anhydrous NMP (6.17 wts, 6.0 vols) is added. To the stirred solution is added N-Methyl-2-pyrrolecarboxaldehyde (0.362 wt, 0.399 vol, 1.2 eq) at rt. The solution is stirred at rt for 1-2 h. Sodium triacetoxyborohydride (0.84 wts, 1.4 eq) is added at once at rt (note: delayed exotherm) The solution is stirred at rt. The reaction progress is monitored by TLC (5% MeOH in DCM) and HPLC. Once the reaction is completed (1-3 h), heat the solution to 65-70° C. then charge sodium bicarbonate saturated solution (10 wts). Cool to 15-20° C. (˜1° C./min) till a white precipitate is formed. Stir for another 1 h then filter, wash the cake with water (2.0 wts). The white solid ER-819762 (1.25 wts, 100%) is dried under house vacuum to a constant weight.

Recrystallization: ER-819924-00 (1 wt, 1 eq) is added to a reaction flask, IPA:Hept (5:5 v/v, 3.92:3.42 wt/wt) is added, the slurry is stirred and heated to 60-70° C. till become solution, cool down (˜1° C./min) to 0-5° C. then stir for another extra 2 h. Filter using Buchner funnel under house vacuum, wash the cake with IPA:Hept (1:1 v/v, 0.78:0.68 wt/wt) and dry under house vacuum (10-30 Torr) for 8-12 h to give ER-819924-00 (1.04 wt, 83:3%).

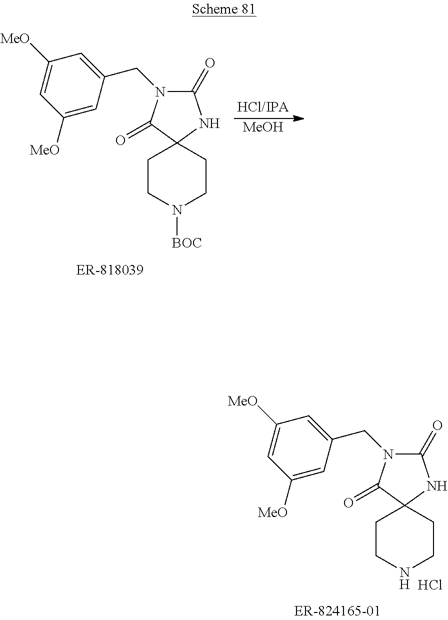

ER-824165-01 As depicted in Scheme 81 above, ER-818039 (1 wt, 1 eq) is charged to reactor. Anhydrous methanol (2.0 wts, 2.5 vols) is added. While stirring charge 5-6 M hydrogen chloride in IPA (1.85 wts, 2.17 vols, 5.0 eq). The reaction is stirred at room temperature and monitored by TLC (EtOAc) and HPLC. The reaction is stirred for 12-16 h Once the reaction is completed, charge MTBE (1.85 wts, 2.5 vols), cool to 0° C. and let stir for 1-2 h then filter, wash the cake with MTBE (1.85 wts, 2.5 vols) then dry the fine white powder at rt using a Buchner funnel under house vacuum overnight to get ER-824165-01 (0.80 wt, 94%).

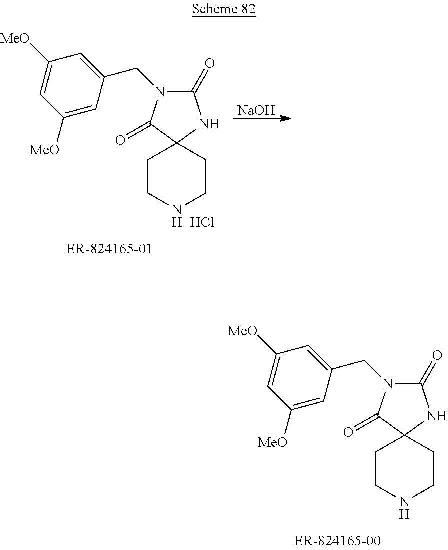

ER-824165-00 As depicted in Scheme 82 above, ER-824217-01 (1 wt, 1 eq) is charged to a reactor. MeOH (wts, 2 vols) is added. To the stirred slurry is added 1 N NaOH (4.0 wts, 4.0 vols). Stir the mixture till all become solution then charge water (4 vols). Stir for 60-90 min then filter the white powder. Dry the white powder at rt using a Buchner funnel under house vacuum for 8-12 h to get ER-824165-00 (0.67 wts, 73.0%)

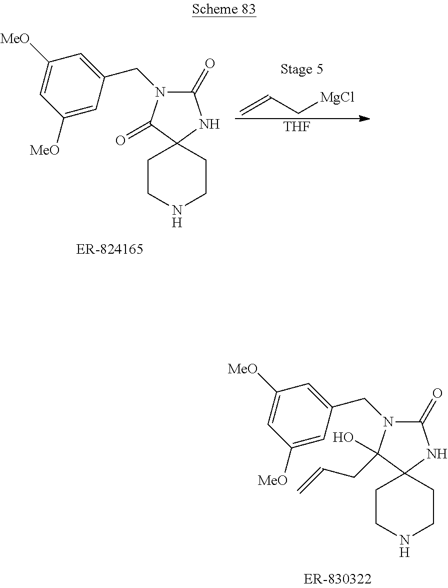

ER-830322 As depicted in Scheme 83 above, ER-824217 (1 wt, 1 eq) is charged to a reactor. Anhydrous THF (7.12 wts, 8.0 vols) is charged under inert atmosphere. 2.0M Allylmagnesium chloride in THF (wts, 4.7 vols, 3.0 eq) is added such a rate by keeping the temperature below 35° C. The progress of the reaction is monitored by TLC (10% methanol in DCM) and HPLC. After the reaction is completed (1-2 h) charge NH4Cl saturated solution (10.0 vols). Stir for 1-2 h, filter and dry the white solid at rt using a Buchner funnel under house vacuum to give ER-830322 (wts, %)

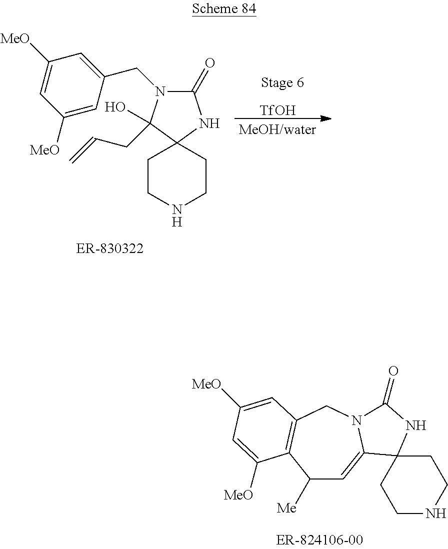

ER-824106-00 As depicted in Scheme 84 above, ER-830322 (1 wt, 1 eq) is charged to a reactor. Methanol (5 vols) is added followed by water (5 vols), the slurry is stirred and heated to 35-45° C. Trifluormethanesulfonic acid (0.48 wt, 1.05 eq) is added. The slurry become clear solution. Check the completion of the reaction by TLC or HPLC. Once the reaction is completed cool to it and charge 1 N NaOH (10 vols), stir for 1-2 h and then filter the white solid, dry at rt using a Buchner funnel under house vacuum to give ER-824106-00 (0.58 wt, 61%)

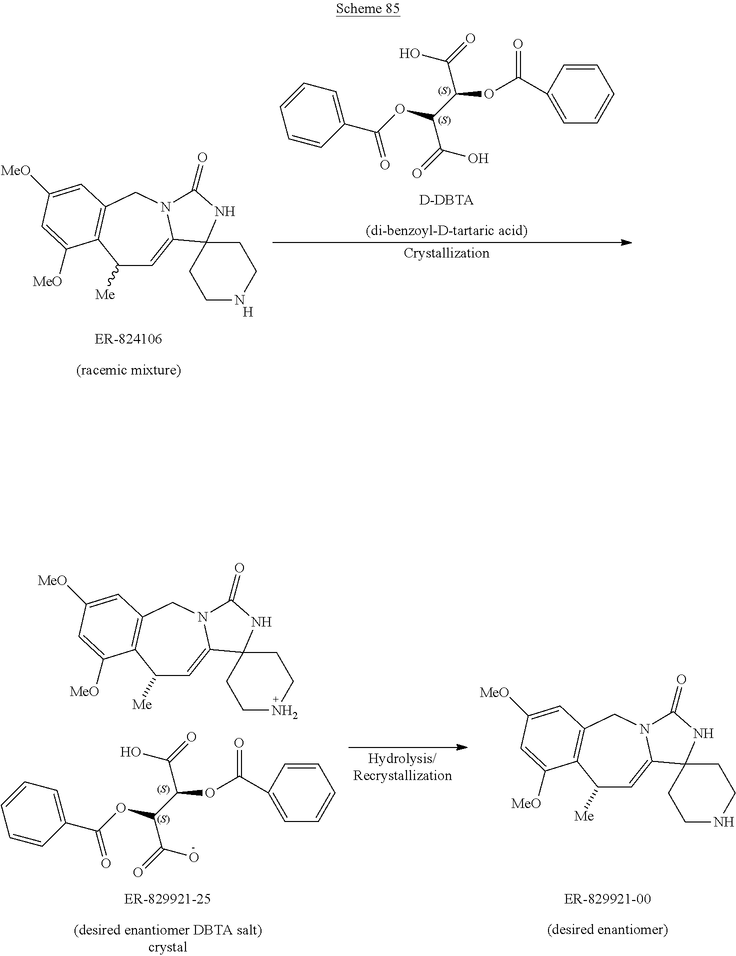

ER-829921-00. As depicted in Scheme 85 above,

Crystallization step: ER-824106 (1.0 eq, 1.0 wt) was slurried in 10 vol of MeOH and stirred at RT. D-DBTA (di-benzoyl-D-tartaric acid, 1.0 eq, 1.1 wt) was dissolved in MeOH (2 vol) and added into ER-824106 at one batch. The mixture stirred for 10 min followed by addition of water (1 vol). The mixture was stirred at RT for 18-24 hours until HPLC shown mother liquor sample with >90% ee of undesired enantiomer.

After crystallization is done, mixture in reactor is filtered. Filter cake containing ER-829921-25 was washed twice with MeOH/water (2/1 vol) mixture (3 volumes each time) on the filter funnel. Wash solution is combined with mother liquor and stored for ER-828098 recovery. Filter cake is dried under high vacuum at room temp for 16 hours then transferred into a reactor for hydrolysis/crystallization.

Hydrolysis/crystallization: Crystal of ER-829921-25 in a flask was slurried in MeOH (20 vol). 5 vol of NaOH (1N aq solution) was added in with stirring. The mixture was stirred for 1 hour and ER-824106 racemic mixture was crystallized. Crystal of ER-824106 racemic mixture was filtered and, 15 vol of water was added into the filtrate and the mixture stirred at RT for 18 hours to let the desired enantiomer to crystallize. The mixture was then concentrated to get rid of methanol then filtered. The filter cake of ER-829921-00 was washed twice with 3 Vol of water then dried at room temp under high vacuum to provide the final product of ER-829921-00.

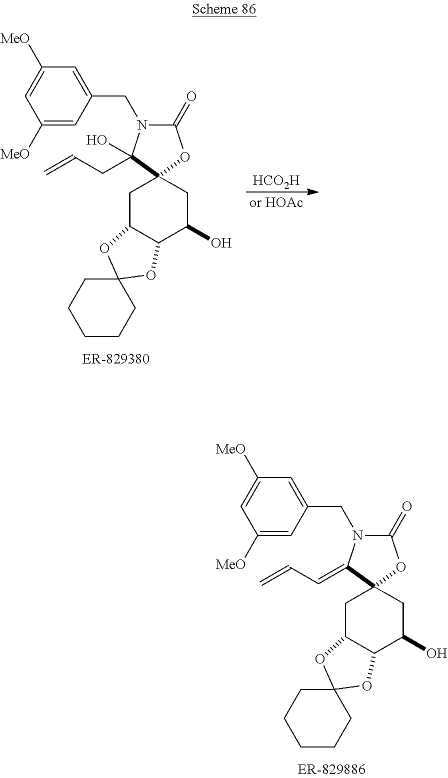

ER-829886. As depicted in Scheme 86 above, ER-829380-00 (1.00 Wt, 1.00 V, 1.00 eq.) was dissolved in acetonitrile (10.0 vols) and treated with formic acid (0.77 vols, 10.0 eq.). The resulting mixture was stirred at r.t. and followed by TLC (TBME, 10% MeOH/DCM). After total 5 h stirring, the mixture was diluted with TBME (100 vols), quenched with saturated aqueous NaHCO₃ (10.0 vols), the separated organic layer was washed with brine (10.0 vols). The organic layer was then concentrated to give crude product as white foam (1.00 wt), which was purified by flash chromatography: Redisep column (40.0 wts silica gel) was pre-conditioned with heptane (200 vols). The crude material was loaded atop the column with minimum amount of DCM and the column was eluted with 1:2 TBME/Heptane (360 vols), 1:1 TBME/Heptane (360 vols), 2:1 TBME/Heptane (360 voids), 3:1 TBME/Heptane (360 vols), 4:1 TBME/Heptane (360 vols), TBME (360 vols). All fractions were collected 20 vols each and analyzed by TLC (TBME). Fractions containing pure product were combined and concentrated to give the desired product as white solid (0.53 wt, yield 54.7%).

ER-829380-00 (1.00 wt, 1.00 v, 1.00 eq.) was dissolved in acetonitrile (10.0 vols) and treated with acetic acid (1.16 vols, 10.0 eq.). The resulting mixture was stirred at r.t. and followed by TLC (TBME, 10% MeOH/DCM). The reaction result is exactly the same as above, but much slower.

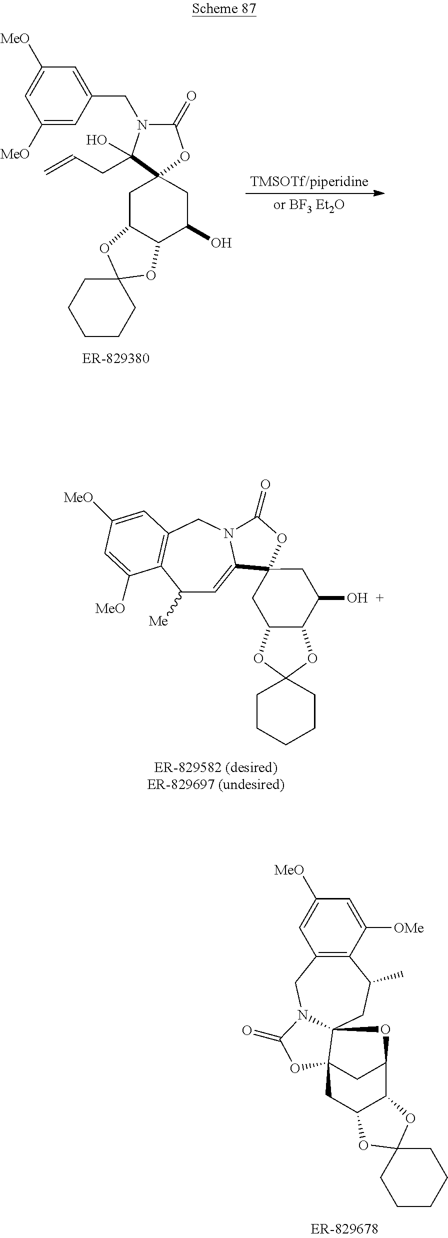

ER-829582 and ER-829678. As depicted in Scheme 87 above, ER-829380-00 (1.00 wt, 1.00 V, 1.00 eq.) was dissolved in acetonitrile (10.0 vols) and piperidine (0.20 vols, 1.00 eq.) was added. The mixture was cooled to 0° C. To the solution, trimethylsilyl trifluoromethanesulfonate (0.39 vols, 1.05 eq.) was added dropwise (Tmax=15° C.). The mixture was then stirred at r.t. and followed by TLC (TBME, 10% MeOH/DCM). Upon completion of the reaction (1 h), the reaction mixture was quenched with saturated aqueous NaHCO₃ (2.00 vols), extracted with TBME (20.0 vols). The separated organic layer was washed with saturated NH4Cl (2.00 vols) and brine (2.00 vols). The organic layer was concentrated to give crude product as white foam (1.25 wts), which was purified by flash chromatography: RediSep column (16.5 wts silica gel) was preconditioned with Heptane (44 vols). The crude product was loaded atop the column with minimum amount of DCM. The column was eluted with 1:2 TBME/Heptane (132 vols), 1:1 TBME/Heptane (132 vols), 2:1 TBME/Heptane (132 vols). All fractions were collected 22.5 vols each and analyzed by TLC (4:1 TBME/Heptane). Fractions containing pure product were combined and concentrated to give the desired product as white solid (0.22 wts, yield 23.0%). Meanwhile, ER-829678 (0.14 wts, 14.7%) was also collected as byproduct, which can be converted to desired product by acid treatment.

ER-829380-00 (1.00 wt, 1.00 V, 1.00 eq.) was dissolved in acetonitrile (10.0 vols) and treated with boron trifluoride etherate (0.025 vols, 0.1 eq.). The resulting mixture was stirred at r.t. and followed by TLC (2:1 TBME/Heptane, TBME, 10% MeOH/DCM). The reaction is exactly the same as TMSOTf catalyzed cyclization.

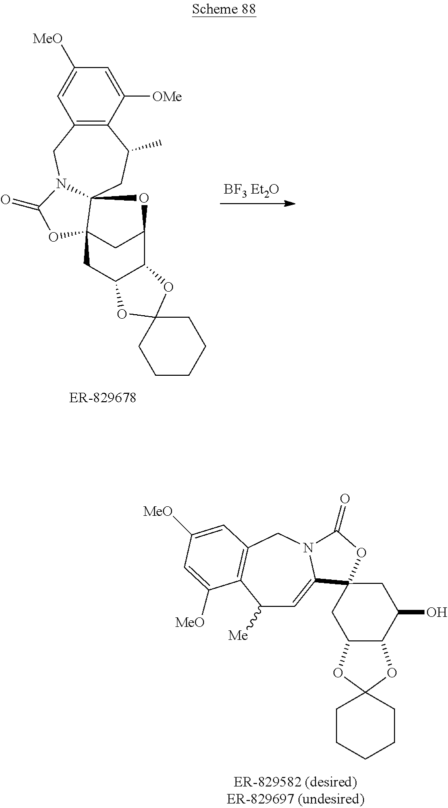

ER-829582. As depicted in Scheme 88 above, ER-829678 (1.00 wt, 1.00 V, 1.00 eq.) was dissolved in acetonitrile (10.0 vols) and treated with boron trifluoride etherate (0.03 vols, 0.10 eq.). The mixture was then stirred at r.t. and monitored by TLC (2:1 TBME/Heptane, 10% MeOH/DCM). After 2.5 h stirring, the reaction was quenched with saturated aqueous NaHCO₃ (5.00 vols), extracted with TBME (50 vols). The separated organic layer was washed with brine (5.00 vols) and concentrated to give crude product as white foam (0.96 wts), which was purified by flash chromatography: RediSep column (15.6 wts silica gel) was preconditioned with Heptane (39 vols). The crude material was loaded atop the column with minimum amount of DCM. The column was eluted with 2:1 Heptane/TBME (117 vols), 1:1 Heptane/TBME (117 vols), 1:2 Heptane/TBME (117 vols), TBME (197 vols). All fractions were collected 13 vols each and analyzed by TLC (TBME). Fractions containing pure product were combined and concentrated to give desired product as white foam (0.61 wt, yield 61.2%). The starting material was also recovered.

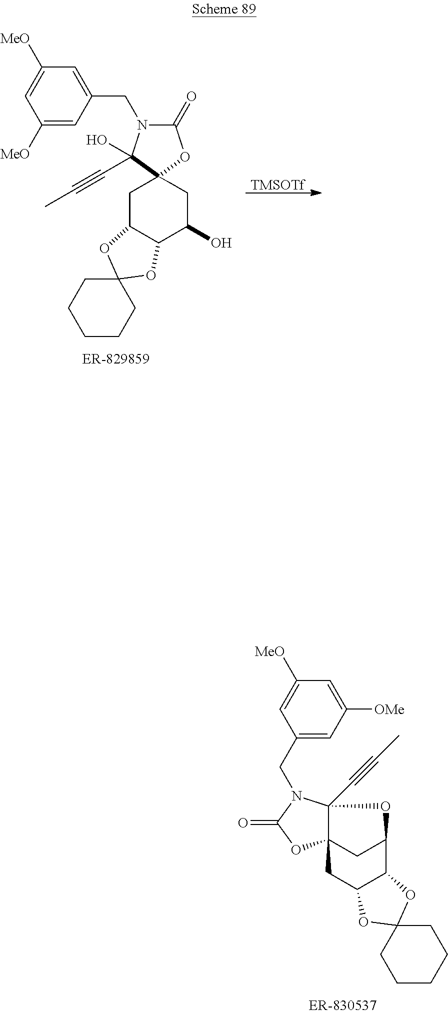

ER-830537. As depicted in Scheme 89 above, ER-829859-00 (1.00 wt, 1.00 V, 1.00 eq.) was dissolved in acetonitrile (10.0 vols) and piperidine (0.20 vols, 1.00 eq.) was added. To the solution, trimethylsilyl trifluoromethanesulfonate (0.39 vols, 1.05 eq.) was added dropwise (Tₘₐₓ=24° C.). The mixture was then stirred at r.t. and followed by TLC (TBME, 10% MeOH/DCM). Upon completion of the reaction (1 h), the mixture was quenched with saturated aqueous NaHCO₃ (10.0 vols), extracted with TBME (500 vols). The separated organic layer was washed with saturated aqueous NaHCO₃ (10.0 vols) and brine (10.0 vols). The organic layer was concentrated to give crude product as yellow foam (0.87 wts), which was purified by flash chromatography: RediSep column (43 wts silica gel) was preconditioned with Heptane (300 vols). The crude material was loaded atop the column with minimum amount of DCM. The column was eluted with 1:2 TBME/Heptane (384 vols), 1:1 TBME/Heptane (384 vols), 2:1 TBME/Heptane (384 vols), TBME (640 vols). All fractions were collected 75 vols each and analyzed by TLC (4:1 TBME/Heptane, TBME). Fractions containing pure product were combined and concentrated to give the desired product as white foam (0.21 wts, yield 22.1%).

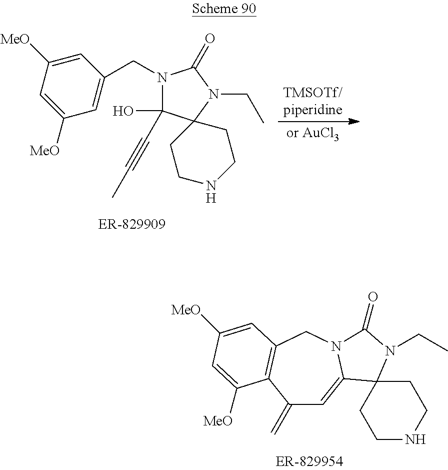

ER-829954. As depicted in Scheme 90 above, ER-829909-00 (1.00 wt, 1.00 V, 1.00 eq.) was dissolved in acetonitrile (10.0 vols). To the solution, trimethylsilyl trifluoromethanesulfonate (0.47 vols, 1.00 eq.) was added dropwise. The mixture was then stirred at r.t. and followed by TLC (20% MeOH/DCM). Upon completion of the reaction, the mixture was quenched with saturated aqueous NaHCO₃ (10 vols), extracted with ethyl acetate (200 vols). The separated organic layer was washed with saturated aqueous NaHCO₃ (10.0 vols) and brine (10.0 vols). The organic layer was concentrated to give crude product as yellow oil (1.5 wts), which was purified by flash chromatography: RediSep column (40.0 wts silica gel) was preconditioned with Heptane (100 vols). The crude material was loaded atop the column with minimum amount of DCM. The column was eluted with 1:1 TBME/Heptane (200 vols), 2:1 TBME/Heptane (200 vols), 4:1 TBME/Heptane (200 vols), TBME (400 vols), 5% MeOH/DCM (200 vols), 10% MeOH/DCM (200 vols), 20% MeOH/DCM (400 vols). All fractions were collected 27 vols each and analyzed by TLC (TBME, 10% MeOH/DCM). Fractions containing pure product were combined and concentrated to give the desired product as yellow oil (0.26 wts, yield 27.4%).

ER-829909-00 (1.00 wt, 1.00 V, 1.00 eq.) was dissolved in toluene (20.0 vols) and treated with GOLD (III) CHLORIDE (0.10 wts, 0.12 eq.). The mixture was then heated to reflux and followed by TLC (10% MeOH/DCM, 20% MeOH/DCM) and MS. After 22 h refluxing, the mixture was diluted with DCM (25.0 vols), and treated with boron trifluoride etherate (0.36 vols, 1.10 eq.). The mixture was stirred at r.t. for 1.5 h and then quenched with saturated aqueous NaHCO₃ (10.0 vols), extracted with ethyl acetate (300 vols) and washed with brine (10.0 vols). The organic layer was concentrated to give the crude product, which was purified by flash chromatography: RediSep column (89 wts silica gel) was preconditioned with DCM (670 vols). The crude material was, loaded atop the column with minimum amount of DCM. The column was eluted with 2% MeOH/DCM (532 vols), 5% MeOH/DCM (532 vols), 10% MeOH/DCM (532 vols). All fractions were collected 111 vols each and analyzed by TLC (10% MeOH/DCM). Fractions containing pure product were combined and concentrated to give the desired product as yellow oil (0.22 wts, yield 23.3%).

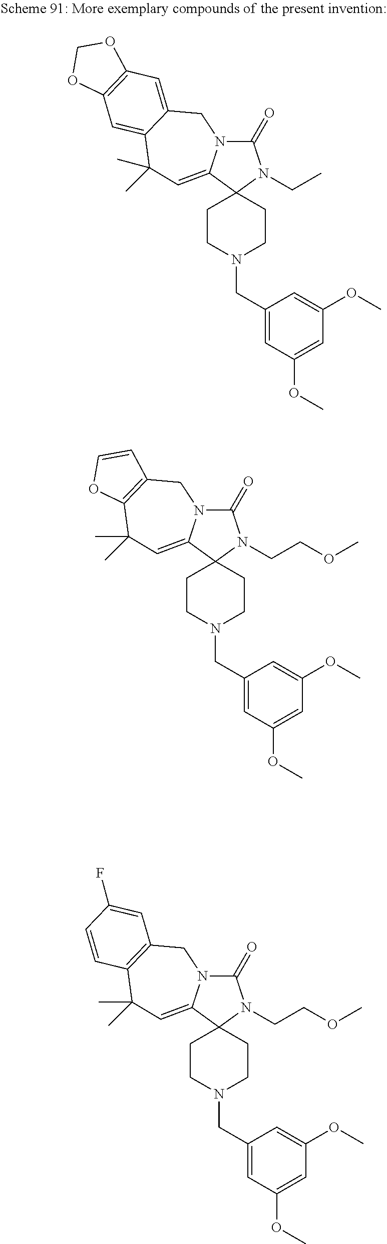

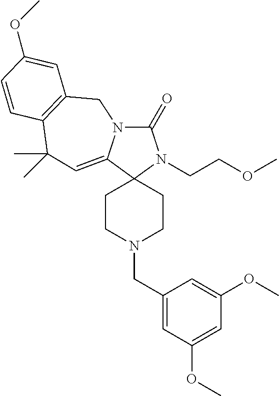

TABLE 4
Acids used in the cyclization reaction:

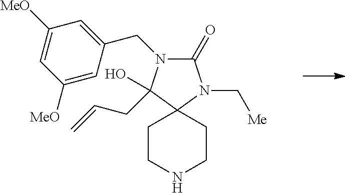

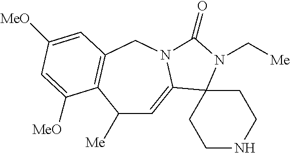

| Lot | | | | | Time | |
|---|---|---|---|---|---|---|
| # | Acid | Eq | Solvent | Temp | h | Results |
| 285 | Camphore sulfonic acid | 1.1 | DCM | rt | 1 | Messy |
| 286 | Di-benzoyl-D-Tartaric acid | 1.1 | DCM | rt | 48 | No pdt |
| 321 | Trifluoroacetic acid | 1-5 | THF | −78° C. | 0.5 | No Pdt |
| 307 | Camphore sulfonic acid | 0.1 | THF | Rt | 1 | Uncompleted |
| 310 | Methanesulfonic acid | 1.1 | THF | −70° C. | 1 h | Uncompleted |
| 304 | Magnesium bromide | 1.0 | THF | Rt | 12 | No reaction |
| 311 | Formic acid | 1-10 | THF | Rt | 12 | No reaction |
| 316 | Acetic acid | 1-5 | THF | Rt | 1 | No reaction |
| 317 | Tetrabutylammonium p- | 1-2 | THF | Rt-50° C. | 1 | No reaction |
| | toluenesulfonate | | | | | |
| 318 | Amberlite-IRP-69 | | THF | Rt | 2 | No reaction |
| 326 | Boron trifluoride diethyl etherate | 1 | THF | rt | 1 | Pdt + imps |
| 332 | Trimethylsilyl | 1.05 | THF | Rt | 0.2 | Pdt |
| | trifluoromethansulfonate | | | | | |
| 351 | Trimethylsilyl chloride | 1.05 | MeCN | Rt | 5 min | Messy |
| 328 | Indium | 1.0 | THF | Rt | 1 h | No reaction |
| | trifluoromethanesulfonate | | | | | |
| 352 | Titanium (IV) tetrachloride | 1.05 | MeCN | Rt | 10 min | OK |
| 358 | Titanium (IV) isopropoxide | 1.1 | MeCN | 50° C. | 12 h | uncompleted |
| 360 | Phosphoric acid | 1.1 | MeCN | rt | 30 min | Messy |

In some embodiments of the present invention, the choice of the acid depends on different substituents of the compound of formula (II), (III), (IIa) or (IIIa). For example, when R⁸ is hydrogen in formula (IIa) or (IIIa), weak acid, such as acetic acid, formic acid, tartic acid, may be used in the cyclization. However, when R⁸ is substituted with alkyl, stronger acid such as trifluoroacetic acid (TFA) may be used in the cyclization.

Other embodiments. While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

1. A method of making a compound of Formula I:
comprising the steps of: 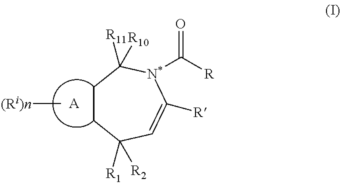
(a) providing a compound of Formula (II) or (III): 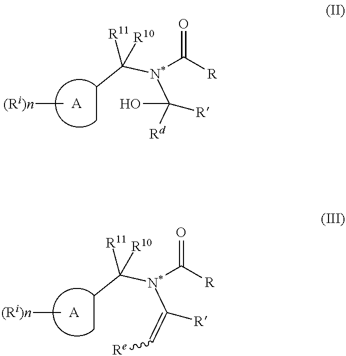
wherein:
ring A is phenyl or furanyl,
n is an integer selected from 0, 1, 2, 3 or 4,
each occurrence of Rⁱ is independently selected from the group consisting of hydrogen, hydroxyl, C₁₋₁₀ alkoxy, benzyloxy, benzyl, halo, amino, (C₁₋₆ alkyl)amino, (C₁₋₆alkyl)(C₁₋₆alkyl)amino, phenoxyl, and phenyl; or two adjacent Rⁱ, taken together, is —O—(CH₂)—O— or —O—CH₂—CH₂—O— and Rⁱ is attached to the A ring as valence permits;
R and R′ are each independently hydrogen, C₁₋₁₀ alkyl, C₂₋₁₀ alkenyl, C₂₋₁₀ alkynyl, C₁₋₁₀ alkoxy, C₁₋₁₀ alkylsulfonyl, C₁₋₄₀ haloalkyl, C₁₋₁₀ aminoalkyl, amino, (C₁₋₆ alkyl)amino, (C₁₋₆alkyl)(C₁₋₆alkyl)amino, C₃₋₁₀ cycloalkyl, C₃₋₁₀ cycloalkenyl, C₃₋₁₀ cycloalkynyl, C₃₋₁₀ heterocycle, C₃₋₁₄ aryl, or C₃₋₁₄ heteroaryl,
or R and R′ taken together form with N* a C₃₋₁₀ cycloalkyl, C₃₋₁₀ cycloalkenyl, C₃₋₁₀ cycloalkynyl, C₄₋₁₀ heterocyclyl, C₃₋₁₄ aryl, or C₃₋₁₄ heteroaryl ring system, which ring system is unsubstituted or substituted from one to four times with substituents independently selected from the group consisting of halo, oxygen, hydroxyl, sulfuryl, amino, nitro, cyano, C₁₋₁₀ haloalkyl, C₁₋₁₀ alkyl, C₃₋₁₀ spirocyclyl, C₃₋₁₀ spiroheterocyclyl, C₂₋₁₀ alkenyl, C₂₋₁₀ alkynyl, C₁₋₁₀ alkoxy, C₁₋₁₀ aminoalkyl, C₁₋₁₀ thioalkyl, C₃₋₁₀ heterocyclyl, C₃₋₁₀cycloalkyl, C₃₋₁₄ aryl, and C₃₋₁₄ heteroaryl,
R¹ and R² are independently hydrogen, C₁₋₁₀alkyl, C₂₋₁₀alkenyl, C₂₋₁₀ alkynyl, or taken together are C₂₋₁₀alkylidene or C₂₋₄₀alkenylidene, or R¹ and R² taken together form C₃₋₁₀ cycloalkyl or C₃₋₁₀heterocyclyl,
R¹⁰ and R¹¹ are independently selected from the group consisting of hydrogen, oxygen, hydroxyl, C₁₋₁₀ alkyl, C₂₋₁₀ alkenyl, C₂₋₁₀ alkynyl, C₁₋₁₀ alkoxy, C₁₋₁₀ alkylsulfonyl, C₁₋₁₀ haloalkyl, C₁₋₁₀ aminoalkyl, amino, (C₁₋₆ alkyl)amino, (C₁₋₆alkyl)(C₁₋₆alkyl)amino, C₃₋₁₀ cycloalkyl, C₃₋₁₀ cycloalkenyl, C₃₋₁₀ cycloalkynyl, C₃₋₁₀ heterocyclyl, C₃₋₁₄ aryl and C₃₋₁₄ heteroaryl, or taken together form C₂₋₁₀ alkenyl, C₃₋₁₀ cycloalkyl, or C₃₋₁₀ heterocyclyl;
R^{d} is C₂₋₁₀ alkenyl or C₂₋₁₀ alkynyl,
R^{e} is C₂₋₁₀ alkenyl or C₂₋₁₀ alkynyl, wherein R^{e} is positioned cis or trans to the double bond; and
(b) combining said compound of Formula (II) or (III) with an acid to produce a compound of Formula I.

2. The method of claim 1, wherein:
n is an integer selected from 0, 1, 2 or 3,
each occurrence of Rⁱ is independently selected from the group consisting of hydrogen, methoxyl, benzyloxy or two adjacent R′, taken together, is —O—(CH₂)—O— or —O—CH₂—CH₂—O—,
R and R′ taken together form with N* a C₄₋₁₀ heterocyclyl, which C₄₋₁₀ heterocyclyl is unsubstituted or substituted from three to sever times with substituents independently selected from the group consisting of C₄₋₆ spirocyclyl, C₃₋₁₀ spiroheterocyclyl,
R and R′ are independently hydrogen, C₁₋₁₀ alkyl, or taken together are C₂₋₆ alkenyl,
R¹⁰ and R¹¹ are hydrogen,
R^{d} is C₂₋₅ alkenyl or C₂₋₅ alkynyl,
R^{e} is C₂₋₅ alkenyl or C₂₋₅ alkynyl, wherein R^{e} is positioned cis or trans to the double bond.

3. A method of claim 1, wherein said compound of Formula I is a compound of Formula (Ia): 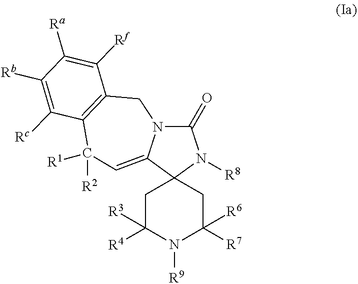
wherein said compounds of Formula (II) or Formula (III) are compounds of Formula (IIa) or (IIIa): 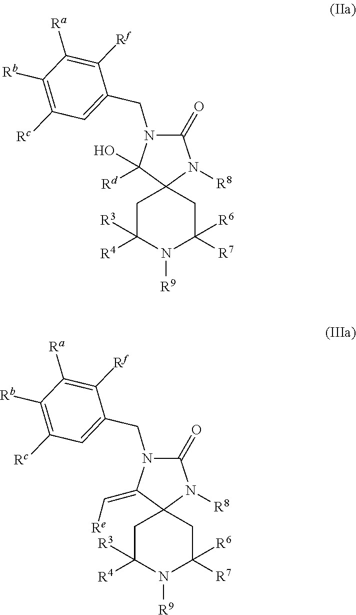
and wherein:
each of R³, R⁴, R⁶, and R⁷ are independently selected from hydrogen and methyl, or R³ and R⁶ taken together is —(CH₂CH₂)—,
R^{d} and R^{e} are independently C₂₋₁₀ alkenyl or C₂₋₁₀ alkynyl, and R^{e} is positioned cis or trans to the double bond,
each of R^{a}, R^{b}, R^{c} and R^{f} is independently selected from the group consisting of hydrogen, hydroxyl, C₁₋₁₀ alkoxy, benzyloxy, benzyl, halo, amino, (C₁₋₆ alkylamino, (C₁₋₆alkyl)(C₁₋₆alkyl)amino, phenoxy, and phenyl; or one pair selected from R^{a} and R^{b}, and R^{b} and R^{c}, taken together, is —O—(CH₂)—O— or —O—CH₂—CH₂—O—,
R⁹ is hydrogen or X—R⁵, wherein X is C₁₋₁₀ alkylene, C₂₋₁₀ alkenylene, C₂₋₁₀ alkynlene, and R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranonyl, thiazolyl, thiadiazolyl, furanyl, thienyl, pyrazolyl, quinoxalinyl, or naphthyl,
wherein said R⁵ substituted with between 0 and 5 substituents independently selected from the group consisting of C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl and halo,
R⁸ is hydrogen, methyl, ethyl, propyl, (C₁₋₃ alkoxy)C₁₋₃ alkyl, (C₁₋₃ alkylthio)C₁₋₃ alkyl, C₁₋₃ hydroxyalkyl, phenyl, benzyl, furanyl, pyrrolyl, imidazolyl, pyrazolyl, pyrrolyl, isothiazolyl, isooxazolyl, pyridyl, and thienyl,
wherein R⁸ is substituted with between 0 and 3 substituents independently selected from methyl, ethyl, halo, hydroxyl, C₁₋₃ alkoxy, C₁₋₃ alkylthio, (C₁₋₃ alkoxy)C₁₋₃ alkyl, (C₁₋₃ alkylthio)C₁₋₃ alkyl, C₁₋₃ hydroxyalkyl, (C₁₋₃ mercaptoalkyl)phenyl, benzyl, furanyl, imidazolyl, pyrazolyl, pyrrolyl, thiazolyl, isothiazolyl, oxazolyl, isooxazolyl, pyridyl, thienyl, indolyl, benzpyrazolyl, benzimidazolyl, benzofuranyl, benzoxazolyl, benzisoxazolyl, isobenzofuranyl, benzothiophenyl, isobenzothiophenyl, indolinyl, quinolinyl, isoquinolinyl, quinazolinyl, or quinoxalinyl.

4. The method of claim 3, wherein:
R¹ and R² are independently hydrogen or C₁₋₁₀ alkyl, or taken together are C₂₋₄ alkenyl,
each of R³, R⁴, R⁶, and R⁷ are independently selected from hydrogen and methyl, or R³ and R⁶ taken together is —(CH₂CH₂)—,
R^{d} is —(CH₂)ₘC(Rᵢ)═C(Rᵢᵢ)(Rᵢᵢᵢ) or —(CH₂)ₘC≡C(Rᵢ), wherein each occurrence of Rᵢ, Rᵢᵢ, Rᵢᵢᵢ are independently hydrogen, C₁₋₆alkyl, and m is 0 or 1,
R^{e} is —(CH₂)ₚC(Rᵢᵥ)═C(Rᵥ)(Rᵥᵢ), wherein Rᵢᵥ, Rᵥ, Rᵥᵢ are independently hydrogen, C₁₋₆alkyl, and p is 0 or 1,
each of R^{a}, R^{b}, R^{c} and R^{f} is independently selected from the group consisting of hydrogen, hydroxyl, methoxyl, benzyloxy, or one pair selected from R^{a} and R^{b}, and R^{b} and R^{c}, taken together, is —O—(CH₂)—O—,
R⁹ is hydrogen or X—R⁵, wherein X is C₁₋₁₀ alkyl, C₁₋₁₀ alkenyl, C₁₋₁₀ alkynyl, and R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranyl, thiazolyl, thiadiazolyl, furanyl, thienyl, pyrazolyl, quinoxalinyl, or naphthyl,
wherein said R⁵ substituted with between 0 and 5 substituents independently selected from the group consisting of C₁₋₄ alkyl, C₁₋₃ alkoxy, hydroxyl, C₁₋₃ alkylthio, cyclopropyl, cyclopropylmethyl, trifluoromethoxy, 5-methylisoxazolyl, pyrazolyl, benzyloxy, acetyl, (cyanyl)C₁₋₃ alkyl, (phenyl)C₂₋₃ alkenyl and halo,
R⁸ is hydrogen, methyl, ethyl, propyl, (C₁₋₃ alkoxy)C₁₋₃ alkyl, (C₁₋₃ alkylthio)C₁₋₃ alkyl, C₁₋₃ hydroxyalkyl, phenyl, benzyl, furanyl, pyrrolyl, imidazolyl, pyrazolyl, pyrrolyl, isothiazolyl, isooxazolyl, pyridyl, and thienyl,
wherein R⁸ is substituted with between 0 and 3 substituents independently selected from methyl, ethyl, halo, hydroxyl, C₁₋₃ alkoxy, C₁₋₃ alkylthio, (C₁₋₃ alkoxy)C₁₋₃ alkyl, (C₁₋₃ alkylthio)C₁₋₃ alkyl, C₁₋₃ hydroxyalkyl, (C₁₋₃ mercaptoalkyl)phenyl, benzyl, furanyl, imidazolyl, pyrazolyl, pyrrolyl, thiazolyl, isothiazolyl, oxazolyl, isooxazolyl, pyridyl, thienyl, indolyl, benzpyrazolyl, benzimidazolyl, benzofuranyl, benzoxazolyl, benzisoxazolyl, isobenzofuranyl, benzothiophenyl, isobenzothiophenyl, indolinyl, quinolinyl, isoquinolinyl, quinazolinyl, or quinoxalinyl.

5. The method of claim 1, wherein said combining step (b) is carried out in a solvent.

6. The method of claim 1, wherein said solvent comprises a solvent selected from the group consisting of tetrahydrofuran, acetonitrile, methylene chloride, ether, methanol, water and combinations thereof.

7. The method of claim 1, wherein said acid is selected from the group consisting of trifluoromethansulfonic acid, trifluoroacetic acid, monofluoroacetic acid, difluoroacetic acid, mono, di-, or trichloroacetic acid, phosphoric acid, sulfuric acid, camphor sulfonic acid, formic acid, acetic acid, tartic acid, haloacetic acid, dibenzoyltartaric acid, hydrochloric acid, hydroiodic acid, hydrofloric acid, hydrobromic acid and combinations thereof.

8. The method of claim 1, wherein said acid is selected from the group consisting of trifluoromethansulfonic acid, trifluoroacetic acid, camphor sulfonic acid, formic acid, acetic acid, tartic acid, dibenzoyltartaric acid, and combinations thereof.

9. The method of claim 1, wherein said acid is a Lewis acid selected from the group consisting of trimethylsilyl trifluoromethanesulfonate, trimethylsilyl chloride, titanium tetrachloride, gold(III) chloride, boron trifluoride, aluminium trichloride, iron(III) chloride, niobium chloride, and combinations thereof.

10. The method of claim 1, wherein said acid is a Lewis acid selected from the group consisting of trimethylsilyl trifluoromethanesulfonate, trimethylsilyl chloride, titanium tetrachloride, dichlorodiisopropoxytitanium, and combinations thereof.

11. The method of claim 4,
wherein R⁸ in the compound of Formula Ia is not H and R⁸ in the compound of Formula (IIa) and (IIIa) is H, said method further comprising the step of:
(c) combining the compound of Formula Ia with a compound of R⁸*—Y and a base to produce said compound of Formula Ia, wherein:
Y is bromo, chloro, iodo, trifluoromethylsulfonyl, 4-methylphenylsulfonyl, or methanesulfonyl; and
R⁸* is hydrogen or X—R⁵, wherein X is C₁₋₁₀ alkyl, C₁₋₁₀ alkenyl, C₁₋₁₀ alkynyl, and R⁵ is phenyl, pyrrolyl, benzimidazolyl, oxazolyl, isoxazolyl, imidazothiazolyl, quinolinyl, isoquinolinyl, indazolyl, pyridinyl, imidazopyridinyl, indolyl, benzotriazolyl, imidazolyl, benzofuranyl, benzothiadiazolyl, pyridimidinyl, benzopyranyl, thiazolyl, thiadiazolyl, furanyl, thienyl, pyrazolyl, quinoxalinyl, or naphthyl.

12. The method of claim 11, wherein:
Y is bromo, chloro, or iodo and R⁸* is hydrogen or X—R⁵, wherein X is C₁₋₁₀ alkyl, C₁₋₁₀ alkenyl, or C₁₋₁₀ alkynyl, and R⁵ is phenyl.

13. The method of claim 11, wherein said base is selected from the group consisting of sodium hydride, lithium hexamethyldisilazide, sodium hexamethyldisilazide, potassium hexamethyldisilazide, potassium tert-butoxide, and combinations thereof.

14. The method of claim 4, wherein
R⁹ in said compound of Formula (Ia) is —X—R⁵ and R⁹ in said compound of Formula (IIa) and Formula (IIIa) is H,
said method further comprising the step of:
(c) combining the compound of Formula (Ia) with Z-X—R⁵ and a base to produce said compound of Formula (Ia),
wherein: Z is bromo, chloro, iodo, trifluoromethylsulfonyl, 4-methylphenylsulfonyl, or methanesulfonyl.

15. The method of claim 14, wherein said base is Diaza(1,3)bicyclo[5.4.0]undecane.

16. The method of claim 4, wherein:
R⁹ in said compound of Formula (Ia) is —X—R⁵ and R⁹ in said compound of Formula (IIa) and Formula (IIIa) is H,
said method further comprising the step of:
(c) combining the compound of formula (Ia) with R⁵—C(═O)H and a reducing agent to produce said compound of Formula (Ia).

17. The method of claim 16, wherein said reducing agent is sodium cyanoborohydride, sodium triacetoxyborohydride, or a combination thereof.

18. The method of claim 16, wherein said step (c) is carried out in a solvent.

19. The method of claim 18, wherein said solvent is selected from the group of consisting of N-methylpyrrolidone, dichloromethane, toluene, dichloroethane, tetrahydrofuran, and combinations thereof.

20. The method of claim 16, wherein:
said reducing agent is sodium triacetoxyborohydride; and
said solvent is N-methylpyrrolidone.

21. The method of claim 4, wherein:
R¹ and R² are independently hydrogen or C₁₋₃ alkyl,
R³, R⁴, R⁶, and R⁷ are hydrogen,
R^{d} is —(CH₂)ₘC(Rᵢ)═C(Rᵢᵢ)(Rᵢᵢᵢ) or —(CH₂)ₘC≡C(Rᵢ), wherein each occurrence of Rᵢ, Rᵢᵢ, Rᵢᵢᵢ are independently hydrogen, C₁₋₃alkyl, and m is 0 or 1,
R^{e} is —(CH₂)ₚC(Rᵢᵥ)═C(Rᵥ)(Rᵥᵢ), wherein Rᵢᵥ, Rᵥ, Rᵥᵢ are independently hydrogen, C₁₋₃alkyl, and p is 0 or 1,
each of R^{a}, R^{b}, R^{c} and R^{f} is independently hydrogen or C₁₋₃ alkoxy,
R⁹ is hydrogen or X—R⁵, wherein X is C₁₋₃ alkylene, and R⁵ is phenyl, pyrrolyl, or pyrazolyl, wherein said R⁵ is substituted with 1 or 2 substituents of C₁₋₃ alkyl,
R⁸ is hydrogen, methyl, ethyl, or propyl.

22. The method of claim 4, wherein said compound of Formula (Ia) is selected from the group consisting of: 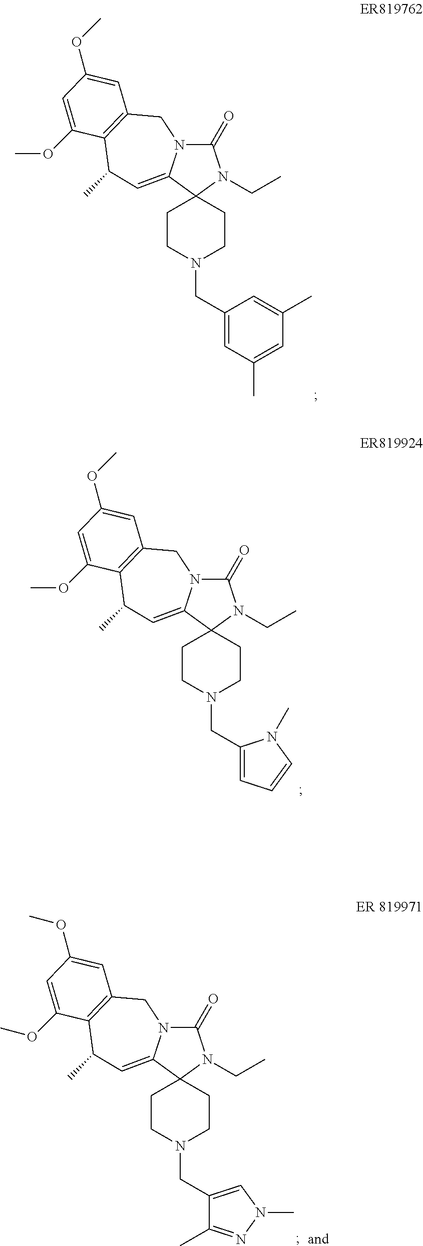 [chemical structure]
